(19) United States
(12) Patent Application Publication
(10) Pub. No.: US 2011/0044943 A1
(43) Pub. Date: Feb. 24, 2011

(54) Anti-Viral Compounds, Compositions, And Methods Of Use

(76) Inventors: Martin Robert Leivers, Durham, NC (US); Jesse Daniel Keicher, Durham, NC (US); Franz Ulrich Schmitz, Mill Valley, CA (US); Roopa Rai, San Carlos, CA (US); Ryan Lauchli, San Mateo, CA (US); Sebastian Reinhard Johannes Liehr, Palo Alto, CA (US); Stephanie Anna Chan, San Francisco, CA (US); Tony Loc Ton, Fremont, CA (US)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(21) Appl. No.: 12/668,543

(22) PCT Filed: Jul. 11, 2008
(86) PCT No.: PCT/US08/08496

§ 371 Date: Aug. 23, 2010

Related U.S. Application Data
(60) Provisional application No. 60/949,758, filed on Jul. 13, 2007.

(51) Int. Cl. A61K 38/21; C07D 487/04; A61K 31/5025; A61P 31/14; A61K 31/5377

(52) U.S. Cl. 424/85.4; 544/236; 514/248; 544/118; 514/234.2

(57) ABSTRACT

Disclosed are compounds of Formula (I), pharmaceutically acceptable salts and solvates thereof, compositions thereof, and methods for their preparation and uses for treating viral infections mediated at least in part by a virus in the Flaviviridae family of viruses.

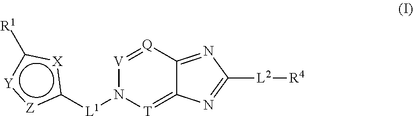

This application claims the benefit of U.S. Provisional Patent Application No. 60/949,758, filed Jul. 13, 2007, which is hereby incorporated by reference.

Compounds and compositions, methods for their preparation, and methods for their use in treating viral infections in patients mediated, at least in part, by a virus in the Flaviviridae family of viruses are disclosed.

The following publications are cited in this application as superscript numbers:
1. Szabo, E. et al., Pathol. Oncol. Res. 2003, 9:215-221.
2. Hoofnagle J. H., Hepatology 1997, 26:15 S-20S.
3. Thomson B. J. and Finch R. G., Clin Microbial Infect. 2005, 11:86-94.
4. Moriishi K. and Matsuura Y., Antivir. Chem. Chemother. 2003, 14:285-297.
5. Fried, M. W., et al. N. Engl. J Med 2002, 347:975-982.
6. Ni, Z. J. and Wagman, A. S. Curr. Opin. Drug Discov. Devel. 2004, 7, 446-459.
7. Beaulieu, P. L. and Tsantrizos, Y. S. Curr. Opin. Investig. Drugs 2004, 5, 838-850.
8. Griffith, R. C. et al., Ann. Rep. Med. Chem. 39, 223-237, 2004.
9. Watashi, K. et al., Molecular Cell, 19, 111-122, 2005
10. Horsmans, Y. et al., Hepatology, 42, 724-731, 2005

Chronic infection with HCV is a major health problem associated with liver cirrhosis, hepatocellular carcinoma, and liver failure. An estimated 170 million chronic carriers worldwide are at risk of developing liver disease.^{1,2} In the United States alone 2.7 million are chronically infected with HCV, and the number of HCV-related deaths in 2000 was estimated between 8,000 and 10,000, a number that is expected to increase significantly over the next years. Infection by HCV is insidious in a high proportion of chronically infected (and infectious) carriers who may not experience clinical symptoms for many years. Liver cirrhosis can ultimately lead to liver failure. Liver failure resulting from chronic HCV infection is now recognized as a leading cause of liver transplantation.

HCV is a member of the Flaviviridae family of RNA viruses that affect animals and humans. The genome is a single ˜9.6-kilobase strand of RNA, and consists of one open reading frame that encodes for a polyprotein of ˜3000 amino acids flanked by untranslated regions at both 5′ and 3′ ends (5′- and 3′-UTR). The polyprotein serves as the precursor to at least 10 separate viral proteins critical for replication and assembly of progeny viral particles. The organization of structural and non-structural proteins in the HCV polyprotein is as follows: C-E1-E2-p7-NS2-NS3-NS4a-NS4b-NS5a-NS5b. Because the replicative cycle of HCV does not involve any DNA intermediate and the virus is not integrated into the host genome, HCV infection can theoretically be cured. While the pathology of HCV infection affects mainly the liver, the virus is found in other cell types in the body including peripheral blood lymphocytes.^{3,4}

At present, the standard treatment for chronic HCV is interferon alpha (IFN-alpha) in combination with ribavirin and this requires at least six (6) months of treatment. IFN-alpha belongs to a family of naturally occurring small proteins with characteristic biological effects such as antiviral, immunoregulatory, and antitumoral activities that are produced and secreted by most animal nucleated cells in response to several diseases, in particular viral infections. IFN-alpha is an important regulator of growth and differentiation affecting cellular communication and immunological control. Treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction. Ribavirin, an inhibitor of inosine 5′-monophosphate dehydrogenase (IMPDH), enhances the efficacy of MN-alpha in the treatment of HCV. Despite the introduction of ribavirin, more than 50% of the patients do not eliminate the virus with the current standard therapy of interferon-alpha (IFN) and ribavirin. By now, standard therapy of chronic hepatitis C has been changed to the combination of pegylated IFN-alpha plus ribavirin. However, a number of patients still have significant side effects, primarily related to ribavirin. Ribavirin causes significant hemolysis in 10-20% of patients treated at currently recommended doses, and the drug is both teratogenic and embryotoxic. Even with recent improvements, a substantial fraction of patients do not respond with a sustained reduction in viral load⁵ and there is a clear need for more effective antiviral therapy of HCV infection.

A number of approaches are being pursued to combat the virus. These include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among the viral targets, the NS3/4a protease/helicase and the NS5b RNA-dependent RNA polymerase are considered the most promising viral targets for new drugs.⁶⁻⁸

Besides targeting viral genes and their transcription and translation products, antiviral activity can also be achieved by targeting host cell proteins that are necessary for viral replication. For example, Watashi et al.⁹ show how antiviral activity can be achieved by inhibiting host cell cyclophilins. Alternatively, a potent TLR7 agonist has been shown to reduce HCV plasma levels in humans.¹⁰

In view of the worldwide epidemic level of HCV and other members of the Flaviviridae family of viruses, and further in view of the limited treatment options, there is a strong need for new effective drugs for treating infections cause by these viruses.

Provided is a compound that is Formula (I):

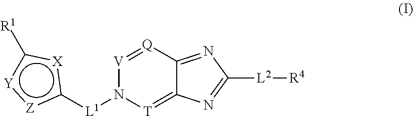

or a pharmaceutically acceptable salt or solvate thereof, wherein:
a) when X is CR² or N, one of Y or Z is O and the other of Y or Z is N; or one of Y or Z is N and the other of Y or Z is NR^{a};
b) when X is O, NR^{a}, or S(O)ₚ wherein p is 0 or 1, one of Y or Z is N and the other of Y or Z is N or CR²;
c) when X is N, one of Y or Z is O and the other of Y or Z is N;
L¹ is L³;
L² is a bond or L³;
L³ is independently C₃₋₆ cycloalkylene or is C₁₋₅ alkylene where one or two —CH₂— groups of said C₁₋₅ alkylene are optionally replaced with —NR^{b}—, —S—, —(C═O)—, or —O— and optionally two —CH₂— groups together form a double bond or triple bond provided that L³ does not contain an —O—O—, —S—O—, or —S—S— group, and wherein said C₁ to C₅ alkylene is optionally substituted with one to three groups independently selected from halo, alkyl, and spirocycloalkyl;
R^{a} and R^{b} are independently H, alkyl, or substituted alkyl;
one of V or T is N and the other of V or T is CR³;
Q is N or CR³;
R¹ and R⁴ are independently selected from aryl, substituted aryl, heteroaryl, substituted heteroaryl, heterocyclyl, substituted heterocyclyl, cycloalkyl, and substituted cycloalkyl;
R² is independently selected from hydrogen, halo, alkyl, substituted alkyl, alkenyl, substituted alkenyl, alkynyl, substituted alkynyl, amino, substituted amino, acylamino, hydroxy, alkoxy, substituted alkoxy, carboxy, carboxy ester, cycloalkyl, substituted cycloalkyl, and cyano; and
R³ is independently selected from hydrogen, halo, amino, substituted amino, acylamino, alkyl, substituted alkyl, alkenyl, substituted alkenyl, alkynyl, substituted alkynyl, carboxy, carboxy ester, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, acyloxy, cyano, thiol, alkylthio, substituted alkylthio, and substituted sulfonyl.

Also provided is a pharmaceutical composition comprising a pharmaceutically acceptable carrier and a therapeutically effective amount of a compound of Formula (I), or a pharmaceutically acceptable salt or solvate thereof.

Also provided are methods for preparing the compounds of Formula (I), or a pharmaceutically acceptable salt or solvate thereof, and compositions thereof and for their therapeutic uses. In some embodiments, provided is a method for treating a viral infection in a patient mediated at least in part by a virus in the Flaviviridae family of viruses, comprising administering to said patient a composition comprising a compound Formula (I), or a pharmaceutically acceptable salt or solvate thereof. In some embodiments, the viral infection is mediated by hepatitis C virus.

Those and other embodiments are further described in the text that follows.

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

It is to be understood that the terminology used herein is for the purpose of describing particular embodiments only and is not intended to limit the scope of the present invention. In this specification and in the claims that follow, reference will be made to a number of terms that shall be defined to have the following meanings:

“Alkyl” refers to monovalent saturated aliphatic hydrocarbyl groups having from 1 to 10 carbon atoms and, in some embodiments, from 1 to 6 carbon atoms. “C_{x-y}alkyl” refers to alkyl groups having from x to y carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5 and, in some embodiments, 1 to 3 or 1 to 2 substituents selected from the group consisting of alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, spirocycloalkyl, SO₃H, substituted sulfonyl, sulfonyloxy, thioacyl, thiocyanate, thiol, alkylthio, and substituted alkylthio, wherein said substituents are as defined herein.

“Alkylidene” or “alkylene” refers to divalent saturated aliphatic hydrocarbyl groups having from 1 to 10 carbon atoms and, in some embodiments, from 1 to 6 carbon atoms. “(Cᵤ₋ᵥ)alkylene” refers to alkylene groups having from u to v carbon atoms. The alkylidene and alkylene groups include branched and straight chain hydrocarbyl groups. For example “(C₁₋₆)alkylene” is meant to include methylene, ethylene, propylene, 2-methypropylene, pentylene, and the like.

“Substituted alkylidene” or “substituted alkylene” refers to an alkylidene group having from 1 to 5 and, in some embodiments, 1 to 3 or 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, SO₃H, substituted sulfonyl, sulfonyloxy, thioacyl, thiocyanate, thiol, alkylthio, and substituted alkylthio, wherein said substituents are as defined herein.

“Alkenyl” refers to a linear or branched hydrocarbyl group having from 2 to 10 carbon atoms and in some embodiments from 2 to 6 carbon atoms or 2 to 4 carbon atoms and having at least 1 site of vinyl unsaturation (>C═C<). For example, (Cₓ-C_{y})alkenyl refers to alkenyl groups having from x to y carbon atoms and is meant to include for example, ethenyl, propenyl, 1,3-butadienyl, and the like.

“Substituted alkenyl” refers to alkenyl groups having from 1 to 3 substituents and, in some embodiments, 1 to 2 substituents selected from the group consisting of alkoxy, substituted alkoxy, acyl, acylamino, acyloxy, alkyl, substituted alkyl, alkynyl, substituted alkynyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₆)alkynyl is meant to include ethynyl, propynyl, and the like.

“Substituted alkynyl” refers to alkynyl groups having from 1 to 3 substituents and, in some embodiments, from 1 to 2 substituents selected from the group consisting of alkoxy, substituted alkoxy, acyl, acylamino, acyloxy, alkyl, substituted alkyl, alkenyl, substituted alkenyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is as defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, substituted hydrazino-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, aryl, substituted aryl, substituted hydrazino, heteroaryl, substituted heteroaryl, heterocyclic, and substituted heterocyclic are as defined herein. Acyl includes the “acetyl” group CH₃C(O)—.

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic wherein R²⁰ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amino” refers to the group —NH₂.

“Substituted amino” refers to the group —NR²¹R²² where R²¹ and R²² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²¹ and R²² are optionally joined together with the nitrogen bound thereto to form a heterocyclic or substituted heterocyclic group, provided that R²¹ and R²² are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. When R²¹ is hydrogen and R²² is alkyl, the substituted amino group is sometimes referred to herein as alkylamino. When R²¹ and R²² are alkyl, the substituted amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Hydroxyamino” refers to the group —NHOH.

“Alkoxyamino” refers to the group —NHO-alkyl wherein alkyl is defined herein.

“Aminocarbonyl” refers to the group —C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, hydroxy, alkoxy, substituted alkoxy, amino, substituted amino, and acylamino, and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonyl” refers to the group —C(S)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonylamino” refers to the group —NR²⁰C(O)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonylamino” refers to the group —NR²⁰C(S)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonyloxy” refers to the group —O—C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyl” refers to the group —SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyloxy” refers to the group —O—SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonylamino” refers to the group —NR²⁰—SO₂NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amidino” refers to the group —C(═NR²⁵)NR²³R²⁴ where R²⁵, R²³, and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aryl” or “Ar” refers to an aromatic group of from 6 to 14 carbon atoms and no ring heteroatoms and having a single ring (e.g., phenyl) or multiple condensed (fused) rings (e.g., naphthyl or anthryl). For multiple ring systems, including fused, bridged, and Spiro ring systems having aromatic and non-aromatic rings that have no ring heteroatoms, the term “Aryl” or “Ar” applies when the point of attachment is at an aromatic carbon atom (e.g., 5,6,7,8 tetrahydronaphthalene-2-yl is an aryl group as its point of attachment is at the 2-position of the aromatic phenyl ring).

“Substituted aryl” refers to aryl groups which are substituted with 1 to 8 and, in some embodiments, 1 to 5, 1 to 3, or 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are defined herein.

“Aryloxy” refers to the group —O-aryl, where aryl is as defined herein, that includes, by way of example, phenoxy and naphthyloxy.

“Substituted aryloxy” refers to the group —O-(substituted aryl) where substituted aryl is as defined herein.

“Arylthio” refers to the group -S-aryl, where aryl is as defined herein.

“Substituted arylthio” refers to the group —S-(substituted aryl), where substituted aryl is as defined herein.

“Azido” refers to the group —N₃.

“Hydrazino” refers to the group —NHNH₂.

“Substituted hydrazino” refers to the group —NR²⁶NR²⁷R²⁸ where R²⁶, R²⁷, and R²⁸ are independently selected from the group consisting of hydrogen, alkyl, substituted alkyl, alkenyl, substituted alkenyl, alkynyl, substituted alkynyl, aryl, substituted aryl, carboxyl ester, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²⁷ and R²⁸ are optionally joined, together with the nitrogen bound thereto to form a heterocyclic or substituted heterocyclic group, provided that R²⁷ and R²⁸ are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cyano” or “carbonitrile” refers to the group —CN.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)β-cycloalkyl, —C(O)O-substituted cycloalkyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)amino” refers to the group —NR²⁰—C(O)O-alkyl, —NR²⁰—C(O)O-substituted alkyl, —NR²⁰—C(O)O-alkenyl, —NR²⁰—C(O)O-substituted alkenyl, —NR²⁰—C(O)O-alkynyl, —NR²⁰—C(O)O-substituted alkynyl, —NR²⁰—C(O)O-aryl, —NR²⁰—C(O)O-substituted aryl, —NR²⁰—C(O)β-cycloalkyl, —NR²⁰—C(O)O-substituted cycloalkyl, —NR²⁰—C(O)O-heteroaryl, —NR²⁰—C(O)O-substituted heteroaryl, —NR²⁰—C(O)O-heterocyclic, and —NR²⁰—C(O)O-substituted heterocyclic wherein R²⁰ is alkyl or hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)β-cycloalkyl, —O—C(O)O-substituted cycloalkyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8,-tetrahydronaphthalene-5-yl). The term “cycloalkyl” includes cycloalkenyl groups. Examples of cycloalkyl groups include, for instance, adamantyl, cyclopropyl, cyclobutyl, cyclopentyl, cyclooctyl, and cyclohexenyl. “Cᵤ₋ᵥcycloalkyl” refers to cycloalkyl groups having u to v carbon atoms.

“Cycloalkenyl” refers to a partially saturated cycloalkyl ring having at least one site of >C═C<ring unsaturation.

“Cycloalkylene” refer to divalent cycloalkyl groups as defined herein. Examples of cycloalkyl groups include those having three to six carbon ring atoms such as cyclopropylene, cyclobutylene, cyclopentylene, and cyclohexylene.

“Substituted cycloalkyl” refers to a cycloalkyl group, as defined herein, having from 1 to 8, or 1 to 5, or in some embodiments 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are as defined herein. The term “substituted cycloalkyl” includes substituted cycloalkenyl groups.

“Cycloalkyloxy” refers to —O-cycloalkyl wherein cycloalkyl is as defined herein.

“Substituted cycloalkyloxy refers to —O-(substituted cycloalkyl) wherein substituted cycloalkyl is as defined herein.

“Cycloalkylthio” refers to —S-cycloalkyl wherein cycloalkyl is as defined herein.

“Substituted cycloalkylthio” refers to —S-(substituted cycloalkyl).

“Guanidino” refers to the group —NHC(═NH)NH₂.

“Substituted guanidino” refers to —NR²⁹C(═NR²⁹)N(R²⁹)₂ where each R²⁹ is independently selected from the group consisting of hydrogen, alkyl, substituted alkyl, aryl, substituted aryl, heteroaryl, substituted heteroaryl, heterocyclyl, and substituted heterocyclyl and two R²⁹ groups attached to a common guanidino nitrogen atom are optionally joined together with the nitrogen bound thereto to form a heterocyclic or substituted heterocyclic group, provided that at least one R²⁹ is not hydrogen, and wherein said substituents are as defined herein.

“Halo” or “halogen” refers to fluoro, chloro, bromo, and iodo.

“Haloalkyl” refers to substitution of alkyl groups with 1 to 5 or in some embodiments 1 to 3 halo groups.

“Haloalkoxy” refers to substitution of alkoxy groups with 1 to 5 or in some embodiments 1 to 3 halo groups.

“Hydroxy” or “hydroxyl” refers to the group —OH.

“Heteroaryl” refers to an aromatic group of from 1 to 14 carbon atoms and 1 to 6 heteroatoms selected from the group consisting of oxygen, nitrogen, and sulfur and includes single ring (e.g. imidazolyl) and multiple ring systems (e.g. benzimidazol-2-yl and benzimidazol-6-yl). For multiple ring systems, including fused, bridged, and Spiro ring systems having aromatic and non-aromatic rings, the term “heteroaryl” applies if there is at least one ring heteroatom and the point of attachment is at an atom of an aromatic ring (e.g. 1,2,3,4-tetrahydroquinolin-6-yl and 5,6,7,8-tetrahydroquinolin-3-yl). In some embodiments, the nitrogen and/or the sulfur ring atom(s) of the heteroaryl group are optionally oxidized to provide for the N-oxide (N→O), sulfinyl, or sulfonyl moieties. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, quinazolinonyl, benzimidazolyl, benzisoxazolyl, or benzothienyl.

“Substituted heteroaryl” refers to heteroaryl groups that are substituted with from 1 to 8 or in some embodiments 1 to 5, or 1 to 3, or 1 to 2 substituents selected from the group consisting of the substituents defined for substituted aryl.

“Heteroaryloxy” refers to —O-heteroaryl wherein heteroaryl is as defined herein.

“Substituted heteroaryloxy” refers to the group —O-(substituted heteroaryl) wherein substituted heteroaryl is as defined herein.

“Heteroarylthio” refers to the group -S-heteroaryl wherein heteroaryl is as defined herein.

“Substituted heteroarylthio” refers to the group —S-(substituted heteroaryl) wherein substituted heteroaryl is as defined herein.

“Heterocyclic” or “heterocycle” or “heterocycloalkyl” or “heterocyclyl” refers to a saturated or partially saturated cyclic group having from 1 to 14 carbon atoms and from 1 to 6 heteroatoms selected from the group consisting of nitrogen, sulfur, or oxygen and includes single ring and multiple ring systems including fused, bridged, and spiro ring systems. For multiple ring systems having aromatic and/or non-aromatic rings, the terms “heterocyclic”, “heterocycle”, “heterocycloalkyl”, or “heterocyclyl” apply when there is at least one ring heteroatom and the point of attachment is at an atom of a non-aromatic ring (e.g. 1,2,3,4-tetrahydroquinoline-3-yl, 5,6,7,8-tetrahydroquinoline-6-yl, and decahydroquinolin-6-yl). In some embodiments, the nitrogen and/or sulfur atom(s) of the heterocyclic group are optionally oxidized to provide for the N-oxide, sulfinyl, sulfonyl moieties. More specifically the heterocyclyl includes, but is not limited to, tetrahydropyranyl, piperidinyl, N-methylpiperidin-3-yl, piperazinyl, N-methylpyrrolidin-3-yl, 3-pyrrolidinyl, 2-pyrrolidon-1-yl, morpholinyl, and pyrrolidinyl. A prefix indicating the number of carbon atoms (e.g., C₃-C₁₀) refers to the total number of carbon atoms in the portion of the heterocyclyl group exclusive of the number of heteroatoms.

“Substituted heterocyclic” or “substituted heterocycle” or “substituted heterocycloalkyl” or “substituted heterocyclyl” refers to heterocyclic groups, as defined herein, that are substituted with from 1 to 5 or in some embodiments 1 to 3 of the substituents as defined for substituted cycloalkyl.

“Heterocyclyloxy” refers to the group —O-heterocycyl wherein heterocyclyl is as defined herein.

“Substituted heterocyclyloxy” refers to the group —O-(substituted heterocycyl) wherein substituted heterocyclyl is as defined herein.

“Heterocyclylthio” refers to the group —S-heterocycyl wherein heterocyclyl is as defined herein.

“Substituted heterocyclylthio” refers to the group —S-(substituted heterocycyl) wherein substituted heterocyclyl is as defined herein.

Examples of heterocycle and heteroaryl groups include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O).

“Oxide” refers to products resulting from the oxidation of one or more heteroatoms. Examples include N-oxides, sulfoxides, and sulfones.

“Spirocycloalkyl” refers to a 3 to 10 member cyclic substituent formed by replacement of two hydrogen atoms at a common carbon atom with an alkylene group having 2 to 9 carbon atoms, as exemplified by the following structure wherein the methylene group shown here attached to bonds marked with wavy lines is substituted with a spirocycloalkyl group:

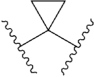

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-alkynyl, —SO₂-substituted alkynyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiol” refers to the group —SH.

“Alkylthio” refers to the group —S-alkyl wherein alkyl is as defined herein.

“Substituted alkylthio” refers to the group —S-(substituted alkyl) wherein substituted alkyl is as defined herein.

“Thiocarbonyl” refers to the divalent group —C(S)— which is equivalent to —C(═S)—.

“Thione” refers to the atom (═S).

“Thiocyanate” refers to the group —SCN.

“Compound” and “compounds” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

“Racemates” refers to a mixture of enantiomers.

“Solvate” or “solvates” of a compound refer to those compounds, where compounds is as defined above, that are bound to a stoichiometric or non-stoichiometric amount of a solvent. Solvates of a compound includes solvates of all forms of the compound. In some embodiments, solvents are volatile, non-toxic, and/or acceptable for administration to humans in trace amounts. Suitable solvents include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

“Patient” refers to mammals and includes humans and non-human mammals.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

Accordingly, provided is a compound that is Formula (I):

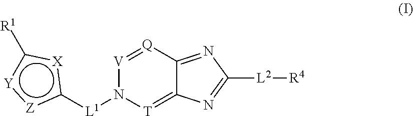

or a pharmaceutically acceptable salt or solvate thereof, wherein:
a) when X is CR² or N, one of Y or Z is O and the other of Y or Z is N; or one of Y or Z is N and the other of Y or Z is NR^{a};
b) when X is O, NR^{a}, or S(O)ₚ wherein p is 0 or 1, one of Y or Z is N and the other of Y or Z is N or CR²;
c) when X is N, one of Y or Z is O and the other of Y or Z is N;
L¹ is L³;
L² is a bond or L³;
L³ is independently C₃₋₆ cycloalkylene or is C₁₋₅ alkylene where one or two —CH₂— groups of said C₁₋₅ alkylene are optionally replaced with —NR^{b}—, —S—, —(C═O)—, or —O— and optionally two —CH₂— groups together form a double bond or triple bond provided that L³ does not contain an —O—O—, —S—O—, or —S—S— group, and wherein said C₁ to C₅ alkylene is optionally substituted with one to three groups independently selected from halo, alkyl, and spirocycloalkyl;
R^{a} and R^{b} are independently H, alkyl, or substituted alkyl;
one of V or T is N and the other of V or T is CR³;
Q is N or CR³;
R¹ and R⁴ are independently selected from aryl, substituted aryl, heteroaryl, substituted heteroaryl, heterocyclyl, substituted heterocyclyl, cycloalkyl, and substituted cycloalkyl;
R² is independently selected from hydrogen, halo, alkyl, substituted alkyl, alkenyl, substituted alkenyl, alkynyl, substituted alkynyl, amino, substituted amino, acylamino, hydroxy, alkoxy, substituted alkoxy, carboxy, carboxy ester, cycloalkyl, substituted cycloalkyl, and cyano; and
R³ is independently selected from hydrogen, halo, amino, substituted amino, acylamino, alkyl, substituted alkyl, alkenyl, substituted alkenyl, alkynyl, substituted alkynyl, carboxy, carboxy ester, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, acyloxy, cyano, thiol, alkylthio, substituted alkylthio, and substituted sulfonyl.

In some embodiments, provided is a compound that is a pharmaceutically acceptable salt of Formula (I).

In some embodiments, provided is a compound that is a solvate of Formula (I). In some embodiments, the solvate is a solvate of a pharmaceutically acceptable salt of Formula (I).

In some embodiments, Q is CR³. In some embodiments, R³ is selected from hydrogen and lower alkyl. In some embodiments, R³ is hydrogen.

In some embodiments, Q is N.

In some embodiments, V is N and T is CR³. In some embodiments when V is N and T is CR³, R³ is selected from hydrogen and lower alkyl. In some embodiments when V is N and T is CR³, R³ is hydrogen.

In some embodiments, V is CR³ and T is N. In some embodiments when V is CR³ and T is N, R³ is selected from hydrogen and lower alkyl. In some embodiments when V is CR³ and T is N, R³ is hydrogen.

In some embodiments, provided is a compound of Formula (I) that is Formula (II)

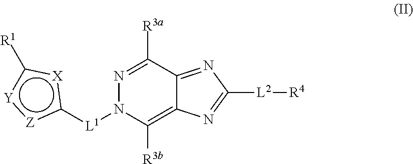

or a pharmaceutically acceptable salt or solvate thereof, wherein R^{3a} and R^{3b} are independently R³ and wherein R¹, R³, R⁴, X, Y, Z, L¹, and L² and are as defined for Formula (I).

Various features relating to the embodiments above are given below. These features when referring to different substituents or variables can be combined with each other or with any other embodiments described in this application. In some embodiments, provided are compounds of Formula (I) or (II) having one or more of the following features below.

In some embodiments, X is CR², Y is O and Z is N. In some embodiments, X is CR², Y is N and Z is O. In some embodiments, Y is N and Z is O. In some embodiments, X is N.

In some embodiment, X is CR². In some embodiments, X is CH.

In some embodiments when X is CR² or N, the ring formed by X, Y, and Z is selected from the following wherein the dashed line indicates the point of attachment to R¹ and the bolded line indicates attachment to the remainder of the compound:

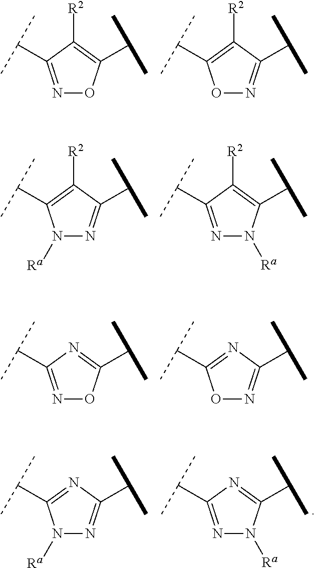

In some embodiments when X is O, NR^{a}, or S(O)ₚ wherein p is 0 or 1, the ring formed by X, Y, and Z is selected from the following:

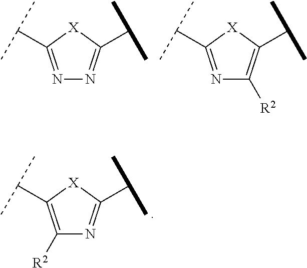

In some embodiments, the ring formed by X, Y, and Z is

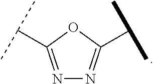

In some embodiments, L¹ is C₁₋₃ alkylene where one or two —CH₂— groups of said C₁₋₃ alkylene are optionally replaced with —NR^{b}—, —S—, —(C═O)—, or —O—, and wherein said C₁ to C₃ alkylene is optionally substituted with one to three groups independently selected from halo and lower alkyl. In some embodiments, L¹ is C₁₋₃ alkylene optionally substituted with one to three halo groups. In some embodiments, L¹ is C₁₋₃ alkylene. In some embodiments, L¹ is CH₂.

In some embodiments, L² is a bond.

In some embodiments, R¹ is substituted phenyl or substituted heteroaryl. In some embodiments, R¹ is phenyl or heteroaryl, each of which is substituted with at least one group selected from alkyl, haloalkyl, and optionally substituted alkoxy. In some embodiments, R¹ is phenyl or heteroaryl, each of which is substituted with at least one group selected from lower alkyl, CF₃, and optionally substituted methoxy. In some embodiments, R¹ is phenyl substituted with at least one group selected from lower alkyl, CF₃, and optionally substituted methoxy. In some embodiments, R¹ is phenyl substituted with at least one group selected from lower alkyl, CF₃, and R⁵—CH₂O— wherein R⁵ is optionally substituted heteroaryl. In some embodiments, R¹ is phenyl substituted with at least one group selected from lower alkyl, CF₃, and R⁵—CH₂O— wherein R⁵ is optionally substituted pyridinyl. In some embodiments, R¹ is phenyl substituted with at least one group selected from lower alkyl, CF₃, and R⁵—CH₂O— wherein R⁵ is pyridinyl.

In some embodiments, R¹ is substituted phenyl or substituted heteroaryl. In some embodiments, R¹ is substituted with at least one haloalkyl group, such as a CF₃ group.

In some embodiments, R⁴ is substituted phenyl or substituted heteroaryl. In some embodiments, R⁴ is substituted with at least one halo group, such as with at least one fluoro group. In some embodiments, R⁴ is phenyl substituted with at least one fluoro group. In some embodiments, R⁴ is 2,3-difluorophenyl.

In some embodiments, R³ or R^{3b} is hydrogen.

In some embodiments, R^{3a} is hydrogen.

Also provided is compound selected from Table 1 or a pharmaceutically acceptable salt or solvate thereof.

TABLE 1

| Com- | | |
|---|---|---|
| pound | | |
| # | Structure | Name |
| 101 | 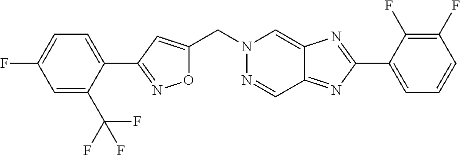 | 2-(2,3-Difluoro-phenyl)-5-[3-(4-fluoro- 2-trifluoromethyl-phenyl)-isoxazol-5- ylmethyl]-5H-imidazo[4,5-d]pyridazine |
| 102 | 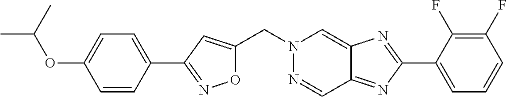 | 2-(2,3-Difluoro-phenyl)-5-[3-(4- isopropoxy-phenyl)-isoxazol-5- ylmethyl]-5H-imidazo[4,5-d]pyridazine |
| 103 | 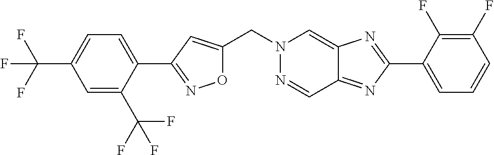 | 5-[3-(2,4-Bis-trifluoromethyl-phenyl)- isoxazol-5-ylmethyl]-2-(2,3-difluoro- phenyl)-5H-imidazo[4,5-d]pyridazine |
| 104 | 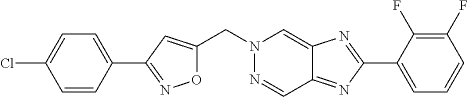 | 5-[3-(4-Chloro-phenyl-isoxazol-5- ylmethyl]-2-(2,3-difluoro-phenyl)-5H- imidazo[4,5-d]pyridazine |
| 105 | 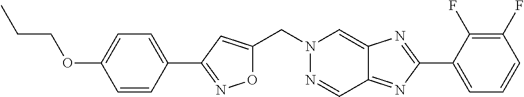 | 2-(2,3-Difluoro-phenyl)-5-[3-(4- propoxy-phenyl)-isoxazol-5-ylmethyl]- 5H-imidazo[4,5-d]pyridazine |
| 106 | 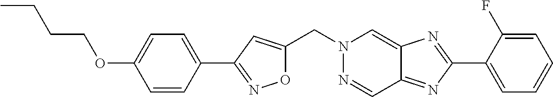 | 5-[3-(4-Butoxy-phenyl)-isoxazol-5- ylmethyl]-2-(2-fluoro-phenyl)-5H- imidazo[4,5-d]pyridazine |
| 107 | 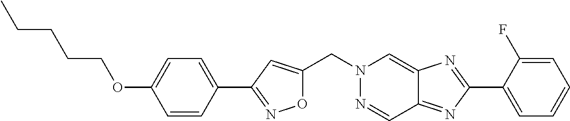 | 2-(2-Fluoro-phenyl)-5-[3-(4-pentyloxy- phenyl)-isoxazol-5-ylmethyl]-5H- imidazo[4,5-d]pyridazine |
| 108 | 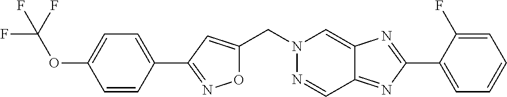 | 2-(2-Fluoro-phenyl)-5-[3-(4- trifluoromethoxy-phenyl)-isoxazol-5- ylmethyl]-5H-imidazo[4,5-d]pyridazine |
| 109 | 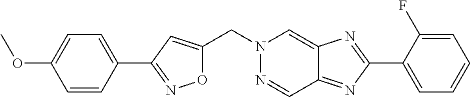 | 2-(2-Fluoro-phenyl)-5-[3-(4-methoxy- phenyl)-isoxazol-5-ylmethyl]-5H- imidazo[4,5-d]pyridazine |
| 110 | 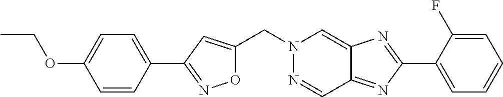 | 5-[3-(4-Ethoxy-phenyl)-isoxazol-5- ylmethyl]-2-(2-fluoro-phenyl)-5H- imidazo[4,5-d]pyridazine |
| 111 | 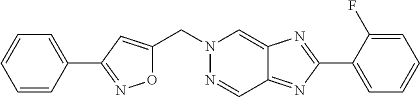 | 2-(2-Fluoro-phenyl)-5-(3-phenyl- isoxazol-5-ylmethyl)-5H-imidazo[4,5- d]pyridazine |
| 112 | 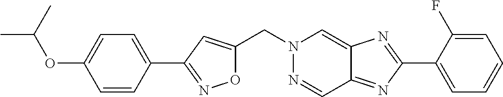 | 2-(2-Fluoro-phenyl)-5-[3-(4- isopropoxy-phenyl)-isoxazol-5- ylmethyl]-5H-imidazo[4,5-d]pyridazine |
| 113 | 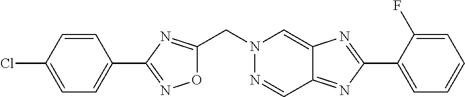 | 5-[3-(4-Chloro-phenyl)- [1,2,4]oxadiazol-5-ylmethyl]-2-(2- fluoro-phenyl)-5H-imidazo[4,5- d]pyridazine |
| 114 | 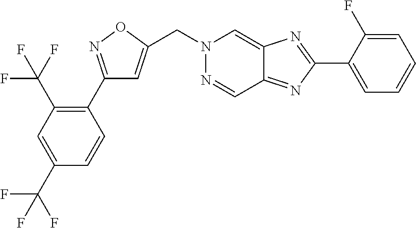 | 5-[3-(2,4-Bis-trifluoromethyl-phenyl)- isoxazol-5-ylmethyl]-2-(2-fluoro- phenyl)-5H-imidazo[4,5-d]pyridazine |
| 115 | 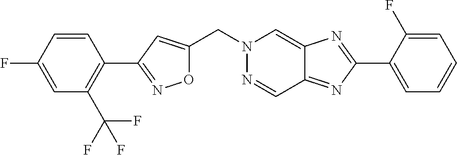 | 2-(2-Fluoro-phenyl)-5-[3-(4-fluoro-2- trifluoromethyl-phenyl)-isoxazol-5- ylmethyl]-5H-imidazo[4,5-d]pyridazine |
| 116 | 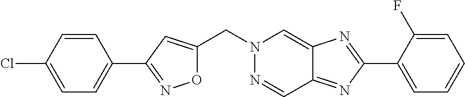 | 5-[3-(4-Chloro-phenyl)-isoxazol-5- ylmethyl]-2-(2-fluoro-phenyl)-5H- imidazo[4,5-d]pyridazine |
| 117 | 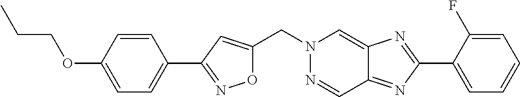 | 2-(2-Fluoro-phenyl)-5-[3-(4-propoxy- phenyl)-isoxazol-5-ylmethyl]-5H- imidazo[4,5-d]pyridazine |
| 119 | 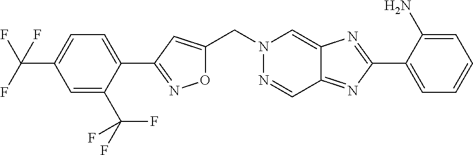 | 2-{5-[3-(2,4-Bis-trifluoromethyl- phenyl)-isoxazol-5-ylmethyl]-5H- imidazo[4,5-d]pyridazin-2-yl}- phenylamine |
| 120 | 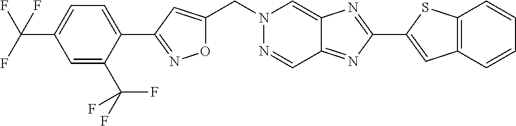 | 2-Benzo[b]thiophen-2-yl-5-[3-(2,4-bis- trifluoromethyl-phenyl)-isoxazol-5- ylmethyl]-5H-imidazo[4,5-d]pyridazine |
| 121 | 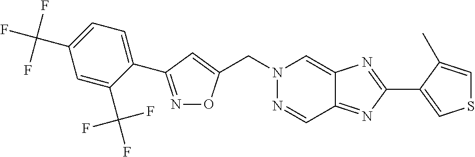 | 5-[3-(2,4-Bis-trifluoromethyl-phenyl)- isoxazol-5-ylmethyl]-2-(4-methyl- thiophen-3-yl)-5H-imidazo[4,5- d]pyridazine |
| 122 | 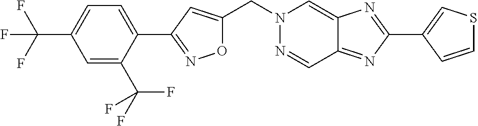 | 5-[3-(2,4-Bis-trifluoromethyl-phenyl)- isoxazol-5-ylmethyl]-2-thiophen-3-yl- 5H-imidazo[4,5-d]pyridazine |
| 123 | 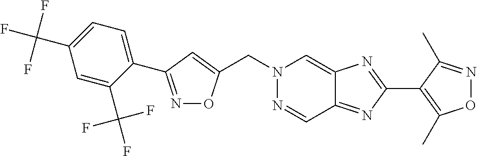 | 5-[3-(2,4-Bis-trifluoromethyl-phenyl)- isoxazol-5-ylmethyl]-2-(3,5-dimethyl- isoxazol-4-yl)-5H-imidazo[4,5- d]pyridazine |
| 124 | 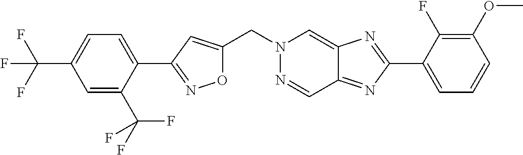 | 5-[3-(2,4-Bis-trifluoromethyl-phenyl)- isoxazol-5-ylmethyl]-2-(2-fluoro-3- methoxy-phenyl)-5H-imidazo[4,5- d]pyridazine |
| 125 | 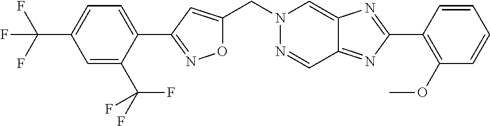 | 5-[3-(2,4-Bis-trifluoromethyl-phenyl)- isoxazol-5-ylmethyl]-2-(2-methoxy- phenyl)-5H-imidazo[4,5-d]pyridazine |
| 126 | 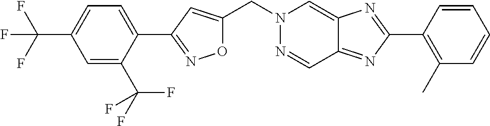 | 5-[3-(2,4-Bis-trifluoromethyl-phenyl)- isoxazol-5-ylmethyl]-2-o-tolyl-5H- imidazo[4,5-d]pyridazine |
| 127 | 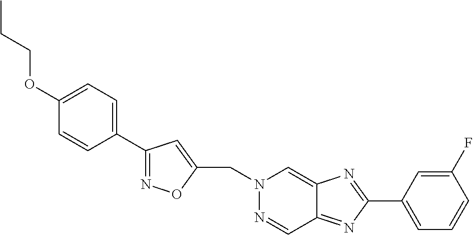 | 2-(3-Fluoro-phenyl)-5-[3-(4-propoxy- phenyl)-isoxazol-5-ylmethyl]-5H- imidazo[4,5-d]pyridazine |
| 128 | 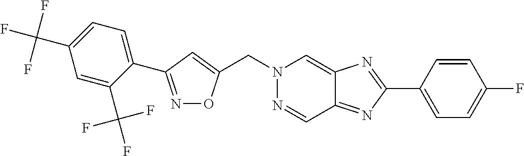 | 5-[3-(2,4-Bis-trifluoromethyl-phenyl)- isoxazol-5-ylmethyl]-2-(4-fluoro- phenyl)-5H-imidazo[4,5-d]pyridazine |
| 129 | 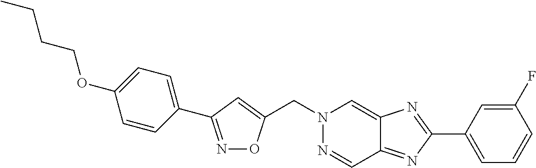 | 5-[3-(4-Butoxy-phenyl)-isoxazol-5- ylmethyl]-2-(3-fluoro-phenyl)-5H- imidazo[4,5-d]pyridazine |
| 130 | 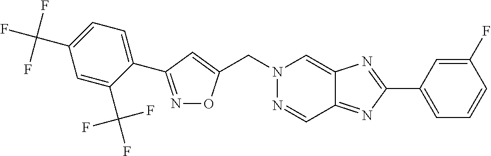 | 5-[3-(2,4-Bis-trifluoromethyl-phenyl)- isoxazol-5-ylmethyl]-2-(3-fluoro- phenyl)-5H-imidazo[4,5-d]pyridazine |
| 131 | 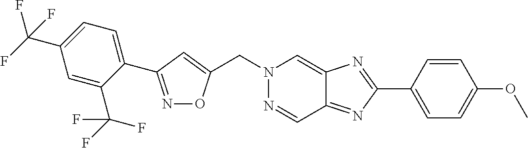 | 5-[3-(2,4-Bis-trifluoromethyl-phenyl)- isoxazol-5-ylmethyl]-2-(4-methoxy- phenyl)-5H-imidazo[4,5-d]pyridazine |
| 132 | 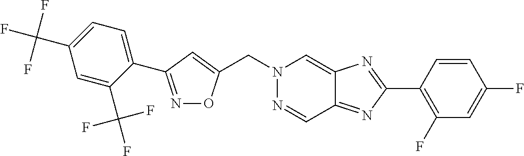 | 5-[3-(2,4-Bis-trifluoromethyl-phenyl)- isoxazol-5-ylmethyl]-2-(2,4-difluoro- phenyl)-5H-imidazo[4,5-d]pyridazine |
| 133 | 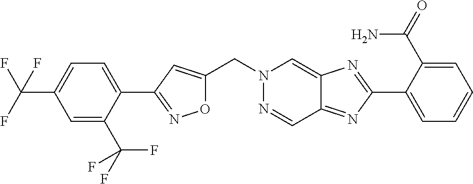 | 2-{5-[3-(2,4-Bis-trifluoromethyl- phenyl)-isoxazol-5-ylmethyl]-5H- imidazo[4,5-d]pyridazin-2-yl}- benzamide |
| 134 | 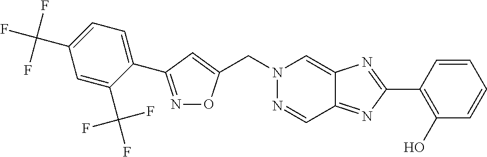 | 2-{5-[3-(2,4-Bis-trifluoromethyl- phenyl)-isoxazol-5-ylmethyl]-5H- imidazo[4,5-d]pyridazin-2-yl}-phenol |
| 135 | 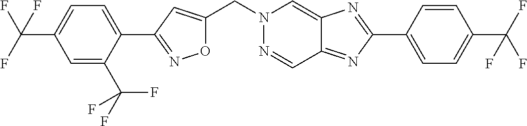 | 5-[3-(2,4-Bis-trifluoromethyl-phenyl)- isoxazol-5-ylmethyl]-2-(4- trifluoromethyl-phenyl)-5H- imidazo[4,5-d]pyridazine |
| 136 | 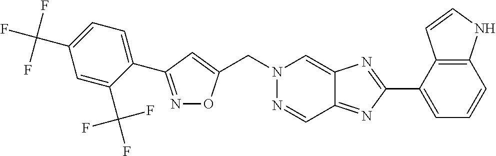 | 5-[3-(2,4-Bis-trifluoromethyl-phenyl)- isoxazol-5-ylmethyl]-2-(1H-indol-4-yl)- 5H-imidazo[4,5-d]pyridazine |
| 137 | 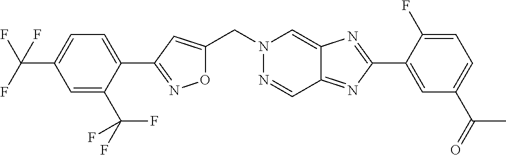 | 1-(3-{5-[3-(2,4-Bis-trifluoromethyl- phenyl)-isoxazol-5-ylmethyl]-5H- imidazo[4,5-d]pyridazin-2-yl}-4-fluoro- phenyl)-ethanone |
| 138 | 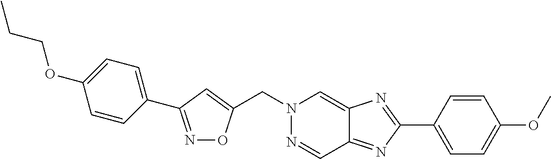 | 2-(4-Methoxy-phenyl)-5-[3-(4-propoxy- phenyl)-isoxazol-5-ylmethyl]-5H- imidazo[4,5-d]pyridazine |
| 139 | 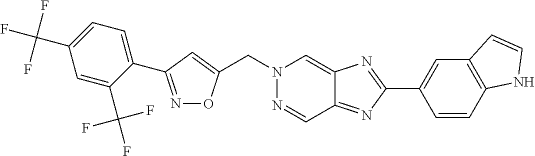 | 5-[3-(2,4-Bis-trifluoromethyl-phenyl)- isoxazol-5-ylmethyl]-2-(1H-indol-5-yl)- 5H-imidazo[4,5-d]pyridazine |
| 140 | 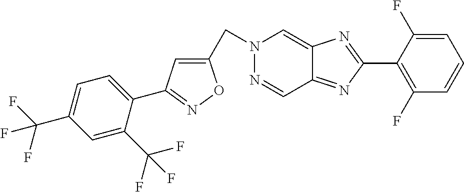 | 5-[3-(2,4-Bis-trifluoromethyl-phenyl)- isoxazol-5-ylmethyl]-2-(2,6-difluoro- phenyl)-5H-imidazo[4,5-d]pyridazine |
| 141 | 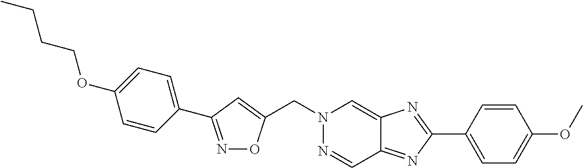 | 5-[3-(4-Butoxy-phenyl)-isoxazol-5- ylmethyl]-2-(4-methoxy-phenyl)-5H- imidazo[4,5-d]pyridazine |
| 142 | 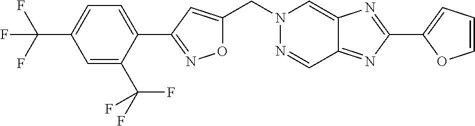 | 5-[3-(2,4-Bis-trifluoromethyl-phenyl)- isoxazol-5-ylmethyl]-2-furan-2-yl-5H- imidazo[4,5-d]pyridazine |
| 143 | 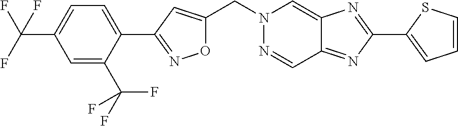 | 5-[3-(2,4-Bis-trifluoromethyl-phenyl)- isoxazol-5-ylmethyl]-2-thiophen-2-yl- 5H-imidazo[4,5-d]pyridazine |
| 144 | 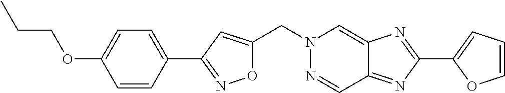 | 2-Furan-2-yl-5-[3-(4-propoxy-phenyl)- isoxazol-5-ylmethyl]-5H-imidazo[4,5- d]pyridazine |
| 145 | 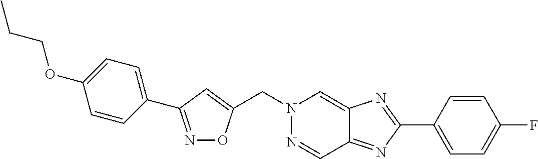 | 2-(4-Fluoro-phenyl)-5-[3-(4-propoxy- phenyl)-isoxazol-5-ylmethyl]-5H- imidazo[4,5-d]pyridazine |
| 146 | 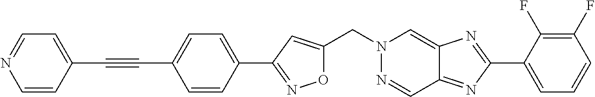 | 2-(2,3-Difluoro-phenyl)-5-[3-(4-pyridin- 4-ylethynyl-phenyl)-isoxazol-5- ylmethyl]-5H-imidazo[4,5-d]pyridazine |
| 147 | 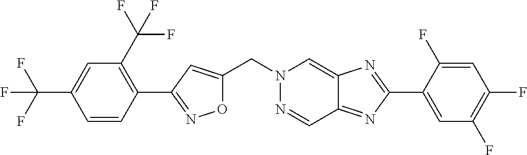 | 5-[3-(2,4-Bis-trifluoromethyl-phenyl)- isoxazol-5-ylmethyl]-2-(2,4,5-trifluoro- phenyl)-5H-imidazo[4,5-d]pyridazine |
| 148 | 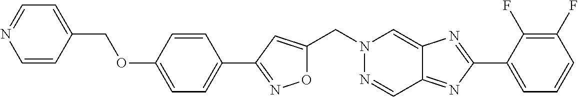 | 2-(2,3-Difluoro-phenyl)-5-{3-[4- (pyridin-4-ylmethoxy)-phenyl]- isoxazol-5-ylmethyl}-5H-imidazo[4,5- d]pyridazine |
| 149 | 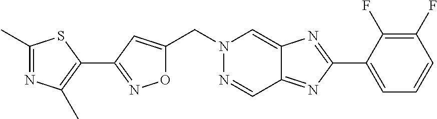 | 2-(2,3-Difluoro-phenyl)-5-[3-(2,4- dimethyl-thiazol-5-yl)-isoxazol-5- ylmethyl]-5H-imidazo[4,5-d]pyridazine |
| 150 | 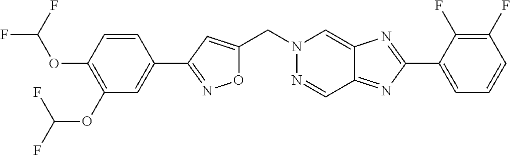 | 5-[3-(3,4-Bis-difluoromethoxy-phenyl)- isoxazol-5-ylmethyl]-2-(2,3-difluoro- phenyl)-5H-imidazo[4,5-d]pyridazine |
| 151 | 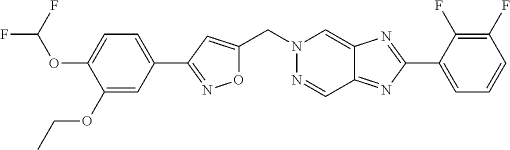 | 5-[3-(4-Difluoromethoxy-3-ethoxy- phenyl)-isoxazol-5-ylmethyl]-2-(2,3- difluoro-phenyl)-5H-imidazo[4,5- d]pyridazine |
| 152 | 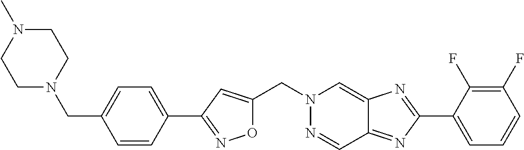 | 2-(2,3-Difluoro-phenyl)-5-{3-[4-(4- methyl-piperazin-1-ylmethyl)phenyl]- isoxazol-5-ylmethyl}-5H-imidazo[4,5- d]pyridazine |
| 153 | 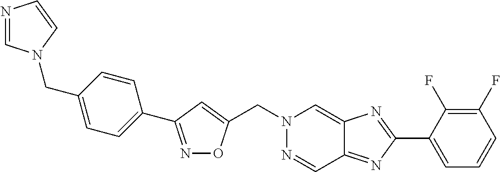 | 2-(2,3-Difluoro-phenyl)-5-[3-(4- imidazol-1-ylmethyl-phenyl)-isoxazol- 5-ylmethyl]-5H-imidazo[4,5- d]pyridazine |
| 154 | 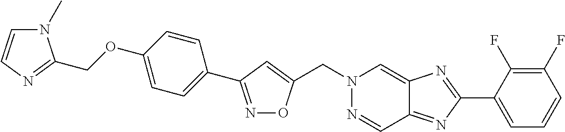 | 2-(2,3-Difluoro-phenyl)-5-{3-[4-(1- methyl-1H-imidazol-2-ylmethoxy)- phenyl]-isoxazol-5-ylmethyl}-5H- imidazo[4,5-d]pyridazine |
| 155 | 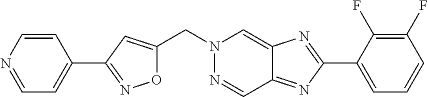 | 2-(2,3-Difluoro-phenyl)-5-(3-pyridin-4- yl-isoxazol-5-ylmethyl)-5H- imidazo[4,5-d]pyridazine |
| 156 | 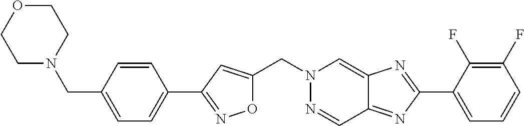 | 2-(2,3-Difluoro-phenyl)-5-[3-(4- morpholin-4-ylmethyl-phenyl)- isoxazol-5-ylmethyl]-5H-imidazo[4,5- d]pyridazine |
| 157 | 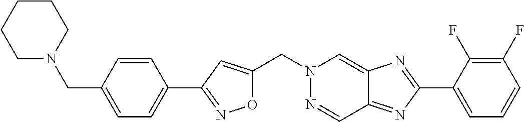 | 2-(2,3-Difluoro-phenyl)-5-[3-(4- piperidin-1-ylmethyl-phenyl)-isoxazol- 5-ylmethyl]-5H-imidazo[4,5- d]pyridazine |
| 158 | 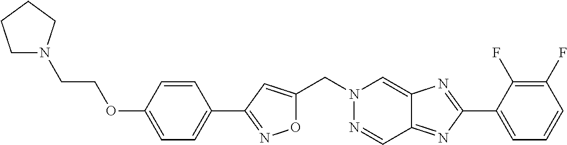 | 2-(2,3-Difluoro-phenyl)-5-{3-[4-(2- pyrrolidin-1-yl-ethoxy)-phenyl]- isoxazol-5-ylmethyl}-5H-imidazo[4,5- d]pyridazine |
| 159 | 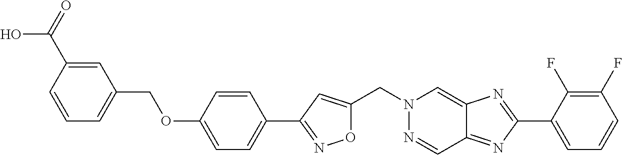 | 3-(4-{5-[2-(2,3-Difluoro-phenyl)- imidazo[4,5-d]pyridazin-5-ylmethyl]- isoxazol-3-yl}-phenoxymethyl)- benzoic acid |
| 160 | 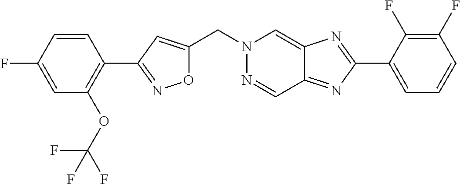 | 2-(2,3-Difluoro-phenyl)-5-[3-(4-fluoro- 2-trifluoromethoxy-phenyl)isoxazol-5- ylmethyl]-5H-imidazo[4,5-d]pyridazine |
| 161 | 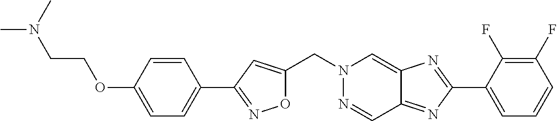 | [2-(4-{5-[2-(2,3-Difluoro-phenyl)- imidazo[4,5-d]pyridazin-5-ylmethyl]- isoxazol-3-yl}-phenoxy)-ethyl]- dimethyl-amine |
| 162 | 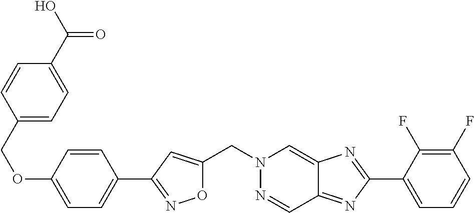 | 4-(4-{5-[2-(2,3-Difluoro-phenyl)- imidazo[4,5-d]pyridazin-5-ylmethyl]- isoxazol-3-yl}-phenoxymethyl)- benzoic acid |
| 163 | 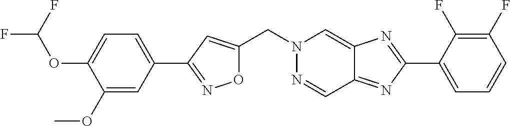 | 5-[3-(4-Difluoromethoxy-3-methoxy- phenyl)-isoxazol-5-ylmethyl]-2-(2,3- difluoro-phenyl)-5H-imidazo[4,5- d]pyridazine |
| 164 | 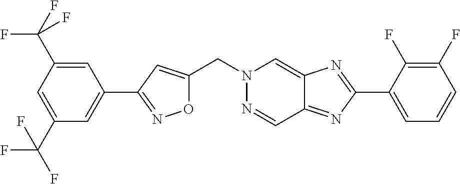 | 5-[3-(3,5-Bis-trifluoromethyl-phenyl)- isoxazol-5-ylmethyl]-2-(2,3-difluoro- phenyl)-5H-imidazo[4,5-d]pyridazine |
| 165 | 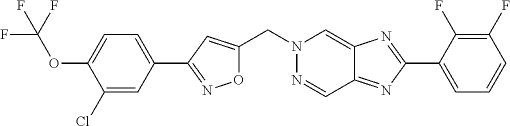 | 5-[3-(3-Chloro-4-trifluoromethoxy- phenyl)-isoxazol-5-ylmethyl]-2-(2,3- difluoro-phenyl)-5H-imidazo[4,5- d]pyridazine |
| 166 | 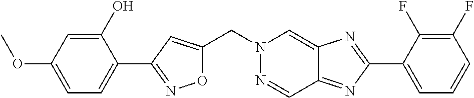 | 2-{5-[2-(2,3-Difluoro-phenyl)- imidazo[4,5-d]pyridazin-5-ylmethyl]- isoxazol-3-yl}-5-methoxy-phenol |
| 167 | 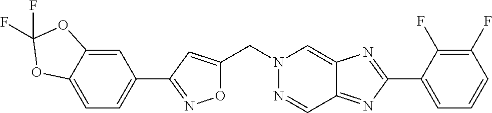 | 5-[3-(2,2-Difluoro-benzo[1,3]dioxol-5- yl)-isoxazol-5-ylmethyl]-2-(2,3-difluoro- phenyl)-5H-imidazo[4,5-d]pyridazine |
| 168 | 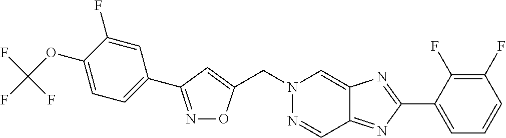 | 2-(2,3-Difluoro-phenyl)-5-[3-(3-fluoro- 4-trifluoromethoxy-phenyl)-isoxazol-5- ylmethyl]-5H-imidazo[4,5-d]pyridazine |
| 169 | 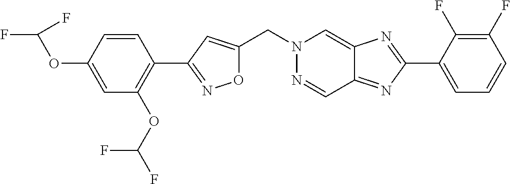 | 5-[3-(2,4-Bis-difluoromethoxy-phenyl)- isoxazol-5-ylmethyl]-2-(2,3-difluoro- phenyl)-5H-imidazo[4,5-d]pyridazine |
| 170 | 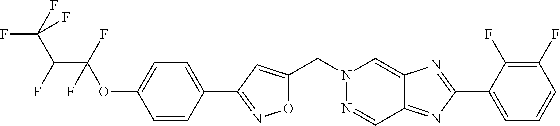 | 2-(2,3-Difluoro-phenyl)-5-{3-[4- (1,1,2,3,3,3-hexafluoro-propoxy)- phenyl]-isoxazol-5-ylmethyl}-5H- imidazo[4, 5-d]pyridazine |
| 171 | 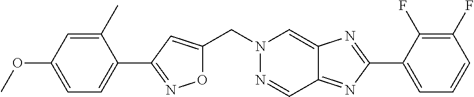 | 2-(2,3-Difluoro-phenyl)-5-[3-(4- methoxy-2-methyl-phenyl)-isoxazol-5- ylmethyl]-5H-imidazo[4,5-d]pyridazine |
| 172 | 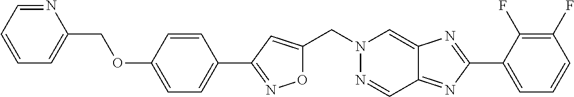 | 2-(2,3-Difluoro-phenyl)-5-{3-[4- (pyridin-2-ylmethoxy)-phenyl]- isoxazol-5-ylmethyl}-5H-imidazo[4,5- d]pyridazine |
| 173 | 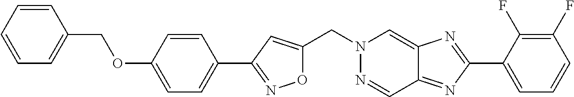 | 5-[3-(4-Benzyloxy-phenyl)-isoxazol-5- ylmethyl]-2-(2,3-difluoro-phenyl)-5H- imidazo[4,5-d]pyridazine |
| 174 | 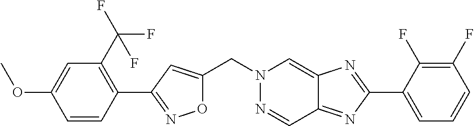 | 2-(2,3-Difluoro-phenyl)-5-[3-(4- methoxy-2-trifluoromethyl-phenyl)- isoxazol-5-ylmethyl]-5H-imidazo[4,5- d]pyridazine |
| 175 | 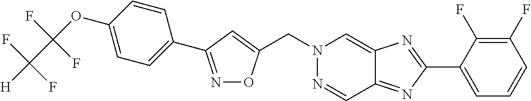 | 2-(2,3-Difluoro-phenyl)-5-{3-[4- (1,1,2,2-tetrafluoro-ethoxy)-phenyl]- isoxazol-5-ylmethyl}-5H-imidazo[4,5- d]pyridazine |
| 176 | 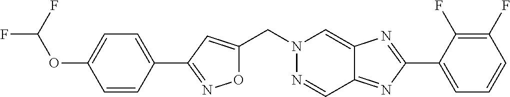 | 5-[3-(4-Difluoromethoxy-phenyl)- isoxazol-5-ylmethyl]-2-(2,3-difluoro- phenyl)-5H-imidazo[4,5-d]pyridazine |
| 177 | 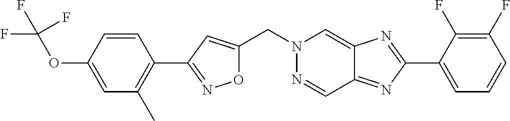 | 2-(2,3-Difluoro-phenyl)-5-[3-(2-methyl- 4-trifluoromethoxy-phenyl)-isoxazol-5- ylmethyl]-5H-imidazo[4,5-d]pyridazine |
| 178 | 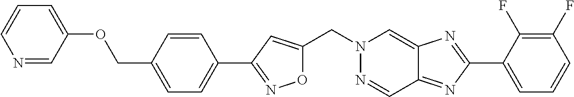 | 2-(2,3-Difluoro-phenyl)-5-{3-[4- (pyridin-3-yloxymethyl)-phenyl]- isoxazol-5-ylmethyl}-5H-imidazo[4,5- d]pyridazine |
| 179 | 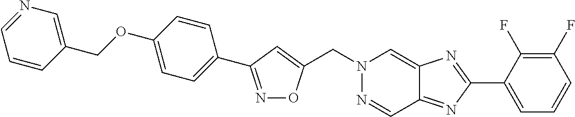 | 2-(2,3-Difluoro-phenyl)-5-{3-[4- (pyridin-3-ylmethoxy)-phenyl]- isoxazol-5-ylmethyl}-5H-imidazo[4,5- d]pyridazine |
| 180 | 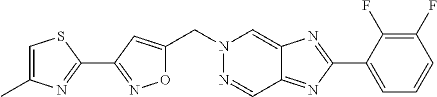 | 2-(2,3-Difluoro-phenyl)-5-[3-(4-methyl- thiazol-2-yl)-isoxazol-5-ylmethyl]-5H- imidazo[4,5-d]pyridazine |
| 181 | 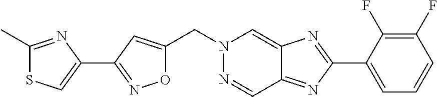 | 2-(2,3-Difluoro-phenyl)-5-[3-(2-methyl- thiazol-4-yl)-isoxazol-5-ylmethyl]-5H- imidazo[4,5-d]pyridazine |
| 182 | 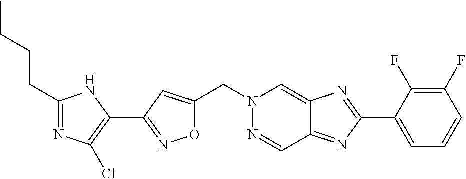 | 5-[3-(2-Butyl-5-chloro-3H-imidazol-4- yl)-isoxazol-5-ylmethyl]-2-(2,3-difluoro- phenyl)-5H-imidazo[4,5-d]pyridazine |
| 183 | 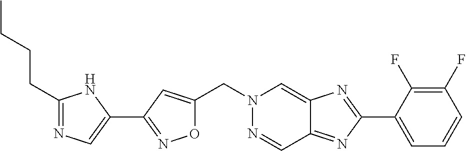 | 5-[3-(2-Butyl-3H-imidazol-4-yl)- isoxazol-5-ylmethyl]-2-(2,3-difluoro- phenyl)-5H-imidazo[4,5-d]pyridazine |
| 184 | 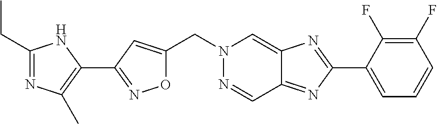 | 2-(2,3-Difluoro-phenyl)-5-[3-(2-ethyl-5- methyl-3H-imidazol-4-yl)-isoxazol-5- ylmethyl]-5H-imidazo[4,5-d]pyridazine |
| 185 | 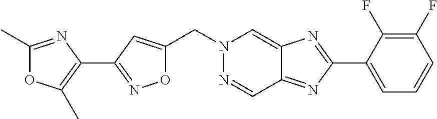 | 2-(2,3-Difluoro-phenyl)-5-[3-(2,5- dimethyl-oxazol-4-yl)-isoxazol-5- ylmethyl]-5H-imidazo[4,5-d]pyridazine |
| 186 | 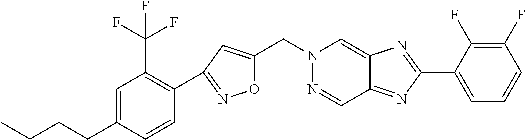 | 5-[3-(4-Butyl-2-trifluoromethyl-phenyl)- isoxazol-5-ylmethyl]-2-(2,3-difluoro- phenyl)-5H-imidazo[4,5-d]pyridazine |
| 187 | 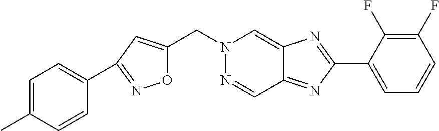 | 2-(2,3-Difluoro-phenyl)-5-(3-p-tolyl- isoxazol-5-ylmethyl)-5H-imidazo[4,5- d]pyridazine |
| 188 | 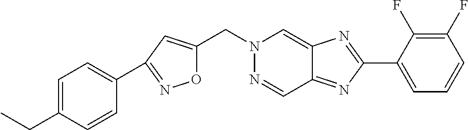 | 2-(2,3-Difluoro-phenyl)-5-[3-(4-ethyl- phenyl)-isoxazol-5-ylmethyl]-5H- imidazo[4,5-d]pyridazine |
| 189 | 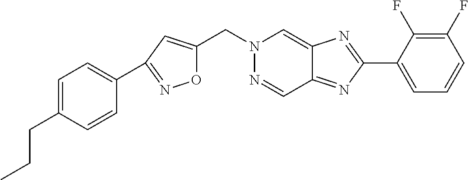 | 2-(2,3-Difluoro-phenyl)-5-[3-(4-propyl- phenyl)-isoxazol-5-ylmethyl]-5H- imidazo[4,5-d]pyridazine |
| 190 | 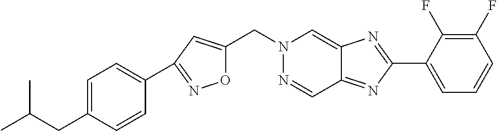 | 2-(2,3-Difluoro-phenyl)-5-[3-(4- isobutyl-phenyl)-isoxazol-5-ylmethyl]- 5H-imidazo[4,5-d]pyridazine |
| 191 | 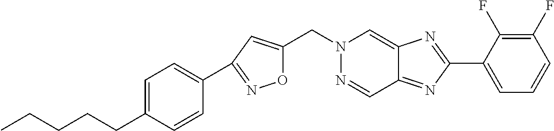 | 2-(2,3-Difluoro-phenyl)-5-[3-(4-pentyl- phenyl)-isoxazol-5-ylmethyl]-5H- imidazo[4,5-d]pyridazine |
| 192 | 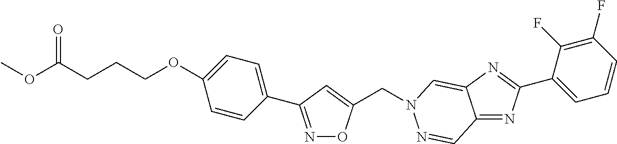 | 4-(4-{5-[2-(2,3-Difluoro-phenyl)- imidazo[4,5-d]pyridazin-5-ylmethyl]- isoxazol-3-yl}-phenoxy)-butyric acid methyl ester |
| 193 | 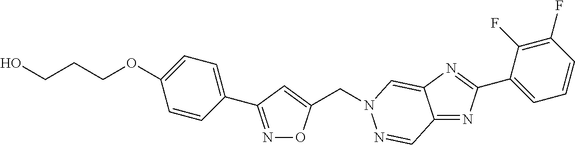 | 3-(4-{5-[2-(2,3-Difluoro-phenyl)- imidazo[4,5-d]pyridazin-5-ylmethyl]- isoxazol-3-yl}-phenoxy)-propan-1-ol |
| 194 | 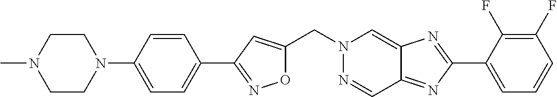 | 2-(2,3-Difluoro-phenyl)-5-{3-[4-(4- methyl-piperazin-1-yl)-phenyl]- isoxazol-5-ylmethyl}-5H-imidazo[4,5- d]pyridazine |
| 195 | 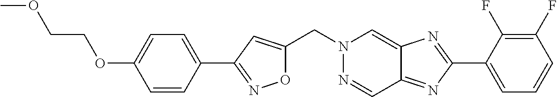 | 2-(2,3-Difluoro-phenyl)-5-{3-[4-(2- methoxy-ethoxy)-phenyl]-isoxazol-5- ylmethyl}-5H-imidazo[4,5-d]pyridazine |
| 196 | 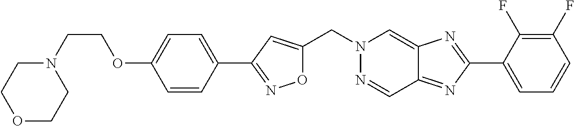 | 2-(2,3-Difluoro-phenyl)-5-{3-[4-(2- morpholin-4-yl-ethoxy)-phenyl]- isoxazol-5-ylmethyl}-5H-imidazo[4,5- d]pyridazine |
| 197 | 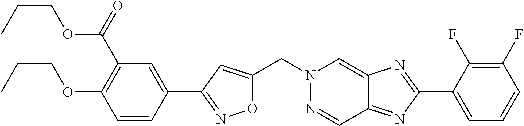 | 5-{5-[2-(2,3-Difluoro-phenyl)- imidazo[4,5-d]pyridazin-5-ylmethyl]- isoxazol-3-yl}-2-propoxy-benzoic acid propyl ester |
| 198 | 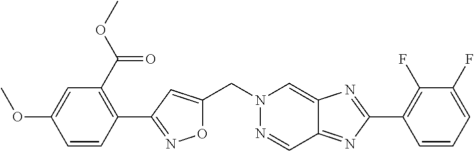 | 2-{5-[2-(2,3-Difluoro-phenyl)- imidazo[4,5-d]pyridazin-5-ylmethyl]- isoxazol-3-yl}-5-methoxy-benzoic acid methyl ester |
| 199 | 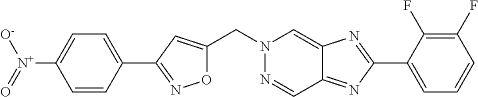 | 2-(2,3-Difluoro-phenyl)-5-[3-(4-nitro- phenyl)-isoxazol-5-ylmethyl]-5H- imidazo[4,5-d]pyridazine |
| 200 | 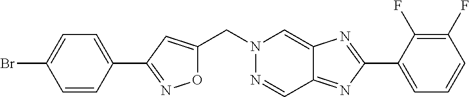 | 5-[3-(4-Bromo-phenyl)-isoxazol-5- ylmethyl]-2-(2,3-difluoro-phenyl)-5H- imidazo[4,5-d]pyridazine |
| 201 | 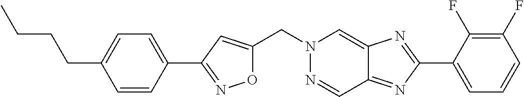 | 5-[3-(4-Butyl-phenyl)-isoxazol-5- ylmethyl]-2-(2,3-difluoro-phenyl)-5H- imidazo[4,5-d]pyridazine |
| 202 | 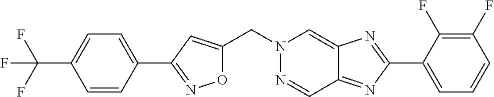 | 2-(2,3-Difluoro-phenyl)-5-[3-(4- trifluoromethyl-phenyl)-isoxazol-5- ylmethyl]-5H-imidazo[4,5-d]pyridazine |
| 203 | 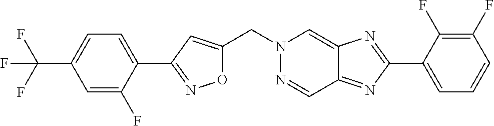 | 2-(2,3-Difluoro-phenyl)-5-[3-(2-fluoro- 4-trifluoromethyl-phenyl)-isoxazol-5- ylmethyl]-5H-imidazo[4,5-d]pyridazine |
| 204 | 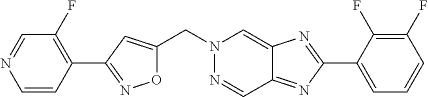 | 2-(2,3-Difluoro-phenyl)-5-[3-(3-fluoro- pyridin-4-yl)-isoxazol-5-ylmethyl]-5H- imidazo[4,5-d]pyridazine |
| 205 | 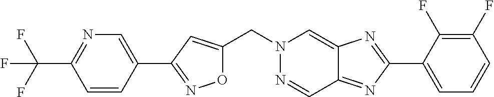 | 2-(2,3-Difluoro-phenyl)-5-[3-(6- trifluoromethyl-pyridin-3-yl)-isoxazol-5- ylmethyl]-5H-imidazo[4,5-d]pyridazine |
| 206 | 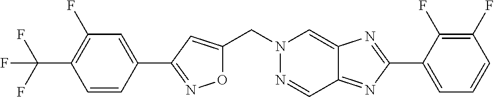 | 2-(2,3-Difluoro-phenyl)-5-[3-(3-fluoro- 4-trifluoromethyl-phenyl)-isoxazol-5- ylmethyl]-5H-imidazo[4,5-d]pyridazine |
| 207 | 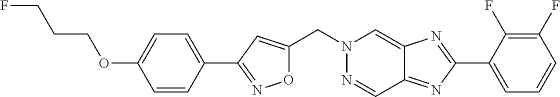 | 2-(2,3-Difluoro-phenyl)-5-{3-[4-(3- fluoro-propoxy)-phenyl]-isoxazol-5- ylmethyl}-5H-imidazo[4,5-d]pyridazine |
| 208 | 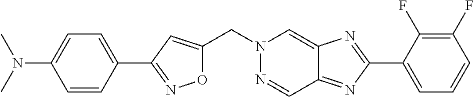 | (4-{5-[2-(2,3-Difluoro-phenyl)- imidazo[4,5-d]pyridazin-5-ylmethyl]- isoxazol-3-yl}-phenyl)-dimethyl-amine |
| 209 | 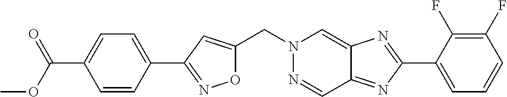 | 4-{5-[2-(2,3-Difluoro-phenyl)- imidazo[4,5-d]pyridazin-5-ylmethyl]- isoxazol-3-yl}-benzoic acid methyl ester |
| 210 | 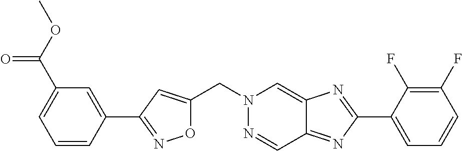 | 3-{5-[2-(2,3-Difluoro-phenyl)- imidazo[4,5-d]pyridazin-5-ylmethyl]- isoxazol-3-yl}-benzoic acid methyl ester |
| 211 | 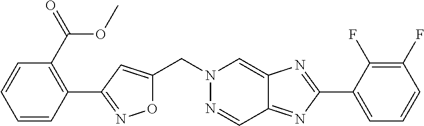 | 2-{5-[2-(2,3-Difluoro-phenyl)- imidazo[4,5-d]pyridazin-5-ylmethyl]- isoxazol-3-yl}-benzoic acid methyl ester |
| 212 | 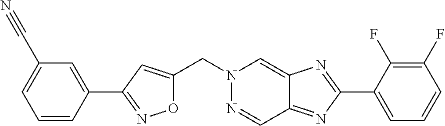 | 3-{5-[2-(2,3-Difluoro-phenyl)- imidazo[4,5-d]pyridazin-5-ylmethyl]- isoxazol-3-yl)-benzonitrile |
| 213 | 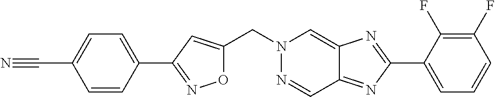 | 4-{5-[2-(2,3-Difluoro-phenyl)- imidazo[4,5-d]pyridazin-5-ylmethyl]- isoxazol-3-yl}-benzonitrile |
| 214 | 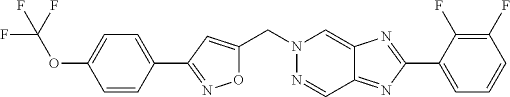 | 2-(2,3-Difluoro-phenyl)-5-[3-(4- trifluoromethoxy-phenyl)-isoxazol-5- ylmethyl]-5H-imidazo[4,5-d]pyridazine |
| 215 | 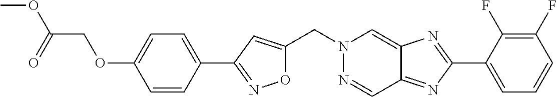 | (4-{5-[2-(2,3-Difluoro-phenyl)- imidazo[4,5-d]pyridazin-5-ylmethyl]- isoxazol-3-yl}-phenoxy)-acetic acid methyl ester |
| 216 | 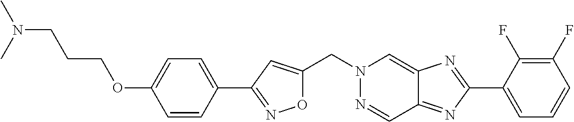 | [3-(4-{5-[2-(2,3-Difluoro-phenyl)- imidazo[4,5-d]pyridazin-5-ylmethyl]- isoxazol-3-yl}-phenoxy)-propyl]- dimethyl-amine |
| 217 | 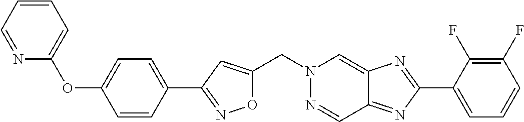 | 2-(2,3-Difluoro-phenyl)-5-{3-[4- (pyridin-2-yloxy)-pheny]-isoxazol-5- ylmethyl}-5H-imidazo[4,5-d]pyridazine |
| 218 | 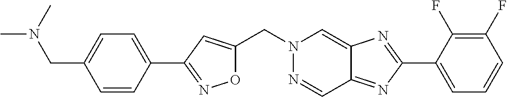 | (4-{5-[2-(2,3-Difluoro-phenyl)- imidazo[4,5-d]pyridazin-5-ylmethyl]- isoxazol-3-yl}-benzyl)-dimethyl-amine |
| 219 | 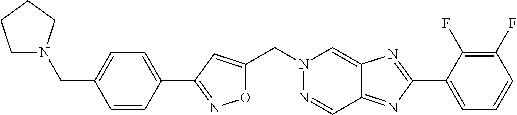 | 2-(2,3-Difluoro-phenyl)-5-[3-(4- pyrrolidin-1-ylmethyl-phenyl)-isoxazol- 5-ylmethyl]-5H-imidazo[4,5- d]pyridazine |
| 220 | 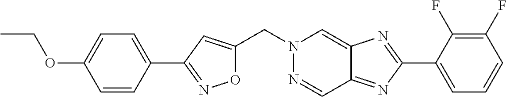 | 2-(2,3-Difluoro-phenyl)-5-[3-(4-ethoxy- phenyl)-isoxazol-5-ylmethyl]-5H- imidazo[4,5-d]pyridazine |
| 221 | 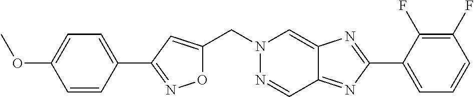 | 2-(2,3-Difluoro-phenyl)-5-[3-(4- methoxy-phenyl)-isoxazol-5-ylmethyl]- 5H-imidazo[4,5-d]pyridazine |
| 222 | 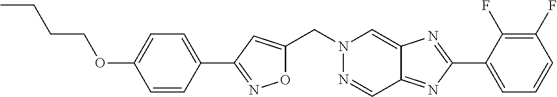 | 5-[3-(4-Butoxy-phenyl)-isoxazol-5- ylmethyl]-2-(2,3-difluoro-phenyl)-5H- imidazo[4,5-d]pyridazine |
| 223 | 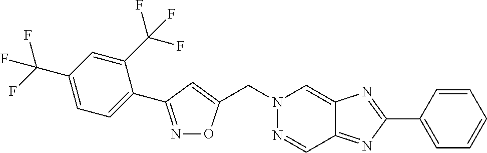 | 5-[3-(2,4-Bis-trifluoromethyl-phenyl)- isoxazol-5-ylmethyl]-2-phenyl-5H- imidazo[4,5-d]pyridazine |
| 224 | 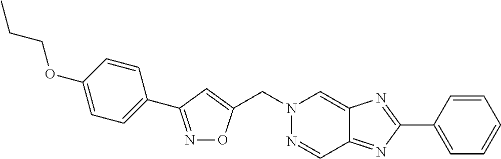 | 2-Phenyl-5-[3-(4-propoxy-phenyl)- isoxazol-5-ylmethyl]-5H-imidazo[4,5- d]pyridazine |
| 225 | 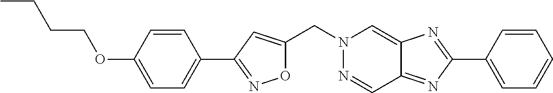 | 5-[3-(4-Butoxy-phenyl)-isoxazol-5- ylmethyl]-2-phenyl-5H-imidazo[4,5- d]pyridazine |
| 226 | 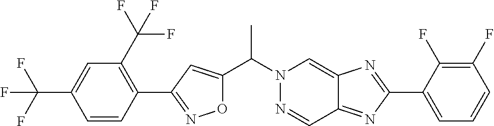 | 5-{1-[3-(2,4-Bis-trifluoromethyl- phenyl)-isoxazol-5-yl]-ethyl}-2-(2,3- difluoro-phenyl)-5H-imidazo[4,5- d]pyridazine |
| 227 | 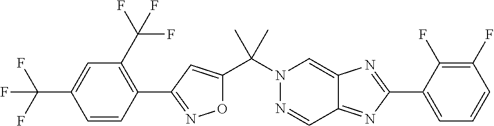 | 5-{1-[3-(2,4-Bis-trifluoromethyl- phenyl)-isoxazol-5-yl]-1-methyl-ethyl}- 2-(2,3-difluoro-phenyl)-5H- imidazo[4,5-d]pyridazine |
| 228 | 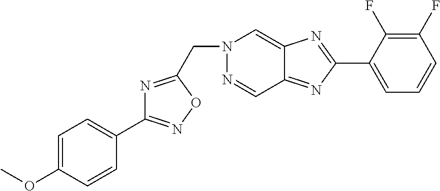 | 2-(2,3-Difluoro-phenyl)-5-[3-(4- methoxy-phenyl)-[1,2,4]oxadiazol-5- ylmethyl]-5H-imidazo[4,5-d]pyridazine |
| 229 | 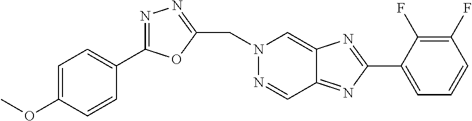 | 2-(2,3-Difluoro-phenyl)-5-[5-(4- methoxy-phenyl)-[1,3,4]oxadiazol-2- ylmethyl]-5H-imidazo[4,5-d]pyridazine |
| 230 | 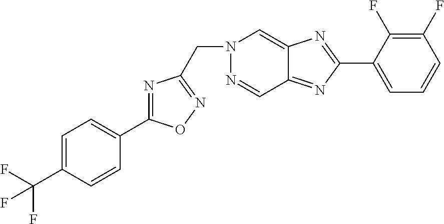 | 2-(2,3-Difluoro-phenyl)-5-[5-(4- trifluoromethyl-phenyl)- [1,2,4]oxadiazol-3-ylmethyl]-5H- imidazo[4,5-d]pyridazine |
| 231 | 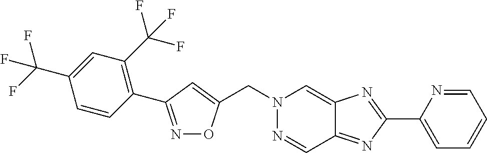 | 5-[3-(2,4-Bis-trifluoromethyl-phenyl)- isoxazol-5-ylmethyl]-2-pyridin-2-yl-5H- imidazo[4,5-d]pyridazine |
| 232 | 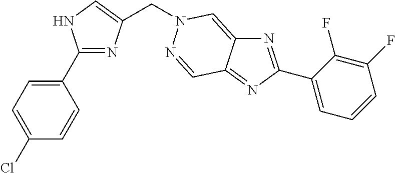 | 5-[2-(4-Chloro-phenyl)-1H-imidazol-4- ylmethyl]-2-(2,3-difluoro-phenyl)-5H- imidazo[4,5-d]pyridazine |
| 233 | 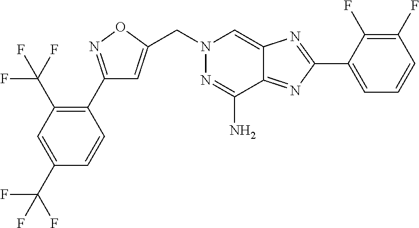 | 6-[3-(2,4-Bis-trifluoromethyl-phenyl)- isoxazol-5-ylmethyl]-2-(2,3-difluoro- phenyl)-6H-imidazo[4,5-d]pyridazin-4- ylamine |
| 234 | 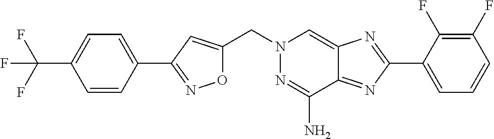 | 2-(2,3-Difluoro-phenyl)-6-[3-(4- trifluoromethyl-phenyl)-isoxazol-5- ylmethyl]-6H-imidazo[4,5-d]pyridazin- 4-ylamine |
| 235 | 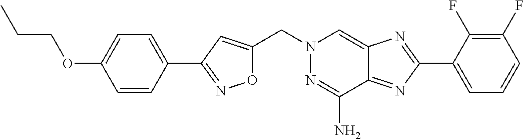 | 2-(2,3-Difluoro-phenyl)-6-[3-(4- propoxy-phenyl)-isoxazol-5-ylmethyl]- 6H-imidazo[4,5-d]pyridazin-4-ylamine |
| 236 | 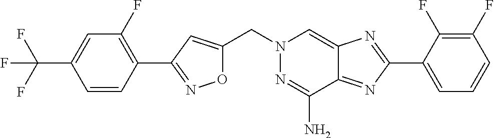 | 2-(2,3-Difluoro-phenyl)-6-[3-(2-fluoro- 4-trifluoromethyl-phenyl)-isoxazol-5- ylmethyl]-6H-imidazo[4,5-d]pyridazin- 4-ylamine |
| 237 | 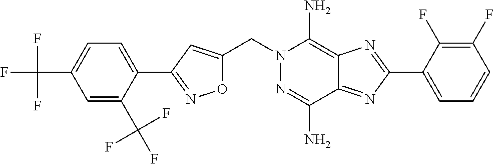 | 5-[3-(2,4-Bis-trifluoromethyl-phenyl)- isoxazol-5-ylmethyl]-2-(2,3-difluoro- phenyl)-5H-imidazo[4,5-d]pyridazine- 4,7-diamine |
| 238 | 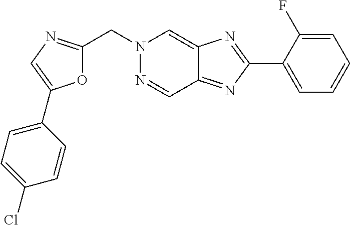 | 5-[5-(4-Chloro-phenyl)-oxazol-2- ylmethyl]-2-(2-fluoro-phenyl)-5H- imidazo[4,5-d]pyridazine |
| 239 | 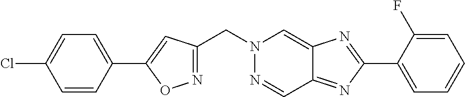 | 5-[5-(4-Chloro-phenyl)-isoxazol-3- ylmethyl]-2-(2-fluoro-phenyl)-5H- imidazo[4,5-d]pyridazine |
| 240 | 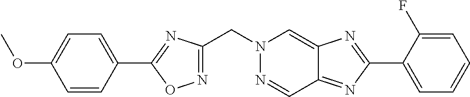 | 2-(2-Fluoro-phenyl)-5-[5-(4-methoxy- phenyl)-[1,2,4]oxadiazol-3-ylmethyl]- 5H-imidazo[4,5-d]pyridazine |
| 241 | 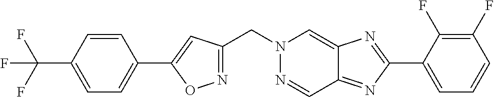 | 2-(2,3-Difluoro-phenyl)-5-[5-(4- trifluoromethyl-phenyl)-isoxazol-3- ylmethyl]-5H-imidazo[4,5-d]pyridazine |
| 242 | 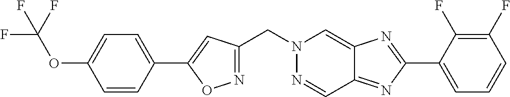 | 2-(2,3-Difluoro-phenyl)-5-[5-(4- trifluoromethoxy-phenyl)-isoxazol-3- ylmethyl]-5H-imidazo[4,5-d]pyridazine |
| 243 | 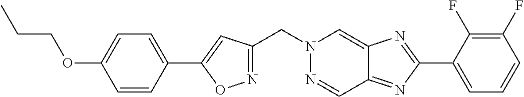 | 2-(2,3-Difluoro-phenyl)-5-[5-(4- propoxy-phenyl)-isoxazol-3-ylmethyl]- 5H-imidazo[4,5-d]pyridazine |
| 244 | 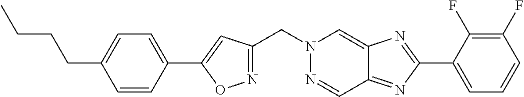 | 5-[5-(4-Butyl-phenyl)-isoxazol-3- ylmethyl]-2-(2,3-difluoro-phenyl)-5H- imidazo[4,5-d]pyridazine |
| 245 | 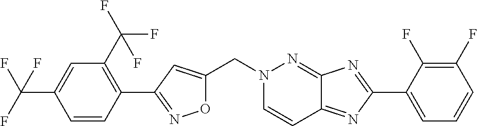 | 2-[3-(2,4-Bis-trifluoromethyl-phenyl)- isoxazol-5-ylmethyl]-6-(2,3-difluoro- phenyl)-2H-imidazo[4,5-c]pyridazine |
| 246 | 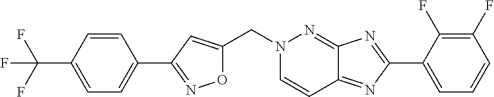 | 6-(2,3-Difluoro-phenyl)-2-[3-(4- trifluoromethyl-phenyl)-isoxazol-5- ylmethyl]-2H-imidazo[4,5-c]pyridazine |
| 247 | 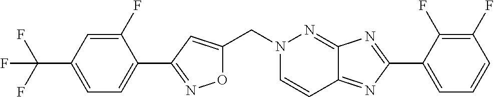 | 6-(2,3-Difluoro-phenyl)-2-[3-(2-fluoro- 4-trifluoromethyl-phenyl)-isoxazol-5- ylmethyl]-2H-imidazo[4,5-c]pyridazine |
| 248 | 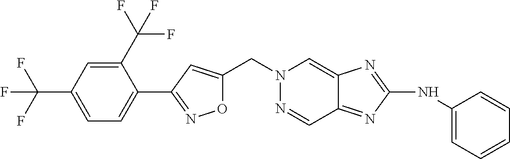 | {5-[3-(2,4-Bis-trifluoromethyl-phenyl)- isoxazol-5-ylmethyl]-5H-imidazo[4,5- d]pyridazin-2-yl}-phenyl-amine |
| 249 | 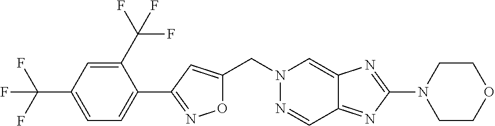 | 5-[3-(2,4-Bis-trifluoromethyl-phenyl)- isoxazol-5-ylmethyl]-2-morpholin-4-yl- 5H-imidazo[4,5-d]pyridazine |
| 250 | 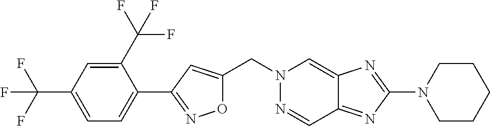 | 5-[3-(2,4-Bis-trifluoromethyl-phenyl)- isoxazol-5-ylmethyl]-2-piperidin-1-yl- 5H-imidazo[4,5-d]pyridazine |
| 251 | 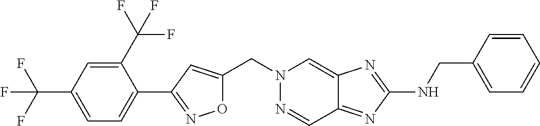 | Benzyl-{5-[3-(2,4-bis-trifluoromethyl- phenyl)-isoxazol-5-ylmethyl]-5H- imidazo[4,5-d]pyridazin-2-yl}-amine |
| 252 | 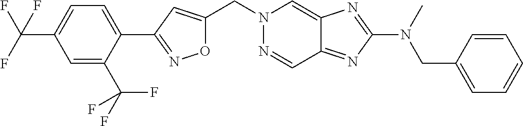 | Benzyl-{5-[3-(2,4-bis-trifluoromethyl- phenyl)-isoxazol-5-ylmethyl]-5H- imidazo[4,5-d]pyridazin-2-yl}-methyl- amine |
| 253 | 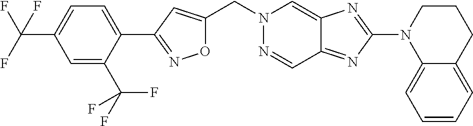 | 1-{5-[3-(2,4-Bis-trifluoromethyl- phenyl)-isoxazol-5-ylmethyl]-5H- imidazo[4,5-d]pyridazin-2-yl}-1,2,3,4- tetrahydro-quinoline |
| 254 | 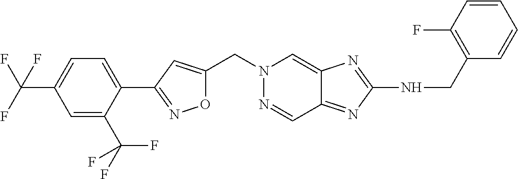 | {5-[3-(2,4-Bis-trifluoromethyl-phenyl)- isoxazol-5-ylmethyl]-5H-imidazo[4,5- d]pyridazin-2-yl)-(2-fluoro-benzyl)- amine |
| 255 | 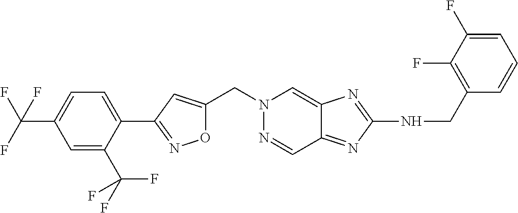 | (5-[3-(2,4-Bis-trifluoromethyl-phenyl)- isoxazol-5-ylmethyl]-5H-imidazo[4,5- d]pyridazin-2-yl)-(2,3-difluoro-benzyl)- amine |
| 256 | 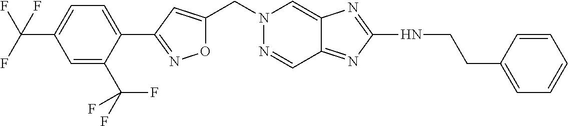 | {5-[3-(2,4-Bis-trifluoromethyl-phenyl)- isoxazol-5-ylmethyl]-5H-imidazo[4,5- d]pyridazin-2-yl}-phenethyl-amine |
| 257 | 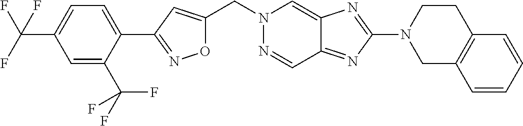 | 2-{5-[3-(2,4-Bis-trifluoromethyl- phenyl)-isoxazol-5-ylmethyl]-5H- imidazo[4,5-d]pyridazin-2-yl}-1,2,3,4- tetrahydro-isoquinoline |
| 258 | 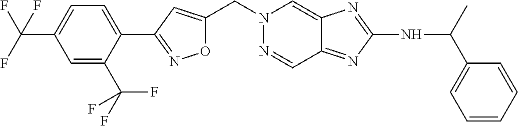 | {5-[3-(2,4-Bis-trifluoromethyl-phenyl)- isoxazol-5-ylmethyl]-5H-imidazo[4,5- d]pyridazin-2-yl}-(1-phenyl-ethyl)- amine |
| 259 | 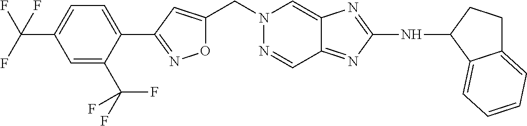 | {5-[3-(2,4-Bis-trifluoromethyl-phenyl)- isoxazol-5-ylmethyl]-5H-imidazo[4,5- d]pyridazin-2-yl}-indan-1-yl-amine |
| 260 | 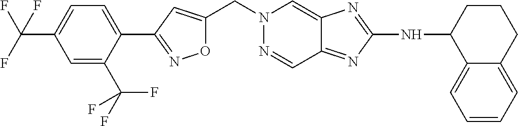 | {5-[3-(2,4-Bis-trifluoromethyl-phenyl)- isoxazol-5-ylmethyl]-5H-imidazo[4,5- d]pyridazin-2-yl}-(1,2,3,4-tetrahydro- naphthalen-1-yl)-amine |
| 261 | 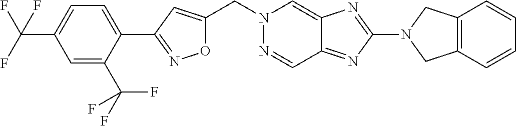 | 5-[3-(2,4-Bis-trifluoromethyl-phenyl)- isoxazol-5-ylmethyl]-2-(1,3-dihydro- isoindol-2-yl)-5H-imidazo[4,5- d]pyridazine |
| 262 | 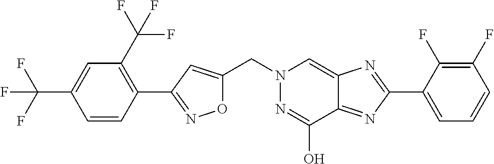 | 6-[3-(2,4-Bis-trifluoromethyl-phenyl)- isoxazol-5-ylmethyl]-2-(2,3-difluoro- phenyl)-6H-imidazo[4,5-d]pyridazin-4- ol |
| 263 | 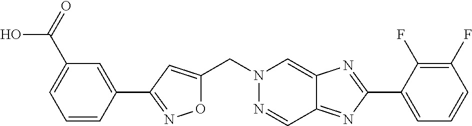 | 3-{5-[2-(2,3-Difluoro-phenyl)- imidazo[4,5-d]pyridazin-5-ylmethyl]- isoxazol-3-yl}-benzoic acid |
| 264 | 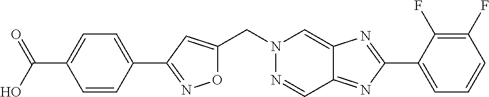 | 4-{5-[2-(2,3-Difluoro-phenyl)- midazo[4,5-d]pyridazin-5-ylmethyl]- isoxazol-3-yl}-benzoic acid |
| 265 | 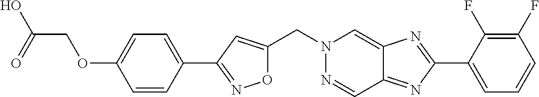 | (4-{5-[2-(2,3-Difluoro-phenyl)- imidazo[4,5-d]pyridazin-5-ylmethyl]- isoxazol-3-yl}-phenoxy)-acetic acid |
| 266 | 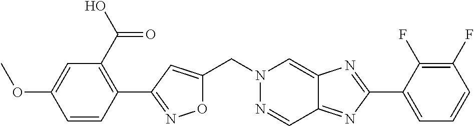 | 2-{5-[2-(2,3-Difluoro-phenyl)- imidazo[4,5-d]pyridazin-5-ylmethyl]- isoxazol-3-yl}-5-methoxy-benzoic acid |
| 267 | 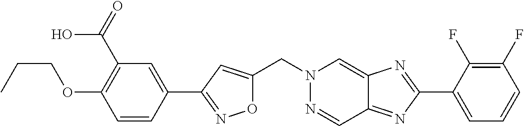 | 5-{5-[2-(2,3-Difluoro-phenyl)- imidazo[4,5-d]pyridazin-5-ylmethyl]- isoxazol-3-yl}-2-propoxy-benzoic acid |
| 268 | 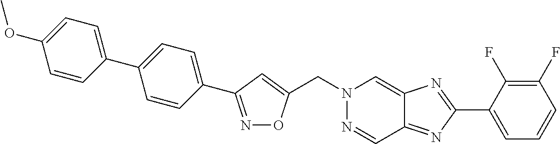 | 2-(2,3-Difluoro-phenyl)-5-[3-(4'- methoxy-biphenyl-4-yl)-isoxazol-5- ylmethyl]-5H-imidazo[4,5-d]pyridazine |
| 269 | 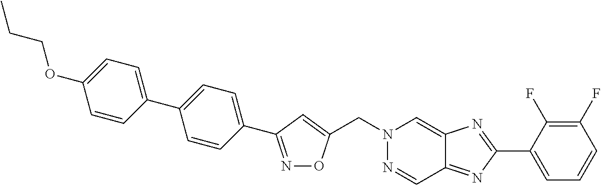 | 2-(2,3-Difluoro-phenyl)-5-[3-(4'- propoxy-biphenyl-4-yl)-isoxazol-5- ylmethyl]-5H-imidazo[4,5-d]pyridazine |
| 270 | 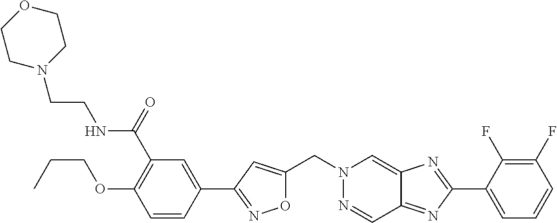 | 5-{5-[2-(2,3-Difluoro-phenyl)- imidazo[4,5-d]pyridazin-5-ylmethyl]- isoxazol-3-yl}-N-(2-morpholin-4-yl- ethyl)-2-propoxy-benzamide |
| 271 | 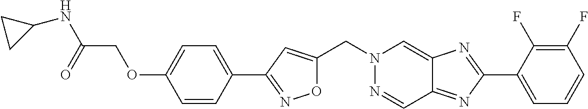 | N-Cyclopropyl-2-(4-{5-[2-(2,3-difluoro- phenyl)-imidazo[4,5-d]pyridazin-5- ylmethyl]-isoxazol-3-yl}-phenoxy)- acetamide |
| 272 | 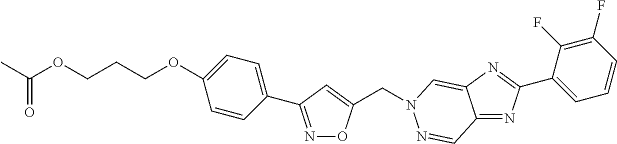 | Acetic acid 3-(4-{5-[2-(2,3-difluoro- phenyl)-imidazo[4,5-d]pyridazin-5- ylmethyl]-isoxazol-3-yl}-phenoxy)- propyl ester |
| 273 | 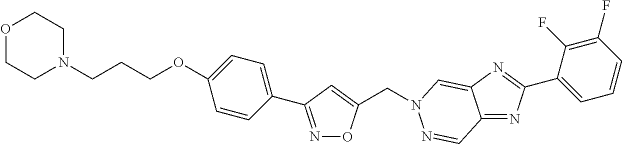 | 2-(2,3-Difluoro-phenyl)-5-{3-[4-(3- morpholin-4-yl-propoxy)-phenyl]- isoxazol-5-ylmethyl}-5H-imidazo[4,5- d]pyridazine |
| 274 | 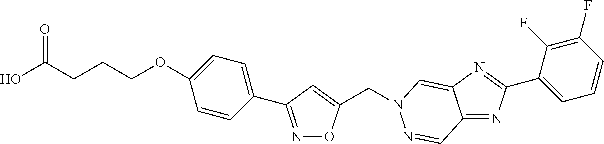 | 4-(4-{5-[2-(2,3-Difluoro-phenyl) imidazo[4,5-d]pyridazin-5-ylmethyl]- isoxazol-3-yl}-phenoxy)-butyric acid |
| 275 | 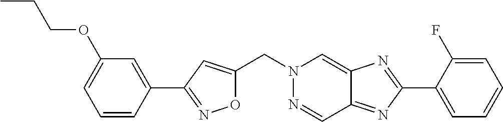 | 2-(2-Fluoro-phenyl)-5-[3-(3-propoxy- phenyl)-isoxazol-5-ylmethyl]-5H- imidazo[4,5-d]pyridazine |
| 276 | 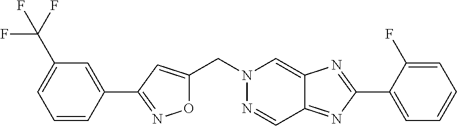 | 2-(2-Fluoro-phenyl)-5-[3-(3- trifluoromethyl-phenyl)-isoxazol-5- ylmethyl]-5H-imidazo[4,5-d]pyridazine |
| 277 | 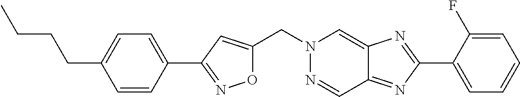 | 5-[3-(4-Butyl-phenyl)-isoxazol-5- ylmethyl]-2-(2-fluoro-phenyl)-5H- imidazo[4,5-d]pyridazine |
| 278 | 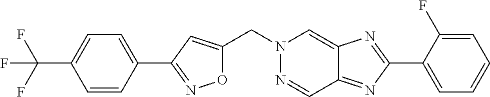 | 2-(2-Fluoro-phenyl)-5-[3-(4- trifluoromethyl-phenyl)-isoxazol-5- ylmethyl]-5H-imidazo[4,5-d]pyridazine |
| 279 | 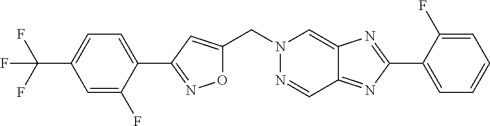 | 2-(2-Fluoro-phenyl)-5-[3-(2-fluoro-4- trifluoromethyl-phenyl)-isoxazol-5- ylmethyl]-5H-imidazo[4,5-d]pyridazine |
| 280 | 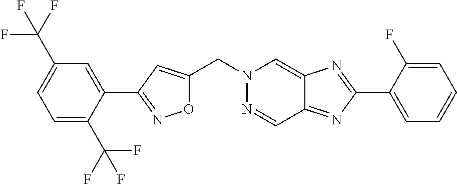 | 5-[3-(2,5-Bis-trifluoromethyl-phenyl)- isoxazol-5-ylmethyl]-2-(2-fluoro- phenyl)-5H-imidazo[4,5-d]pyridazine |
| 281 | 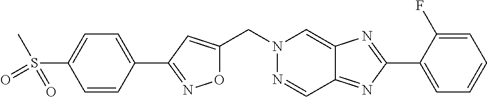 | 2-(2-Fluoro-phenyl)-5-[3-(4- methanesulfonyl-phenyl)-isoxazol-5- ylmethyl]-5H-imidazo[4,5-d]pyridazine |
| 282 | 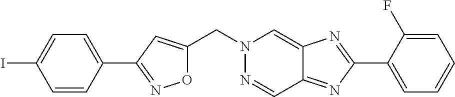 | 2-(2-Fluoro-phenyl)-5-[3-(4-iodo- phenyl)-isoxazol-5-ylmethyl]-5H- imidazo[4,5-d]pyridazine |
| 283 | 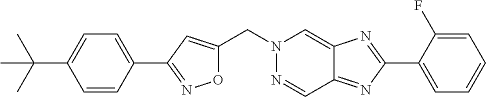 | 5-[3-(4-tert-Butyl-phenyl)-isoxazol-5- ylmethyl]-2-(2-fluoro-phenyl)-5H- imidazo[4,5-d]pyridazine |
| 284 | 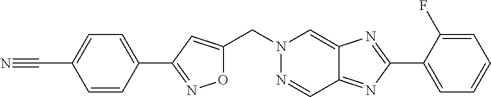 | 4-{5-[2-(2-Fluoro-phenyl)-imidazo[4,5- d]pyridazin-5-ylmethyl]-isoxazol-3-yl}- benzonitrile |
| 285 | 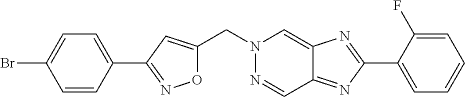 | 5-[3-(4-Bromo-phenyl)-isoxazol-5- ylmethyl]-2-(2-fluoro-phenyl)-5H- imidazo[4,5-d]pyridazine |
| 286 | 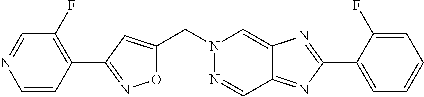 | 2-(2-Fluoro-phenyl)-5-[3-(3-fluoro- pyridin-4-yl)-isoxazol-5-ylmethyl]-5H- imidazo[4,5-d]pyridazine |
| 287 | 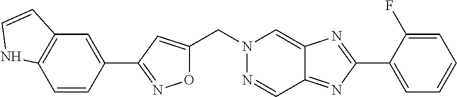 | 2-(2-Fluoro-phenyl)-5-[3-(1H-indol-5- yl)-isoxazol-5-ylmethyl]-5H- imidazo[4,5-d]pyridazine |
| 288 | 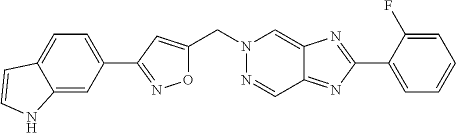 | 2-(2-Fluoro-phenyl)-5-[3-(1H-indol-6- yl)-isoxazol-5-ylmethyl]-5H- imidazo[4,5-d]pyridazine |
| 289 | 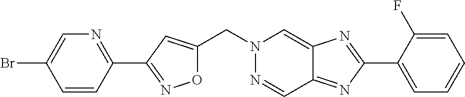 | 5-[3-(5-Bromo-pyridin-2-yl)-isoxazol-5- ylmethyl]-2-(2-fluoro-phenyl)-5H- imidazo[4,5-d]pyridazine |
| 290 | 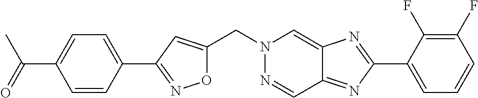 | 1-(4-{5-[2-(2,3-Difluoro-phenyl)- imidazo[4,5-d]pyridazin-5-ylmethyl]- isoxazol-3-yl}-phenyl)-ethanone |
| 291 | 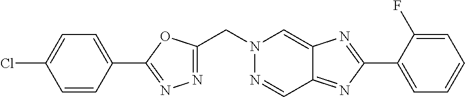 | 5-[5-(4-Chloro-phenyl)- [1,3,4]oxadiazol-2-ylmethyl]-2-(2- fluoro-phenyl)-5H-imidazo[4,5- d]pyridazine |
| 292 | 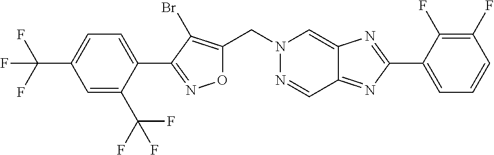 | 5-[3-(2,4-Bis-trifluoromethyl-phenyl)-4- bromo-isoxazol-5-ylmethyl]-2-(2,3- difluoro-phenyl)-5H-imidazo[4,5- d]pyridazine |

In some embodiments, provided are pharmaceutical compositions comprising a pharmaceutically acceptable diluent and a therapeutically effective amount of one of the compounds, or pharmaceutically acceptable salts or solvates, described herein or mixtures of one or more of such compounds, or pharmaceutically acceptable salts or solvates.

In some embodiments, provided are methods for treating in patients a viral infection mediated at least in part by a virus in the Flaviviridae family of viruses, such as HCV, which methods comprise administering to a patient that has been diagnosed with said viral infection or is at risk of developing said viral infection a pharmaceutical composition comprising a pharmaceutically acceptable diluent and a therapeutically effective amount of one of the compounds, or pharmaceutically acceptable salts or solvates, described herein or mixtures of one or more of such compounds, or pharmaceutically acceptable salts or solvates. In some embodiments, present provided are use of the compounds of Formula (I), or pharmaceutically acceptable salts or solvates, for the preparation of a medicament for treating or preventing said infections. In some embodiments, the patient is a human.

In some embodiments, provided are methods of treating or preventing viral infections in patients in combination with the administration of a therapeutically effective amount of one or more agents active against HCV. Active agents against HCV include ribavirin, levovirin, viramidine, thymosin alpha-1, an inhibitor of NS3 serine protease, and inhibitor of inosine monophosphate dehydrogenase, interferon-alpha, pegylated interferon-alpha, alone or in combination with ribavirin or viramidine. In one example, the additional agent active against HCV is interferon-alpha or pegylated interferon-alpha alone or in combination with ribavirin or viramidine. In another example, the active agent is interferon.

### General Synthetic Methods

The compounds disclosed herein can be prepared by following the general procedures and examples set forth below. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

If the compounds, or pharmaceutically acceptable salts or solvates, described herein contain one or more chiral centers, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

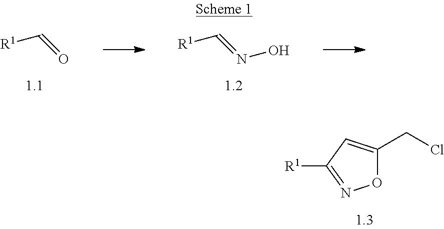

Scheme 1 shows the synthesis of 3-substituted chloromethylisoxazoles intermediates wherein R¹ is as defined for Formula (I). Aldehyde 1.1 is treated with hydroxylamine under oxime forming conditions to give 1.2 that is then cyclized to isoxazole 1.3 through treatment with propargyl chloride and an oxidizing agent such as NaOCl.

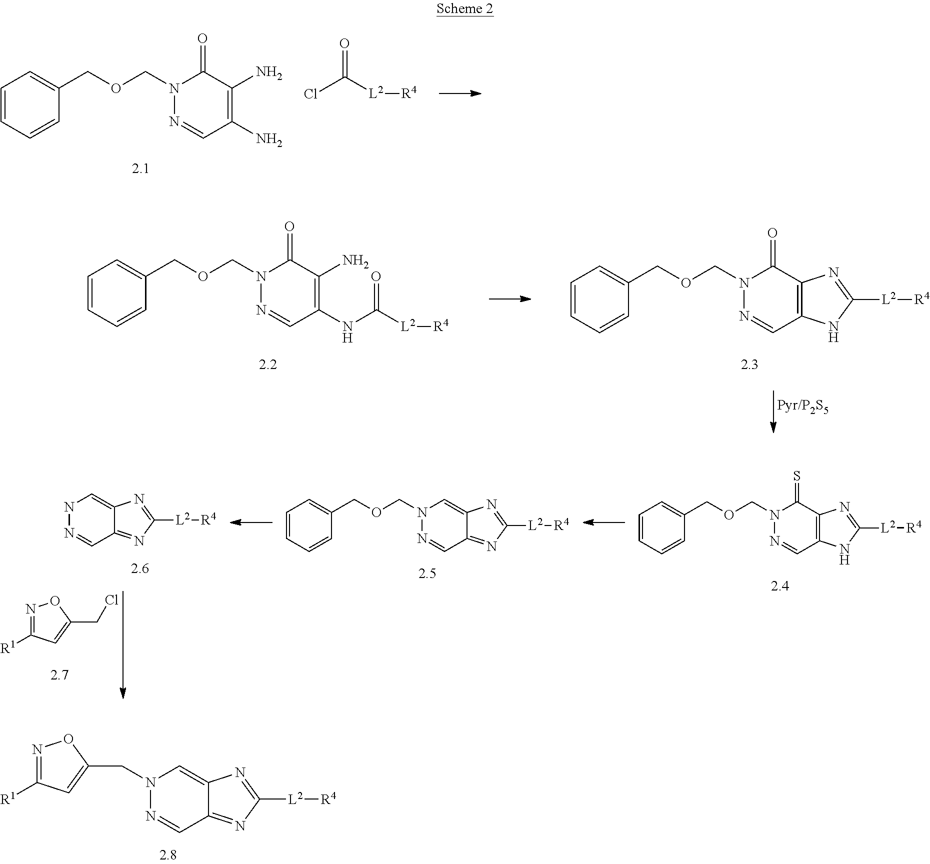

Scheme 2 shows the synthesis of the compounds of Formula (I) where for illustrative purposes Q and T are CH, V is N, X is CH, Y is N, Z is O, L¹ is CH₂, and R¹ and L²-R⁴ are previously defined. Diamine 2.1 (J. Het. Chem. 21, 481, 1984) is condensed with an acid chloride in a solvent such as pyridine to give amide 2.2 or its regioisomer. Exposure of 2.2 or its regioisomer to dehydration conditions such as treatment with an acid catalyst such as acetic acid gives 1,5-dihydro-imidazo[4,5-d]pyridazin-4-one 2.3. Reduction of the keto group can be accomplished via the corresponding thione 2.4 through treatment with a sufurizing reagent such as P₂S₅ in pyridine. The sulfur is then removed with Raney Nickel in a solvent such as ethanol giving the protected 5H-imidazo[4,5-d]pyridazines 2.5. The benzyloxymethyl protecting group is removed with a Lewis acid such as BCl₃ to give the unprotected 5H-imidazo[4,5-d]pyridazine 2.6. Alkylation of 2.6 with electrophiles such as chloromethyl isoxazole 2.7 in the presence of base gives the final product 2.8.

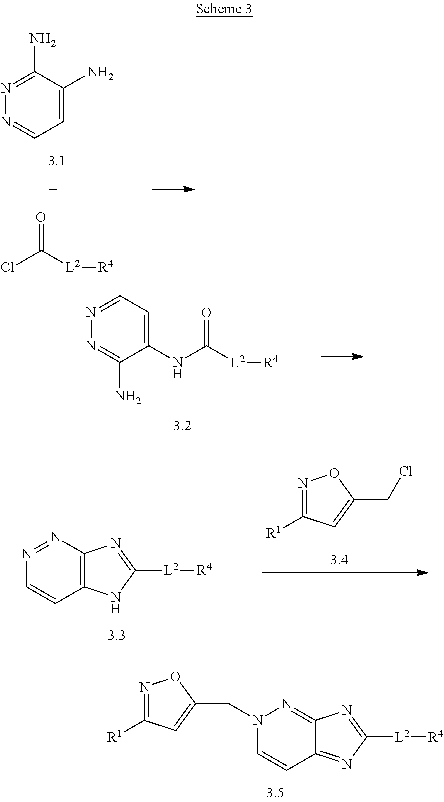

Scheme 3 shows the synthesis of the compounds of Formula (I) where for illustrative purposes Q and V are CH, T is N, X is CH, Y is N, Z is O, L¹ is CH₂, and R¹ and L²-R⁴ are previously defined. Diamine 3.1 (J. Het. Chem. 2, 67, 1965) is acylated with an acid chloride in a solvent such as pyridine to give amide 3.2 or its regioisomer. Treatment of 3.2 or its regioisomer with an acid catalyst such as acetic acid gives the 6-substituted-5H-imidazo[4,5-c]pyridazine 3.3 that is then alkylated with electrophiles such as chloromethyl isoxazole 3.4 in the presence of base to give isoxazole 3.5.

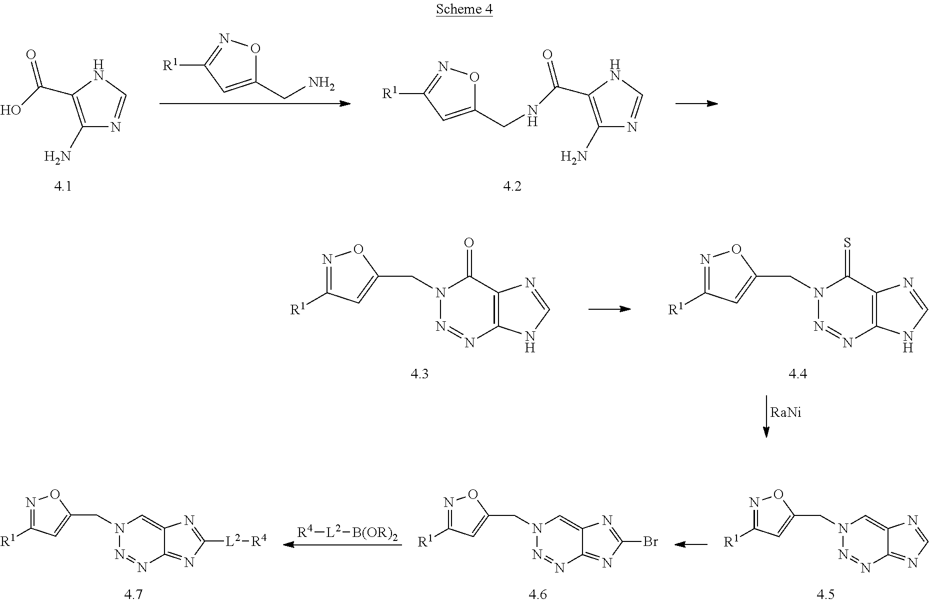

Scheme 4 shows the synthesis of the compounds of Formula (I) where for illustrative purposes T is CH, Q and V are N, X is CH, Y is N, Z is O, L¹ is CH₂, and R¹ and L²-R⁴ are previously defined. Carboxy amino imidazole 4.1 is condensed with an aminomethyl isoazole in the presence of standard amide coupling reagents such as N-[(dimethylamino)-1H-1,2,3-triazolo[4,5-b]pyridine-1-ylmethylene]-N-methylmethanaminium hexafluorophosphate N-oxide (HATU) to give amide 4.2. Diazotization of 4.2 and cyclization gives 3-substituted-3,7-dihydro-imidazo[4,5-d][1,2,3]triazin-4-one 4.3 that is next treated with P₂S₅ to give thione 4.4. Reduction of 4.4 with Raney Nickel gives the 3-substituted-3H-imidazo[4,5-d][1,2,3]triazine 4.5. Bromination of 4.5 gives 4.6 that is coupled under Suzuki conditions with boronic acid or ester R⁴L²-B(OR)₂ to afford 4.7.

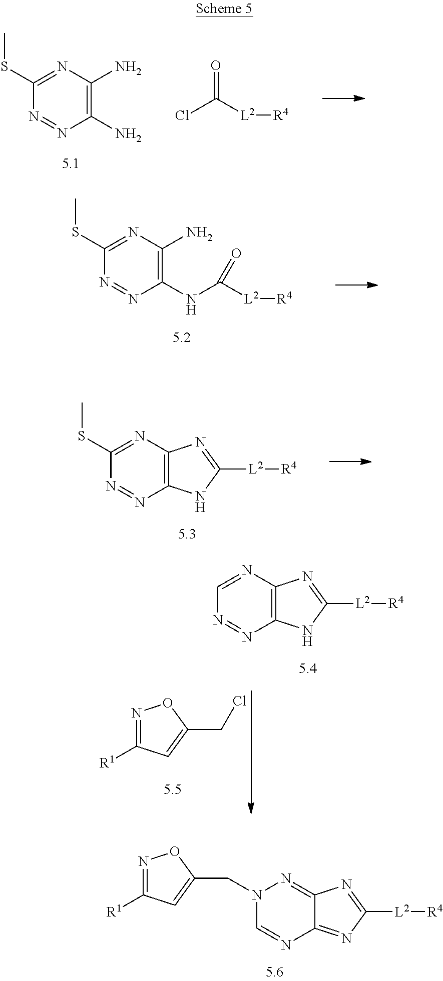

Scheme 5 shows the synthesis of the compounds of Formula (I) where for illustrative purposes Q and T are N, V is CH, X is CH, Y is N, Z is O, L¹ is CH₂, and R¹ and L²-R⁴ are previously defined. Diamine 5.1 (J. Org. Chem. 48, 8, 1271, 1983) is condensed with an acid chloride in a solvent such as pyridine to give amide 5.2 or its regioisomer. Amide 5.2 or its regioisomercan be cyclized in the presence of an acid catalyst such as acetic acid to give the 6-substituted-3-methylsulfanyl-7H-imidazo[4,5-e][1,2,4]triazine 5.3. The sulfur is then removed with Raney Nickel in a solvent such as ethanol giving the 6-substituted-7H-imidazo[4,5-e][1,2,4]triazine 5.4 that is then alkylated with electrophiles such as chloromethyl isoxazole 5.5 in the presence of base to afford 5.6.

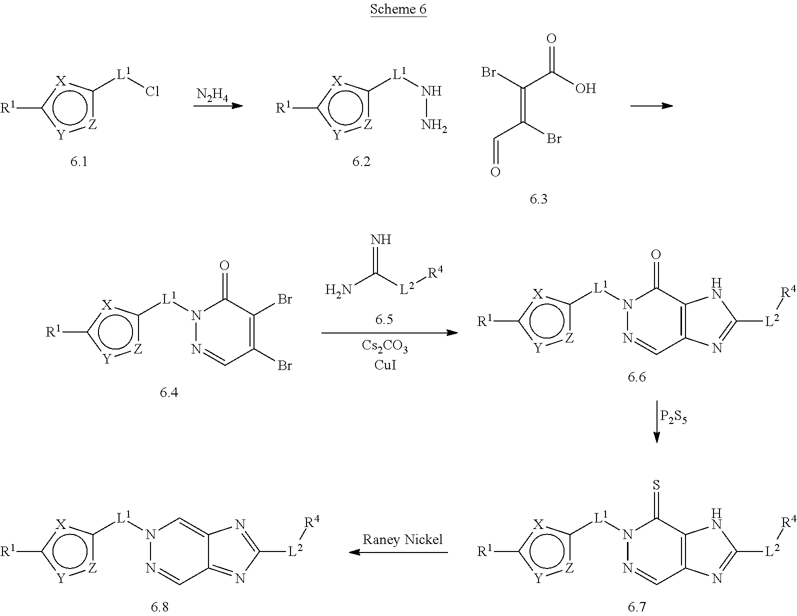

Scheme 6 shows the synthesis of the compounds of Formula (I) where for illustrative purposes Q and T are CH, V is N, and R¹, R⁴, L¹, L², X, Y and Z are previously defined. The substituted hydrazine 6.2 is formed from displacement of the corresponding electrophiles such as chloroalkyl heterocycles 6.1 with hydrazine. The compounds 6.2 are then cyclized with mucobromic acid 6.3, which are in turn cyclized with amidines 6.5 giving 2,5-disubstituted-3,5-dihydro-imidazo[4,5-d]pyridazin-4-ones 6.6. These are then converted to the final products 6.8 through treatment with reagents such as P₂S₅ followed by reduction with Raney Nickel.

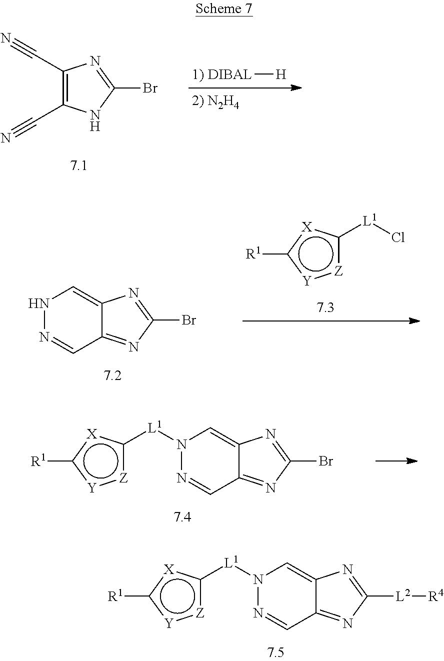

Scheme 7 shows the synthesis of the compounds of Formula (I) where for illustrative purposes Q and T are CH, V is N, and R¹, R⁴, L¹, L², X, Y and Z are previously defined. The dinitrile 7.1 (Heterocycles, 29, 1325, 1989) is reduced with reagents such as DIBAL-H in a solvent such as THF and subsequently cyclized with hydrazine or its derivatives to give 2-bromo-5H-imidazo[4,5-d]pyridazine 7.2. These are then alkylated with electrophiles such as chloroalkyl heterocycles 7.3 in the presence of base giving the 2-bromo-5-substituted-imidazo[4,5-d]pyridazines 7.4. They can be converted into the corresponding final products 7.5 through cross coupling reactions such as the Suzuki reaction.

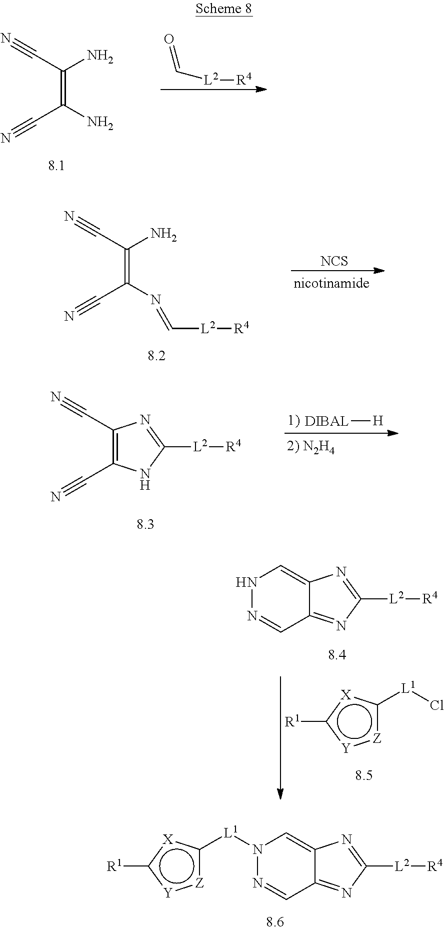

Scheme 8 shows the synthesis of the compounds of Formula (I) where for illustrative purposes Q and T are CH, V is N, and R¹, R⁴, L¹, L², X, Y and Z are previously defined. The dinitrile 8.1 is condensed with aldehydes of formula H(O)C-L²R⁴ and oxidatively cyclized to the 2-substituted imidazole 4,5 dinitrile 8.3. This is then reduced with reagents such as DIBAL-H in a solvent such as THF and subsequently cyclized with hydrazine or its derivatives to give 2-substituted-5H-imidazo[4,5-d]pyridazine 8.4. These are then alkylated with electrophiles such as chloroalkyl heterocycles 8.5 in the presence of base giving the final products 8.6.

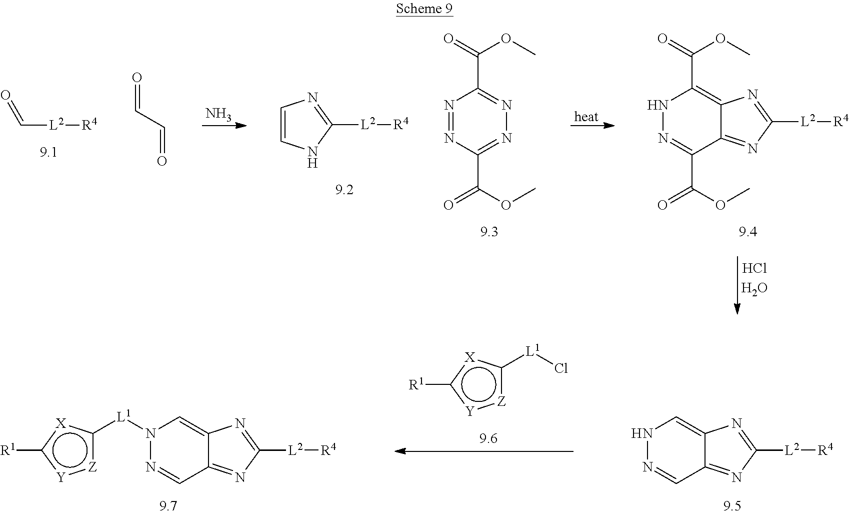

Scheme 9 shows the synthesis of the compounds of Formula (I) where for illustrative purposes Q and T are CH, V is N, and R¹, R⁴, L¹, L², X, Y and Z are previously defined. The imidazole 9.2 is formed in one step from the corresponding aldehyde 9.1 through condensation with glyoxal and ammonia. The 2-substituted imidazole 9.2 is condensed with reagents such as [1,2,4,5]Tetrazine-3,6-dicarboxylic acid dimethyl ester 9.3 (Org. Syn. Coll. Vol. 9, p 335, 1998). The intermediate 9.4 is then saponified and decarboxylated giving the 2-substituted-5H-imidazo[4,5-d]pyridazine 9.5 which is finally alkylated with electrophiles such as chloroalkyl heterocycles 9.6 in the presence of base giving the final products 9.7.

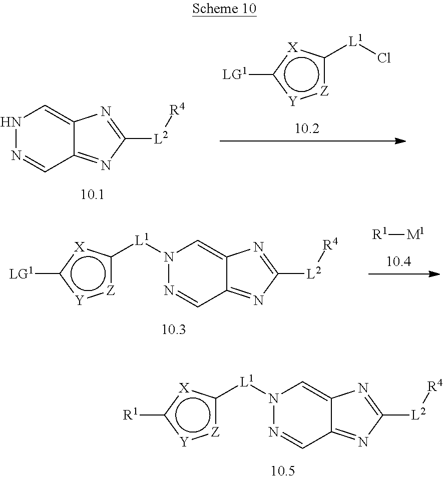

Scheme 10 shows the synthesis of the compounds of Formula (I) where for illustrative purposes Q and T are CH, V is N, and R¹, R⁴, L¹, L², X, Y and Z are previously defined. The 2-substituted-5H-imidazo[4,5-d]pyridazine 10.1 is alkylated with electrophiles such as chloroalkyl heterocycles 10.2 in the presence of base giving the products 10.3 which can then be converted to final products 10.5.

The foregoing and other aspects of the present invention may be better understood in connection with the following representative examples.

### EXAMPLES

In the examples below and the synthetic schemes above, the following abbreviations have the following meanings. If an abbreviation is not defined, it has its generally accepted meaning.
aq.=aqueous
μL=microliters
μM=micromolar
NMR=nuclear magnetic resonance
br=broad
d=doublet
δ=chemical shift
° C.=degrees celsius
dd=doublet of doublets
DMEM=Dulbeco's Modified Eagle's Medium
DMF=N,N-dimethylformamide
DMSO=dimethylsulfoxide
DTT=dithiothreotol
EDTA=ethylenediaminetetraacetic acid
EtOH=ethanol
g=gram
h or hr=hours
HCV=hepatitus C virus
HPLC=high performance liquid chromatography
Hz=hertz
IU=International Units
IC₅₀=inhibitory concentration at 50% inhibition
J=coupling constant (given in Hz unless otherwise indicated)
m=multiplet
M=molar
M+H⁺=parent mass spectrum peak plus H⁺
MeOH=methanol
mg=milligram
mL=milliliter
mM=millimolar
mmol=millimole
MS=mass spectrum
nm=nanomolar
ng=nanogram
ppm=parts per million
s=Singlet
t=triplet
wt %=weight percent

### General Procedure A: Synthesis of 2-Substituted 5H-imidazo[4,5-d]pyridazines

4,5-Diamino-2-benzyloxymethyl-2H-pyridazin-3-one (5.0 g, from J. Het. Chem. 21, 481, 1984) was dissolved in pyridine (25 mL) and an acid chloride (1.1 eq) was added dropwise at room temperature. The mixture was allowed to stir at ambient temperature for 2 hours. The solvent was removed, yielding the amide as a mixture of regioisomers.

The dried amide was dissolved in HOAc (5 mL/gram) and heated to 170° C. for 30 minutes to give 2-substituted 5-benzyloxymethyl-1,5-dihydro-imidazo[4,5-d]pyridazin-4-ones. The products can be purified by trituration with MeOH.

The products and P₂S₅ (1 g/mmol) were then dissolved in pyridine (30 mL/gram) and water (0.75%). The reactions were refluxed overnight. More P₂S₅ was added if the reaction was incomplete. The reaction mixture was cooled and the solution decanted. The solids were washed with hot pyridine and the organic solvent removed. The resulting oil was partitioned between chloroform (100 mL) and NaHCO₃ (sat. aq. 50 mL). The organics were dried (Na₂SO₄) and purified by silica gel chromatography (CH₂Cl₂/MeOH) giving 2-substituted 5-benzyloxymethyl-1,5-dihydro-imidazo[4,5-d]pyridazine-4-thiones.

The thiones were then dissolved in EtOH (20 mL/gram) and treated with Raney Nickel (unwashed, 1 g/1 g thione) and heated to 70° C. If the reaction was incomplete after 1 hour more Nickel was added. The reactions were then cooled, filtered, the solids were thoroughly washed with hot EtOH and the organics combine and removed yielding the 2-substituted 5-benzyloxymethyl-5H-imidazo[4,5-d]pyridazines.

The products were dissolved in CH₂Cl₂ (35 mL/mmol) and cooled to −78° C. A solution of BCl₃ (1M in CH₂Cl₂, 8 mL/mmol) was added and the mixture stirred for 30 minutes. Upon completion, MeOH (5 mL) was added and the mixture warmed to room temperature. The solvents were removed yielding the pure 2-substituted 5H-imidazo[4,5-d]pyridazines. They can be further purified by tritruation with MeOH.

### General Procedure B: Synthesis of Chloromethyl Aryl Isoxazole

The aldehyde (20 mmol) was dissolved in ethanol (15 mL) and hydroxyl amine (50% aq. solution, 3 mL) was added. The mixture was allowed to stir at ambient temperature for 2 hours. The solvent was removed, and no further purification steps were taken.

The oxime (7.65 mmol) was dissolved in dichloromethane (8 mL), and the solution was cooled to 0° C. Propargyl chloride (0.548 mL, 7.65 mmol) was added followed by the dropwise addition of NaOCl (6.5% aq. solution, 13 mL). The reaction was stirred at 0° C. for 15 minutes and then heated to 50° C. for 3 hours. After cooling, the reaction was partitioned between dichloromethane and water, and the aqueous layer was extracted with dichloromethane (3×20 mL). The organic layers were combined, washed with brine (40 mL), dried with anhydrous magnesium sulfate, and filtered. The solvent was removed to give the desired product, and no further purification steps were taken.

### General Procedure C: Synthesis of Compounds 101-105

2-(2,3-Difluoro-phenyl)-5H-imidazo[4,5-d]pyridazine (23.8 mg, 0.10 mmol), chloromethyl aryl isoxazole (1 equivalent), and cesium carbonate (66.7 mg, 0.20 mmol) were dissolved in DMF (3 mL) and microwaved at 120° C. for 10 minutes. The reaction was filtered and purified by reverse phase HPLC to give the desired product. The product was converted to the HCl salt by the addition of 1N HCl before concentration.

### Example 1

### 2-(2,3-Difluoro-phenyl)-5-[3-(4-fluoro-2-trifluoromethyl-phenyl)-isoxazol-5-ylmethyl]-5H-imidazo[4,5-d]pyridazine (Compound 101)

From 1 equivalent of 5-chloromethyl-3-(4-fluoro-2-trifluoromethyl-phenyl)-isoxazole. Yield 17.3 mg. MS 476.0 (M+H⁺); ¹H NMR (DMSO-d₆): δ(ppm) 10.17 (d, 1H), 9.55 (d, 1H), 8.12-8.18 (m, 1H), 7.84-7.90 (m, 1H), 7.55-7.77 (m, 3H), 7.34-7.41 (m, 1H), 6.98 (s, 1H), 6.24 (s, 2H).

### Example 2

### 2-(2,3-Difluoro-phenyl)-5-[3-(4-isopropoxy-phenyl)-isoxazol-5-ylmethyl]-5H-imidazo[4,5-d]pyridazine (Compound 102)

From 1 equivalent of 5-chloromethyl-3-(4-isopropoxy-phenyl)-isoxazole. Yield 16.7 mg. MS 448.1 (M+H⁺); ¹H NMR (DMSO-d₆): δ(ppm) 10.18 (d, 1H), 9.55 (d, 1H), 8.12-8.19 (m, 1H), 7.72-7.78 (m, 2H), 7.55-7.65 (m, 1H), 7.34-7.42 (m, 1H), 7.13 (s, 1H), 6.97-7.03 (m, 2H), 6.18 (s, 2H), 4.64-4.73 (m, 1H), 1.27 (d, 6H).

### Example 3

### 5-[3-(2,4-Bis-trifluoromethyl-phenyl)-isoxazol-5-ylmethyl]-2-(2,3-difluoro-phenyl)-5H-imidazo[4,5-d]pyridazine (Compound 103)

From 1 equivalent of 3-(2,4-bis-trifluoromethyl-phenyl)-5-chloromethyl-isoxazole. Yield 12.2 mg. MS 526.0 (M+H⁺); ¹H NMR (DMSO-d₆): δ(ppm) 10.17 (d, 1H), 9.55 (d, 1H), 8.12-8.26 (m, 3H), 7.93 (d, 1H), 7.54-7.65 (m, 1H), 7.33-7.41 (m, 1H), 7.06 (s, 1H), 6.26 (s, 2H).

### Example 4

### 5-[3-(4-Chloro-phenyl)-isoxazol-5-ylmethyl]-2-(2,3-difluoro-phenyl)-5H-imidazo[4,5-d]pyridazine (Compound 104)

From 1 equivalent of 5-chloromethyl-3-(4-chloro-phenyl)-isoxazole. Yield 16.9 mg. MS: 424.0 (M+H⁺); ¹H NMR (DMSO-d₆): δ(ppm) 10.21 (d, 1H), 9.57 (d, 1H), 8.12-8.18 (m, 1H), 7.85-7.91 (m, 2H), 7.54-7.67 (m, 3H), 7.35-7.42 (m, 1H), 7.24 (s, 1H), 6.23 (s, 2H).

### Example 5

### 2-(2,3-Difluoro-phenyl)-5-[3-(4-propoxy-phenyl)-isoxazol-5-ylmethyl]-5H-imidazo[4,5-d]pyridazine (Compound 105)

From 1 equivalent of 5-chloromethyl-3-(4-propoxy-phenyl)-isoxazole. Yield 21.9 mg. MS 448.1 (M+H⁺); ¹H NMR (DMSO-d₆): δ(ppm) 10.36 (s, 1H), 9.67 (d, 1H), 8.13-8.18 (m, 1H), 7.63-7.79 (m, 3H), 7.40-7.47 (m, 1H), 7.16 (s, 1H), 7.00-7.05 (m, 2H), 6.26 (s, 2H), 3.95-4.00 (t, 2H), 1.67-1.80 (m, 2H), 0.95-1.02 (t, 3H).

### General Procedure D: Synthesis of Compounds 106-118

2-(2-Fluoro-phenyl)-5H-imidazo[4,5-d]pyridazine (30.0 mg, 0.14 mmol), chloromethyl aryl heterocycle (1 equivalent), and cesium carbonate (91.3 mg, 0.28 mmol) were dissolved in DMF (3 mL) and heated in a microwave reactor at 120° C. for 10 minutes. The reaction was filtered and purified by reverse phase HPLC to give the desired product. The product was converted to the HCl salt by the addition of 1N HCl before concentration.

### Example 6

### 5-[3-(4-Butoxy-phenyl)-isoxazol-5-ylmethyl]-2-(2-fluoro-phenyl)-5H-imidazo[4,5-d]pyridazine (Compound 106)

From 1 equivalent of 3-(4-butoxy-phenyl)-5-chloromethyl-isoxazole. Yield 8.8 mg. MS 444.1 (M+H⁺); ¹H NMR (DMSO-d₆): δ(ppm) 10.45 (s, 1H), 9.74 (s, 1H), 8.32-8.39 (m, 1H), 7.67-7.78 (m, 3H), 7.44-7.56 (m, 2H), 7.17 (s, 1H), 7.03 (d, 2H), 6.31 (s, 2H), 3.98-4.04 (t, 2H), 1.65-1.74 (m, 2H), 1.36-1.49 (m, 2H), 0.90-0.97 (t, 3H).

### Example 7

### 2-(2-Fluoro-phenyl)-5-[3-(4-pentyloxy-phenyl)-isoxazol-5-ylmethyl]-5H-imidazo[4,5-d]pyridazine (Compound 107)

From 1 equivalent of 5-chloromethyl-3-(4-pentyloxy-phenyl)-isoxazole. Yield 10.1 mg. MS 458.1 (M+H⁺); ¹H NMR (DMSO-d₆): δ(ppm) 10.42 (s, 1H), 9.72 (s, 1H), 8.32-8.40 (m, 1H), 7.65-7.79 (m, 3H), 7.43-7.54 (m, 2H), 7.16 (s, 1H), 7.03 (d, 2H), 6.30 (s, 2H), 3.97-4.03 (t, 2H), 1.65-1.78 (m, 2H), 1.27-1.45 (m, 4H), 0.86-0.92 (t, 3H).

### Example 8

### 2-(2-Fluoro-phenyl)-5-[3-(4-trifluoromethoxy-phenyl)-isoxazol-5-ylmethyl]-5H-imidazo[4,5-d]pyridazine (Compound 108)

From 1 equivalent of 5-chloromethyl-3-(4-trifluoromethoxy-phenyl)-isoxazole. Yield 10.4 mg. MS 456.1 (M+H⁺); ¹H NMR (DMSO-d₆): δ(ppm) 10.37 (s, 1H), 9.68 (s, 1H), 8.31-8.38 (m, 1H), 7.95-8.02 (m, 2H), 7.61-7.71 (m, 1H), 7.41-7.54 (m, 4H), 7.28 (s, 1H), 6.31 (s, 2H).

### Example 9

### 2-(2-Fluoro-phenyl)-5-[3-(4-methoxy-phenyl)-isoxazol-5-ylmethyl]-5H-imidazo[4,5-d]pyridazine (Compound 109)

From 1 equivalent of 5-chloromethyl-3-(4-methoxy-phenyl)-isoxazole. Yield 12.1 mg. MS 402.1 (M+H⁺); ¹H NMR (DMSO-d₆): δ(ppm) 10.59 (s, 1H), 9.80 (d, 1H), 8.33-8.40 (m, 1H), 7.70-7.80 (m, 3H), 7.46-7.59 (m, 2H), 7.19 (s, 1H), 7.01-7.07 (m, 2H), 6.37 (s, 2H), 3.80 (s, 3H).

### Example 10

### 5-[3-(4-Ethoxy-phenyl)-isoxazol-5-ylmethyl]-2-(2-fluoro-phenyl)-5H-imidazo[4,5-d]pyridazine (Compound 110)

From 1 equivalent of 5-chloromethyl-3-(4-ethoxy-phenyl)-isoxazole. Yield 10.2 mg. MS 416.1 (M+H⁺); ¹H NMR (DMSO-d₆): δ(ppm) 10.39 (s, 1H), 9.70 (s, 1H), 8.31-8.39 (m, 1H), 7.64-7.79 (m, 3H), 7.42-7.54 (m, 2H), 7.16 (s, 1H), 7.00-7.05 (m, 2H), 6.28 (s, 2H), 4.02-4.12 (q, 2H), 1.30-1.38 (t, 3H).

### Example 11

### 2-(2-Fluoro-phenyl)-5-(3-phenyl-isoxazol-5-ylmethyl)-5H-imidazo[4,5-d]pyridazine (Compound 111)

From 1 equivalent of 5-chloromethyl-3-phenyl-isoxazole. Yield 15.2 mg. MS 372.1 (M+H⁺); ¹H NMR (DMSO-d₆): δ(ppm) 10.59 (s, 1H), 9.80 (d, 1H), 8.33-8.40 (m, 1H), 7.70-7.87 (m, 3H), 7.46-7.59 (m, 5H), 7.26 (s, 1H), 6.39 (s, 2H).

### Example 12

### 2-(2-Fluoro-phenyl)-5-[3-(4-isopropoxy-phenyl)-isoxazol-5-ylmethyl]-5H-imidazo[4,5-d]pyridazine (Compound 112)

From 1 equivalent of 5-chloromethyl-3-(4-isopropoxy-phenyl)-isoxazole. Yield 34.6 mg. MS 430.1 (M+H⁺); ¹H NMR (DMSO-d₆): δ(ppm) 10.51 (s, 1H), 9.77 (d, 1H), 8.32-8.39 (m, 1H), 7.69-7.77 (m, 3H), 7.45-7.59 (m, 2H), 7.16 (s, 1H), 6.96-7.04 (m, 2H), 6.33 (s, 2H), 4.63-4.72 (m, 1H), 1.28 (d, 6H).

### Example 13

### 5-[3-(4-Chloro-phenyl)-[1,2,4]oxadiazol-5-ylmethyl]-2-(2-fluoro-phenyl)-5H-imidazo[4,5-d]pyridazine (Compound 113)

From 5-chloromethyl-3-(4-chloro-phenyl)-[1,2,4]oxadiazole. MS: 407.8 (M+H⁺); ¹H NMR (DMSO-d₆): δ(ppm) 10.3 (s, 1H), 9.6 (s, 1H), 8.3 (m, 1H), 7.9 (m, 2H), 7.6 (m, 3H), 7.4 (m, 2H), 6.5 (s, 2H).

### Example 14

### 5-[3-(2,4-Bis-trifluoromethyl-phenyl)-isoxazol-5-ylmethyl]-2-(2-fluoro-phenyl)-5H-imidazo[4,5-d]pyridazine (Compound 114)

From 3-(2,4-bis-trifluoromethyl-phenyl)-5-chloromethyl-isoxazole. MS: 508.4 (M+H⁺); ¹H NMR (DMSO-d₆): δ(ppm) 10.3 (s, 1H), 9.6 (s, 1H), 8.3 (m, 1H), 8.2 (m, 2H), 7.9 (d, 1H), 7.6 (m, 1H), 7.4 (m, 2H), 7.0 (s, 1H), 6.3 (s, 2H).

### Example 15

### 2-(2-Fluoro-phenyl)-5-[3-(4-fluoro-2-trifluoromethyl-phenyl)-isoxazol-5-ylmethyl]-5H-imidazo[4,5-d]pyridazine (Compound 115)

From 5-chloromethyl-3-(4-fluoro-2-trifluoromethyl-phenyl)-isoxazole. MS: 458.4 (M+H⁺); ¹H NMR (DMSO-d₆): δ(ppm) 10.3 (s, 1H), 9.6 (s, 1H), 8.2 (m, 1H), 7.8 (m, 1H), 7.6 (m, 3H), 7.4 (m, 2H), 6.9 (s, 1H), 6.3 (s, 2H).

### Example 16

### 5-[3-(4-Chloro-phenyl)-isoxazol-5-ylmethyl]-2-(2-fluoro-phenyl)-5H-imidazo[4,5-d]pyridazine (Compound 116)

From 5-chloromethyl-3-(4-chloro-phenyl)-isoxazole. MS: 406 (M+H⁺); ¹H NMR (DMSO-d₆): δ(ppm) 10.4 (s, 1H), 9.7 (s, 1H), 8.3 (m, 1H), 7.8 (m, 2H), 7.7 (m, 1H), 7.6-7.4 (m, 4H), 7.2 (s, 1H), 6.3 (s, 2H).

### Example 17

### 2-(2-Fluoro-phenyl)-5-[3-(4-propoxy-phenyl)-isoxazol-5-ylmethyl]-5H-imidazo[4,5-d]pyridazine (Compound 117)

From 5-chloromethyl-3-(4-propoxy-phenyl)-isoxazo le. MS: 430 (M+H⁺); ¹H NMR (DMSO-d₆): δ(ppm) 10.4 (s, 1H), 9.7 (s, 1H), 8.3 (m, 1H), 7.8-7.7 (m, 3H), 7.6-7.4 (m, 2H), 7.1 (m, 1H), 7.0 (m, 2H), 6.3 (s, 2H), 3.9 (t, 2H), 1.7 (m, 2H), 0.9 (t. 3H).

### Example 18

### 5-[5-(4-Chloro-phenyl)-[1,3,4]oxadiazol-2-ylmethyl]-2-(2-fluoro-phenyl)-5H-imidazo[4,5-d]pyridazine (Compound 118)

2-(2-Fluoro-phenyl)-5H-imidazo[4,5-d]pyridazine (30.0 mg, 0.14 mmol), 2-chloromethyl-5-phenyl-[1,3,4]oxadiazole (32.1 mg, 0.14 mmol), and cesium carbonate (91.3 mg, 0.28 mmol) were dissolved in DMF and microwaved at 120° C. for 10 minutes. The reaction was filtered and purified by reverse phase HPLC to give the desired product. Yield 12.7 mg. MS 407.0 (M+H⁺); ¹H NMR (DMSO-d₆): δ(ppm) 10.22 (s, 1H), 9.58 (d, 1H), 8.31-8.38 (m, 1H), 7.96-8.01 (m, 2H), 7.57-7.70 (m, 3H), 7.37-7.47 (m, 2H), 6.40 (s, 2H).

### General Procedure E: Synthesis of Compounds 119-145

### General Procedure E

### Synthesis of 2-Aryl-5-substituted-imidazo[4,5-d]pyridazines (Compounds 119-145) 2-Bromo-5H-imidazo[4,5-d]pyridazine

A solution of 2-Bromo-1H-imidazole-4,5-dicarbonitrile (2 g, 10 mmol from Heterocycles 29, 1325, 1989) in THF (100 mL) was cooled to −78° C. and treated with DIBALH (50 mL of 1M solution in THF, 5 eq.) over 10 minutes. The mixture was stirred for 15 minutes then quenched with potassium tartrate (aq. 10% w/vol, 80 mL) stirred for 15 minutes at 15° C. then treated with hydrazine (anhydrous, 5 mL) and stirred at room temperature for 1 hr. The reaction was then cooled to 0° C. overnight, then filtered. The solids were washed with MeOH (2×100 mL) and the organic fraction concentrated. The crude product was then purified on silica gel eluting with 0-60% CH₂Cl₂: MeOH (w/ 10% NH₄OH). Yield 350 mg, (18%) MS 199/201 (M+H⁺).

### 5-Substituted-2-bromo-imidazo[4,5-d]pyridazine

To a solution of 2-Bromo-5H-imidazo[4,5-d]pyridazine (1 eq) in DMF (5 mL/mmol) was added an excess of K₂CO₃ and 3-aryl-chloromethyl-isoxazole (1 eq) and heated to 40° C. for 1 hr. The mixture was then cooled and poured into H₂O (30 mL/mmol) and the precipitate collected and dried to give the products.

### 2-Aryl-5-substituted-imidazo[4,5-d]pyridazines

A solution of the 5-substituted-2-bromo-imidazo[4,5-d]pyridazine (1 eq.), aryl boronic acid (1.3 eq.) tetrakistriphenylphosphine palladium (5 mol %), K₂CO₃ (3 eq. 1M, aq) in isopropanol (10 mL/mmol) was degassed and heated to 120° C. with microwave irradiation for 20 minutes. The reaction was filtered and purified by reverse phase HPLC to give the desired product. The product was converted to the HCl salt by the addition of 1N HCl before concentration.

### Example 19

### 2-{5-[3-(2,4-Bis-trifluoromethyl-phenyl)-isoxazol-5-ylmethyl]-5H-imidazo[4,5-d]pyridazine-2-yl}-phenylamine (Compound 119)

From 5-[3-(2,4-Bis-trifluoromethyl-phenyl)-isoxazol-5-ylmethyl]-2-bromo-5H-imidazo[4,5-d]pyridazine and 2-amino-phenylboronic acid. MS 505.1 (M+H⁺); H¹ NMR (DMSO-d₆): δ(ppm) 10.22 (s, 1H), 9.57 (s, 1H), 8.18-8.09 (m, 3H), 7.86-7.83 (d, 1H), 7.21 (t, 1H), 7.02 (s, 1H), 6.84-6.82 (d, 1H), 6.63 (t, 1H), 6.30 (s, 2H).

### Example 20

### 2-Benzo[b]thiophen-2-yl-5-[3-(2,4-bis-trifluoromethyl-phenyl)-isoxazol-5-ylmethyl]-5H-imidazo[4,5-d]pyridazine (Compound 120)

From 5-[3-(2,4-Bis-trifluoromethyl-phenyl)-isoxazol-5-ylmethyl]-2-bromo-5H-imidazo[4,5-d]pyridazine and 2-benzo[b]thiophene boronic acid. MS 546.1 (M+H⁺); H¹ NMR (DMSO-d₆): δ(ppm) 10.20 (s, 1H), 9.54 (s, 1H), 8.39 (s, 1H), 8.17-8.13 (m, 2H), 8.02-7.94 (m, 2H), 7.87-7.84 (d, 1H), 7.41-7.38 (m, 2H), 7.00 (s, 1H), 6.26 (s, 2H).

### Example 21

### 5-[3-(2,4-Bis-trifluoromethyl-phenyl)-isoxazol-5-ylmethyl]-2-(4-methyl-thiophen-3-yl)-5H-imidazo[4,5-d]pyridazine (Compound 121)

From 5-[3-(2,4-Bis-trifluoromethyl-phenyl)-isoxazol-5-ylmethyl]-2-bromo-5H-imidazo[4,5-d]pyridazine and 4-methyl-thiophene 3-boronic acid. MS 510.0 (M+H⁺); H¹ NMR (DMSO-d₆): δ(ppm) 10.45 (s, 1H), 9.74 (s, 1H), 8.62-6.61 (d, 1H), 8.24-8.20 (m, 2H), 7.91-7.89 (d, 1H), 7.62-7.45 (m, 1H), 7.09 (s, 1H), 6.37 (s, 2H), 2.65 (s, 3H).

### Example 22

### 5-[3-(2,4-Bis-trifluoromethyl-phenyl)-isoxazol-5-ylmethyl]-2-thiophen-3-yl-5H-imidazo[4,5-d]pyridazine (Compound 122)

From 5-[3-(2,4-Bis-trifluoromethyl-phenyl)-isoxazol-5-ylmethyl]-2-bromo-5H-imidazo[4,5-d]pyridazine and thiophene 3-boronic acid. MS 496.0 (M+H⁺); H¹ NMR (DMSO-d₆): δ(ppm) 10.39 (s, 1H), 9.67 (s, 1H), 8.66 (s, 1H), 8.18-8.14 (m, 2H), 7.91-7.76 (m, 3H), 7.02 (s, 1H), 6.32 (s, 2H).

### Example 23

### 5-[3-(2,4-Bis-trifluoromethyl-phenyl)-isoxazol-5-ylmethyl]-2-(3,5-dimethyl-isoxazol-4-yl)-5H-imidazo[4,5-d]pyridazine (Compound 123)

From 5-[3-(2,4-Bis-trifluoromethyl-phenyl)-isoxazol-5-ylmethyl]-2-bromo-5H-imidazo[4,5-d]pyridazine and 3,5-dimethyl-isoxazole 4-boronic acid. MS 509.1 (M+H⁺); H¹ NMR (DMSO-d₆): δ(ppm) 10.37 (s, 1H), 9.66 (s, 1H), 8.23-8.19 (m, 2H), 7.91-7.89 (d, 1H), 7.08 (s, 1H), 6.36 (s, 2H), 2.84 (s, 3H), 2.56 (s, 3H).

### Example 24

### 5-[3-(2,4-Bis-trifluoromethyl-phenyl)-isoxazol-5-ylmethyl]-2-(2-fluoro-3-methoxy-phenyl)-5H-imidazo[4,5-d]pyridazine (Compound 124)

From 5-[3-(2,4-Bis-trifluoromethyl-phenyl)-isoxazol-5-ylmethyl]-2-bromo-5H-imidazo[4,5-d]pyridazine and 2-Fluoro-3-methoxy-phenylboronic acid. MS 538.1 (M+H⁺); H¹ NMR (DMSO-d₆): δ(ppm) 10.11 (s, 1H), 9.50 (s, 1H), 8.23-8.19 (m, 2H), 7.93-7.81 (m, 2H), 7.31-7.26 (m, 2H), 7.04 (s, 1H), 6.24 (s, 2H), 3.85 (s, 3H).

### Example 25

### 5-[3-(2,4-Bis-trifluoromethyl-phenyl)-isoxazol-5-ylmethyl]-2-(2-methoxy-phenyl)-5H-imidazo[4,5-d]pyridazine (Compound 125)

From 5-[3-(2,4-Bis-trifluoromethyl-phenyl)-isoxazol-5-ylmethyl]-2-bromo-5H-imidazo[4,5-d]pyridazine and 2-methoxyphenyl boronic acid. MS 520.1 (M+H⁺); H¹ NMR (DMSO-d₆): δ(ppm) 9.80 (s, 1H), 8.43-8.40 (d, 1H), 8.25-8.21 (m, 3H), 7.91-7.88 (d, 1H), 7.34-7.68 (t, 1H), 7.40-7.37 (d, 1H), 7.26-7.22 (t, 1H), 7.11 (s, 1H), 6.47 (s, 2H), 4.10 (s, 3H).

### Example 26

### 5-[3-(2,4-Bis-trifluoromethyl-phenyl)-isoxazol-5-ylmethyl]-2-o-tolyl-5H-imidazo[4,5-d]pyridazine (Compound 126)

From 5-[3-(2,4-Bis-trifluoromethyl-phenyl)-isoxazol-5-ylmethyl]-2-bromo-5H-imidazo[4,5-d]pyridazine and 2-methylphenylboronic acid. MS 504.1 (M+H⁺); H¹ NMR (DMSO-d₆): δ(ppm) 10.53 (s, 1H), 9.80 (s, 1H), 8.25-8.21 (m, 2H), 8.01-7.97 (d, 1H), 7.92-7.89 (d, 1H), 7.53-7.44 (m, 3H), 7.10 (s, 1H), 6.40 (s, 2H), 2.70 (s, 3H).

### Example 27

### 2-(3-Fluoro-phenyl)-5-[3-(4-propoxy-phenyl)-isoxazol-5-ylmethyl]-5H-imidazo[4,5-d]pyridazine (Compound 127)

From 2-bromo-5-[3-(4-propoxy-phenyl)-isoxazol-5-ylmethyl]-5H-imidazo[4,5-d]pyridazine and 3-fluorophenyl boronic acid. MS 430.1 (M+H⁺); H¹ NMR (DMSO-d₆): δ(ppm) 10.20 (s, 1H), 9.54 (s, 1H), 8.19-8.17 (d, 1H), 8.08-8.05 (d, 1H), 7.71-7.68 (d, 2H), 7.59-7.57 (m, 1H), 7.37 (t, 1H), 7.08 (s, 1H), 6.97-6.94 (d, 2H), 6.15 (s, 2H), 3.92-3.88 (t, 2H), 1.70-1.63 (m, 2H), 0.93-0.88 (t, 3H).

### Example 28

### 5-[3-(2,4-Bis-trifluoromethyl-phenyl)-isoxazol-5-ylmethyl]-2-(4-fluoro-phenyl)-5H-imidazo[4,5-d]pyridazine (Compound 128)

From 5-[3-(2,4-Bis-trifluoromethyl-phenyl)-isoxazol-5-ylmethyl]-2-bromo-5H-imidazo[4,5-d]pyridazine and 4-fluorophenyl boronic acid. MS 508.1 (M+H⁺); H¹ NMR (DMSO-d₆): δ(ppm) 10.27 (s, 1H), 9.62 (s, 1H), 8.46-8.42 (q, 2H), 8.24-8.20 (m, 2H), 7.92-7.89 (d, 1H), 7.47-7.41 (t, 2H), 6.03 (s, 2H).

### Example 29

### 5-[3-(4-Butoxy-phenyl)-isoxazol-5-ylmethyl]-2-(3-fluoro-phenyl)-5H-imidazo[4,5-d]pyridazine (Compound 129)

From 2-bromo-5-[3-(4-butoxy-phenyl)-isoxazol-5-ylmethyl]-5H-imidazo[4,5-d]pyridazine and 3-fluorophenyl boronic acid. MS 444.2 (M+H⁺); H¹ NMR (DMSO-d₆): δ(ppm) 10.22 (s, 1H), 9.55 (s, 1H), 8.20-8.17 (d, 1H), 8.10-8.09 (d, 1H), 7.70-7.67 (d, 2H), 7.60-7.57 (m, 1H), 7.37 (t, 1H), 7.08 (s, 1H) 6.97-6.94 (d, 2H), 6.16 (s, 2H), 3.96-3.92 (t, 2H), 1.66-1.61 (m, 2H), 1.40-1.33 (m, 2H), 0.89-0.84 (t, 3H).

### Example 30

### 5-[3-(2,4-Bis-trifluoromethyl-phenyl)-isoxazol-5-ylmethyl]-2-(3-fluoro-phenyl)-5H-imidazo[4,5-d]pyridazine (Compound (Compound 130)

From 5-[3-(2,4-Bis-trifluoromethyl-phenyl)-isoxazol-5-ylmethyl]-2-bromo-5H-imidazo[4,5-d]pyridazine and 3-fluorophenyl boronic acid. MS 508.1 (M+H⁺); H¹ NMR (DMSO-d₆): δ(ppm) 10.50 (s, 1H), 9.76 (s, 1H), 8.29-8.20 (m, 4H), 7.91-7.89 (d, 1H), 7.70-7.67 (m, 1H), 7.05 (t, 1H), 7.09 (s, 1H), 6.39 (s, 2H).

### Example 31

### 5-[3-(2,4-Bis-trifluoromethyl-phenyl)-isoxazol-5-ylmethyl]-2-(4-methoxy-phenyl)-5H-imidazo[4,5-d]pyridazine (Compound 131)

From 5-[3-(2,4-Bis-trifluoromethyl-phenyl)-isoxazol-5-ylmethyl]-2-bromo-5H-imidazo[4,5-d]pyridazine and 4-methoxyphenyl boronic acid. MS 520.1 (M+H⁺); H¹ NMR (DMSO-d₆): δ(ppm) 10.45 (s, 1H), 9.73 (s, 1H), 8.39-8.36 (d, 2H), 8.25-8.20 (m, 2H), 7.91-7.89 (d, 1H), 7.22-7.19 (d, 2H), 7.09 (s, 1H), 6.39 (s, 2H), 3.88 (s, 3H).

### Example 32

### 5-[3-(2,4-Bis-trifluoromethyl-phenyl)-isoxazol-5-ylmethyl]-2-(2,4-difluoro-phenyl)-5H-imidazo[4,5-d]pyridazine (Compound 132)

From 5-[3-(2,4-Bis-trifluoromethyl-phenyl)-isoxazol-5-ylmethyl]-2-bromo-5H-imidazo[4,5-d]pyridazine and 2,4 difluorophenyl boronic acid. MS 526.1 (M+H⁺); H¹ NMR (DMSO-d₆): δ(ppm) 10.10 (s, 1H), 9.49 (s, 1H), 8.44-8.39 (m, 1H), 8.23-8.19 (m, 2H), 7.93-7.90 (d, 1H), 7.42 (t, 1H), 7.26 (t, 1H), 7.04 (s, 1H), 6.24 (s, 2H).

### Example 33

### 2-{5-[3-(2,4-Bis-trifluoromethyl-phenyl)-isoxazol-5-ylmethyl]-5H-imidazo[4,5-d]pyridazin-2-yl}-benzamide (Compound 133)

From 5-[3-(2,4-Bis-trifluoromethyl-phenyl)-isoxazol-5-ylmethyl]-2-bromo-5H-imidazo[4,5-d]pyridazine and benzamide 2-boronic acid. MS 533.1 (M+H⁺); H¹ NMR (DMSO-d₆): δ(ppm) 10.64 (s, 1H), 9.86 (s, 1H), 8.24-8.13 (m, 4H), 7.92-7.88 (m, 2H), 7.77-7.68 (m, 3H), 7.13 (s, 1H), 6.43 (s, 2H).

### Example 34

### 2-{5-[3-(2,4-Bis-trifluoromethyl-phenyl)-isoxazol-5-ylmethyl]-5H-imidazo[4,5-d]pyridazin-2-yl}-phenol (Compound 134)

From 5-[3-(2,4-Bis-trifluoromethyl-phenyl)-isoxazol-5-ylmethyl]-2-bromo-5H-imidazo[4,5-d]pyridazine and phenol 2-boronic acid. MS 506.1 (M+H⁺); H¹ NMR (DMSO-d₆): δ(ppm) 10.16 (s, 1H), 9.55 (s, 1H), 8.30-8.27 (dd, 1H), 8.18-8.14 (m, 2H), 7.87-7.84 (d, 1H), 7.38 (t, 1H), 7.03-6.96 (m, 3H), 6.26 (s, 2H).

### Example 35

### 5-[3-(2,4-Bis-trifluoromethyl-phenyl)-isoxazol-5-ylmethyl]-2-(4-trifluoromethyl-phenyl)-5H-imidazo[4,5-d]pyridazine (Compound 135)

From 5-[3-(2,4-Bis-trifluoromethyl-phenyl)-isoxazol-5-ylmethyl]-2-bromo-5H-imidazo[4,5-d]pyridazine and 4-trifluoromethylphenyl boronic acid. MS 558.1 (M+H⁺); H¹ NMR (DMSO-d₆): δ(ppm) 10.45 (s, 1H), 9.73 (s, 1H), 8.63-8.61 (d, 2H), 8.23-8.19 (m, 2H), 8.01-7.89 (m, 3H), 7.08 (s, 1H), 6.37 (s, 2H).

### Example 36

### 5-[3-(2,4-Bis-trifluoromethyl-phenyl)-isoxazol-5-ylmethyl]-2-(1H-indol-4-yl)-5H-imidazo[4,5-d]pyridazine (Compound 136)

From 5-[3-(2,4-Bis-trifluoromethyl-phenyl)-isoxazol-5-ylmethyl]-2-bromo-5H-imidazo[4,5-d]pyridazine and 1H indole 4-boronic acid. MS 529.1 (M+H⁺); H¹ NMR (DMSO-d₆): δ(ppm) 10.68 (s, 1H), 9.97 (s, 1H), 8.75 (s, 1H), 8.47-8.42 (m, 3H), 8.29-8.26 (d. 1H), 8.14-8.11 (d, 1H), 8.01-7.99 (d, 1H), 7.87 (m, 1H), 7.32 (s, 1H), 6.80 (s, 1H), 6.64 (s, 2H).

### Example 37

### 1-(3-{5-[3-(2,4-Bis-trifluoromethyl-phenyl)-isoxazol-5-ylmethyl]-5H-imidazo[4,5-d]pyridazin-2-yl}-4-fluoro-phenyl)-ethanone (Compound 137)

From 5-[3-(2,4-Bis-tri fluoromethyl-phenyl)-isoxazol-5-ylmethyl]-2-bromo-5H-imidazo[4,5-d]pyridazine and 2-fluoro 5-acetylphenyl boronic acid. MS 550.1 (M+H⁺); H¹ NMR (DMSO-d₆): δ(ppm) 10.35 (s, 1H), 9.68 (s, 1H), 8.92-8.89 (dd, 1H), 8.24-8.19 (m, 3H), 7.93-7.90 (d, 1H), 7.58 (m, 1H), 7.08 (s, 1H), 6.35 (s, 2H), 2.67 (s, 3H).

### Example 38

### 2-(4-Methoxy-phenyl)-5-[3-(4-propoxy-phenyl)-isoxazol-5-ylmethyl]-5H-imidazo[4,5-d]pyridazine (Compound 138)

From 2-bromo-5-[3-(4-propoxy-phenyl)-isoxazol-5-ylmethyl]-5H-imidazo[4,5-d]pyridazine and 4-methoxyphenyl boronic acid. MS 442.2 (M+H⁺); H¹ NMR (DMSO-d₆): δ(ppm) 10.31 (s, 1H), 9.63 (s, 1H), 8.31-8.28 (d, 2H), 7.71-7.68 (d, 2H), 7.16-7.13 (d, 2H), 7.09 (s, 1H), 6.70-6.95 (d, 2H), 6.22 (s, 2H), 3.93-3.88 (t, 2H), 3.18 (s, 3H), 1.70-1.63 (m, 2H), 0.94-0.89 (t, 3H).

### Example 39

### 5-[3-(2,4-Bis-trifluoromethyl-phenyl)-isoxazol-5-ylmethyl]-2-(1H-indol-5-yl)-5H-imidazo[4,5-d]pyridazine (Compound 139)

From 5-[3-(2,4-Bis-trifluoromethyl-phenyl)-isoxazol-5-ylmethyl]-2-bromo-5H-imidazo[4,5-d]pyridazine and 1H indole 5-boronic acid. MS 529.1 (M+H⁺); H¹ NMR (DMSO-d₆): δ(ppm) 10.81 (s, 1H), 10.11 (s, 1H), 9.07 (s, 1H), 8.61 (m, 3H), 8.28-8.26 (d, 1H), 8.01-7.88 (m, 3H), 7.46 (s, 1H), 7.02 (s, 1H), 6.77 (s, 2H).

### Example 40

### 5-[3-(2,4-Bis-trifluoromethyl-phenyl)-isoxazol-5-ylmethyl]-2-(2,6-difluoro-phenyl)-5H-imidazo[4,5-d]pyridazine (Compound 140)

From 5-[3-(2,4-Bis-trifluoromethyl-phenyl)-isoxazol-5-ylmethyl]-2-bromo-5H-imidazo[4,5-d]pyridazine and 2,6 difluorophenyl boronic acid. MS 526.1 (M+H⁺); H¹ NMR (DMSO-d₆): δ(ppm) 10.42 (s, 1H), 9.72 (s, 1H), 8.25 (m, 3H), 7.92 (d, 1H), 7.38-7.33 (m, 2H), 7.08 (s, 1H), 6.35 (s, 2H).

### Example 41

### 5-[3-(4-Butoxy-phenyl)-isoxazol-5-ylmethyl]-2-(4-methoxy-phenyl)-5H-imidazo[4,5-d]pyridazine (Compound 141)

From 2-bromo-5-[3-(4-butoxy-phenyl)-isoxazol-5-ylmethyl]-5H-imidazo[4,5-d]pyridazine and 4-methoxyphenyl boronic acid. MS 456.2 (M+H⁺); H¹ NMR (DMSO-d₆): δ(ppm) 10.35 (s, 1H), 9.71 (s, 1H), 8.40-8.37 (d, 2H), 7.76-7.73 (d, 2H), 7.22-7.19 (d, 2H), 7.15 (s, 1H), 7.03-7.00 (d, 2H), 6.30 (s, 2H), 4.02-3.98 (t, 2H), 3.88 (s, 3H), 1.69 (m, 2H), 1.43-1.41 (m, 2H), 0.94-0.90 (t, 3H).

### Example 42

### 5-[3-(2,4-Bis-trifluoromethyl-phenyl)-isoxazol-5-ylmethyl]-2-furan-2-yl-5H-imidazo[4,5-d]pyridazine (Compound 142)

From 5-[3-(2,4-Bis-trifluoromethyl-phenyl)-isoxazol-5-ylmethyl]-2-bromo-5H-imidazo[4,5-d]pyridazine and furan 2-boronic acid. MS 480.1 (M+H⁺); H¹ NMR (DMSO-d₆): δ(ppm) 10.24 (s, 1H), 9.58 (s, 1H), 8.24-8.20 (m, 2H), 8.06 (s, 1H), 7.92 (d, 1H), 7.51-7.50 (d, 1H), 7.06 (s, 1H), 6.81-6.80 (m, 1H), 6.31 (s, 2H).

### Example 43

### 5-[3-(2,4-Bis-trifluoromethyl-phenyl)-isoxazol-5-ylmethyl]-2-thiophen-2-yl-5H-imidazo[4,5-d]pyridazine (Compound 143)

From 5-[3-(2,4-Bis-trifluoromethyl-phenyl)-isoxazol-5-ylmethyl]-2-bromo-5H-imidazo[4,5-d]pyridazine and thiophene 2-boronic acid. MS 496.0 (M+H⁺); H¹ NMR (DMSO-d₆): δ(ppm) 10.34 (s, 1H), 9.65 (s, 1H), 8.23-8.20 (m, 3H), 7.80-7.89 (m, 2H), 7.34-7.31 (t, 1H), 7.07 (s, 1H), 6.35 (s, 2H).

### Example 44

### 2-Furan-2-yl-5-[3-(4-propoxy-phenyl)-isoxazol-5-ylmethyl]-5H-imidazo[4,5-d]pyridazine (Compound 144)

From 2-bromo-5-[3-(4-propoxy-phenyl)-isoxazol-5-ylmethyl]-5H-imidazo[4,5-d]pyridazine and furan 2-boronic acid. MS 402.2 (M+H⁺); H¹ NMR (DMSO-d₆): δ(ppm) 10.270 (s, 1H), 9.57 (s, 1H), 8.05 (s, 1H), 7.70-7.67 (d, 2H), 7.54-7.53 (d, 1H), 7.09 (s, 1H), 6.97-6.94 (d, 2H), 6.78-6.76 (m, 1H), 6.19 (s, 2H), 3.92-3.88 (t, 2H), 1.68-1.65 (m, 2H), 0.93-0.88 (t, 3H).

### Example 45

### 2-(4-Fluoro-phenyl)-5-[3-(4-propoxy-phenyl)-isoxazol-5-ylmethyl]-5H-imidazo[4,5-d]pyridazine (Compound 145)

From 2-bromo-5-[3-(4-propoxy-phenyl)-isoxazol-5-ylmethyl]-5H-imidazo[4,5-d]pyridazine and 4-fluorophenyl boronic acid. MS 430.2 (M+H⁺); H¹ NMR (DMSO-d₆): δ(ppm) 10.21 (s, 1H), 9.55 (s, 1H), 8.46-8.41 (m, 2H), 7.78-7.74 (m, 2H), 7.45-7.39 (m, 2H), 7.13 (s, 1H), 7.03-7.00 (d, 2H), 6.19 (s, 2H), 3.98-3.94 (m, 2H), 1.74-1.71 (q, 2H), 0.99-0.94 (m, 3H).

### General Procedure F

### Synthesis of 2-Aryl-5H-imidazo[4,5-d]pyridazines

To a solution of diaminomaleonitrile in THF (1 mL/mmol) was added aryl aldehyde (1 eq) and then catalytic H₂SO₄ (1 drop/20 mmol) and stirred at room temperature for 90 minutes. The solvent was evaporated to dryness then the solid washed with 1:1 ethyl ether and hexane giving the pure product: 2-amino-3-aryl-but-2-enedinitrile.

The 2-amino-3-aryl-but-2-enedinitrile is dissolved in DMF (3 mL/mmol) and then treated with NCS (1.5 eq) followed by nicotinamide (1.5 eq). The solution turned to dark brown in 2 minutes. After 1 hour the precipitated nicotinamide HCl salt was filtered off and the solution concentrated to oil. The reaction mixture was then poured into cold water with the product oiling out. Ethyl acetate was added to dissolve the oil and the organics were washed with brine. The organics were dried with MgSO₄ and evaporated to give a black oil. The oil was dissolved in a minimum amount of DCM and filtered through silica gel (3 g/mmol) with DCM: MeOH (4:1). The solvent was evaporated to give the product-2-aryl-1H-imidazole-4,5-dicarbonitrile.

The 2-aryl-1H-imidazole-4,5-dicarbonitrile was dissolved in THF (1.5 mL/mmol), cooled to −78° C. and treated with DIBAL-H (6.5 eq, 1M in THF) dropwise. Water was carefully added to the cold mixture until the excess DIBAL-H was fully quenched. Hydrazine (3 eq. hydrate) was added to the solution and then the reaction was warmed to room temperature. MeOH (1 mL/mmol) was added and the aluminum salts were filtered. The solid was washed with another 50 mL of MeOH. The filtrate was evaporated and purified by silica column with the gradient from 10% to 30% DCM/MeOH (with 10% v/v NH₄OH) to provide 2-aryl-5H-imidazo[4,5-d]pyridazines.

### General Procedure G

### Synthesis of Methanesulfonic Acid 5-Aryl-isoxazol-3-ylmethyl Esters

A mixture of phenyl isocyanate (2.2 eq, 1.1 g), 2-(2-Nitro-ethoxy)-tetrahydro-pyran (1 eq, 875 mg) and aryl alkyne (1 eq, 5 mmol) in benzene (20 mL) was treated with DIEA (20 drops, excess) then heated to 75° C. overnight in a sealed vial. The mixture was cooled, the solution decanted, concentrated and purified on silica gel eluting with EtOAc: hexanes 0-40% to give the 3-(Tetrahydro-pyran-2-yloxymethyl)-aryl-isoxazole.

A solution of the 3-(Tetrahydro-pyran-2-yloxymethyl)-5-aryl-isoxazole (725 mg) in HOAc: H₂O: THF (4:2:1, 10 mL) was heated to 75° C. for 5 hrs. The mixture was cooled to room temperature, concentrated and the product, 5-aryl-isoxazol-3-yl-methanol, which was used directly.

To a solution of the 5-aryl-isoxazol-3-yl-methanol (2.2 mmol) in DCM (20 mL) was added triethylamine (0.5 mL, 2 eq.) and mesyl chloride (1.5 eq, 0.26 mL) and stirred at room temperature for 1 hr. The reaction was then quenched with water (10 mL) and the organics partitioned and concentrated to give the crude product methanesulfonic acid 5-aryl-isoxazol-3-ylmethyl ester.

### General Procedure H

### Synthesis of Compounds 146-244 and 275-289

A solution of 2-aryl-5H-imidazo[4,5-d]pyridazine (0.10 mmol), chloromethyl-, or methanesulfonic acid methyl ester- of aryl isoxazole compound (1 equivalent), and alkali carbonate (0.20 mmol) in DMF (3 mL) was heated under microwave irradiation at 60-120° C. for 10 minutes. The reaction was filtered and purified by reverse phase HPLC to give the desired product. The product was converted to the HCl salt by the addition of 1N HCl before concentration.

### Example 46

### 2-(2,3-Difluoro-phenyl)-5-[3-(4-pyridin-4-ylethynyl-phenyl)-isoxazol-5-ylmethyl]-5H-imidazo[4,5-d]pyridazine (Compound 146)

### (4-Pyridin-4-ylethynyl-phenyl)-methanol

A mixture of 4-ethynylpyridine hydrochloride (210 mg, 1.5 mmol), 4-iodobenzyl alcohol (351 mg, 1.5 mmol), and triethylamine (4 mL) was sparged with Ar for 2 min in a microwave vial. To this mixture was added Cu(I) I (29 mg, 0.15 mmol) and tetrakis(triphenylphosphine)palladium (92 mg, 0.08 mmol). The vial was sealed, and the contents were heated to 130° C. in a microwave for 10 min. The cooled reaction mixture was heterogeneous with a heavy black ppt. The reaction mixture was partitioned between EtOAc and water. The organic layer was washed with brine, dried over sodium sulfate, and adsorbed onto Celite. The product was purified by SiO₂ flash chromatography using EtOAc in hexanes (50-100%) to give the product as a white flaky solid. Yield: 110 mg.

### 4-Pyridin-4-ylethynyl-benzaldehyde

(4-Pyridin-4-ylethynyl-phenyl)-methanol (100 mg) was suspended in DCM (20 mL) and excess MnO₂ (ca. 1 g) was added. The reaction mixture was stirred for 1 h, filtered, and concentrated onto Celite. The product was isolated by SiO₂ flash chromatography using EtOAc in hexanes (30-100%) to give the product as a white crystalline solid. Yield: 57 mg.

### 4-(2-Pyridin-4-yl-ethyl)-benzaldehyde

(4-Pyridin-4-ylethynyl-phenyl)-methanol (180 mg) was dissolved in EtOH (50 mL) and the solution was sparged with Ar. Pd (10% on carbon (50 mg) was added and mixture was stirred for 1 h under a balloon filled with H₂. The reaction mixture was filtered through Celite, and was then concentrated onto Celite. The product was isolated by SiO₂ flash chromatography using 50-100% EtOAc in hexanes to give the product as a flaky solid. This material was dissolved in DCM (25 mL) and a large excess of MnO₂ (ca. 1 g) was added. The reaction mixture was stirred for 30 min, and then was filtered through Celite and concentrated onto Celite. The product was purified by SiO₂ flash chromatography using EtOAc in hexanes (50-100%) to give the product as white crystals. Yield: 57 mg.

### 2-(2,3-Difluoro-phenyl)-5-[3-(4-pyridin-4-ylethynyl-phenyl)-isoxazol-5-ylmethyl]-5H-imidazo[4,5-d]pyridazine (Compound 146)

From 2-(2,3-difluoro-phenyl)-5H-imidazo[4,5-d]pyridazine and 4-[4-(5-chloromethyl-isoxazol-3-yl)-phenylethynyl]-pyridine (General Procedure B, from 4-(2-pyridin-4-yl-ethyl)-benzaldehyde). 491.0 (M+H⁺); H¹ NMR (DMSO-d₆): δ(ppm) 10.54 (s, 1H), 9.76 (s, 1H), 8.83-8.81 (dd, 2H), 8.19-8.15 (m, 1H), 7.99-7.91 (m, 4H), 7.81-7.68 (m, 3H), 7.50-7.43 (m, 1H), 7.33 (s, 1H), 6.38 (s, 2H).

### Example 47

### 5-[3-(2,4-Bis-trifluoromethyl-phenyl)-isoxazol-5-ylmethyl]-2-(2,4,5-trifluoro-phenyl)-5H-imidazo[4,5-d]pyridazine (Compound 147)

From 3-(2,4-bis-trifluoromethyl-phenyl)-5-chloromethyl-isoxazole and 2-(2,4,5-trifluoro-phenyl)-5H-imidazo[4,5-d]pyridazine. 544.0 (M+H⁺); H¹ NMR (DMSO-d₆): δ(ppm) 10.45 (s, 1H), 9.71 (s, 1H), 8.42-8.33 (m, 1H), 8.24-8.20 (m, 2H), 7.93-7.82 (m, 2H), 7.08 (s, 1H), 6.37 (s, 2H).

### Example 48

### 2-(2,3-Difluoro-phenyl)-5-{3-[4-(pyridin-4-ylmethoxy)-phenyl]-isoxazol-5-ylmethyl}-H-imidazo[4,5-d]pyridazine (Compound 148)

From 2-(2,3-difluoro-phenyl)-5H-imidazo[4,5-d]pyridazine and 4-[4-(5-chloromethyl-isoxazol-3-yl)-phenoxymethyl]-pyridine. MS 497.1 (M+H⁺); H¹ NMR (DMSO-d₆): δ(ppm) 10.61 (s, 1H), 9.79 (s, 1H), 8.92-8.90 (d, 2H), 8.19-8.16 (d, 1H), 8.06-8.04 (d, 2H), 7.84-7.69 (m, 3H), 7.51-7.45 (t, 1H), 7.19-7.16 (m, 3H), 5.53 (s, 2H), 6.36 (s, 2H).

### Example 49

### 2-(2,3-Difluoro-phenyl)-5-[3-(2,4-dimethyl-thiazol-5-yl)-isoxazol-5-ylmethyl]-5H-imidazo[4,5-d]pyridazine (Compound 149)

From 2-(2,3-difluoro-phenyl)-5H-imidazo[4,5-d]pyridazine and 5-chloromethyl-3-(2,4-dimethyl-thiazol-5-yl)-isoxazole. 425.0 (M+H⁺); H¹ NMR (DMSO-d₆): δ(ppm) 10.62 (s, 1H), 9.82 (s, 1H), 8.16-8.14 (t, 1H), 7.79-7.75 (q, 1H), 7.5 (m, 1H), 7.14 (s, 1H), 6.37 (s, 2H), 2.62 (s, 3H), 2.49 (s, 3H).

### Example 50

### 5-[3-(3,4-Bis-difluoromethoxy-phenyl)-isoxazol-5-ylmethyl]-2-(2,3-difluoro-phenyl)-5H-imidazo[4,5-d]pyridazine (Compound 150)

From 2-(2,3-difluoro-phenyl)-5H-imidazo[4,5-d]pyridazine and 3-(3,4-Bis-difluoromethoxy-phenyl)-5-chloromethyl-isoxazole. 522.0 (M+H⁺); H¹ NMR (DMSO-d₆): δ(ppm) 10.50 (s, 1H), 9.74 (s, 1H), 8.18-8.13 (t, 1H), 7.15-7.71 (m, 3H), 7.54-7.45 (m, 2H), 7.30 (s, 2H), 7.05 (s, 1H), 6.35 (s, 2H).

### Example 51

### 5-[3-(4-Difluoromethoxy-3-ethoxy-phenyl)-isoxazol-5-ylmethyl]-2-(2,3-difluoro-phenyl)-5H-imidazo[4,5-d]pyridazine (Compound 151)

From 2-(2,3-difluoro-phenyl)-5H-imidazo[4,5-d]pyridazine and 5-chloromethyl-3-(4-difluoromethoxy-3-ethoxy-phenyl)-isoxazole. 500.7 (M+H⁺); H¹ NMR (DMSO-d₆): δ(ppm) 10.12 (s, 1H), 9.50 (s, 1H), 8.16 (t, 1H), 7.56-7.52 (m, 2H), 7.46-7.43 (dd, 1H), 7.38-7.34 (m, 1H), 7.27-7.23 (m, 2H), 7.13 (s, 1H), 6.18 (s, 2H), 4.17-4.10 (q, 2H), 1.36-1.32 (t, 3H).

### Example 52

### 2-(2,3-Difluoro-phenyl)-5-{3-[4-(4-methyl-piperazin-1-ylmethyl)-phenyl]-isoxazol-5-ylmethyl}-5H-imidazo[4,5-d]pyridazine (Compound 152)

From 2-(2,3-difluoro-phenyl)-5H-imidazo[4,5-d]pyridazine and 1-[4-(5-chloromethyl-isoxazol-3-yl)-benzyl]-4-methyl-piperazine. 502.1 (M+H⁺); H¹ NMR (DMSO-d₆): δ(ppm) 10.58 (s, 1H), 9.76 (s, 1H), 8.18-8.14 (t, 1H), 7.93-7.90 (d, 2H), 7.78-7.71 (m, 3H), 7.50-7.46 (m, 1H), 7.29 (s, 1H), 6.36 (s, 2H), 4.40 (b, 2H), 3.59-3.51 (d, 8H), 2.79 (s, 3H).

### Example 53

### 2-(2,3-Difluoro-phenyl)-5-[3-(4-imidazol-1-ylmethyl-phenyl)-isoxazol-5-ylmethyl]-5H-imidazo[4,5-d]pyridazine (Compound 153)

From 2-(2,3-difluoro-phenyl)-5H-imidazo[4,5-d]pyridazine and 5-chloromethyl-3-(4-imidazol-1-ylmethyl-phenyl)-isoxazole. MS 470.1 (M+H⁺); H¹ NMR (DMSO-d₆): δ(ppm) 10.49 (s, 1H), 9.71 (s, 1H), 9.33 (s, 1H), 8.17-8.15 (d, 1H), 7.89-7.86 (d, 2H), 7.80 (s, 1H), 7.71-7.70 (m, 2H), 7.53-7.44 (m, 3H), 7.25 (d, 1H), 6.33 (s, 2H), 5.49 (s, 2H).

### Example 54

### 2-(2,3-Difluoro-phenyl)-5-{3-[4-(1-methyl-1H-imidazol-2-ylmethoxy)-phenyl]-isoxazol-5-ylmethyl}-5H-imidazo[4,5-d]pyridazine (Compound 154)

From 2-(2,3-difluoro-phenyl)-5H-imidazo[4,5-d]pyridazine 5-chloromethyl-3-[4-(1-methyl-1H-imidazol-2-ylmethoxy)-phenyl]-isoxazole. MS 500.1 (M+H⁺); H¹ NMR (DMSO-d₆): δ(ppm) 10.59 (s, 1H), 9.78 (s, 1H), 8.19-8.15 (m, 1H), 7.85-7.70 (m, 5H), 7.50-7.47 (t, 1H), 7.26-7.21 (m, 3H), 6.36 (s, 2H), 5.55 (s, 2H), 3.87 (s, 3H).

### Example 55

### 2-(2,3-Difluoro-phenyl)-5-(3-pyridin-4-yl-isoxazol-5-ylmethyl)-5H-imidazo[4,5-d]pyridazine (Compound 155)

From 2-(2,3-difluoro-phenyl)-5H-imidazo[4,5-d]pyridazine and 4-(5-chloromethyl-isoxazol-3-yl)-pyridine. MS 391.1 (M+H⁺); H¹ NMR (DMSO-d₆): δ(ppm) 10.42 (s, 1H), 9.70 (s, 1H), 8.86-8.84 (d, 2H), 8.17-8.12 (t, 1H), 8.07-8.05 (dd, 2H), 7.72-7.68 (m, 1H), 7.48-7.44 (m, 2H), 6.37 (s, 2H).

### Example 56

### 2-(2,3-Difluoro-phenyl)-5-[3-(4-morpholin-4-ylmethyl-phenyl)-isoxazol-5-ylmethyl]-5H-imidazo[4,5-d]pyridazine (Compound 156)

From 2-(2,3-difluoro-phenyl)-5H-imidazo[4,5-d]pyridazine and 4-[4-(5-chloromethyl-isoxazol-3-yl)-benzyl]-morpholine. MS 489.2 (M+H⁺); H¹ NMR (DMSO-d₆): δ(ppm) 10.43 (s, 1H), 9.70 (s, 1H), 8.17-8.14 (m, 1H), 7.94-7.91 (d, 2H), 7.75-7.72 (m, 3H), 7.48-7.41 (m, 1H), 7.27 (s, 1H), 6.32 (s, 2H), 4.37 (s, 2H), 3.94-3.74 (m, 4H), 3.24-3.10 (m, 4H).

### Example 57

### 2-(2,3-Difluoro-phenyl)-5-[3-(4-piperidin-1-ylmethyl-phenyl)-isoxazol-5-ylmethyl]-5H-imidazo[4,5-d]pyridazine (Compound 157)

From 2-(2,3-difluoro-phenyl)-5H-imidazo[4,5-d]pyridazine and 1-[4-(5-chloromethyl-isoxazol-3-yl)-benzyl]-piperidine. MS 487.2 (M+H⁺); H¹ NMR (DMSO-d₆): δ(ppm) 10.87 (s, 1H), 9.66 (s, 1H), 8.17-8.14 (m, 1H), 7.94-7.91 (d, 2H), 7.72-7.66 (m, 3H), 7.46-7.39 (m, 1H), 7.26 (s, 1H), 6.30 (s, 2H), 4.03-4.29 (d, 2H), 3.29-3.25 (d, 2H), 2.84 (b, 2H), 1.75-1.66 (m, 6H).

### Example 58

### 2-(2,3-Difluoro-phenyl)-5-{3-[4-(2-pyrrolidin-1-yl-ethoxy)-phenyl]-isoxazol-5-ylmethyl}-5H-imidazo[4,5-d]pyridazine (Compound 158)

From 2-(2,3-difluoro-phenyl)-5H-imidazo[4,5-d]pyridazine and 5-chloromethyl-3-[4-(2-pyrrolidin-1-yl-ethoxy)-phenyl]-isoxazole. MS 503.1 (M+H⁺); H¹ NMR (DMSO-d₆): δ(ppm) 10.32 (s, 1H), 9.62 (s, 1H), 8.17-8.13 (m, 1H), 7.83-7.80 (d, 2H), 7.66-7.63 (m, 1H), 7.43-7.37 (m, 1H), 7.16-7.09 (m, 3H), 6.24 (s, 2H), 4.39-4.36 (m, 2H), 3.60-3.56 (m, 4H), 3.12-3.06 (b, 2H), 2.01-1.86 (m, 4H).

### Example 59

### 3-(4-{5-[2-(2,3-Difluoro-phenyl)-imidazo[4,5-d]pyridazin-5-ylmethyl]-isoxazol-3-yl}-phenoxymethyl)-benzoic acid (Compound 159)

From 2-(2,3-difluoro-phenyl)-5H-imidazo[4,5-d]pyridazine and 3-[4-(5-chloromethyl-isoxazol-3-yl)-phenoxymethyl]-benzoic acid. MS 540.2 (M+H⁺); H¹ NMR (DMSO-d₆): δ(ppm) 10.27 (s, 1H), 9.60 (s, 1H), 8.13-8.09 (m, 1H), 7.80 (s, 1H), 7.86-7.83 (d, 1H), 7.76-7.73 (d, 2H), 7.66-7.60 (m, 2H), 7.49-7.37 (m, 2H), 7.10-7.07 (d, 3H), 6.19 (s, 2H), 5.19 (s, 2H).

### Example 60

### 2-(2,3-Difluoro-phenyl)-5-[3-(4-fluoro-2-trifluoromethoxy-phenyl)-isoxazol-5-ylmethyl]-5H-imidazo[4,5-d]pyridazine (Compound 160)

### 4-Fluoro-2-trifluoromethoxy-benzaldehyde

To a solution of 1-fluoro-3-trifluoromethoxy-benzene (1.73 g, 9.6 mmol) in THF (20 mL) at −78° C. was added nBuLi (1.2 eq, 4.6 mL of 2.5M in hexanes). The mixture was stirred for 180 minutes and quenched with DMF (2 mL) and allowed to warm to room temperature. Solvents were removed, the reaction was washed with H₂O (10 mL) and the organics concentrated giving the crude product.

### 2-(2,3-Difluoro-phenyl)-5-[3-(4-fluoro-2-trifluoromethoxy-phenyl)-isoxazol-5-ylmethyl]-5H-imidazo[4,5-d]pyridazine (Compound 160)

From 2-(2,3-difluoro-phenyl)-5H-imidazo[4,5-d]pyridazine and 5-chloromethyl-3-(4-fluoro-2-trifluoromethoxy-phenyl)-isoxazole (General Procedure B, from 4-fluoro-2-trifluoromethoxy-benzaldehyde). MS 492.0 (M+H⁺); H¹ NMR (DMSO-d₆): δ(ppm) 10.67 (s, 1H), 9.67 (s, 1H), 8.16-8.12 (m, 1H), 7.76-7.66 (m, 2H), 7.53-7.39 (m, 3H), 7.05 (s, 1H), 6.34 (s, 2H).

### Example 61

### [2-(4-{5-[2-(2,3-Difluoro-phenyl)-imidazo[4,5-d]pyridazin-5-ylmethyl]-isoxazol-3-yl}-phenoxy)-ethyl]-dimethyl-amine (Compound 161)

From 2-(2,3-difluoro-phenyl)-5H-imidazo[4,5-d]pyridazine and {2-[4-(5-chloromethyl-isoxazol-3-yl)-phenoxy]-ethyl}-dimethyl-amine. MS 477.2 (M+H⁺); H¹ NMR (DMSO-d₆): δ(ppm) 10.47 (s, 1H), 9.72 (s, 1H), 8.18-8.15 (m, 1H), 7.83-7.66 (m, 3H), 7.49-7.42 (m, 1H), 7.18 (s, 1H), 7.12-7.08 (m, 2H), 6.31 (s, 2H), 4.43-4.39 (t, 2H), 3.53-3.45 (q, 2H), 2.84-2.82 (d, 6H).

### Example 62

### 4-(4-{5-[2-(2,3-Difluoro-phenyl)-imidazo[4,5-d]pyridazin-5-ylmethyl]-isoxazol-3-yl}-phenoxymethyl)-benzoic acid (Compound 162)

From 2-(2,3-difluoro-phenyl)-5H-imidazo[4,5-d]pyridazine and 4-[4-(5-chloromethyl-isoxazol-3-yl)-phenoxymethyl]-benzoic acid. MS 540.7 (M+H⁺); H¹ NMR (DMSO-d₆): δ(ppm) 10.41 (s, 1H), 9.69 (s, 1H), 8.18-8.14 (m, 1H), 7.95-7.93 (m, 2H), 7.79-7.76 (m, 2H), 7.70-7.67 (m, 1H), 7.56-7.54 (d, 2H), 7.47-7.40 (m, 1H), 7.15-7.11 (m, 3H), 6.47 (s, 2H), 5.24 (s, 2H).

### Example 63

### 5-[3-(4-Difluoromethoxy-3-methoxy-phenyl)-isoxazol-5-ylmethyl]-2-(2,3-difluoro-phenyl)-5H-imidazo[4,5-d]pyridazine (Compound 163)

From 2-(2,3-difluoro-phenyl)-5H-imidazo[4,5-d]pyridazine and 5-chloromethyl-3-(4-difluoromethoxy-3-methoxy-phenyl)-isoxazole. MS 486.0 (M+H⁺); H¹ NMR (DMSO-d₆): δ(ppm) 10.27 (s, 1H) 9.61 (s, 1H), 8.17-8.13 (m, 1H), 7.45-7.62 (m, 1H), 7.54 (s, 1H), 7.47-7.37 (m, 2H), 7.28-7.28 (m, 2H), 7.14 (s, 1H), 6.24 (s, 2H), 3.87 (s, 3H).

### Example 64

### 5-[3-(3,5-Bis-trifluoromethyl-phenyl)-isoxazol-5-ylmethyl]-2-(2,3-difluoro-phenyl)-5H-imidazo[4,5-d]pyridazine (Compound 164)

From 2-(2,3-difluoro-phenyl)-5H-imidazo[4,5-d]pyridazine and 3-(3,5-Bis-trifluoromethyl-phenyl)-5-chloromethyl-isoxazole. MS 526.0 (M+H⁺); H¹ NMR (DMSO-d₆): δ(ppm) 10.18 (s, 1H), 9.55 (s, 1H), 8.48 (s, 2H), 8.26 (s, 1H), 8.16-8.12 (m, 1H), 7.61-7.57 (m, 1H), 7.45 (s, 1H), 7.40-7.33 (m, 1H), 6.26 (s, 2H).

### Example 65

### 5-[3-(3-Chloro-4-trifluoromethoxy-phenyl)-isoxazol-5-ylmethyl]-2-(2,3-difluoro-phenyl)-5H-imidazo[4,5-d]pyridazine (Compound 165)

From 2-(2,3-difluoro-phenyl)-5H-imidazo[4,5-d]pyridazine and 5-chloromethyl-3-(3-chloro-4-trifluoromethoxy-phenyl)-isoxazole. MS 508.0 (M+H⁺); H¹ NMR (DMSO-d₆): δ(ppm) 10.50 (s, 1H), 9.75 (s, 1H), 8.17-8.13 (t, 2H), 7.98-7.94 (dd, 1H), 7.73-7.69 (m, 2H), 7.48-7.45 (m, 1H), 7.34 (s, 1H), 6.36 (s, 2H).

### Example 66

### 2-{5-[2-(2,3-Difluoro-phenyl)-imidazo[4,5-d]pyridazin-5-ylmethyl]-isoxazol-3-yl}-5-methoxy-phenol (Compound 166)

From 2-(2,3-difluoro-phenyl)-5H-imidazo[4,5-d]pyridazine and 2-(5-chloromethyl-isoxazol-3-yl)-5-methoxy-phenol.MS 436.1 (M+H⁺); H¹ NMR (DMSO-d₆): δ(ppm) 10.45 (s, 1H), 10.30 (s, 1H), 9.71 (s, 1H), 8.17-8.14 (m, 1H), 7.74-7.61 (m, 2H), 7.47-7.41 (m, 1H), 7.17 (s, 1H), 6.57-6.56 (d, 1H), 6.50-6.46 (dd, 1H), 6.27 (s, 2H), 3.72 (s, 3H).

### Example 67

### 5-[3-(2,2-Difluoro-benzo[1,3]dioxol-5-yl)-isoxazol-5-ylmethyl]-2-(2,3-difluoro-phenyl)-5H-imidazo[4,5-d]pyridazine (Compound 167)

From 2-(2,3-difluoro-phenyl)-5H-imidazo[4,5-d]pyridazine and 5-chloromethyl-3-(2,2-difluoro-benzo[1,3]dioxol-5-yl)-isoxazole. MS 470.1 (M+H⁺); H¹ NMR (DMSO-d₆): δ(ppm) 10.62 (s, 1H), 9.79 (s, 1H), 8.18-8.14 (m, 1H), 7.89 (d, 1H), 7.75-7.70 (m, 2H), 7.55-7.44 (m, 2H), 7.27 (s, 1H), 6.40 (s, 2H).

### Example 68

### 2-(2,3-Difluoro-phenyl)-5-[3-(3-fluoro-4-trifluoromethoxy-phenyl)-isoxazol-5-ylmethyl]-5H-imidazo[4,5-d]pyridazine (Compound 168)

From 2-(2,3-difluoro-phenyl)-5H-imidazo[4,5-d]pyridazine and 5-chloromethyl-3-(3-fluoro-4-trifluoromethoxy-phenyl)-isoxazole. MS 492.1 (M+H⁺); H¹ NMR (DMSO-d₆): δ(ppm) 10.26 (s, 1H), 9.61 (s, 1H), 8.16-8.12 (m, 1H), 8.03-7.99 (dd, 1H), 7.84-7.81 (d, 1H), 7.74-7.62 (m, 2H), 7.43-7.38 (m, 1H), 7.28 (s, 1H), 6.26 (s, 2H).

### Example 69

### 5-[3-(2,4-Bis-difluoromethoxy-phenyl)-isoxazol-5-ylmethyl]-2-(2,3-difluoro-phenyl)-5H-imidazo[4,5-d]pyridazine (Compound 169)

From 2-(2,3-difluoro-phenyl)-5H-imidazo[4,5-d]pyridazine and 3-(2,4-Bis-difluoromethoxy-phenyl)-5-chloromethyl-isoxazole. MS 522.0 (M+H⁺); H¹ NMR (DMSO-d₆): δ(ppm) 10.09 (s, 1H), 9.57 (s, 1H), 8.17-8.13 (m, 1H), 7.90-7.87 (d, 1H), 7.62-7.56 (m, 1H), 7.38-7.32 (m, 2H), 7.20-7.10 (m, 1H), 7.05 (s, 1H), 6.19 (s, 2H), 3.32 (s, 2H).

### Example 70

### 2-(2,3-Difluoro-phenyl)-5-{3-[4-(1,1,2,3,3,3-hexafluoro-propoxy)-phenyl]-isoxazol-5-ylmethyl}-5H-imidazo[4,5-d]pyridazine (Compound 170)

From 2-(2,3-difluoro-phenyl)-5H-imidazo[4,5-d]pyridazine and 5-chloromethyl-3-[4-(1,1,2,3,3,3-hexafluoro-propoxy)-phenyl]-isoxazole. MS 556.0 (M+H⁺); H¹ NMR (DMSO-d₆): δ(ppm) 10.48 (s, 1H), 9.23 (s, 1H), 8.17-8.13 (m, 1H), 7.96-7.93 (d, 2H), 7.75-7.66 (m, 1H), 7.48-7.38 (m, 3H), 7.27 (s, 1H), 6.54-6.52 (m, 1H), 6.40-6.34 (m, 2H).

### Example 71

### 2-(2,3-Difluoro-phenyl)-5-[3-(4-methoxy-2-methyl-phenyl)-isoxazol-5-ylmethyl]-5H-imidazo[4,5-d]pyridazine (Compound 171)

From 2-(2,3-difluoro-phenyl)-5H-imidazo[4,5-d]pyridazine and 5-chloromethyl-3-(4-methoxy-2-methyl-phenyl)-isoxazole. MS 434.1 (M+H⁺); H¹ NMR (DMSO-d₆): δ(ppm) 10.43 (s, 1H), 9.71 (s, 1H), 8.18-8.13 (m, 1H), 7.71-7.67 (m, 1H), 7.48-7.41 (m, 2H), 7.03 (s, 1H), 6.91-6.83 (m, 2H), 6.28 (s, 2H), 3.76 (s, 3H), 2.40 (s, 3H).

### Example 72

### 2-(2,3-Diffluoro-phenyl)-5-{3-[4-(pyridin-2-ylmethoxy)-phenyl]-isoxazol-5-ylmethyl}-5H-imidazo[4,5-d]pyridazine (Compound 172)

From 2-(2,3-difluoro-phenyl)-5H-imidazo[4,5-d]pyridazine and 2-[4-(5-chloromethyl-isoxazol-3-yl)-phenoxymethyl]-pyridine. MS 497.0 (M+H⁺); H¹ NMR (DMSO-d₆): δ(ppm) 10.57 (s, 1H), 9.79 (s, 1H), 8.72-8.71 (d, 1H), 8.19-8.12 (m, 2H), 7.82-7.74 (m, 4H), 7.64-7.60 (m, 1H), 7.51-7.47 (m, 1H), 7.19-7.16 (d, 3H), 6.34 (s, 2H), 5.38 (s, 2H).

### Example 73

### 5-[3-(4-Benzyloxy-phenyl)-isoxazol-5-ylmethyl]-2-(2,3-difluoro-phenyl)-5H-imidazo[4,5-d]pyridazine (Compound 173)

From 2-(2,3-difluoro-phenyl)-5H-imidazo[4,5-d]pyridazine and 3-(4-benzyloxy-phenyl)-5-chloromethyl-isoxazole. MS 496.0 (M+H⁺); H¹ NMR (DMSO-d₆): δ(ppm) 10.10 (s, 1H), 9.48 (s, 1H), 8.19-8.14 (m, 1H), 7.80-7.56 (m, 2H), 7.60-7.51 (m, 1H), 7.46-7.31 (m, 6H), 7.14-7.07 (m, 3H), 6.14 (s, 2H), 5.15 (s, 2H).

### Example 74

### 2-(2,3-Difluoro-phenyl)-5-[3-(4-methoxy-2-trifluoromethyl-phenyl)-isoxazol-5-ylmethyl]-5H-imidazo[4,5-d]pyridazine (Compound 174)

From 2-(2,3-difluoro-phenyl)-5H-imidazo[4,5-d]pyridazine and 5-chloromethyl-3-(4-methoxy-2-trifluoromethyl-phenyl)-isoxazole. MS 488.2 (M+H⁺); H¹ NMR (DMSO-d₆): δ(ppm) 10.58 (s, 1H), 9.8 (s, 1H), 8.18-8.14 (m, 1H), 7.75-7.73 (q, 1H), 7.58-7.56 (d, 1H), 7.50-7.44 (m, 1H), 7.37-7.31 (m, 2H), 6.95 (s, 1H), 6.38 (s, 2H), 3.87 (s, 3H).

### Example 75

### 2-(2,3-Difluoro-phenyl)-5-{3-[4-(1,1,2,2-tetrafluoro-ethoxy)-phenyl]-isoxazol-5-ylmethyl}-5H-imidazo[4,5-d]pyridazine (Compound 175)

From 2-(2,3-difluoro-phenyl)-5H-imidazo[4,5-d]pyridazine and 5-chloromethyl-3-[4-(1,1,2,2-tetrafluoro-ethoxy)-phenyl]-isoxazole. MS 506.1 (M+H⁺); H¹ NMR (DMSO-d₆): δ(ppm) 10.55 (s, 1H), 9.76 (s, 1H), 8.17-8.14 (m, 1H), 7.97-7.92 (m, 2H), 7.74-7.71 (m, 1H), 7.49-7.39 (m, 3H), 7.27 (s, 1H), 7.00-6.66 (t, 1H), 6.37 (s, 2H).

### Example 76

### 5-[3-(4-Difluoromethoxy-phenyl)-isoxazol-5-ylmethyl]-2-(2,3-difluoro-phenyl)-5H-imidazo[4,5-d]pyridazine (Compound 176)

From 2-(2,3-difluoro-phenyl)-5H-imidazo[4,5-d]pyridazine and 5-chloromethyl-3-(4-difluoromethoxy-phenyl)-isoxazole. MS 456.0 (M+H⁺); H¹ NMR (DMSO-d₆): δ(ppm) 10.40 (s, 1H), 9.72 (s, 1H), 8.17-8.13 (t, 1H), 7.91-7.88 (m, 2H), 7.75-7.66 (m, 1H), 7.49-7.41 (m, 1H), 7.30-7.23 (t, 3H), 7.58, 7.33, 7.09,(t, 1H), 6.31 (s, 2H).

### Example 77

### 2-(2,3-Difluoro-phenyl)-5-[3-(2-methyl-4-trifluoromethoxy-phenyl)-isoxazol-5-ylmethyl]-5H-imidazo[4,5-d]pyridazine (Compound 177)

### 2-Methyl-4-trifluoromethoxy-benzaldehyde

To a solution of 1-bromo-2-methyl-4-trifluoromethoxy-benzene (1.25 g, 5 mmol) in THF (20 mL) at −78° C. was added nBuLi (1.2 eq, 2.4 mL of 2.5M in hexanes). The mixture was stirred for 180 minutes and quenched with DMF (2 mL) and allowed to warm to room temperature. Solvents were removed, the reaction was washed with H₂O (10 mL) and the organics concentrated giving the crude product.

### 2-(2,3-Difluoro-phenyl)-5-[3-(2-methyl-4-trifluoromethoxy-phenyl)-isoxazol-5-ylmethyl]-5H-imidazo[4,5-d]pyridazine (Compound 177)

From 2-(2,3-difluoro-phenyl)-5H-imidazo[4,5-d]pyridazine and 5-chloromethyl-3-(2-methyl-4-trifluoromethoxy-phenyl)-isoxazole (General Procedure B, from 2-methyl-4-trifluoromethoxy-benzaldehyde). MS 488.0 (M+H⁺); H¹ NMR (DMSO-d₆): δ(ppm) 10.47 (s, 1H), 9.73 (s, 1H), 8.17-8.13 (m, 1H), 7.76-7.63 (m, 2H), 7.48-7.39 (m, 2H), 7.32-7.28 (d, 1H), 7.11 (s, 1H), 6.34 (s, 2H), 2.45 (s, 3H).

### Example 78

### 2-(2,3-Difluoro-phenyl)-5-{3-[4-(pyridin-3-yloxymethyl)-phenyl]-isoxazol-5-ylmethyl}-5H-imidazo[4,5-d]pyridazine (Compound 178)

From 2-(2,3-difluoro-phenyl)-5H-imidazo[4,5-d]pyridazine and 3-[4-(5-chloromethyl-isoxazol-3-yl)-benzyloxy]-pyridine. MS 497.2 (M+H⁺); H¹ NMR (DMSO-d₆): δ(ppm) 10.52 (s, 1H), 9.75 (s, 1H), 8.71-8.70 (d, 1H), 8.47-8.45 (d, 1H), 8.19-8.10 (m, 2H), 7.91-7.84 (m, 3H), 7.34-7.71 (m, 1H), 7.61-7.59 (d, 2H), 7.49-7.45 (m, 1H), 7.26 (s, 1H), 6.34 (s, 2H), 5.37 (s, 2H).

### Example 79

### 2-(2,3-Difluoro-phenyl)-5-{3-[4-(pyridin-3-ylmethoxy)-phenyl]-isoxazol-5-ylmethyl}-5H-imidazo[4,5-d]pyridazine (Compound 179)

From 2-(2,3-difluoro-phenyl)-5H-imidazo[4,5-d]pyridazine and 3-[4-(5-chloromethyl-isoxazol-3-yl)-phenoxymethyl]-pyridine. MS 497.2 (M+H⁺); H¹ NMR (DMSO-d₆): δ(ppm) 10.62 (s, 1H), 9.78 (s, 1H), 8.90 (s, 1H), 8.87-8.85 (d, 1H), 8.58-8.55 (d, 1H), 8.18-8.16 (d, 1H), 8.04-7.99 (t, 1H), 7.82-7.73 (m, 3H), 7.50-7.47 (m, 1H), 7.20-7.16 (m, 3H), 6.36 (s, 2H), 5.38 (s, 2H).

### Example 80

### 2-(2,3-Difluoro-phenyl)-5-[3-(4-methyl-thiazol-2-yl)-isoxazol-5-ylmethyl]-5H-imidazo[4,5-d]pyridazine (Compound 180)

From 2-(2,3-difluoro-phenyl)-5H-imidazo[4,5-d]pyridazine and 5-chloromethyl-3-(4-methyl-thiazol-2-yl)-isoxazole. MS 411.0 (M+H⁺); H¹ NMR (DMSO-d₆): δ(ppm) 10.41 (s, 1H), 9.71 (s, 1H), 8.17-8.12 (t, 1H), 7.74-7.69 (m, 1H), 7.53 (s, 1H), 7.45-7.41 (m, 1H), 7.22 (s, 1H), 6.33 (s, 2H), 2.42 (s, 3H).

### Example 81

### 2-(2,3-Difluoro-phenyl)-5-[3-(2-methyl-thiazol-4-yl)-isoxazol-5-ylmethyl]-5H-imidazo[4,5-d]pyridazine (Compound 181)

From 2-(2,3-difluoro-phenyl)-5H-imidazo[4,5-d]pyridazine and 5-chloromethyl-3-(2-methyl-thiazol-4-yl)-isoxazole. MS 411.0 (M+H⁺); H¹ NMR (DMSO-d₆): δ(ppm) 10.67 (s, 1H), 9.83 (s, 1H), 8.19-8.13 (m, 2H), 7.79-7.76 (m, 1H), 7.53-7.49 (m, 1H), 7.13 (s, 1H), 6.40 (s, 2H), 2.68 (s, 3H).

### Example 82

### 5-[3-(2-Butyl-5-chloro-1H-imidazol-4-yl)-isoxazol-5-ylmethyl]-2-(2,3-difluoro-phenyl)-5H-imidazo[4,5-d]pyridazine (Compound 182)

From 2-(2,3-difluoro-phenyl)-5H-imidazo[4,5-d]pyridazine and 3-(2-butyl-5-chloro-1H-imidazol-4-yl)-5-chloromethyl-isoxazole. MS 470.2 (M+H⁺); H¹ NMR (DMSO-d₆): δ(ppm) 10.59 (s, 1H), 9.80 (s, 1H), 8.18-8.13 (t, 1H), 7.80-7.71 (m, 1H), 7.52-7.45 (m, 1H), 7.21 (s, 1H), 6.37 (s, 2H), 2.64-2.59 (t, 2H), 1.63-1.55 (m, 2H), 1.30-1.21 (m, 2H), 0.87-0.83 (t, 3H).

### Example 83

### 5-[3-(2-Butyl-1H-imidazol-4-yl)-isoxazol-5-ylmethyl]-2-(2,3-difluoro-phenyl)-5H-imidazo[4,5-d]pyridazine (Compound 183)

From 2-(2,3-difluoro-phenyl)-5H-imidazo[4,5-d]pyridazine and 3-(2-butyl-1H-imidazol-4-yl)-5-chloromethyl-isoxazole. MS 436.2 (M+H⁺); H¹ NMR (DMSO-d₆): δ(ppm) 10.43 (s, 1H), 9.68 (s, 1H), 8.23 (s, 1H), 8.17-8.13 (m, 1H), 7.69-7.66 (m, 1H), 7.46-7.40 (m, 1H), 7.24 (s, 1H), 6.35 (s, 2H), 2.96-2.91 (m, 21-1), 1.75-1.67 (m, 2H), 1.30-1.21 (m, 2H), 0.89-0.83 (m, 3H).

### Example 84

### 2-(2,3-Difluoro-phenyl)-5-[3-(2-ethyl-5-methyl-1H-imidazol-4-yl)-isoxazol-5-ylmethyl]-5H-imidazo[4,5-d]pyridazine (Compound 184)

From 2-(2,3-difluoro-phenyl)-5H-imidazo[4,5-d]pyridazine and 5-chloromethyl-3-(2-ethyl-5-methyl-1H-imidazol-4-yl)-isox azole. MS 422.2 (M+H⁺); H¹ NMR (DMSO-d₆): δ(ppm) 10.50 (s, 1H), 9.71 (s, 1H), 8.18-8.14 (m, 1H), 7.71-7.68 (m, 1H), 7.47-7.42 (m, 1H), 7.36 (s, 1H), 6.38 (s, 2H), 2.96-2.87 (m, 2H), 2.45 (s, 3H), 1.33-1.28 (m, 3H).

### Example 85

### 2-(2,3-Difluoro-phenyl)-5-[3-(2,5-dimethyl-oxazol-4-yl)-isoxazol-5-ylmethyl]-5H-imidazo[4,5-d]pyridazine (Compound 185)

From 2-(2,3-difluoro-phenyl)-5H-imidazo[4,5-d]pyridazine and 5-chloromethyl-3-(2,5-dimethyl-oxazol-4-yl)-isoxazole. MS 409.1 (M+H⁺); H¹ NMR (DMSO-d₆): δ(ppm) 10.66 (s, 1H), 9.81 (s, 1H), 8.19-8.14 (m, 1H), 7.81-7.72 (m, 1H), 7.52-7.46 (m, 1H), 6.70 (s, 1H), 6.39 (s, 2H), 2.49 (s, 3H), 2.38 (s, 3H).

### Example 86

### 5-[3-(4-Butyl-2-trifluoromethyl-phenyl)-isoxazol-5-ylmethyl]-2-(2,3-difluoro-phenyl)-5H-imidazo[4,5-d]pyridazine (Compound 186)

### 4-Butyl-2-fluoro-benzaldehyde

4-Bromo-2-fluoro-benzaldehyde (Tetrahedron 61, 6590, 2005) (253 mg, 1.0 mmol), butaneboronic acid (165 mg, 1.6 mmol), potassium carbonate (1.0 mL, 2 M, 2.0 mmol), and toluene (2.0 mL) were combined in a vial and sparged with argon. Tetrakis(triphenylphosphine)palladium (58 mg, 0.05 mmol) was added, and the vial was sealed. The reaction was magnetically stirred at 100° C. overnight. The cooled reaction mixture was extracted with ether (3×4 mL), and the combined extract was concentrated onto celite. The product was isolated by silica gel flash chromatography (EtOAc in hexanes, 0-15%). The product was collected as colorless oil. Yield 165 mg, 72%.

### 5-[3-(4-Butyl-2-trifluoromethyl-phenyl)-isoxazol-5-ylmethyl]-2-(2,3-difluoro-phenyl)-5H-imidazo[4,5-d]pyridazine (Compound 186)

From 2-(2,3-difluoro-phenyl)-5H-imidazo[4,5-d]pyridazine and 3-(4-butyl-2-trifluoromethyl-phenyl)-5-chloromethyl-isoxazole (General Procedure B, from 4-butyl-2-fluoro-benzaldehyde). MS 514.3 (M+H⁺); H¹ NMR (DMSO-d₆): δ(ppm) 10.40 (s, 1H), 10.22 (s, 1H), 9.70 (s, 1H), 8.17-8.13 (m, 1H), 7.72-7.40 (m, 4H), 6.32 (s, 2H), 6.95 (s, 1H), 2.74-2.69 (t, 2H), 1.60-1.52 (m, 2H), 1.33-1.25 (m, 2H), 0.90-0.85 (t, 3H).

### Example 87

### 2-(2,3-Difluoro-phenyl)-5-(3-p-tolyl-isoxazol-5-ylmethyl)-5H-imidazo[4,5-d]pyridazine (Compound 187)

From 2-(2,3-difluoro-phenyl)-5H-imidazo[4,5-d]pyridazine and 5-chloromethyl-3-p-tolyl-isoxazole. MS 404.1 (M+H⁺); H¹ NMR (DMSO-d₆): δ(ppm) 10.53 (s, 1H), 9.75 (s, 1H), 8.18-8.14 (m, 1H), 7.28-7.70 (q, 3H), 7.49-7.42 (m, 1H), 7.30-7.28 (d, 2H), 7.19 (s, 1H), 6.33 (s, 2H), 2.32 (s, 3H).

### Example 88

### 2-(2,3-Difluoro-phenyl)-5-[3-(4-ethyl-phenyl)-isoxazol-5-ylmethyl]-5H-imidazo[4,5-d]pyridazine (Compound 188)

From 2-(2,3-difluoro-phenyl)-5H-imidazo[4,5-d]pyridazine and 5-chloromethyl-3-(4-ethyl-phenyl)-isoxazole. MS 418.1 (M+H⁺); H¹ NMR (DMSO-d₆): δ(ppm) 10.53 (s, 1H) 9.75 (s, 1H), 8.18-8.14 (m, 1H), 7.76-7.67 (m, 3H), 7.49-7.42 (m, 1H), 7.33-7.31 (d, 2H), 7.19 (s, 1H), 6.33 (s, 2H), 2.66-2.59 (q, 2H), 1.19-1.14 (t, 3H).

### Example 89

### 2-(2,3-Difluoro-phenyl)-5-[3-(4-propyl-phenyl)-isoxazol-5-ylmethyl]-5H-imidazo[4,5-d]pyridazine (Compound 189)

From 2-(2,3-difluoro-phenyl)-5H-imidazo[4,5-d]pyridazine and 5-chloromethyl-3-(4-propyl-phenyl)-isoxazole. MS 432.1 (M+H⁺); H¹ NMR (DMSO-d₆): δ(ppm) 10.51 (s, 1H), 9.74 (s, 1H), 8.18-8.14 (m, 1H), 7.74-7.67 (m, 3H), 7.46-7.42 (m, 1H), 7.31-7.28 (d, 2H), 7.19 (s, 1H), 6.32 (s, 2H), 2.60-2.46 (t, 2H), 1.61-1.54 (m, 2H), 0.89-0.84 (t, 3H).

### Example 90

### 2-(2,3-Difluoro-phenyl)-5-[3-(4-isobutyl-phenyl)-isoxazol-5-ylmethyl]-5H-imidazo[4,5-d]pyridazine (Compound 190)

From 2-(2,3-difluoro-phenyl)-5H-imidazo[4,5-d]pyridazine and 5-chloromethyl-3-(4-isobutyl-phenyl)-isoxazole. MS 446.1 (M+H⁺); H¹ NMR (DMSO-d₆): δ(ppm) 10.48 (s, 1H), 9.73 (s, 1H), 8.18-8.14 (m, 1H), 7.75-7.66 (m, 3H), 7.48-7.40 (m, 1H), 7.28-7.23 (d, 2H), 7.19 (s, 1H), 6.31 (s, 2H), 2.45 (m, 2H), 1.90-1.73 (m, 1H), 0.85-0.83 (d, 6H).

### Example 91

### 2-(2,3-Difluoro-phenyl)-5-[3-(4-pentyl-phenyl)-isoxazol-5-ylmethyl]-5H-imidazo[4,5-d]pyridazine (Compound 191)

From 2-(2,3-difluoro-phenyl)-5H-imidazo[4,5-d]pyridazine and 5-chloromethyl-3-(4-pentyl-phenyl)-isoxazole. MS 460.1 (M+H⁺); H¹ NMR (DMSO-d₆): δ(ppm) 10.54 (s, 1H), 9.76 (s, 1H), 8.18-8.15 (m, 1H), 7.74-7.67 (m, 3H), 7.48-7.42 (m, 1H), 7.31-7.28 (d, 2H), 7.19 (s, 1H), 6.34 (s, 2H), 2.61-2.50 (t, 2H), 1.58-1.51 (m, 2H), 1.29-1.20 (m, 4H), 0.85-0.80 (t, 3H).

### Example 92

### 4-(4-{5-[2-(2,3-Difluoro-phenyl)-imidazo[4,5-d]pyridazin-5-ylmethyl]-isoxazol-3-yl}-phenoxy)-butyric acid methyl ester (Compound 192)

A flask was charged with 4-hydroxybenzaldehyde (1.22 g, 10 mmol) and DMF (10 mL). The resulting solution was cooled in an ice bath and treated with NaH (380 mg, 60% in mineral oil, 9.5 mmol). After 5 min, methyl 4-bromobutyrate (1.4 mL, 11 mmol) was added dropwise. The reaction was treated with ultrasound for 15 min, and then stirred overnight at room temp. The reaction mixture was partitioned between ethyl acetate (250 mL) and water (100 mL). The organic layer was washed with water and brine. The organic layer was dried over sodium sulfate and concentrated onto celite. The product was isolated via SiO₂ flash chromatography using 0-5% methanol in dichloromethane to give an oil (1.4 g).

From 2-(2,3-difluoro-phenyl)-5H-imidazo[4,5-d]pyridazine and 3-(4-butyl-2-trifluoromethyl-phenyl)-5-chloromethyl-isoxazole (General Procedure B, from 4-(4-Formyl-phenoxy)-butyric acid methyl ester. MS 506.1 (M+H⁺); H¹ NMR (DMSO-d₆): δ(ppm) 10.09 (d, 1H), 9.48 (d, 1H), 8.14-8.19 (m, 1H), 7.77 (d, 2H), 7.52-7.60 (m, 1H), 7.31-7.37 (m, 1H), 7.12 (s, 1H), 7.01 (d, 2H), 6.14 (s, 2H), 4.03 (t, 2H), 3.59 (s, 3H), 2.44-2.50 (m, 2H), 1.97 (quintet, 2H).

### Example 93

### 3-(4-{5-[2-(2,3-Difluoro-phenyl)-imidazo[4,5-d]pyridazin-5-ylmethyl]-isoxazol-3-yl}-phenoxy)-propan-1-ol (Compound 193)

A flask was charged with 4-hydroxybenzaldehyde (0.96 g, 7.9 mmol) and DMF (10 mL). The resulting solution was cooled in an ice bath and treated with NaH (350 mg, 60% in mineral oil, 8.7 mmol). After 5 min, (3-Bromopropoxy)-tert-butyldimethylsilane (2.0 mL, 8.7 mmol) was added dropwise. The reaction was treated with ultrasound for 15 min, and then stirred overnight at room temp. The reaction mixture was partitioned between ethyl acetate (250 mL) and water (100 mL). The organic layer was washed with water and brine. The organic layer was dried over sodium sulfate and concentrated to give the crude TBS-alcohol. The TBS-alcohol was suspended in 120 mL of a 1:1 mixture of acetonitrile and 1 N HCl. The reaction was stirred at room temp for 1.5 h, and then the solvents were removed in vacuo. The residue was adsorbed onto celite and purified via SiO₂ flash chromatography using 1:1 hexanes:ethyl acetate to give the product alcohol (1.0 g) as a colorless oil.

From 2-(2,3-difluoro-phenyl)-5H-imidazo[4,5-d]pyridazine and 3-(4-butyl-2-trifluoromethyl-phenyl)-5-chloromethyl-isoxazole (General Procedure B, from 4-(3-Hydroxy-phenoxy)-benzaldehyde. MS 464.0 (M+H⁺); H¹ NMR (DMSO-d₆): δ(ppm) 10.08 (d, 1H), 9.47 (d, 1H), 8.13-8.18 (m, 1H), 7.76 (d, 2H), 7.51-7.60 (m, 1H), 7.31-7.38 (m, 1H), 7.12 (s, 1H), 7.02 (d, 2H), 6.14 (s, 2H), 4.56 (t, 1H), 4.07 (t, 2H), 3.55 (q, 2H), 1.86 (m, 2H).

### Example 94

### 2-(2,3-Difluoro-phenyl)-5-{3-[4-(4-methyl-piperazin-1-yl)-phenyl]-isoxazol-5-ylmethyl}-5H-imidazo[4,5-d]pyridazine (Compound 194)

From 2-(2,3-difluoro-phenyl)-5H-imidazo[4,5-d]pyridazine and 1-[4-(5-chloromethyl-isoxazol-3-yl)-phenyl]-4-methyl-piperazine. MS: 488.1 (M+H⁺); H¹-NMR (DMSO-d₆): δ(ppm) 11.0 (bs, 1H), 10.5 (s, 1H), 9.7 (s, 1H), 8.1-8.2 (m, 1H), 7.7 (m, 3H), 7.4 (m, 1H), 7.1 (s, 1H), 7.1 (d, 2H), 6.3 (s, 2H), 3.9 (d, 2H), 3.4 (d, 2H), 3.0-3.2 (m, 4H), 2.8 (d, 3H).

### Example 95

### 2-(2,3-Difluoro-phenyl)-5-{3-[4-(4-methyl-piperazin-1-yl)-phenyl]-isoxazol-5-ylmethyl}-5H-imidazo[4,5-d]pyridazine (Compound 195)

From 2-(2,3-difluoro-phenyl)-5H-imidazo[4,5-d]pyridazine and 5-chloromethyl-3-[4-(2-methoxy-ethoxy)-phenyl]-isoxazole. MS: 464.2 (M+H⁺); H¹-NMR (DMSO-d₆): δ(ppm) 10.3 (s, 1H), 9.7 (s, 1H), 8.1-8.2 (m, 1H), 7.6-7.8 (m, 3H), 7.4 (m, 1H), 7.2 (s, 1H), 7.0 (d, 2H), 6.3 (s, 2H), 4.2 (m, 2H), 3.6 (m, 2H), 3.3 (s, 1H).

### Example 96

### 2-(2,3-Difluoro-phenyl)-5-{3-[4-(2-morpholin-4-yl-ethoxy)-phenyl]-isoxazol-5-ylmethyl}-5H-imidazo[4,5-d]pyridazine (Compound 196)

From 2-(2,3-difluoro-phenyl)-5H-imidazo[4,5-d]pyridazine and 4-{2-[4-(5-chloromethyl-isoxazol-3-yl)-phenoxy]-ethyl}-morpholine. MS: 519.2 (M+H⁺); H¹-NMR (DMSO-d₆): δ(ppm) 10.4 (s, 1H), 9.7 (s, 1H), 8.1-8.2 (m, 1H), 7.8 (d, 2H), 7.7 (m, 1H), 7.4 (m, 1H), 7.2 (s, 1H), 7.0 (d, 2H), 6.3 (s, 2H), 4.4 (m, 2H), 3.9 (m, 2H), 3.8 (m, 2H), 3.4-3.6 (m, 4H), 3.2 (m, 2H).

### Example 97

### 5-{5-[2-(2,3-Difluoro-phenyl)-imidazo[4,5-d]pyridazin-5-ylmethyl]-isoxazol-3-yl}-2-propoxy-benzoic acid propyl ester (Compound 197)

From 2-(2,3-difluoro-phenyl)-5H-imidazo[4,5-d]pyridazine and 5-(5-chloromethyl-isoxazol-3-yl)-2-propoxy-benzoic acid propyl ester. MS: 534.2 (M+H⁺); H¹ -NMR (DMSO-d₆): δ(ppm) 10.3 (s, 1H), 9.7 (s, 1H), 8.1-8.2 (m, 1H), 8.1 (s, 1H), 7.9-8.0 (m, 1H), 7.7 (m, 1H), 7.4 (m, 1H), 7.2 (m, 2H), 7.0 (d, 2H), 6.3 (s, 2H), 4.2 (tr, 2H), 4.0 (tr, 2H), 1.6-1,8 (m, 4H), 3.4-3.6 (m, 4H), 0.9-1.0 (m, 6H).

### Example 98

### 2-{5-[2-(2,3-Difluoro-phenyl)-imidazo[4,5-d]pyridazin-5-ylmethyl]-isoxazol-3-yl}-5-methoxy-benzoic acid methyl ester (Compound 198)

From 2-(2,3-difluoro-phenyl)-5H-imidazo[4,5-d]pyridazine and

5-(5-chloromethyl-isoxazol-3-yl)-2-methoxy-benzoic acid methyl ester. MS: 478.1 (M+H⁺); H¹-NMR (DMSO-d₆): δ(ppm) 10.3 (s, 1H), 9.6 (s, 1H), 8.2 (m, 1H), 7.6-7.7 (m, 2H), 7.4 (m, 1H), 7.3 (d, 1H), 7.2 (dd, 1H), 6.9 (s, 1H), 6.3 (s, 2H), 3.8 (s, 3H), 3.7 (s, 3H).

### Example 99

### 2-(2,3-Difluoro-phenyl)-5-[3-(4-nitro-phenyl)-isoxazol-5-ylmethyl]-5H-imidazo[4,5-d]pyridazine (Compound 199)

From 2-(2,3-difluoro-phenyl)-5H-imidazo[4,5-d]pyridazine and 5-chloromethyl-3-(4-nitro-phenyl)-isoxazole. MS: 435.1 (M+H⁺); H¹-NMR (DMSO-d₆):δ(ppm) 10.2 (s, 1H), 9.6 (s, 1H), 8.8 (m, 1H), 8.3 (d, 2H), 8.1 (d, 2H), 7.8 (m, 1H), 7.6 (m, 1H), 7.4 (m, 2H), 6.3 (s, 2H).

### Example 100

### 5-[3-(4-Bromo-phenyl)-isoxazol-5-ylmethyl]-2-(2,3-difluoro-phenyl)-5H-imidazo[4,5-d]pyridazine (Compound 200)

From 2-(2,3-difluoro-phenyl)-5H-imidazo[4,5-d]pyridazine and 3-(4-bromo-phenyl)-5-chloromethyl-isoxazole. MS: 468.0 (M+H⁺); H¹-NMR (DMSO-d₆): δ(ppm) 10.3 (d, 1H), 9.6 (d, 1H), 8.1 (m, 1H), 7.8 (d, 2H), 7.7 (d, 2H), 7.6 (m, 1H), 7.5 (m, 1H), 7.2 (s, 1H), 6.2 (s, 2H).

### Example 101

### 5-[3-(4-Butyl-phenyl)-isoxazol-5-ylmethyl]-2-(2,3-difluoro-phenyl)-5H-imidazo[4,5-d]pyridazine (Compound 201)

From 2-(2,3-difluoro-phenyl)-5H-imidazo[4,5-d]pyridazine and 3-(4-butyl-phenyl)-5-chloromethyl-isoxazole. MS: 446.1 (M+H⁺); H¹ NMR (DMSO-d₆): δ(ppm) 10.26 (s, 1H), 9.60 (s, 1H), 8.11-8.20 (m, 1H), 7.71-7.77 (m, 2H), 7.55-7.69 (m, 1H), 7.27-7.45 (m, 3H), 7.18 (m, 1H), 6.23 (s, 2H), 2.62 (t, 2H), 1.48-1.63 (m, 2H), 1.21-1.36 (m, 2H), 0.90 (t, 3H).

### Example 102

### 2-(2,3-Difluoro-phenyl)-5-[3-(4-trifluoromethyl-phenyl)-isoxazol-5-ylmethyl]-5H-imidazo[4,5-d]pyridazine (Compound 202)

From 2-(2,3-difluoro-phenyl)-5H-imidazo[4,5-d]pyridazine and 5-chloromethyl-3-(4-trifluoromethyl-phenyl)-isoxazoleMS: 458.1 (M+H⁺); H¹ NMR (DMSO-d₆): δ(ppm) 10.46 (s, 1H), 9.73 (s, 1H), 8.04-8.20 (m, 3H), 7.84-7.91 (m, 2H), 7.64-7.77 (m, 1H), 7.34-7.50 (m, 2H), 6.35 (s, 1H).

### Example 103

### 2-(2,3-Difluoro-phenyl)-5-[3-(2-fluoro-4-trifluoromethyl-phenyl)-isoxazol-5-ylmethyl]-5H-imidazo[4,5-d]pyridazine (Compound 203)

From 2-(2,3-difluoro-phenyl)-5H-imidazo[4,5-d]pyridazine and 5-chloromethyl-3-(2-fluoro-4-trifluoromethyl-phenyl)-isoxazole. MS: 476.1 (M+H⁺); H¹ NMR (DMSO-d₆): δ(ppm) 10.39 (s, 1H), 9.68 (s, 1H), 8.07-8.20 (m, 2H), 7.90-7.98 (m, 1H), 7.62-7.77 (m, 2H), 7.38-7.48 (m, 1H), 7.24-7.29 (m, 1H), 6.34 (s, 1H).

### Example 104

### 2-(2,3-Difluoro-phenyl)-5-[3-(3-fluoro-pyridin-4-yl)-isoxazol-5-ylmethyl]-5H-imidazo[4,5-d]pyridazine (Compound 204)

From 2-(2,3-difluoro-phenyl)-5H-imidazo[4,5-d]pyridazine and 4-(5-chloromethyl-isoxazol-3-yl)-3-fluoro-pyridine. MS: 409.1 (M+H⁺); H¹ NMR (DMSO-d₆): δ(ppm) 10.60 (s, 1H), 9.81 (s, 1H), 8.78-8.83 (m, 1H), 8.55-8.61 (m, 1H), 8.12-8.20 (m, 1H), 7.87-7.95 (m, 1H), 7.73-7.82 (m, 1H), 7.45-7.55 (m, 1H), 7.31-7.36 (m, 1H), 6.44 (s, 2H).

### Example 105

### 2-(2,3-Difluoro-phenyl)-5-[3-(6-trifluoromethyl-pyridin-3-yl)-isoxazol-5-ylmethyl]-5H-imidazo[4,5-d]pyridazine (Compound 205)

From 2-(2,3-difluoro-phenyl)-5H-imidazo[4,5-d]pyridazine and 5-(5-chloromethyl-isoxazol-3-yl)-2-trifluoromethyl-pyridine. MS: 459.1 (M+H⁺); H¹ NMR (DMSO-d₆): δ(ppm) 10.24 (s, 1H), 9.58 (s, 1H), 9324 (s, 1H), 8.50-8.57 (m, 1H), 8.11-8.19 (m, 1H), 8.02-8.09 (m, 1H), 7.56-7.68 (m, 1H), 7.34-7.44 (m, 2H), 6.29 (s, 2H).

### Example 106

### 2-(2,3-Difluoro-phenyl)-5-[3-(3-fluoro-4-trifluoromethyl-phenyl)-isoxazol-5-ylmethyl]-5H-imidazo[4,5-d]pyridazine (Compound 206)

From 2-(2,3-difluoro-phenyl)-5H-imidazo[4,5-d]pyridazine and 5-chloromethyl-3-(3-fluoro-4-trifluoromethyl-phenyl)-isoxazole. MS: 476.1 (M+H⁺); H¹ NMR (DMSO-d₆): δ(ppm) 10.18 (s, 1H), 9.56 (s, 1H), 8.10-8.20 (m, 1H), 7.88-8.07 (m, 3H), 7.55-7.65 (m, 1H), 7.30-7.42 (m, 2H), 6.25 (s, 2H).

### Example 107

### 2-(2,3-Difluoro-phenyl)-5-{3-[4-(3-fluoro-propoxy)-phenyl]-isoxazol-5-ylmethyl}-5H-imidazo[4,5-d]pyridazine (Compound 207)

From 2-(2,3-difluoro-phenyl)-5H-imidazo[4,5-d]pyridazine and 5-chloromethyl-3-[4-(3-fluoro-propoxy)-phenyl]-isoxazole. MS: 466.1 (M+H⁺); H¹ NMR (DMSO-d₆): δ(ppm) 10.17 (s, 1H), 9.54 (s, 1H), 8.11-8.19 (s, 1H), 7.73-7.81 (m, 2H), 7.53-7.66 (m, 1H), 7.32-7.43 (m, 1H), 7.14 (s, 1H), 7.00-7.09 (m, 2H), 6.18 9s, 2H), 4.68 (t, 1H), 4.53 (t, 1H), 4.12 (t, 2H), 2.01-2.22 (m, 2H).

### Example 108

### (4-{5-[2-(2,3-Difluoro-phenyl)-imidazo[4,5-d]pyridazin-5-ylmethyl]-isoxazol-3-yl}-phenyl)-dimethyl-amine (Compound 208)

From 2-(2,3-difluoro-phenyl)-5H-imidazo[4,5-d]pyridazine and [4-(5-chloromethyl-isoxazol-3-yl)-phenyl]-dimethyl-amine. MS: 433.2 (M+H⁺); H¹ NMR (DMSO-d₆): δ(ppm) 10.21 (s, 1H), 9.57 (s, 1H), 8.11-8.19 (m, 1H), 7.60-7.68 (m, 3H), 7.34-7.44 (m, 1H), 7.07 (s, 1H), 6.71-6.79 (m, 2H), 6.17 (s, 2H), 2.96 (s, 6H).

### Example 109

### 4-{5-[2-(2,3-Difluoro-phenyl)-imidazo[4,5-d]pyridazin-5-ylmethyl]-isoxazol-3-yl}-benzoic acid methyl ester (Compound 209)

From 2-(2,3-difluoro-phenyl)-5H-imidazo[4,5-d]pyridazine and 4-(5-chloromethyl-isoxazol-3-yl)-benzoic acid methyl ester. MS: 448.1 (M+H⁺); H¹ NMR (DMSO-d₆): δ(ppm) 10.12 (s, 1H), 9.51 (s, 1H), 8.11-8.20 (m, 1H), 7.77-7.84 (m, 1H), 7.52-7.73 (m, 4H), 7.30-7.40 (m, 1H), 6.94 (s, 1H), 6.18 (s, 2H), 3.68 (s, 3H).

### Example 110

### 3-{5-[2-(2,3-Difluoro-phenyl)-imidazo[4,5-d]pyridazin-5-ylmethyl]-isoxazol-3-yl}-benzoic acid methyl ester (Compound 210)

From 2-(2,3-difluoro-phenyl)-5H-imidazo[4,5-d]pyridazine and 3-(5-chloromethyl-isoxazol-3-yl)-benzoic acid methyl ester. MS: 448.1 (M+H⁺); H¹ NMR (DMSO-d₆): δ(ppm) 10.43 (s, 1H), 9.71 (s, 1H), 8.34-8.39 (m, 1H), 8.02-8.20 (m, 3H), 7.64-7.74 (m, 2H), 7.39-7.50 (m, 1H), 7.36 (s, 1H), 6.33 (s, 2H), 3.88 (s, 3H).

### Example 111

### 2-{5-[2-(2,3-Difluoro-phenyl)-imidazo[4,5-d]pyridazin-5-ylmethyl]-isoxazol-3-yl}-benzoic acid methyl ester (Compound 211)

From 2-(2,3-difluoro-phenyl)-5H-imidazo[4,5-d]pyridazine and 2-(5-chloromethyl-isoxazol-3-yl)-benzoic acid methyl ester. MS: 448.1 (M+H⁺); H¹ NMR (DMSO-d₆): δ(ppm) 10.26 (s, 1H), 9.62 (s, 1H), 8.12-8.18 (m, 1H), 7.77-7.84 (m, 1H), 7.57-7.72 (m, 4H), 7.35-7.44 (m, 1H), 6.95 (s, 1H), 6.24 (s, 2H), 6.69 (s, 3H).

### Example 112

### 3-{5-[2-(2,3-Difluoro-phenyl)-imidazo[4,5-d]pyridazin-5-ylmethyl]-isoxazol-3-yl}-benzonitrile (Compound 212)

From 2-(2,3-difluoro-phenyl)-5H-imidazo[4,5-d]pyridazine and 3-(5-chloromethyl-isoxazol-3-yl)-benzonitrile. MS: 415.1 (M+H⁺); H¹ NMR (DMSO-d₆): δ(ppm) 10.38 (s, 1H), 9.69 (s, 1H), 8.29-8.34 (m, 1H), 8.11-8.23 (m, 2H), 7.94-8.01 (m, 1H), 7.62-7.76 (m, 2H), 7.38-7.66 (m, 1H), 7.32 (s, 1H), 6.33 (s, 2H).

### Example 113

### 4-{5-[2-(2,3-Difluoro-phenyl)-imidazo[4,5-d]pyridazin-5-ylmethyl]-isoxazol-3-yl}-benzonitrile (Compound 213)

From 2-(2,3-difluoro-phenyl)-5H-imidazo[4,5-d]pyridazine and 4-(5-chloromethyl-isoxazol-3-yl)-benzonitrile. MS: 415.1 (M+H⁺); H¹ NMR (DMSO-d₆): δ(ppm) 10.23 (s, 1H), 9.58 (s, 1H), 8.12-8.18 (m, 1H), 7.94-8.09 (m, 4H), 7.56-7.68 (m, 1H), 7.31-7.44 (m, 2H), 6.26 (s, 2H).

### Example 114

### 2-(2,3-Difluoro-phenyl)-5-[3-(4-trifluoromethoxy-phenyl)-isoxazol-5-ylmethyl]-5H-imidazo[4,5-d]pyridazine (Compound 214)

From 2-(2,3-difluoro-phenyl)-5H-imidazo[4,5-d]pyridazine and 5-chloromethyl-3-(4-trifluoromethoxy-phenyl)-isoxazole. MS: 474.1 (M+H⁺); H¹ NMR (DMSO-d₆): δ(ppm) 10.21 (s, 1H), 9.57 (s, 1H), 8.11-8.19 (m, 1H), 7.95-8.02 (m, 2H), 7.34-7.67 (m, 4H), 7.25 (s, 1H), 6.24 (s, 2H).

### Example 115

### (4-{5-[2-(2,3-Difluoro-phenyl)-imidazo[4,5-d]pyridazin-5-ylmethyl]-isoxazol-3-yl}-phenoxy)-acetic acid methyl ester (Compound 215)

From 2-(2,3-difluoro-phenyl)-5H-imidazo[4,5-d]pyridazine and [4-(5-chloromethyl-isoxazol-3-yl)-phenoxy]-acetic acid methyl ester. MS: 478.1 (M+H⁺); H¹ NMR (DMSO-d₆): δ(ppm) 10.09 (s, 1H), 9.47 (s, 1H), 8.11-8.20 (m, 1H), 7.74-7.81 (m, 2H), 7.48-7.61 (m, 1H), 7.29-7.40 (m, 1H), 7.13 (s, 1H), 7.00-7.06 (m, 2H), 6.07 (s, 2H), 4.69 (s, 2H), 3.70 (s, 3H).

### Example 116

### [3-(4-{5-[2-(2,3-Difluoro-phenyl)-imidazo[4,5-d]pyridazin-5-ylmethyl]-isoxazol-3-yl}-phenoxy)-propyl]-dimethyl-amine (Compound 216)

From 2-(2,3-difluoro-phenyl)-5H-imidazo[4,5-d]pyridazine and {3-[4-(5-chloromethyl-isoxazol-3-yl)-phenoxy]-propyl}-dimethyl-amine. MS: 491.2 (M+H⁺); H¹ NMR (DMSO-d₆): δ(ppm) 10.18 (s, 1H), 9.54 (s, 1H), 8.11-8.20 (m, 1H), 7.76-7.83 (m, 2H), 7.52-7.68 (m, 1H), 7.33-7.43 (m, 1H), 7.14 (s, 1H), 7.00-7.07 (m, 2H), 6.19 (s, 2H), 4.10 (t, 2H), 3.15-3.26 (m, 2H), 7.76-2.82 (m, 6H), 2.06-2.18 (m, 2H).

### Example 117

### 2-(2,3-Difluoro-phenyl)-5-[3-(4-pyridin-2-ylmethyl-phenyl)-isoxazol-5-ylmethyl]-5H-imidazo[4,5-d]pyridazine (Compound 217)

From 2-(2,3-difluoro-phenyl)-5H-imidazo[4,5-d]pyridazine and 2-[4-(5-chloromethyl-isoxazol-3-yl)-benzyl]-pyridine. MS: 483.1 (M+H⁺); H¹ NMR (DMSO-d₆): δ(ppm) 10.70 (s, 1H), 9.88 9s, 1H), 8.11-8.20 (m, 2H), 7.73-7.92 (m, 4H), 7.46-7.57 (m, 1H), 7.04-7.50 (m, 5H), 6.42 (s, 2H).

### Example 118

### (4-{5-[2-(2,3-Difluoro-phenyl)-imidazo[4,5-d]pyridazin-5-ylmethyl]-isoxazol-3-yl}-benzyl)-dimethyl-amine (Compound 218)

From 2-(2,3-difluoro-phenyl)-5H-imidazo[4,5-d]pyridazine and [4-(5-chloromethyl-isoxazol-3-yl)-benzyl]-dimethyl-amine. Additional purification by silica gel chromatography gave the desired product. MS: 447.1 (M+H⁺); H¹ NMR (DMSO-d₆): δ(ppm) 10.09 (s, 1H), 9.48 (s, 1H), 8.11-8.20 (m, 1H), 7.77-7.84 (m, 2H), 7.48-7.62 (m, 1H), 7.29-7.44 (m, 3H), 7.18 (s, 1H), 6.17 (s, 2H), 3.50 (s, 2H), 2.20 (s, 6H).

### Example 119

### 2-(2,3-Difluoro-phenyl)-5-[3-(4-pyrrolidin-1-ylmethyl-phenyl)-isoxazol-5-ylmethyl]-5H-imidazo[4,5-d]pyridazine (Compound 219)

From 2-(2,3-difluoro-phenyl)-5H-imidazo[4,5-d]pyridazine and 5-chloromethyl-3-(4-pyrrolidin-1-ylmethyl-phenyl)-isoxazole. MS: 473.2 (M+H⁺); H¹ NMR (DMSO-d₆): δ(ppm) 10.44 (s, 1H), 9.70 (s, 1H), 8.12-8.20 (m, 1H), 7.88-7.94 (m, 2H), 7.63-7.76 (m, 3H), 7.39-7.50 (m, 1H), 7.28 (s, 1H), 6.33 (s, 2H), 4.34-4.40 (m, 2H), 3.26-3.40 (m, 1H), 2.96-3.10 (m, 1H), 1.82-2.05 (m, 4H).

### Example 120

### 2-(2,3-Difluoro-phenyl)-5-[3-(4-ethoxy-phenyl)-isoxazol-5-ylmethyl]-5H-imidazo[4,5-d]pyridazine (Compound 220)

From 2-(2,3-difluoro-phenyl)-5H-imidazo[4,5-d]pyridazine and 5-chloromethyl-3-(4-ethoxy-phenyl)-isoxazole. MS: 434.1 (M+H⁺); H¹ NMR (DMSO-d₆): δ(ppm) 10.42 (s, 1H), 9.71 (s, 1H), 8.13-8.22 (m, 1H), 7.64-7.81 (m, 3H), 7.40-7.51 (m, 1H), 7.17 (s, 1H), 6.97-7.06 (m, 2H), 6.28 (s, 2H), 4.07 (q, 2H), 1.33 (t, 3H).

### Example 121

### 2-(2,3-Difluoro-phenyl)-5-[3-(4-methoxy-phenyl)-isoxazol-5-ylmethyl]-5H-imidazo[4,5-d]pyridazine (Compound 221)

From 2-(2,3-difluoro-phenyl)-5H-imidazo[4,5-d]pyridazine and 5-chloromethyl-3-(4-methoxy-phenyl)-isoxazole. MS: 420.2 (M+H⁺); H¹ NMR (DMSO-d₆): δ(ppm) 10.48 (s, 1H), 9.74 (s, 1H), 8.13-8.22 (m, 1H), 7.66-7.84 (m, 3H), 7.41-7.52 (m, 1H), 7.18 (s, 1H), 7.00-7.09 (m, 2H), 6.31 (s, 2H), 3.80 (s, 3H).

### Example 122

### 5-[3-(4-Butoxy-phenyl)-isoxazol-5-ylmethyl]-2-(2,3-difluoro-phenyl)-5H-imidazo[4,5-d]pyridazine (Compound 222)

From 2-(2,3-difluoro-phenyl)-5H-imidazo[4,5-d]pyridazine and 3-(4-butoxy-phenyl)-5-chloromethyl-isoxazole. MS: 462.3 (M+H⁺); H¹ NMR (DMSO-d₆): δ(ppm) 10.43 (s, 1H), 9.72 (s, 1H), 8.13-8.21 (m, 1H), 7.64-7.81 (m, 3H), 7.40-7.53 (m, 1H), 7.17 (s, 1H), 7.00-7.07 (m, 2H), 6.29 (s, 2H), 4.02 (t, 2H), 1.63-1.76 (m, 2H), 1.34-1.51 (m, 2H), 0.93 (t, 3H).

### Example 123

### 5-[3-(2,4-Bis-trifluoromethyl-phenyl)-isoxazol-5-ylmethyl]-2-phenyl-5H-imidazo[4,5-d]pyridazine (Compound 223)

From 3-(2,4-bis-trifluoromethyl-phenyl)-5-chloromethyl-isoxazole and 2-phenyl-5H-imidazo[4,5-d]pyridazine. MS 490 (M+H⁺); H¹ NMR (DMSO-d₆): δ(ppm) 10.45 (s, 1H), 9.74 (s, 1H), 8.4 (m, 2H), 8.2 (m, 2H), 7.91 (m, 1H), 7.65 (s, 3H), 7.03 (s, 1H), 6.38 (s, 2H).

### Example 124

### 2-Phenyl-5-[3-(4-propoxy-phenyl)-isoxazol-5-ylmethyl]-5H-imidazo[4,5-d]pyridazine (Compound 224)

From 3 5-chloromethyl-3-(4-propoxy-phenyl)-isoxazole and 2-phenyl-5H-imidazo[4,5-d]pyridazine. MS 412.1 (M+H⁺); H¹ NMR (DMSO-d₆): δ(ppm) 10.37 (s, 1H), 9.69 (s, 1H), 8.4 (m, 2H), 7.76 (m, 2H), 7.63 (m, 3H), 7.15 (s, 1H), 7.03 (d, 2H), 6.26 (s, 2H), 3.98 (t, 2H), 1.74 (m, 2H), 0.99 (q, 3H).

### Example 125

### 5-[3-(4-Butoxy-phenyl)-isoxazol-5-ylmethyl]-2-phenyl-5H-imidazo[4,5-d]pyridazine (Compound 225)

From 3-(4-butoxy-phenyl)-5-chloromethyl-isoxazole and 2-phenyl-5H-imidazo[4,5-d]pyridazine. MS 426.1 (M+H⁺); H¹ NMR (DMSO-d₆): δ(ppm) 10.23 (s, 1H), 9.57 (s, 1H), 8.34 (m, 2H), 7.7 (m, 2H), 7.54 (m, 3H), 7.08 (s, 1H), 6.97 (m, 2H), 6.17 (s, 2H), 3.94 (t, 2H), 1.63 (m, 2H), 1.37 (m, 2H), 0.88 (t, 3H).

### Example 126

### 5-{1-[3-(2,4-Bis-trifluoromethyl-phenyl)-isoxazol-5-yl]-ethyl}-2-(2,3-difluoro-phenyl)-5H-imidazo[4,5-d]pyridazine (Compound 226)

From 2-(2,3-Difluoro-phenyl)-5H-imidazo[4,5-d]pyridazine and methanesulfonic acid 1-[3-(2,4-bis-trifluoromethyl-phenyl)-isoxazol-5-yl]-ethyl ester. MS 540.1 (M+H¹); H¹ NMR (DMSO-d₆): δ(ppm) 10.33 (s, 1H), 9.65 (s, 1H), 8.11-8.26 (m, 3H), 7.90 (m, 1H), 7.6 (m, 1H), 7.40 (m, 1H), 7.01 (s, 1H), 6.64 (q, 1H), 2.1 (d, 3H).

### Example 127

### 5-{1-[3-(2,4-Bis-trifluoromethyl-phenyl)-isoxazol-5-yl]-1-methyl-ethyl}-2-(2,3-difluoro-phenyl)-5H-imidazo[4,5-d]pyridazine (Compound 227)

From 2-(2,3-Difluoro-phenyl)-5H-imidazo[4,5-d]pyridazine and 3-(2,4-Bis-trifluoromethyl-phenyl)-5-(1-chloro-1-methyl-ethyl)-isoxazole. MS 554.1 (M+H⁺); H¹ NMR (DMSO-d₆): δ(ppm) 10.44 (s, 1H), 9.69 (s, 1H), 8.24 (m, 2H), 8.10 (m, 2H), 7.99 (m, 1H), 7.71 (m, 1H), 7.44 (m, 1H), 7.12 (s, 1H), 2.3 (s, 6H).

### Example 128

### 2-(2,3-Difluoro-phenyl)-5-[3-(4-methoxy-phenyl)-[1,2,4]oxadiazol-5-ylmethyl]-5H-imidazo[4,5-d]pyridazine (Compound 228)

From 2-(2,3-difluoro-phenyl)-5H-imidazo[4,5-d]pyridazine and 5-chloromethyl-3-(4-methoxy-phenyl)-[1,2,4]oxadiazole. MS 421.1 (M+H⁺); H¹ NMR (DMSO-d₆): δ(ppm) 10.11 (d, 1H), 9.50 (d, 1H), 8.14-8.20 (m, 1H), 7.87 (d, 2H), 7.51-7.60 (m, 1H), 7.31-7.38 (m, 1H), 7.06 (d, 2H), 6.40 (s, 1H), 3.80 (s, 3H).

### Example 129

### 2-(2,3-Difluoro-phenyl)-5-[5-(4-methoxy-phenyl)-[1,3,4]oxadiazol-2-ylmethyl]-5H-imidazo[4,5-d]pyridazine (Compound 229)

From 2-(2,3-difluoro-phenyl)-5H-imidazo[4,5-d]pyridazine and 2-chloromethyl-5-(4-methoxy-phenyl)-[1,3,4]oxadiazole. MS 421.1 (M+H⁺); H¹ NMR (DMSO-d₆): δ(ppm) 10.10 (d, 1H), 9.47 (d, 1H), 8.14-8.19 (m, 1H), 7.91 (d, 2H), 7.52-7.60 (m, 1H), 7.31-7.37 (m, 1H), 7.11 (d, 2H), 6.30 (s, 1H), 3.82 (s, 3H).

### Example 130

### 2-(2,3-Difluoro-phenyl)-5-[5-(4-trifluoromethyl-phenyl)-[1,2,4]oxadiazol-3-ylmethyl]-5H-imidazo[4,5-d]pyridazine (Compound 230)

From 2-(2,3-difluoro-phenyl)-5H-imidazo[4,5-d]pyridazine and 3-chloromethyl-5-(4-trifluoromethyl-phenyl)-[1,2,4]oxadiazole. MS 458.9 (M+H⁺); H¹ NMR (DMSO-d₆): δ(ppm) 10.13 (d, 1H), 9.48 (d, 1H), 8.28 (d, 2H), 8.14-8.19 (m, 1H), 7.96 (d, 2H), 7.51-7.59 (m, 1H), 7.32-7.37 (m, 1H), 6.24 (s, 2H).

### Example 131

### 5-[3-(2,4-Bis-trifluoromethyl-phenyl)-isoxazol-5-ylmethyl]-2-pyridin-2-yl-5H-imidazo[4,5-d]pyridazine (Compound 231)

### 2-(1H-Imidazol-2-yl)-pyridine

A round bottom flask was charged with 2-pyridinecarboxaldehyde (5.0 g), glyoxal (10.7 mL, 40% in water), and methanol (100 mL). This mixture was stirred at room temp as 26 mL of concentrated aqueous ammonia was added portion-wise. After 1 h, the solvents were removed, and the remaining brown residue was recrystallized in acetonitrile (ca. 40 mL). The product 2-(1H-Imidazol-2-yl)-pyridine was collected as brown crystals.

### 2-Pyridin-2-yl-5H-imidazo[4,5-d]pyridazine

A portion of 2-(1H-Imidazol-2-yl)-pyridine (61 mg, 0.42 mmol) and dimethyl 1,2,4,5-tetrazine-3,6-dicarboxylate (165 mg, 0.84 mmol) were ground together and heated slowly with a heat gun (caution!) in a vial until vigorous evolution of gas was observed. The cooled crude product was combined with DMF (ca. 3 mL) and a few drops of TFA were added. An off-white solid precipitated and was collected. This solid was dissolved in 7 mL of a 2:1 mixture of acetic acid and conc. HCl, and this solution was heated to 95° C. for 3 h. The solvents were removed in vacuo to give 237 mg of the crude 2-pyridin-2-yl-5H-imidazo[4,5-d]pyridazine.

### 5-[3-(2,4-Bis-trifluoromethyl-phenyl)-isoxazol-5-ylmethyl]-2-pyridin-2-yl-5H-imidazo[4,5-d]pyridazine (Compound 231)

2-Pyridin-2-yl-5H-imidazo[4,5-d]pyridazine was coupled to 3-(2,4-bis-trifluoromethyl-phenyl)-5-chloromethyl-isoxazole according General Procedure H.

MS 491.1 (M+H⁺); H¹ NMR (DMSO-d₆): δ(ppm) 10.08 (d, 1H), 9.46 (d, 1H), 8.71-8.73 (m, 1H), 8.43-8.46 (m, 1H), 8.24 (s, 1H), 8.21 (d, 1H), 7.91-7.97 (m, 2H), 7.45-7.49 (m, 1H), 7.05 (s, 1H), 6.24 (s, 2H).

### Example 132

### 5-[2-(4-Chloro-phenyl)-3H-imidazol-4-ylmethyl]-2-(2,3-difluoro-phenyl)-5H-imidazo[4,5-d]pyridazine (Compound 232)

4-Chloro-benzonitrile was dissolved in ethanol and HCl was bubbled through the solution for 1 h. The reaction flask was sealed and stored in the freezer overnight. The solvents were removed in vacuo to give 4-chloro-benzimidic acid ethyl ester. The 4-chloro-benzimidic acid ethyl ester was placed in a Parr high-pressure apparatus and 1 equivalent of 1,3-dihydroxyacetone (as the dimer) was added. Liquid NH₃ (ca. 20 mL) was introduced, and the apparatus was sealed and heated to 60° C. overnight. The NH₃ was allowed to evaporate, and the remaining residue was triturated with isopropanol. The isopropanol was concentrated to give [2-(4-chloro-phenyl)-3H-imidazol-4-yl]-methanol. The [2-(4-chloro-phenyl)-3H-imidazol-4-yl]-methanol (45 mg, 0.22 mmol) was suspended in benzene (2 mL) and SOCl₂ (0.05 mL) was added. The reaction was stirred at 78° C. for 3 h, and then the solvents were removed in vacuo.

The crude chloromethyl derivative was coupled to 2-(2,3-difluoro-phenyl)-5H-imidazo[4,5-d]pyridazine according to General Procedure H. MS 423.1 (M+H⁺); H¹ NMR (DMSO-d₆): δ(ppm) 10.60 (s, 1H), 9.76 (s, 1H), 8.12-8.20 (m, 3H), 7.96 (s, 1H), 7.67-7.80 (m, 3H), 7.45-7.52 (m, 1H), 6.21 (s, 1H).

### Example 133

### 6-[3-(2,4-Bis-trifluoromethyl-phenyl)-isoxazol-5-ylmethyl]-2-(2,3-difluoro-phenyl)-6H-imidazo[4,5-d]pyridazin-4-ylamine (Compound 233)

### 2-(2,3-Difluoro-phenyl)-5H-imidazo[4,5-d]pyridazin-4-ylamine

To 2-(2,3-difluoro-phenyl)-1H-imidazole-4,5-dicarbonitrile (1.15 g) in THF (50 mL) at −78° C. was added DIBALH (12.5 mL of 1 M in THF, 2.5 eq.) dropwise and warmed to RT. Hydrazine (5 mL, excess) was added and the mixture stirred for 1 hr. The solvents were removed and the product purified on silica gel 0-20% MeOH CH₂Cl₂. By ¹H NMR the product appeared to be the uncyclized hydrazone. The intermediate was dissolved in MeOH (½ mL) and heated to 145° C. under μ-wave irradiation for 15 min. The MeOH was removed and the product purified on silica gel 0-10% MeOH CH₂Cl₂ yielding the desired product.

### 6-[3-(2,4-Bis-trifluoromethyl-phenyl)-isoxazol-5-ylmethyl]-2-(2,3-difluoro-phenyl)-6H-imidazo[4,5-d]pyridazine-4-ylamine (Compound 233)

Following a procedure similar to General Procedure H, from 3-(2,4-bis-trifluoromethyl-phenyl)-5-chloromethyl-isoxazole and 2-(2,3-difluoro-phenyl)-5H-imidazo[4,5-d]pyridazine-4-ylamine (in place of 2-(2,3-difluoro-phenyl)-5H-imidazo[4,5-d]pyridazine). MS 541.1 (M+H⁺); H¹ NMR (DMSO-d₆): δ(ppm) 9.53 (s, 1H), 8.22 (m, 2H), 8.05 (m, 1H), 7.94 (m, 1H), 7.49 (m, 1H), 7.31 (m, 1H), 7.14 (br s, 2H), 6.98 (s, 1H), 5.94 (s, 2H).

### Example 134

### 2-(2,3-Difluoro-phenyl)-6-[3-(4-trifluoromethyl-phenyl)-isoxazol-5-ylmethyl]-6H-imidazo[4,5-d]pyridazin-4-ylamine (Compound 234)

Following a procedure similar to General Procedure H, from 5-chloromethyl-3-(4-trifluoromethyl-phenyl)-isoxazole and 2-(2,3-difluoro-phenyl)-6H-imidazo[4,5-d]pyridazin-4-ylamine (in place of 2-(2,3-difluoro-phenyl)-5H-imidazo[4,5-d]pyridazine). MS 473.1 (M+H⁺); H¹ NMR (DMSO-d₆): δ(ppm) 9.55 (s, 1H), 8.01-8.1 (m, 3H), 7.86 (d, 2H), 7.41-7.50 (m, 1H), 7.28-7.32 (m, 2H), 7.12 (s, 2H), 5.92 (s, 2H).

### Example 135

### 2-(2,3-Difluoro-phenyl)-6-[3-(4-propoxy-phenyl)-isoxazol-5-ylmethyl]-6H-imidazo[4,5-d]pyridazin-4-ylamine (Compound 235)

Following a procedure similar to General Procedure H, from 2-(2,3-difluoro-phenyl)-6H-imidazo[4,5-d]pyridazin-4-ylamine (in place of 2-(2,3-difluoro-phenyl)-5H-imidazo[4,5-d]pyridazine) and 5-chloromethyl-3-(4-propoxy-phenyl)-isoxazole. MS 463.1 (M+H⁺); H¹ NMR (DMSO-d₆): δ(ppm) 9.53 (s, 1H), 8.01-8.07 (m, 1H), 7.74-7.79 (m, 2H), 7.40-7.47 (m, 1H), 7.25-7.32 (m, 1H), 7.11 (s, 2H), 7.09 (s, 1H), 6.99-7.04 (m, 2H), 5.86 (s, 2H), 3.97 (t, 2H), 1.73 (sext., 2H), 0.98 (t, 3H).

### Example 136

### 2-(2,3-Difluoro-phenyl)-6-[3-(2-fluoro-4-trifluoromethyl-phenyl)-isoxazol-5-ylmethyl]-6H-imidazo[4,5-d]pyridazin-4-ylamine (Compound 236)

Following a procedure similar to General Procedure H, from 2-(2,3-difluoro-phenyl)-6H-imidazo[4,5-d]pyridazin-4-ylamine (in place of 2-(2,3-difluoro-phenyl)-5H-imidazo[4,5-d]pyridazine) and 5-chloromethyl-3-(2-fluoro-4-trifluoromethyl-phenyl)-isoxazole. MS 491.1 (M+H⁺); H¹ NMR (DMSO-d₆): δ(ppm) 9.55 (s, 1H), 8.12 (t, 1H), 8.00-8.06 (m, 1H), 7.92 (d, 1H), 7.72 (d, 1H), 7.41-7.50 (m, 1H), 7.25-7.32 (m, 1H), 7.18 (d, 1H), 7.12 (s, 2H), 5.94 (s, 2H).

### Example 137

### 5-[3-(2,4-Bis-trifluoromethyl-phenyl)-isoxazol-5-ylmethyl]-2-(2,3-difluoro-phenyl)-5H-imidazo[4,5-d]pyridazine-4,7-diamine (Compound 237)

### 2-(2,3-Difluoro-phenyl)-5H-imidazo[4,5-d]pyridazine-4,7-diamine

To 2-(2,3-Difluoro-phenyl)-1H-imidazole-4,5-dicarbonitrile (100 mg) was added hydrazine (anhydrous, 1 mL) and stirred at room temperature for 16 hrs. The hydrazine was removed and the product purified by HPLC.

### 5-[3-(2,4-Bis-trifluoromethyl-phenyl)-isoxazol-5-ylmethyl]-2-(2,3-difluoro-phenyl)-5H-imidazo[4,5-d]pyridazine-4,7-diamine (Compound 237)

Following a procedure similar to General Procedure H, from 3-(2,4-bis-trifluoromethyl-phenyl)-5-chloromethyl-isoxazole and 2-(2,3-difluoro-phenyl)-5H-imidazo[4,5-d]pyridazine-4,7-diamine (in place of 2-(2,3-difluoro-phenyl)-5H-imidazo[4,5-d]pyridazine). MS 556 (M+H⁺); H¹ NMR (DMSO-d₆): δ(ppm) 8.96 (br s, 2H), 8.22 (m, 2H), 8.00 (m, 1H), 7.90 (m, 1H), 7.77 (m, 1H), 7.45 (m, 1H), 7.31 (br s, 2H), 6.95 (s, 1H), 5.71 (s, 1H).

### Example 138

### 5-[5-(4-Chloro-phenyl)-oxazol-2-ylmethyl]-2-(2-fluoro-phenyl)-5H-imidazo[4,5-d]pyridazine (Compound 238)

From 2-(2-Fluoro-phenyl)-5H-imidazo[4,5-d]pyridazine and 2-chloromethyl-5-(4-chloro-phenyl)-oxazole (similar to General Procedure B, using corresponding oxazole derivatives in place of isoxazole derivatives). MS: 406.1 (M+H⁺); H¹ NMR (DMSO-d₆): δ(ppm) 10.56 (s, 1H), 9.77-9.79 (m, 1H), 8.32-8.42 (m, 1H), 7.64-7.83 (m, 4H), 7.44-7.59 (m, 4H), 6.37 (s, 2H).

### Example 139

### 5-[5-(4-Chloro-phenyl)-isoxazol-3-ylmethyl]-2-(2-fluoro-phenyl)-5H-imidazo[4,5-d]pyridazine (Compound 239)

From 2-(2-Fluoro-phenyl)-5H-imidazo[4,5-d]pyridazine and methanesulfonic acid 5-(4-chloro-phenyl)-isoxazol-3-ylmethyl ester. MS: 406.1 (M+H⁺); H¹ NMR (DMSO-d₆): δ(ppm) 10.45 (s, 1H), 9.73 (s, 1H), 8.30-8.40 (m, 1H), 7.81-7.90 (m, 2H), 7.65-7.76 (m, 1H), 7.42-7.64 (m, 4H), 7.19 (s, 1H), 6.23 (s, 2H).

### Example 140

### 2-(2-Fluoro-phenyl)-5-[5-(4-methoxy-phenyl)-[1,2,4]oxadiazol-3-ylmethyl]-5H-imidazo[4,5-d]pyridazine (Compound 240)

From 2-(2-Fluoro-phenyl)-5H-imidazo[4,5-d]pyridazine and 3-chloromethyl-5-(4-methoxy-phenyl)-[1,2,4]oxadiazole. MS: 403.1 (M+H⁺); H¹ NMR (DMSO-d₆): δ(ppm) 10.57 (s, 1H), 9.79 (s, 1H), 8.31-8.40 (m, 1H), 7.97-8.04 (m, 2H), 7.68-7.79 (m, 1H), 7.44-7.58 (m, 2H), 7.09-7.17 (m, 2H), 6.37 (s, 1H), 3.85 (s, 3H).

### Example 141

### 2-(2,3-Difluoro-phenyl)-5-[5-(4-trifluoromethyl-phenyl)-isoxazol-3-ylmethyl]-5H-imidazo[4,5-d]pyridazine (Compound 241)

From methanesulfonic acid 3-(4-trifluoromethyl-phenyl)-isoxazol-5-ylmethyl ester and 2-(2,3-difluoro-phenyl)-5H-imidazo[4,5-d]pyridazine MS 458.1 (M+H⁺); H¹ NMR (DMSO-d₆): δ(ppm) 10.34 (s, 1H), 9.6 (s, 1H), 8.08-8.16 (m, 3H), 7.78 (m, 2H), 7.67 (m, 1H), 7.44 (s, 1H), 7.32 (s, 1H), 6.18 (s, 2H).

### Example 142

### 2-(2,3-Difluoro-phenyl)-5-[5-(4-trifluoromethoxy-phenyl)-isoxazol-3-ylmethyl]-5H-imidazo[4,5-d]pyridazine (Compound 242)

From methanesulfonic acid 5-(4-trifluoromethoxy-phenyl)-isoxazol-3-ylmethyl ester and 2-(2,3-difluoro-phenyl)-5H-imidazo[4,5-d]pyridazine. MS 474.1 (M+H⁺); H¹ NMR (DMSO-d₆): δ(ppm) 10.27 (s, 1H), 9.6 (s, 1H), 8.16 (m, 1H), 7.98 (m, 2H), 7.67 (m, 1H), 7.54 (m, 2H), 7.42 (m, 1H), 7.2 (s, 1H), 6.13 (s, 2H).

### Example 143

### 2-(2,3-Difluoro-phenyl)-5-[5-(4-propoxy-phenyl)-isoxazol-3-ylmethyl]-5H-imidazo[4,5-d]pyridazine (Compound 243)

From methanesulfonic acid 5-(4-propoxy-phenyl)-isoxazol-3-ylmethyl ester and 2-(2,3-difluoro-phenyl)-5H-imidazo[4,5-d]pyridazine. MS 448.1 (M+H⁺); H¹ NMR (DMSO-d₆): δ(ppm) 10.34 (s, 1H), 9.6 (s, 1H), 8.08-8.16 (m, 1H), 7.75 (m, 3H), 7.02 (m, 3H), 6.13 (s, 2H), 3.96 (t, 2H), 1.7 (m, 2H), 0.97 (t, 3H).

### Example 144

### 5-[5-(4-Butyl-phenyl)-isoxazol-3-ylmethyl]-2-(2,3-difluoro-phenyl)-5H-imidazo[4,5-d]pyridazine (Compound 244)

From methanesulfonic acid 5-(4-butyl-phenyl)-isoxazol-3-ylmethyl ester and 2-(2,3-difluoro-phenyl)-5H-imidazo[4,5-d]pyridazine. MS 446.1 (M+H⁺); H¹ NMR (DMSO-d₆): δ(ppm) 10.54 (s, 1H), 9.7 (s, 1H), 8.08-8.16 (m, 1H), 7.75 (m, 3H), 7.5 (m, 1H), 7.32 (m, 2H), 7.06 (s, 1H), 6.21 (s, 2H), 2.61 (m, 2H), 1.54 (m, 2H), 1.3 (m. 2H), 0.85 (t, 3H).

### General Procedure J

### Synthesis of Compounds 245-247

### 6-(2,3-difluoro-phenyl)-2H-imidazo[4,5-c]pyridazine

Pyridazine-3,4-diamine was synthesized as described by Kuraishi et al. in J. Het. Chem. 1964, 1, 42-47. MS: 111.1 (M+H⁺); H¹-NMR (DMSO-d₆): δ(ppm) 8.2-8.3 (m, 3H), 7.31 (s, 2H), 6.73 (d, 1H, 6.1 Hz).

2,3-Difluoro-benzoic acid (100 mg), HATU (345.6 mg), and diisopropylethylamine (3 eq.) were added to DMF (900 uL) and stirred for 15 minutes. Pyridazine-3,4-diamine was added and the reaction mixture was stirred at room temperature overnight. The reaction mixture was evaporated, partitioned between water and ethyl acetate. The organic fraction was dried with sodium sulfate and concentrated in vacuo. The residue was then heated in acetic acid at reflux for one day. The mixture was evaporated and purified via reverse-phase HPLC to give 136 mg of 6-(2,3-difluoro-phenyl)-2H-imidazo[4,5-c]pyridazine. MS: 233.1 (M+H⁺) H¹-NMR (DMSO-d₆): δ(ppm) 9.04 (d, 1H, 5.8 Hz), 8.08 (m, 1H), 7.96 (d, 1H, 5.3 Hz) 7.70 (m, 1H) 7.44 (m, 1H)

### Compounds 245-247

A solution of 6-(2,3-difluoro-phenyl)-2H-imidazo[4,5-c]pyridazine and a 5-chloromethyl-2-aryl-isoxazole compound (1 equivalent), and cesium carbonate (66.7 mg, 0.20 mmol) in DMF (3 mL) was heated under microwave irradiation at 120° C. for 10 minutes. The reaction was filtered and purified by reverse phase HPLC to give the desired product. The product was converted to the HCl salt by the addition of 1N HCl before concentration.

### Example 145

### 2-[3-(2,4-Bis-trifluoromethyl-phenyl)-isoxazol-5-ylmethyl]-6-(2,3-difluoro-phenyl)-2H-imidazo[4,5-c]pyridazine (Compound 245)

From 3-(2,4-bis-trifluoromethyl-phenyl)-5-chloromethyl-isoxazole and 6-(2,3-difluoro-phenyl)-2H-imidazo[4,5-c]pyridazine. MS: 526.1 (M+H⁺); H¹-NMR (DMSO-d₆): δ(ppm) 9.4 (d, 1H), 8.4 (d, 1H), 8.2 (m, 3H), 7.9 (d, 2H), 7.7 (m, 1H), 7.4 (m, 1H), 7.1 (s, 1H), 6.4 (s, 2H).

### Example 146

### 6-(2,3-Difluoro-phenyl)-2-[3-(4-trifluoromethyl-phenyl)-isoxazol-5-ylmethyl]-2H-imidazo[4,5-c]pyridazine (Compound 246)

From 5-chloromethyl-3-(4-trifluoromethyl-phenyl)-isoxazole and 6-(2,3-difluoro-phenyl)-2H-imidazo[4,5-c]pyridazine. MS: 458.0 (M+H⁺); H¹-NMR (DMSO-d₆): δ(ppm) 9.4 (d, 1H), 8.4 (d, 1H), 8.2 (m, 1H), 8.1 (d, 2H), 7.9 (d, 2H), 7.7 (m, 1H), 7.4 (m, 1H), 7.4 (s, 1H), 6.3 (s, 2H).

### Example 147

### 6-(2,3-Difluoro-phenyl)-2-[3-(2-fluoro-4-trifluoromethyl-phenyl)-isoxazol-5-ylmethyl]-2H-imidazo[4,5-c]pyridazine (Compound 247)

From 5-chloromethyl-3-(2-fluoro-4-trifluoro-phenyl)-isoxazole and 6-(2,3-difluoro-phenyl)-2H-imidazo[4,5-c]pyridazine. MS: 476.1 (M+H⁺); H¹-NMR (DMSO-d₆): δ(ppm) 9.4 (d, 1H), 9.4 (d, 1H), 8.1-8.2 (m, 2H), 7.9 (m, 1H), 7.6-7.8 (m, 2H), 7.4-7.5 (m, 1H), 7.4 (d, 1H), 6.4 (s, 1H).

### General Procedure K

### Synthesis of 2-(Substituted amino)-5-substituted-imidazo[4,5-d]pyridazines Compounds 248-261

### 5-[3-(2,4-Bis-trifluoromethyl-phenyl)-isoxazol-5-ylmethyl]-2-bromo-5H-imidazo[4,5-d]pyridazine

To a solution of 2-bromo-5H-imidazo[4,5-d]pyridazine (350 mg) in DMF (5 mL) was added an excess of K₂CO₃ (500 mg) and 3-(2,4-Bis-trifluoromethyl-phenyl)-5-chloromethyl-isoxazole (1 eq, 600 mg) and heated to 40 C for 1 hr. The mixture was then cooled and poured into H₂O (30 mL) and the precipitate collected and dried to give the product (590 mg, 70%). MS 492.1, 494.1 (M+H⁺); H¹ NMR (DMSO-d₆): δ(ppm) 10.08 (s, 1H), 9.41 (s, 1H), 8.22 (m, 2H), 7.91 (m, 1H), 7.01 (s, 1H), 6.21 (s, 1H).

### 2-(Substituted amino)-5-substituted-imidazo[4,5-d]pyridazines

5-[3-(2,4-Bis-trifluoromethyl-phenyl)-isoxazol-5-ylmethyl]-2-bromo-5H-imidazo[4,5-d]pyridazine (70 mg) was dissolved in a substituted amino compound (0.5 mL) and heated under microwave irradiation to 160° C. for 10 minutes. The mixture was cooled and the solvent was removed, yielding the amine after HPLC purification.

### Example 148

### {5-[3-(2,4-Bis-trifluoromethyl-phenyl)-isoxazol-5-ylmethyl]-5H-imidazo[4,5-d]pyridazin-2-yl}-phenyl-amine (Compound 248)

From aniline. MS 505.1 (M+H⁺); H¹ NMR (DMSO-d₆): δ(ppm) 11.1 (br s, 1H), 9.8 (s, 1H), 9.31 (s, 1H), 8.2 (m, 2H), 7.91 (m, 1H), 7.77 (m, 2H), 7.4 (m, 2H), 7.11 (m, 1H), 7.05 (s, 1H), 6.27 (s, 2H).

### Example 149

### 5-[3-(2,4-Bis-trifluoromethyl-phenyl)-isoxazol-5-ylmethyl]-2-morpholin-4-yl-5H-imidazo[4,5-d]pyridazine (Compound 249)

From morpholine. MS 499.1 (M+H⁺); H¹ NMR (DMSO-d₆): δ(ppm) 9.86 (s, 1H), 9.26 (s, 1H), 8.23 (m, 2H), 7.91 (m, 1H), 7.03 (s, 1H), 6.28 (s, 2H), 3.77 (m, 8H).

### Example 150

### 5-[3-(2,4-Bis-trifluoromethyl-phenyl)-isoxazol-5-ylmethyl]-2-piperidin-1-yl-5H-imidazo[4,5-d]pyridazine (Compound 250)

From piperidine. MS 497.1 (M+H⁺); H¹ NMR (DMSO-d₆): δ(ppm) 9.74 (s, 1H), 9.2 (s, 1H), 8.23 (m, 2H), 7.91 (m, 1H), 7.02 (s, 1H), 6.25 (s, 2H), 3.80 (m, 4H) 1.65 (m, 6H).

### Example 151

### Benzyl-{5-[3-(2,4-bis-trifluoromethyl-phenyl)-isoxazol-5-ylmethyl]-5H-imidazo[4,5-d]pyridazin-2-yl}-amine (Compound 251)

From benzylamine. MS 519 (M+H⁺); H¹ NMR (DMSO-d₆): δ(ppm) 9.7 (s, 1H), 9.37 (br s, 1H), 9.2 (s, 1H), 8.23 (m, 2H), 7.90 (m, 1H), 7.23 (m, 5H), 7.02 (s, 1H), 6.24 (s, 2H), 4.7 (d, 2H).

### Example 152

### Benzyl-{5-[3-(2,4-bis-trifluoromethyl-phenyl)-isoxazol-5-ylmethyl]-5H-imidazo[4,5-d]pyridazin-2-yl}-methyl-amine (Compound 252)

From benzyl-methyl-amine. 533.1 (M+H⁺); H¹ NMR (DMSO-d₆): δ(ppm) 9.71 (s, 1H), 9.18 (s, 1H), 8.18-8.14 (m, 2H), 7.85-7.83 (d, 1H), 7.29-7.25 (m, 5H), 6.98 (s, 1H), 6.20 (s, 2H), 4.88 (s, 2H), 3.17 (s, 3H).

### Example 153

### 1-{5-[3-(2,4-Bis-trifluoromethyl-phenyl)-isoxazol-5-ylmethyl]-5H-imidazo[4,5-d]pyridazin-2-yl}-1,2,3,4-tetrahydro-quinoline (Compound 253)

From 1,2,3,4-tetrahydro-quinoline. 545.1 (M+H⁺); H¹ NMR (DMSO-d₆): δ(ppm) 9.82 (s, 1H), 9.32 (s, 1H), 8.24-8.20 (m, 2H), 7.93-7.89 (t, 2H), 7.30-7.24 (m, 2H), 7.16-7.11 (m, 1H), 7.05 (s, 1H), 6.29 (s, 2H), 4.06 (t, 2H), 2.83-2.79 (t, 2H), 2.05-2.01 (m, 2H).

### Example 154

### {5-[3-(2,4-Bis-trifluoromethyl-phenyl)-isoxazol-5-ylmethyl]-5H-imidazo[4,5-d]pyridazin-2-yl}-(2-fluoro-benzyl)-amine (Compound 254)

From 2-fluoro-benzylamine. 537.1 (M+H⁺); H¹ NMR (DMSO-d₆): δ(ppm) 9.69 (s, 1H), 9.34 (s, 1H), 9.17 (s, 1H), 8.17-8.13 (m, 2H), 7.84-7.82 (d, 1H), 7.40-7.08 (m, 4H), 6.97 (s, 1H), 6.20 (s, 2H), 4.70-4.68 (d, 2H).

### Example 155

### {5-[3-(2,4-Bis-trifluoromethyl-phenyl)-isoxazol-5-ylmethyl]-5H-imidazo[4,5-d]pyridazin-2-yl}-(2,3-difluoro-benzyl)-amine (Compound 255)

From 2,3-difluoro-benzylamine 555.1 (M+H⁺); H¹ NMR (DMSO-d₆): δ (ppm), 9.71 (s, 1H), 9.37 (s, 1H), 9.18 (s, 1H), 8.17-8.13 (m, 2H), 7.84-7.82 (d, 1H), 7.31-7.10 (m, 3H), 6.97 (s, 1H), 6.20 (s, 2H), 4.71-4.72 (d, 2H).

### Example 156

### {5-[3-(2,4-Bis-trifluoromethyl-phenyl)-isoxazol-5-ylmethyl]-5H-imidazo[4,5-d]pyridazin-2-yl}-phenethyl-amine (Compound 256)

From phenethylamine. 533.1 (M+H⁺); H¹ NMR (DMSO-d₆): δ(ppm) 9.69 (s, 1H), 9.20 (s, 1H), 8.96 (b, 1H), 8.23-8.20 (m, 2H), 7.91-7.88 (d, 1H), 7.31-7.16 (m, 5H), 7.02 (s, 1H), 6.24 (s, 2H), 3.71-3.67 (m, 2H), 2.96-2.91 (t, 2H).

### Example 157

### 2-{5-[3-(2,4-Bis-trifluoromethyl-phenyl)-isoxazol-5-ylmethyl]-5H-imidazo[4,5-d]pyridazin-2-yl}-1,2,3,4-tetrahydro-isoquinoline (Compound 257)

From 1,2,3,4-tetrahydro-isoquinoline. 545.1 (M+H⁺); H¹ NMR (DMSO-d₆): δ(ppm) 9.75 (s, 1H), 9.19 (s, 1H), 8.17-8.13 (m, 2H), 7.85-7.82 (d, 1H), 7.19-7.16 (m, 4H), 6.97 (s, 1H), 6.21 (s, 2H), 4.92 (s, 2H), 4.01-3.97 (t, 2H), 2.97-2.93 (t, 2H).

### Example 158

### {5-[3-(2,4-Bis-trifluoromethyl-phenyl)-isoxazol-5-ylmethyl]-5H-imidazo[4,5-d]pyridazin-2-yl}-(1-phenyl-ethyl)-amine (Compound 258)

From 1-phenyl-ethylamine. 533.1 (M+H⁺); H¹ NMR (DMSO-d₆): δ(ppm) 9.70-9.56 (m, 2H), 9.18 (s, 1H), 8.22-8.18 (m, 2H), 7.89-7.87 (d, 1H), 7.47-7.44 (d, 2H), 7.35-7.02 (m, 3H), 7.00 (s, 1H), 6.24 (s, 2H), 5.22-5.17 (q, 1H), 1.57-1.55 (d, 3H).

### Example 159

### 5-[3-(2,4-Bis-trifluoromethyl-phenyl)-isoxazol-5-ylmethyl]-5H-imidazo[4,5-d]pyridazin-2-yl}-indan-1-yl-amine (Compound 259)

From indan-1-ylamine. 545.1 (M+H⁺); H¹ NMR (DMSO-d₆): δ(ppm) 9.75 (s, 1H), 9.40-9.38 (d, 1H), 9.24 (s, 1H), 8.24-8.20 (d, 2H), 7.92-7.89 (d, 1H), 7.31-7.15 (m, 4H), 6.28 (s, 2H), 7.05 (s, 1H), 5.56-5.54 (q, 1H), 3.07-2.84 (m, 2H), 2.62-2.58 (m, 1H), 2.06-1.99 (m, 1H).

### Example 160

### {5-[3-(2,4-Bis-trifluoromethyl-phenyl)-isoxazol-5-ylmethyl]-5H-imidazo[4,5-d]pyridazin-2-yl}-(1,2,3,4-tetrahydro-naphthalen-1-yl)-amine (Compound 260)

From 1,2,3,4-tetrahydro-naphthalen-1-ylamine. 559.2 (M+H⁺); H¹ NMR (DMSO-d₆): δ(ppm) 9.59 (s, 1H), 9.27 (s, 1H), 9.16 (s, 1H), 8.18-8.14 (d, 2H), 7.86-7.83 (d, 1H), 7.21-7.02 (m, 4H), 6.98 (s, 1H), 6.21 (s, 2H), 5.17 (s, 1H), 2.80-2.66 (m, 2H), 2.02-1.68 (m, 5H).

### Example 161

### 5-[3-(2,4-Bis-trifluoromethyl-phenyl)-isoxazol-5-ylmethyl]-2-(1,3-dihydro-isoindol-2-yl)-5H-imidazo[4,5-d]pyridazine (Compound 261)

From 2,3-dihydro-1H-isoindole. 531.1 (M+H⁺); H¹ NMR (DMSO-d₆): δ(ppm) 9.24 (s, 1H), 8.84 (s, 1H), 8.22-8.18 (m, 2H), 7.93-7.91 (d, 1H), 7.42-7.29 (m, 4H), 6.94 (s, 1H), 6.05 (s, 2H), 4.92 (s, 4H).

### Example 162

### 6-[3-(2,4-Bis-trifluoromethyl-phenyl)-isoxazol-5-ylmethyl]-2-(2,3-difluoro-phenyl)-6H-imidazo[4,5-d]pyridazin-4-ol (Compound 262)

To 6-[3-(2,4-Bis-trifluoromethyl-phenyl)-isoxazol-5-ylmethyl]-2-(2,3-difluoro-phenyl)-6H-imidazo[4,5-d]pyridazin-4-ylamine (compound 233, 54 mg) in HOAc (1 mL) was added NOBF₄ (32 mg, 2 eq.) and stirred at RT for 2 hrs. The solvent was removed and the crude product purified by HPLC. MS 542.1 (M+H⁺); H¹ NMR (DMSO-d₆): δ(ppm) 9.87 (s, 1H), 8.23 (m, 2H), 7.93 (m, 2H), 7.77 (m, 1H), 7.43 (m, 1H), 7.06 (s, 1H), 6.08 (s, 2H).

### Example 163

### 3-{5-[2-(2,3-Difluoro-phenyl)-imidazo[4,5-d]pyridazin-5-ylmethyl]-isoxazol-3-yl}-benzoic acid (Compound 263)

To a solution of 3-{5-[2-(2,3-difluoro-phenyl)-imidazo[4,5-d]pyridazin-5-ylmethyl]-isoxazol-3-yl}-benzoic acid methyl ester (compound 210, 138.5 mg, 0.31 mmol) in dichloromethane (3.8 mL), boron tribromide (1.0M in dichloromethane, 2.79 mL) was added. The mixture was heated at 42° C. until completion. The reaction was quenched by the addition of 1N HCl, and the solvent was removed. The resulting residue was purified by reverse phase HPLC to give the desired product. The product was converted to the HCl salt by the addition of 1N HCl before concentration. MS: 434.1 (M+H⁺); H¹ NMR (DMSO-d₆): δ(ppm) 10.26 (s, 1H), 9.61 (s, 1H), 8.33-8.38 (m, 1H), 8.00-8.19 (m, 3H), 7.59-7.68 (m, 2H), 7.35-7.45 (m, 1H), 7.32 (s, 1H), 6.26 (s, 2H).

### Example 164

### 4-{5-[2-(2,3-Difluoro-phenyl)-imidazo[4,5-d]pyridazin-5-ylmethyl]-isoxazol-3-yl}-benzoic acid (Compound 264)

To a solution of 4-{5-[2-(2,3-difluoro-phenyl)-imidazo[4,5-d]pyridazin-5-ylmethyl]-isoxazol-3-yl}-benzoic acid methyl ester (compound 209, 50.0 mg, 0.11 mmol) in dichloromethane (1.4 mL), boron tribromide (1.0M in dichloromethane, 1.00 mL) was added. The mixture was heated at 42° C. until completion. The reaction was quenched by the addition of 1N HCl, and the solvent was removed. The resulting residue was purified by reverse phase HPLC to give the desired product. The product was converted to the HCl salt by the addition of 1N HCl before concentration. MS: 434.1 (M+H⁺); H¹ NMR (DMSO-d₆): δ(ppm) 10.23 (s, 1H), 9.58 (s, 1H), 8.11-8.20 (m, 1H), 7.94-8.07 (m, 4H), 7.56-7.68 (m, 1H), 7.34-7.44 (m, 1H), 7.29 (s, 1H), 6.25 (s, 2H).

### Example 165

### (4-{5-[2-(2,3-Difluoro-phenyl)-imidazo[4,5-d]pyridazin-5-ylmethyl]-isoxazol-3-yl}-phenoxy)-acetic acid (Compound 265)

To a solution of (4-{5-[2-(2,3-difluoro-phenyl)-imidazo[4,5-d]pyridazin-5-ylmethyl]-isoxazol-3-yl}-phenoxy)-acetic acid methyl ester (compound 215, 150 mg, 0.31 mmol) in acetonitrile (3 mL), 2M HCl (3 mL) was added. The mixture was heated allowed to stir at 50° C. overnight. The acetonitrile was removed, and the resulting residue was purified by reverse phase HPLC to give the desired product. The product was converted to the HCl salt by the addition of 1N HCl before concentration. MS: 434.1 (M+H⁺); H¹ NMR (DMSO-d₆): δ(ppm) 10.39 (s, 1H), 9.67 (s, 1H), 8.12-8.20 (m, 1H), 7.60-7.80 (m, 3H), 7.38-7.48 (m, 1H), 7.16 (s, 1H), 6.97-7.07 (m, 2H), 6.27 (s, 2H), 4.74 (s, 2H).

### Example 166

### 2-{5-[2-(2,3-Difluoro-phenyl)-imidazo[4,5-d]pyridazin-5-ylmethyl]-isoxazol-3-yl}-5-methoxy-benzoic acid (Compound 266)

2-{5-[2-(2,3-Difluoro-phenyl)-imidazo[4,5-d]pyridazin-5-ylmethyl]-isoxazol-3-yl}-5-methoxy-benzoic acid methyl ester (compound 198, 100 mg) are heated in 6 mL of 1:1 6N HCl/4MHCl in dioxane for three hours at 95° C. The reaction is cooled, evaporated and purified via reverse phase HPLC to give 2-{5-[2-(2,3-difluoro-phenyl)-imidazo[4,5-d]pyridazin-5-ylmethyl]-isoxazol-3-yl}-5-methoxy-benzoic acid. The product was converted to the HCl salt by the addition of 1N HCl before concentration.

MS: 464.1 (M+H⁺); H¹-NMR (DMSO-d₆): δ(ppm) 10.4 (s, 1H), 9.7 (s, 1H), 8.2 (m, 1H), 8.0 (s, 1H), 7.7 (m, 1H), 7.4-7.5 (m, 2H), 7.3 (d, 1H), 7.2 (m, 1H), 6.9 (s, 1H), 6.3 (s, 2H), 3.8 (s, 3H).

### Example 167

### 5-{5-[2-(2,3-Difluoro-phenyl)-imidazo[4,5-d]pyridazin-5-ylmethyl]-isoxazol-3-yl}-2-propoxy-benzoic acid (Compound 267)

5-{5-[2-(2,3-Difluoro-phenyl)-imidazo[4,5-d]pyridazin-5-ylmethyl]-isoxazol-3-yl}-2-propoxy-benzoic acid propyl ester (compound 197, 110 mg) are heated in 6 mL of 1:1 6N HCl aq./4M HCl in dioxane for three hours at 95° C. The reaction is cooled, evaporated and purified via reverse phase HPLC to give 43 mg of 5-{5-[2-(2,3-Difluoro-phenyl)-imidazo[4,5-d]pyridazin-5-ylmethyl]-isoxazol-3-yl}-2-propoxy-benzoic acid. The product was converted to the HCl salt by the addition of 1N HCl before concentration. MS: 492.1 (M+H⁺); H¹-NMR (DMSO-d₆): δ(ppm) 10.2 (s, 1H), 9.6 (s, 1H), 8.2 (m, 1H), 8.0 (s, 1H), 7.9 (dd, 1H), 7.6 (m, 1H), 7.4 (m, 1H), 7.2 (m, 2H), 6.2 (s, 2H), 4.0 (t, 2H), 1.7 (m, 2H), 1.0 (t, 3H).

### Example 168

### 2-(2,3-Difluoro-phenyl)-5-[3-(4′-methoxy-biphenyl-4-yl)-isoxazol-5-ylmethyl]-5H-imidazo[4,5-d]pyridazine (Compound 268)

A reaction vessel is charged with 5-[3-(4-bromo-phenyl)-isoxazol-5-ylmethyl]-2-(2,3-difluoro-phenyl)-5H-imidazo[4,5-d]pyridazine (compound 200, 50 mg, 0.1 mmol), 4-methoxy-phenyl-boronic acid (24.3 mg, 1.5 eq.), tetrakis(triphenylphosphine)-palladium(0) (6 mg, 0.05 eq.), evacuated in vacuo and filled with argon three times. A 2N sodium carbonate solution (107 μl, 2 eq.) and toluene (427 μL) are added and the solution is degassed for 5 minutes. The sealed reaction vessel is then heated to 80° C. for 3 hr. After cooling the reaction mixture is concentrated and purified via reverse phase HPLC to give 17 mg of 2-(2,3-difluoro-phenyl)-5-[3-(4′-methoxy-biphenyl-4-yl)-isoxazol-5-ylmethyl]-5H-imidazo[4,5-d]pyridazine. The product was converted to the HCl salt by the addition of 1N HCl before concentration. MS: 496.2 (M+H⁺); H¹-NMR (DMSO-d₆): δ(ppm) 10.4 (s, 1H), 9.9 (s, 1H), 8.2 (m, 1H), 7.9 (d, 2H), 7.7 (d, 2H), 7.4-7.7 (m, 6H), 7.3 (s, 1H), 7.0 (d, 2H), 6.3 (s, 2H), 3.8 (s, 3H).

### Example 169

### 2-(2,3-Difluoro-phenyl)-5-[3-(4′-propoxy-biphenyl-4-yl)-isoxazol-5-ylmethyl]-5H-imidazo[4,5-d]pyridazine (Compound 269)

A reaction vessel is charged with 5-[3-(4-bromo-phenyl)-isoxazol-5-ylmethyl]-2-(2,3-difluoro-phenyl)-5H-imidazo[4,5-d]pyridazine (compound 200, 50 mg, 0.1 mmol), 4-propoxy-phenyl-boronic acid (28.8 mg, 1.5 eq.), tetrakis(triphenylphosphine)-palladium(0) (6 mg, 0.05 eq.), evacuated in vacuo and filled with argon three times. A 2N sodium carbonate solution (107 μL, 2 eq.) and toluene (427 μL) are added and the solution is degassed for 5 minutes. The sealed reaction vessel is then heated to 80° C. for 3 hr. After cooling the reaction mixture is concentrated and purified via reverse phase HPLC to give 2-(2,3-Difluoro-phenyl)-5-[3-(4′-propoxy-biphenyl-4-yl)-isoxazol-5-ylmethyl]-5H-imidazo[4,5-d]pyridazine. The product was converted to the HCl salt by the addition of 1N HCl before concentration. MS: 524.2 (M+H⁺); H¹-NMR (DMSO-d₆): δ(ppm) 10.3 (d, 1H), 9.6 (d, 1H), 8.1-8.2 (m, 1H), 7.9 (m, 1H), 7.7-7.8 (m, 2H), 7.6-7.7 (m, 3H), 7.4 (m, 1H), 7.2 (s, 1H), 7.0 (m, 2H), 6.2 (s, 2H), 4.0 (t, 2H), 1.7-1.8 (m, 2H), 1.0 (t, 3H).

### Example 170

### 5-{5-[2-(2,3-Difluoro-phenyl)-imidazo[4,5-d]pyridazin-5-ylmethyl]-isoxazol-3-yl}-N-(2-morpholin-4-yl-ethyl)-2-propoxy-benzamide (Compound 270)

5-{5-[2-(2,3-Difluoro-phenyl)-imidazo[4,5-d]pyridazin-5-ylmethyl]-isoxazol-3-yl}-2-propoxy-benzoic acid (compound 267, 30 mg), HATU (23.4 mg), and diisopropylethylamine (21.8 uL) are dissolved in 0.5 mL DMF and stirred for 5 minutes. 2-Aminoethyl morpholine (6 uL) is added and the reaction stirred for 2 hours at room temperature. The reaction is then evaporated and purified via reverse phase-HPLC to give 21 mg of 5-{5-[2-(2,3-Difluoro-phenyl)-imidazo[4,5-d]pyridazine-5-ylmethyl]-isoxazol-3-yl}-N-(2-morpholin-4-yl-ethyl)-2-propoxy-benzamide. The product was converted to the HCl salt by the addition of 1N HCl before concentration. MS: 604.1 (M+H⁺); H¹-NMR (DMSO-d₆): δ(ppm) 10.9 (bs, 1H), 10.4 (s, 1H), 9.7 (s, 1H), 8.5 (tr, 1H), 8.1-8.2 (m, 2H), 7.9 (m, 1H), 7.7 (m, 1H), 7.4 (m, 1H), 7.2-7.3 (m, 2H), 6.3 (s, 2H), 4.1 (tr, 2H), 3.7-4.0 (m, 6H), 3.5 (m, 2H), 3.2 (m, 2H), 3.1 (m, 2H), 1.8 (m, 2H), 1.0 (t, 3H).

### Example 171

### N-Cyclopropyl-2-(4-{5-[2-(2,3-difluoro-phenyl)-imidazo[4,5-d]pyridazin-5-ylmethyl]-isoxazol-3-yl}-phenoxy)-acetamide (Compound 271)

The (4-{5-[2-(2,3-difluoro-phenyl)-imidazo[4,5-d]pyridazin-5-ylmethyl]-isoxazol-3-yl}-phenoxy)-acetic acid (compound 265, 39 mg, 0.084 mmol), cyclopropyamine (7 μL, 0.10 mmol), diisopropylethylamine (30 μL) and HATU (35 mg, 0.92 mmol) were combined under Ar in a vial and stirred at room temp for 1 h. The reaction mixture was portioned between ethyl acetate and 1 N HCl. The organic layer was washed sequentially with saturated aqueous NaHCO₃, water, and brine. After drying over sodium sulfate, the organics were concentrated onto celite. The product was purified via SiO₂ flash chromatography using 0-20% methanol in ethyl aceate. MS 503.1 (M+H⁺); H¹ NMR (DMF-d₇): δ(ppm) 10.96 (s, 1H), 10.02 (s, 1H), 8.51-8.56 (m, 1H), 8.42 (s, 1H), 7.88-7.97 (m, 1H), 7.65-7.72 (m, 1H), 7.50 (s, 1H), 7.27 (s, 2H), 6.72 (s, 2H), 4.76 (s, 2H), 4.14 (t, 1H), 0.81-0.88 (m, 2H), 0.71-0.76 (m, 2H).

### Example 172

### Acetic acid 3-(4-{5-[2-(2,3-difluoro-phenyl)-imidazo[4,5-d]pyridazin-5-ylmethyl]-isoxazol-3-yl}-phenoxy)-propyl ester (Compound 272)

The 3-(4-{5-[2-(2,3-difluoro-phenyl)-imidazo[4,5-d]pyridazin-5-ylmethyl]-isoxazol-3-yl}-phenoxy)-propan-1-ol (compound 193, 20 mg) was dissolved in 1 mL of acetic anhydride, and excess triethylamine (ca. 0.1 mL) was added. The reaction was warmed to 85° C. for 1 h, and then the volatile components were removed. The residue was portioned between ethyl acetate and water. The organic layer was concentrated to give the pure product. MS 506.0 (M+H⁺); H¹ NMR (CDCl₃): δ(ppm) 9.35 (d, 1H), 9.27 (d, 1H), 8.14-8.19 (m, 1H), 7.68 (d, 2H), 7.19-7.29 (m, 2H), 6.95 (d, 2H), 6.69 (d, 1H), 5.90 (s, 2H), 4.26 (t, 2H), 4.08 (t, 2H), 2.13 (quintet, 2H), 2.06 (s, 3H).

### Example 173

### 2-(2,3-Difluoro-phenyl)-5-(3-[4-(3-morpholin-4-yl-propoxy)-phenyl]isoxazol-5-ylmethyl)-5H-imidazo[4,5-d]pyridazine (Compound 273)

A solution of 3-(4-{5-[2-(2,3-difluoro-phenyl)-imidazo[4,5-d]pyridazin-5-ylmethyl]-isoxazol-3-yl}-phenoxy)-propan-1-ol (compound 193, 40 mg) in DMF (1 mL) was treated with triethylamine (0.1 mL) then methanesulfonyl chloride (0.1 mL). After 10 min, 0.20 mL of morpholine was added and the mixture was heated to 90° C. for 1 h. The reaction mixture was purified by reverse-phase HPLC to give the product, which was converted to the HCl salt and collected as a white powder. MS 533.0 (M+H⁺); H¹ NMR (DMSO-d₆): δ(ppm) 10.49 (s, 1H), 9.75 (s, 1H), 8.18-8.23 (m, 1H), 7.82 (d, 2H), 7.70-7.79 (m, 1H), 7.45-7.52 (m, 1H), 7.20 (s, 1H), 7.08 (d, 2H), 6.33 (s, 2H), 4.15 (t, 2H), 3.98 (dd, 2H), 3.84 (t, 2H), 3.46 (d, 2H), 3.23-3.31 (m, 2H), 3.03-3.14 (m, 2H), 2.21-2.29 (m, 2H).

### Example 174

### 4-(4-{5-[2-(2,3-Difluoro-phenyl)-imidazo[4,5-d]pyridazin-5-ylmethyl]-isoxazol-3-yl}-phenoxy)-butyric acid (Compound 274)

The 4-(4-{5-[2-(2,3-difluoro-phenyl)-imidazo[4,5-d]pyridazin-5-ylmethyl]-isoxazol-3-yl}-phenoxy)-butyric acid methyl ester (compound 192, 60 mg) was suspended in ethanol and magnetically stirred in an ice bath as 5 mL of KOH (20%, aq.) was added. The reaction was stirred at room temp overnight, and then most of the ethanol was removed under vacuum. The remaining liquid was diluted with 50 mL of water, and the pH was adjusted to 3 using concentrated HCl. The product precipitated and was isolated by filtration. MS 492.1 (M+H⁺); H¹ NMR (DMSO-d₆): δ(ppm) 10.37 (s, 1H), 9.66 (s, 1H), 8.14-8.17 (m, 1H), 7.76 (d, 2H), 7.67 (quartet, 1H), 7.40-7.47 (m, 1H), 7.15 (s, 1H), 7.02 (d, 2H), 6.25 (s, 2H), 4.02 (t, 2H), 2.37 (t, 2H), 1.93 (quintet, 2H).

### Example 175

### 2-(2-Fluoro-phenyl)-5-[3-(3-propoxy-phenyl)-isoxazol-5-ylmethyl]-5H-imidazo[4,5-d]pyridazine (Compound 275)

Following General Procedure H, from 2-(2-fluoro-phenyl)-5H-imidazo[4,5-d]pyridazine and 5-chloromethyl-3-(3-propoxy-phenyl)-isoxazole. MS: 430.1 (M+H⁺); H¹ NMR (DMSO-d₆): δ(ppm) 10.59 (s, 1H), 9.82 (s, 1H), 8.32-8.42 (m, 1H), 7.69-7.80 (m, 1H), 7.45-7.60 (m, 2H), 7.32-7.43 (m, 3H), 7.27 (s, 1H), 7.01-7.09 (m, 1H), 6.39 (s, 2H), 3.97 (t, 3H), 1.65-1.80 (m, 2H), 0.98 (t, 3H).

### Example 176

### 2-(2-Fluoro-phenyl)-5-[3-(3-trifluoromethyl-phenyl)-isoxazol-5-ylmethyl]-5H-imidazo[4,5-d]pyridazine (Compound 276)

Following General Procedure H, from 2-(2-fluoro-phenyl)-5H-imidazo[4,5-d]pyridazine and 5-chloromethyl-3-(3-trifluoromethyl-phenyl)-isoxazole. MS: 440.1 (M+H⁺); H¹ NMR (DMSO-d₆): δ(ppm) 10.15 (s, 1H), 9.77 (s, 1H), 8.31-8.40 (m, 1H), 8.12-8.21 (m, 2H), 7.85-7.92 (m, 1H), 7.65-7.80 (m, 2H), 7.38-7.57 (m, 3H), 6.39 (s, 2H).

### Example 177

### 5-[3-(4-Butyl-phenyl)-isoxazol-5-ylmethyl]-2-(2-fluoro-phenyl)-5H-imidazo[4,5-d]pyridazine (Compound 277)

Following General Procedure H, from 2-(2-fluoro-phenyl)-5H-imidazo[4,5-d]pyridazine and 3-(4-butyl-phenyl)-5-chloromethyl-isoxazole. MS: 428.1 (M+H⁺); H¹ NMR (DMSO-d₆): δ(ppm) 10.40 (s, 1H), 9.71 (s, 1H), 8.30-8.40 (m, 1H), 7.63-7.77 (m, 3H), 7.41-7.55 (m, 2H), 7.28-7.34 (m, 2H), 7.20 (s, 1H), 6.30 (s, 2H), 2.62 (t, 2H), 1.49-1.63 (m, 2H), 1.22-1.38 (m, 2H), 0.90 (t, 3H).

### Example 178

### 2-(2-Fluoro-phenyl)-5-[3-(4-trifluoromethyl-phenyl)-isoxazol-5-ylmethyl]-5H-imidazo[4,5-d]pyridazine (Compound 278)

Following General Procedure H, from 2-(2-fluoro-phenyl)-5H-imidazo[4,5-d]pyridazine and 5-chloromethyl-3-(4-trifluoromethyl-phenyl)-isoxazole. MS: 440.1 (M+H⁺); H¹ NMR (DMSO-d₆): δ(ppm) 10.43 (s, 1H), 9.72 (s, 1H), 8.30-8.39 (m, 1H), 8.04-8.11 (m, 2H), 7.84-7.91 (m, 2H), 7.60-7.74 (m, 1H), 7.41-7.55 (m, 2H), 7.36 (s, 1H), 6.35 (s, 2H).

### Example 179

### 2-(2-Fluoro-phenyl)-5-[3-(2-fluoro-4-trifluoromethyl-phenyl)-isoxazol-5-ylmethyl]-5H-imidazo[4,5-d]pyridazine (Compound 279)

Following General Procedure H, from 2-(2-fluoro-phenyl)-5H-imidazo[4,5-d]pyridazine and 5-chloromethyl-3-(2-fluoro-4-trifluoromethyl-phenyl)-isoxazole. MS: 458.0 (M+H⁺); H¹ NMR (DMSO-d₆): δ(ppm) 10.37 (s, 1H), 9.68 (s, 1H), 8.30-8.39 (m, 1H), 8.06-8.16 (m, 1H), 7.91-7.98 (m, 1H), 7.58-7.77 (m, 2H), 7.40-7.53 (m, 2H), 7.24-7.29 (m, 1H), 6.35 (s, 2H).

### Example 180

### 5-[3-(2,5-Bis-trifluoromethyl-phenyl)-isoxazol-5-ylmethyl]-2-(2-fluoro-phenyl)-5H-imidazo[4,5-d]pyridazine (Compound 280)

Following General Procedure H, from 2-(2-fluoro-phenyl)-5H-imidazo[4,5-d]pyridazine and 3-(2,5-bis-trifluoromethyl-phenyl)-5-chloromethyl-isoxazole. MS: 508.1 (M+H⁺); H¹ NMR (DMSO-d₆): δ(ppm) 10.34 (s, 1H), 9.68 (s, 1H), 8.29-8.39 (m, 1H), 8.11-8.22 (m, 2H), 8.04 (s, 1H), 7.60-7.70 (m, 1H), 7.39-7.52 (m, 2H), 7.10 (s, 1H), 6.35 (s, 2H).

### Example 181

### 2-(2-Fluoro-phenyl)-5-[3-(4-methanesulfonyl-phenyl)-isoxazol-5-ylmethyl]-5H-imidazo[4,5-d]pyridazine (Compound 281)

Following General Procedure H, from 2-(2-fluoro-phenyl)-5H-imidazo[4,5-d]pyridazine and 5-chloromethyl-3-(4-methanesulfonyl-phenyl)-isoxazole. MS: 450.1 (M+H⁺); H¹ NMR (DMSO-d₆): δ(ppm) 10.47 (s, 1H), 9.74 (s, 1H), 8.31-8.40 (m, 1H), 8.01-8.15 (m, 4H), 7.64-7.76 (m, 1H), 7.35-7.56 (m, 3H), 6.38 (s, 1H), 3.28 (s, 3H).

### Example 182

### 2-(2-Fluoro-phenyl)-5-[3-(4-iodo-phenyl)-isoxazol-5-ylmethyl]-5H-imidazo[4,5-d]pyridazine (Compound 282)

Following General Procedure H, from 2-(2-fluoro-phenyl)-5H-imidazo[4,5-d]pyridazine and 5-chloromethyl-3-(4-iodo-phenyl)-isoxazole. MS: 498.0 (M+H⁺); H¹ NMR (DMSO-d₆): δ(ppm) 10.46 (s, 1H), 9.74 (s, 1H), 8.30-8.45 (m, 1H), 7.84-7.91 (m, 2H), 7.43-7.72 (m, 5H), 7.25 (s, 1H), 6.34 (s, 2H).

### Example 183

### 5-[3-(4-tert-Butyl-phenyl)-isoxazol-5-ylmethyl]-2-(2-fluoro-phenyl)-5H-imidazo[4,5-d]pyridazine (Compound 283)

Following General Procedure H, from m 2-(2-fluoro-phenyl)-5H-imidazo[4,5-d]pyridazine and 3-(4-tert-butyl-phenyl)-5-chloromethyl-isoxazole. MS: 428.2 (M+H⁺); H¹ NMR (DMSO-d₆): δ(ppm) 10.41-10.45 (m, 1H), 9.71-9.76 (m, 1H), 8.29-8.43 (m, 1H), 7.62-7.79 (m, 3H), 7.42-7.56 (m, 4H), 7.21 (s, 1H), 6.32 (s, 2H), 1.30 (s, 9H).

### Example 184

### 4-{5-[2-(2-Fluoro-phenyl)-imidazo[4,5-d]pyridazin-5-ylmethyl]-isoxazol-3-yl}-benzonitrile (Compound 284)

Following General Procedure H, from 2-(2-fluoro-phenyl)-5H-imidazo[4,5-d]pyridazine and 4-(5-chloromethyl-isoxazol-3-yl)-benzonitrile. MS: 397.2 (M+H⁺); H¹ NMR (DMSO-d₆): δ(ppm) 10.56 (s, 1H), 9.79 (s, 1H), 8.31-8.40 (m, 1H), 7.94-8.08 (m, 4H), 7.68-7.78 (m, 1H), 7.44-7.58 (m, 1H), 7.38 (s, 1H), 6.41 (s, 2H).

### Example 185

### 5-[3-(4-Bromo-phenyl)-isoxazol-5-ylmethyl]-2-(2-fluoro-phenyl)-5H-imidazo[4,5-d]pyridazine (Compound 285)

Following General Procedure H, from 2-(2-fluoro-phenyl)-5H-imidazo[4,5-d]pyridazine and 3-(4-bromo-phenyl)-5-chloromethyl-isoxazole. MS: 451.0 (M+H⁺); H¹ NMR (DMSO-d₆): δ(ppm) 10.35 (s, 1H), 9.67 (s, 1H), 8.30-8.40 (m, 1H), 7.64-7.84 (m, 5H), 7.39-7.53 m, 2H), 7.26 (s, 1H), 6.30 (s, 2H).

### Example 186

### 2-(2-Fluoro-phenyl)-5-[3-(3-fluoro-pyridin-4-yl)-isoxazol-5-ylmethyl]-5H-imidazo[4,5-d]pyridazine (Compound 286)

Following General Procedure H, from 2-(2-fluoro-phenyl)-5H-imidazo[4,5-d]pyridazine and 4-(5-chloromethyl-isoxazol-3-yl)-3-fluoro-pyridine. MS: 391.1 (M+H⁺); H¹ NMR (DMSO-d₆): δ(ppm) 10.77 (s, 1H), 9.88 (s, 1H), 8.83 (s, 1H), 8.59 (d, 1H), 8.38 (t, 1H), 7.88-7.96 (m, 1H), 7.71-7.84 (m, 1H), 7.47-7.63 (m, 2H), 7.37 (s, 1H), 6.53 (s, 2H).

### Example 187

### 2-(2-Fluoro-phenyl)-5-[3-(1H-indol-5-yl)-isoxazol-5-ylmethyl]-5H-imidazo[4,5-d]pyridazine (Compound 287)

Following General Procedure H, from 2-(2-fluoro-phenyl)-5H-imidazo[4,5-d]pyridazine and 5-(5-chloromethyl-isoxazol-3-yl)-1H-indole. MS: 411.1 (M+H⁺); H¹ NMR (DMSO-d₆): δ(ppm) 11.34 (s, 1H), 10.31 (s, 1H), 9.65 (s, 1H), 8.30-8.40 (m, 1H), 8.03 (s, 1H), 7.38-7.70 (m, 6H), 7.19 (s, 1H), 6.46-6.52 (s, 1H), 6.26 (s, 2H).

### Example 188

### 2-(2-Fluoro-phenyl)-5-[3-(1H-indol-6-yl)-isoxazol-5-ylmethyl]-5H-imidazo[4,5-d]pyridazin (Compound 288)

Following General Procedure H, from 2-(2-fluoro-phenyl)-5H-imidazo[4,5-d]pyridazine and 5-(5-chloromethyl-isoxazol-3-yl)-1H-indole. MS: 411.2 (M+H⁺); H¹ NMR (DMSO-d₆): δ(ppm) 11.43 (s, 1H), 10.63 (s, 1H), 9.82 (s, 1H), 8.32-8.41 (m, 1H), 7.86 (s, 1H), 7.42-7.79 (m, 6H), 7.25 (s, 1H), 6.47 (s, 1H), 6.39 (s, 2H).

### Example 189

### 5-[3-(5-Bromo-pyridin-2-yl)-isoxazol-5-ylmethyl]-2-(2-fluoro-phenyl)-5H-imidazo[4,5-d]pyridazine (Compound 289)

Following General Procedure H, from 2-(2-fluoro-phenyl)-5H-imidazo[4,5-d]pyridazine and 5-bromo-2-(5-chloromethyl-isoxazol-3-yl)-pyridine. MS: 451.0 (M+H⁺); H¹ NMR (DMSO-d₆): δ(ppm) 10.56 (s, 1H), 9.78 (s, 1H), 8.80-8.86 (m, 1H), 8.31-8.40 (m, 1H), 8.18-8.26 (m, 1H), 7.93-8.00 (m, 1H), 7.68-7.80 (m, 1H), 7.43-7.59 (m, 2H), 7.28 (s, 1H), 6.42 (s, 2H).

### Example 190

### 1-(4-{5-[2-(2,3-Difluoro-phenyl)-imidazo[4,5-d]pyridazin-5-ylmethyl]-isoxazol-3-yl}-phenyl)-ethanone (Compound 290)

### 1-(4-{5-[2-(2,3-Difluoro-phenyl)-imidazo[4,5-d]pyridazin-5-ylmethyl]-isoxazol-3-yl}-phenyl)-ethanone oxime

Following General Procedure H, from 2-(2,3-difluoro-phenyl)-5H-imidazo[4,5-d]pyridazine and 1-[4-(5-chloromethyl-isoxazol-3-yl)-phenyl]-ethanone oxime.

### 1-(4-{5-[2-(2,3-Difluoro-phenyl)-imidazo[4,5-d]pyridazin-5-ylmethyl]-isoxazol-3-yl}-phenyl)-ethanone (Compound 290)

1-(4-{5-[2-(2,3-Difluoro-phenyl)-imidazo[4,5-d]pyridazin-5-ylmethyl]-isoxazol-3-yl}-phenyl)-ethanone oxime (171.5 mg, 0.38 mmol) was dissolved in glyoxylic acid (50% aq. solution, 3 mL) and stirred at ambient temperature for two hours. After removal of the solvent, purification by reverse phase HPLC gave the desired product. The product was converted to the HCl salt by the addition of 1N HCl before concentration. MS: 432.1 (M+H⁺); H¹ NMR (DMSO-d₆): δ(ppm) 10.57 (s, 1H), 9.78 (s, 1H), 8.12-8.21 (m, 1H), 8.02 (q, 4H), 7.66-7.80 (m, 1H), 7.41-7.52 (m, 1H), 7.36 (s, 1H), 6.40 (s, 2H), 2.61 (s, 3H).

### Example 191

### 5-[5-(4-Chloro-phenyl)-[1,3,4]oxadiazol-2-ylmethyl]-2-(2-fluoro-phenyl)-5H-imidazo[4,5-d]pyridazine (Compound 291)

2-(2-Fluoro-phenyl)-5H-imidazo[4,5-d]pyridazine (30.0 mg, 0.14 mmol), 2-chloromethyl-5-phenyl-[1,3,4]oxadiazole, and cesium carbonate (91.3 mg, 0.28 mmol) were dissolved in DMF and microwaved at 120° C. for 10 minutes. The reaction was filtered and purified by reverse phase HPLC to give the desired product. Yield 12.7 mg. MS 407.0 (M+H⁺); H¹ NMR (DMSO-d₆): δ(ppm) 10.22 (s, 1H), 9.58 (d, 1H), 8.31-8.38 (m, 1H), 7.96-8.01 (m, 2H), 7.57-7.70 (m, 3H), 7.37-7.47 (m, 2H), 6.40 (s, 2H).

### Example 192

### 5-[3-(2,4-Bis-trifluoromethyl-phenyl)-4-bromo-isoxazol-5-ylmethyl]-2-(2,3-difluoro-phenyl)-5H-imidazo[4,5-d]pyridazine (Compound 292)

To a solution of 5-[3-(2,4-Bis-trifluoromethyl-phenyl)-isoxazol-5-ylmethyl]-2-(2,3-difluoro-phenyl)-5H-imidazo[4,5-d]pyridazine (compound 103, 535 mg, 1 mmol) in HOAc (10 mL) was added H₂SO₄ (5 drops) and NBS (725 mg, 4 eq.) and the mixture heated in a sealed vial to 115° C. for 18 hrs. The solvent was removed and the crude product purified by HPLC. MS 604.1/606.1 (M+H⁺); H¹ NMR (DMSO-d₆): δ(ppm) 10.1 (s, 1H), 9.51 (s, 1H), 8.3 (m, 2H), 8.16 (m, 2H), 7.8 (m, 1H), 7.56 (m, 1H), 7.36 (m, 1H), 6.28 (s, 2H).

### Administration and Pharmaceutical Composition

Also provided are compounds possessing antiviral activity, including Flaviviridae family viruses such as hepatitis C virus. The compounds, or pharmaceutically acceptable salts or solvates, described herein inhibit viral replication by inhibiting the enzymes involved in replication, including RNA dependent RNA polymerase. They may also inhibit other enzymes utilized in the activity or proliferation of Flaviviridae viruses.

In general, the compounds, or pharmaceutically acceptable salts or solvates, described herein will be administered in a therapeutically effective amount by any of the accepted modes of administration for agents that serve similar utilities. The actual amount of the compound, or pharmaceutically acceptable salt or solvate, described herein, i.e., the active ingredient, will depend upon numerous factors such as the severity of the disease to be treated, the age and relative health of the subject, the potency of the compound used, the route and form of administration, and other factors. The drug can be administered more than once a day, such as once or twice a day.

Therapeutically effective amounts of compounds, or pharmaceutically acceptable salts or solvates, described herein may range from approximately 0.01 to 50 mg per kilogram body weight of the recipient per day; such as about 0.01-25 mg/kg/day, for example, from about 0.1 to 10 mg/kg/day. Thus, in some embodiments, for administration to a 70 kg person, the dosage range would be about 7-70 mg per day.

This invention is not limited to any particular composition or pharmaceutical carrier, as such may vary. In general, compounds, or pharmaceutically acceptable salts or solvates, described herein will be administered as pharmaceutical compositions by any one of the following routes: oral, systemic (e.g., transdermal, intranasal or by suppository), or parenteral (e.g., intramuscular, intravenous or subcutaneous) administration. In some embodiments, the manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another manner for administering compounds of described herein is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound, or pharmaceutically acceptable salt or solvate, described herein in combination with at least one pharmaceutically acceptable excipient. Acceptable excipients are non-toxic, aid administration, and do not adversely affect the therapeutic benefit of the claimed compounds. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols.

Compressed gases may be used to disperse a compound, or pharmaceutically acceptable salt or solvate, described herein in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound, or pharmaceutically acceptable salt or solvate, described herein based on the total formulation, with the balance being one or more suitable pharmaceutical excipients. In some embodiments, the compound is present at a level of about 1-80 wt %. Representative pharmaceutical formulations are described in the Formulation Examples section below.

Also provided is a pharmaceutical composition comprising a therapeutically effective amount of a compound, or pharmaceutically acceptable salt or solvate, described herein in combination with a therapeutically effective amount of another active agent against RNA-dependent RNA virus and, in particular, against HCV. Agents active against HCV include, but are not limited to, ribavirin, levovirin, viramidine, thymosin alpha-1, an inhibitor of HCV NS3 serine protease, or an inhibitor of inosine monophosphate dehydrognease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as ROFERON interferon available from Hoffman-LaRoche, Nutley, N.J.), interferon-α2b (such as Intron-A interferon available from Schering Corp., Kenilworth, N.J., USA), a consensus interferon, and a purified interferon-α product. For a discussion of ribavirin and its activity against HCV, see J. O, Saunders and S. A. Raybuck, “Inosine Monophosphate Dehydrogenase: Consideration of Structure, Kinetics and Therapeutic Potential,” Ann. Rep. Med. Chem., 35:201-210 (2000).

The agents active against hepatitis C virus also include agents that inhibit HCV proteases, HCV polymerase, HCV helicase, HCV NS4B protein, HCV entry, HCV assembly, HCV egress, HCV NS5A protein, and inosine 5′-monophosphate dehydrogenase. Other agents include nucleoside analogs for the treatment of an HCV infection. Still other compounds include those disclosed in WO 2004/014313 and WO 2004/014852 and in the references cited therein. The patent applications WO 2004/014313 and WO 2004/014852 are hereby incorporated by references in their entirety.

Specific antiviral agents include Omega IFN (BioMedicines Inc.), BILN-2061 (Boehringer Ingelheim), Summetrel (Endo Pharmaceuticals Holdings Inc.), Roferon A (F. Hoffman-La Roche), Pegasys (F. Hoffman-La Roche), Pegasys/Ribaravin (F. Hoffman-La Roche), CellCept (F. Hoffman-La Roche), Wellferon (GlaxoSmithKline), Albuferon-α (Human Genome Sciences Inc.), Levovirin (ICN Pharmaceuticals), IDN-6556 (Idun Pharmaceuticals), IP-501 (Indevus Pharmaceuticals), Actimmune (InterMune Inc.), Infergen A (InterMune Inc.), ISIS 14803 (ISIS Pharamceuticals Inc.), JTK-003 (Japan Tobacco Inc.), Pegasys/Ceplene (Maxim Pharmaceuticals), Ceplene (Maxim Pharmaceuticals), Civacir (Nabi Biopharmaceuticals Inc.), Intron A/Zadaxin (RegeneRx), Levovirin (Ribapharm Inc.), Viramidine(Ribapharm Inc.), Heptazyme (Ribozyme Pharmaceuticals), Intron A (Schering-Plough), PEG-Intron (Schering-Plough), Rebetron (Schering-Plough), Ribavirin (Schering-Plough), PEG-Intron/Ribavirin (Schering-Plough), Zadazim (SciClone), Rebif (Serono), IFN-β/EMZ701 (Transition Therapeutics), T67 (Tularik Inc.), VX-497 (Vertex Pharmaceuticals Inc.), VX-950/LY-570310 (Vertex Pharmaceuticals Inc.), Omniferon (Viragen Inc.), XTL-002 (XTL Biopharmaceuticals), SCH 503034 (Schering-Plough), isatoribine and its prodrugs ANA971 and ANA975 (Anadys), R1479 (Roche Biosciences), Valopicitabine (Idenix), NIM811 (Novartis), and Actilon (Coley Pharmaceuticals).

In some embodiments, the compositions and methods described herein contain a compound, or pharmaceutically acceptable salt or solvate, described herein and interferon. In some embodiments, the interferon is selected from the group consisting of interferon alpha 2B, pegylated interferon alpha, consensus interferon, interferon alpha 2A, and lymphoblastiod interferon tau.

In other embodiments, the compositions and methods described herein contain a compound, or pharmaceutically acceptable salt or solvate, described herein and a compound having anti-HCV activity is selected from the group consisting of interleukin 2, interleukin 6, interleukin 12, a compound that enhances the development of a type 1 helper T cell response, interfering RNA, anti-sense RNA, Imiquimod, ribavirin, an inosine 5′-monophospate dehydrogenase inhibitor, amantadine, and rimantadine.

In some embodiments, the compound having anti-HCV activity is Ribavirin, levovirin, viramidine, thymosin alpha-1, an inhibitor of NS3 serine protease, and inhibitor of inosine monophosphate dehydrogenase, interferon-alpha, or pegylated interferon-alpha alone or in combination with Ribavirin or viramidine.

In some embodiments, the compound having anti-HCV activity is said agent active against HCV is interferon-alpha or pegylated interferon-alpha alone or in combination with Ribavirin or viramidine.

### Biological Examples

### Biological Example 1. Anti-Hepatitis C Activity

Compounds can exhibit anti-hepatitis C activity by inhibiting viral and host cell targets required in the replication cycle. A number of assays have been published to assess these activities. A general method that assesses the gross increase of HCV virus in culture is disclosed in U.S. Pat. No. 5,738,985 to Miles et al. In vitro assays have been reported in Ferrari et al J. of Vir., 73:1649-1654, 1999; Ishii et al., Hepatology, 29:1227-1235, 1999; Lohmann et al., J. of Bio. Chem., 274:10807-10815, 1999; and Yamashita et al., J. of Bio. Chem., 273:15479-15486, 1998.

### Replicon Assay

A cell line, ET (Huh-lucubineo-ET) was used for screening of compounds, or pharmaceutically acceptable salts or solvates, described herein for inhibition of HCV RNA dependent RNA polymerase. The ET cell line was stably transfected with RNA transcripts harboring a I₃₈₉luc-ubi-neo/NS3-3′/ET; replicon with firefly luciferase-ubiquitin-neomycin phosphotransferase fusion protein and EMCV-IRES driven NS3-5B polyprotein containing the cell culture adaptive mutations (E1202G; T1280I; K1846T) (Krieger at al, 2001 and unpublished). The ET cells were grown in DMEM, supplemented with 10% fetal calf serum, 2 mM Glutamine, Penicillin (100 IU/mL)/Streptomycin (100 μg/mL), 1× nonessential amino acids, and 250 μg/mL G418 (“Geneticin”). They were all available through Life Technologies (Bethesda, Md.). The cells were plated at 0.5-1.0×10⁴ cells/well in the 96 well plates and incubated for 24 hrs before adding the test compounds. The compounds were then added to the cells to achieve a final concentration of 5 or 50 μM. Luciferase activity was measured 48-72 hours later by adding a lysis buffer and the substrate (Catalog number Glo-lysis buffer E2661 and Bright-Glo luciferase system E2620 Promega, Madison, Wis.). Cells should not be too confluent during the assay. Percent inhibition of replication was plotted relative to no compound control. Under the same condition, cytotoxicity of the compounds was determined using cell proliferation reagent, WST-1 (Roche, Germany). The compounds showing antiviral activities, but no significant cytotoxicities were chosen to determine the EC₅₀ and TC₅₀, the effective concentration and toxic concentration at which 50% of the maximum inhibition is observed. For these determinations, 6 dilutions of each compound were used. Compounds were typically diluted 3 fold to span a concentration range of 250 fold. EC₅₀ and similarly TC₅₀ values were calculated by fitting % inhibition at each concentration to the following equation:

% inhibition=100%/[EC₅₀/[I])^{b}+1]

where b is Hill's coefficient.

Certain of the compounds of Formula (I) exhibited a % inhibition of at least 80% when tested at 5 μM. For certain of the compounds of Formula (I), the % inhibition was at least 50% when tested at 5 μM. For certain of the compounds, the % inhibition was at least 10% when tested at 5 μM.

When tested at 5 μM, the following compounds where found respectively to have the following % inhibition values:

| Compound | % inhibition |
|---|---|
| 101 | 80.3 |
| 102 | 99.8 |
| 103 | 100.0 |
| 104 | 99.7 |
| 105 | 99.9 |
| 106 | 99.3 |
| 107 | 98.4 |
| 108 | 99.9 |
| 109 | 98.0 |
| 110 | 99.4 |
| 111 | 16.0 |
| 112 | 80.9 |
| 113 | 24.9 |
| 114 | 99.3 |
| 115 | 67.6 |
| 116 | 92.1 |
| 117 | 99.8 |

Certain of the compounds of Formula (I) exhibited a % inhibition of at least 80% when tested at 10 μM. For certain of the compounds of Formula (I), the % inhibition was at least 50% when tested at 10 μM. For certain of the compounds, the % inhibition was at least 10% when tested at 10 μM.

When tested at 10 μM, the following compounds where found respectively to have the following % inhibition values:

| Compound No. | % inhibition at 10 μM |
|---|---|
| 101 | 92.92 |
| 102 | 99.71 |
| 103 | 99.99 |
| 104 | 99.73 |
| 105 | 99.69 |
| 106 | 99.70 |
| 107 | 99.39 |
| 108 | 99.98 |
| 109 | 95.44 |
| 110 | 97.39 |
| 112 | 85.89 |
| 113 | 41.67 |
| 114 | 98.50 |
| 115 | 75.31 |
| 116 | 92.85 |
| 117 | 99.13 |
| 119 | 0.00 |
| 133 | 10.13 |
| 134 | 0.51 |
| 135 | 0.79 |
| 136 | 5.84 |
| 137 | 40.35 |
| 138 | 48.78 |
| 139 | 51.15 |
| 140 | 82.72 |
| 142 | 66.31 |
| 143 | 96.11 |
| 144 | 92.22 |
| 145 | 85.63 |
| 148 | 99.97 |
| 149 | 43.67 |
| 157 | 9.99 |
| 158 | 27.85 |
| 159 | 49.44 |
| 160 | 61.50 |
| 161 | 54.64 |
| 162 | 66.90 |
| 163 | 62.02 |
| 164 | 90.14 |
| 165 | 79.37 |
| 166 | 70.68 |
| 167 | 93.11 |
| 168 | 99.24 |
| 169 | 83.64 |
| 170 | 97.91 |
| 171 | 91.49 |
| 172 | 95.39 |
| 173 | 98.74 |
| 174 | 99.71 |
| 175 | 99.77 |
| 176 | 99.81 |
| 177 | 99.64 |
| 178 | 98.70 |
| 179 | 99.89 |
| 180 | 83.33 |
| 185 | 47.81 |
| 186 | 100.00 |
| 187 | 99.67 |
| 190 | 99.90 |
| 191 | 99.78 |
| 192 | 54.09 |
| 193 | 76.34 |
| 194 | 57.47 |
| 195 | 98.82 |
| 196 | 89.03 |
| 197 | 54.08 |
| 198 | 50.31 |
| 199 | 98.51 |
| 200 | 99.38 |
| 201 | 99.95 |
| 202 | 99.17 |
| 203 | 99.60 |
| 205 | 98.74 |
| 206 | 98.06 |
| 207 | 99.47 |
| 208 | 69.66 |
| 209 | 93.56 |
| 213 | 98.11 |
| 214 | 98.55 |
| 216 | 79.10 |
| 217 | 85.33 |
| 219 | 34.98 |
| 220 | 99.69 |
| 221 | 98.91 |
| 222 | 98.19 |
| 226 | 99.74 |
| 227 | 97.90 |
| 228 | 40.76 |
| 230 | 35.76 |
| 231 | 41.95 |
| 233 | 96.83 |
| 235 | 56.01 |
| 236 | 76.82 |
| 237 | 1.73 |
| 239 | 95.29 |
| 240 | 16.39 |
| 241 | 99.68 |
| 242 | 96.96 |
| 243 | 99.44 |
| 245 | 72.79 |
| 246 | 62.14 |
| 247 | 80.02 |
| 248 | 15.46 |
| 251 | 97.73 |
| 255 | 4.67 |
| 256 | 9.34 |
| 257 | 0.87 |
| 258 | 40.00 |
| 259 | 50.60 |
| 260 | 53.17 |
| 261 | 73.92 |
| 262 | 96.63 |
| 266 | 10.03 |
| 268 | 93.74 |
| 269 | 36.56 |
| 271 | 2.27 |
| 272 | 91.14 |
| 273 | 94.00 |
| 274 | 54.88 |
| 276 | 60.05 |
| 277 | 99.87 |
| 278 | 99.71 |
| 279 | 98.89 |
| 282 | 83.42 |
| 283 | 66.67 |
| 284 | 77.38 |
| 285 | 90.11 |
| 289 | 57.32 |
| 290 | 95.15 |
| 291 | 22.48 |
| 292 | 64.06 |

### Formulation Examples

The following are representative pharmaceutical formulations containing a compound of Formula (I), or a pharmaceutically acceptable salt or solvate.

### Formulation Example 1

### Tablet Formulation

The following ingredients are mixed intimately and pressed into single scored tablets.

| | Quantity per |
|---|---|
| Ingredient | tablet, mg |
| compound | 400 |
| cornstarch | 50 |
| croscarmellose sodium | 25 |
| lactose | 120 |
| magnesium stearate | 5 |

### Formulation Example 2

### Capsule formulation

The following ingredients are mixed intimately and loaded into a hard-shell gelatin capsule.

| | Quantity per |
|---|---|
| Ingredient | capsule, mg |
| compound | 200 |
| lactose, spray-dried | 148 |
| magnesium stearate | 2 |

### Formulation Example 3

### Suspension Formulation

The following ingredients are mixed to form a suspension for oral administration.

| Ingredient | Amount | |
|---|---|---|
| compound | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. (quantity |
| | sufficient) to 100 mL |

### Formulation Example 4

### Injectable formulation

The following ingredients are mixed to form an injectable formulation.

| Ingredient | Amount |
|---|---|
| compound | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Formulation Example 5

### Suppository Formulation

A suppository of total weight 2.5 g is prepared by mixing the compound with Witepsol® H-15 (triglycerides of saturated vegetable fatty acid; Riches-Nelson, Inc., New York), and has the following composition:

| Ingredient | Amount |
|---|---|
| compound | 500 mg |
| Witepsol ® H-15 | balance |

1. A compound that is Formula (I) 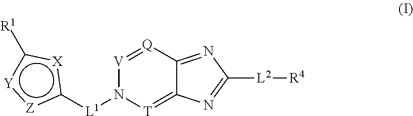
or a pharmaceutically acceptable salt or solvate thereof, wherein:
a) when X is CR² or N, one of Y or Z is O and the other of Y or Z is N; or one of Y or Z is N and the other of Y or Z is NR^{a};
b) when X is O, NR^{a}, or S(O)ₚ wherein p is 0 or 1, one of Y or Z is N and the other of Y or Z is N or CR²;
c) when X is N, one of Y or Z is O and the other of Y or Z is N;
L¹ is L³;
L² is a bond or L³;
L³ is independently C₃₋₆ cycloalkylene or is C₁₋₅ alkylene where one or two —CH₂— groups of said C₁₋₅ alkylene are optionally replaced with —NR^{b}—, —S—, —(C═O)—, or —O— and optionally two —CH₂— groups together form a double bond or triple bond provided that L³ does not contain an —O—O—, —S—O—, or —S—S— group, and wherein said C₁ to C₅ alkylene is optionally substituted with one to three groups independently selected from halo, alkyl, and spirocycloalkyl;
R^{a} and R^{b} are independently H, alkyl, or substituted alkyl;
one of V or T is N and the other of V or T is CR³;
Q is N or CR³;
R¹ and R⁴ are independently selected from aryl, substituted aryl, heteroaryl, substituted heteroaryl, heterocyclyl, substituted heterocyclyl, cycloalkyl, and substituted cycloalkyl;
R² is independently selected from hydrogen, halo, alkyl, substituted alkyl, alkenyl, substituted alkenyl, alkynyl, substituted alkynyl, amino, substituted amino, acylamino, hydroxy, alkoxy, substituted alkoxy, carboxy, carboxy ester, cycloalkyl, substituted cycloalkyl, and cyano; and
R³ is independently selected from hydrogen, halo, amino, substituted amino, acylamino, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, carboxy, carboxy ester, acyloxy, cyano, thiol, alkylthio, substituted alkylthio, and substituted sulfonyl.

2. A compound of claim 1 wherein Q is CR³.

3. A compound of claim 2 wherein R³ is selected from hydrogen and lower alkyl.

4. A compound of claim 3 wherein R³ is hydrogen.

5. A compound of claim 1 wherein Q is N.

6. A compound of claim 1 wherein V is N and T is CR³.

7. A compound of claim 6 wherein R³ is selected from hydrogen and lower alkyl.

8. A compound of claim 7 wherein R³ is hydrogen.

9. A compound of claim 1 wherein V is CR³ and T is N.

10. A compound of claim 9 wherein R³ is selected from hydrogen and lower alkyl.

11. A compound of claim 10 wherein R³ is hydrogen.

12. A compound of claim 1 that is Formula (II) 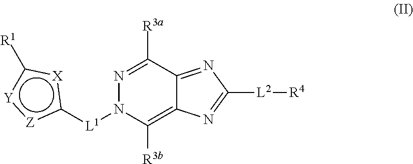
wherein R^{3a} and R^{3b} are independently selected from hydrogen, halo, amino, substituted amino, acylamino, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, carboxy, carboxy ester, acyloxy, cyano, thiol, alkylthio, substituted alkylthio, and substituted sulfonyl.

13. A compound of claim 1 wherein X is CR², Y is O and Z is N.

14. A compound of claim 1 wherein X is CR², Y is N and Z is O.

15. A compound of claim 1 wherein Y is N and Z is O.

16. A compound of claim 1 wherein X is N.

17. A compound of claim 1 wherein X is CR².

18. A compound of claim 1 wherein X is CH.

19. A compound of claim 1 wherein L¹ is C₁₋₃ alkylene where one or two —CH₂— groups of said C₁₋₃ alkylene are optionally replaced with —NR^{b}—, —S—, —(C═O)—, or —O—, and wherein said C₁ to C₃ alkylene is optionally substituted with one to three groups independently selected from halo and lower alkyl.

20. A compound of claim 19 wherein L¹ is C₁₋₃ alkylene optionally substituted with one to three halo groups.

21. A compound of claim 20 wherein L¹ is C₁₋₃ alkylene.

22. A compound of claim 21 wherein L¹ is CH₂.

23. A compound of claim 1 wherein L² is a bond.

24. A compound of claim 1 wherein R¹ is substituted phenyl or substituted heteroaryl.

25. A compound of claim 24 wherein R¹ is phenyl or heteroaryl, each of which is substituted with at least one group selected from alkyl, haloalkyl, and optionally substituted alkoxy.

26. A compound of claim 25 wherein R¹ is phenyl or heteroaryl, each of which is substituted with at least one group selected from lower alkyl, CF₃, and optionally substituted methoxy.

27. A compound of claim 26 wherein R¹ is phenyl substituted with at least one group selected from lower alkyl, CF₃, and optionally substituted methoxy.

28. A compound of claim 27 wherein R¹ is phenyl substituted with at least one group selected from lower alkyl, CF₃, and R⁵—CH₂O— wherein R⁵ is optionally substituted heteroaryl.

29. A compound of claim 28 wherein R¹ is phenyl substituted with at least one group selected from lower alkyl, CF₃, and R⁵—CH₂O— wherein R⁵ is optionally substituted pyridinyl.

30. A compound of claim 29 wherein R¹ is phenyl substituted with at least one group selected from lower alkyl, CF₃, and R⁵—CH₂O— wherein R⁵ is pyridinyl.

31. A compound of claim 24 wherein R¹ is phenyl or heteroaryl, each of which is substituted with at least one haloalkyl group.

32. A compound of claim 31 wherein R¹ is phenyl or heteroaryl, each of which is substituted with at least one CF₃ group.

33. A compound of claim 1 wherein R⁴ is substituted phenyl or substituted heteroaryl.

34. A compound of claim 33 wherein R⁴ is phenyl or heteroaryl, each of which is substituted with at least one halo group.

35. A compound of claim 34 wherein R⁴ is phenyl or heteroaryl, each of which is substituted with at least one fluoro group.

36. A compound of claim 35 wherein R⁴ is phenyl substituted with at least one fluoro group.

37. A compound of claim 36 wherein R⁴ is 2,3-difluorophenyl.

38. A compound of claim 12 wherein R^{3b} is hydrogen.

39. A compound of claim 12 wherein R^{3a} is hydrogen.

40. A compound selected from any one or more of the compounds from the Table below or a pharmaceutically acceptable salt or solvate thereof:

| Com- | | |
|---|---|---|
| pound | | |
| # | Structure | Name |
| 101 | 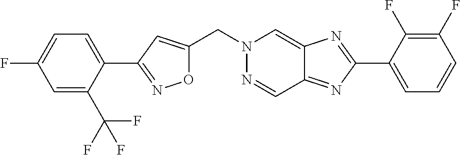 | 2-(2,3- Difluoro- phenyl)- 5-[3-(4-fluoro- 2-trifluoro- methyl- phenyl)- isoxazol- 5-ylmethyl]- 5H- imidazo [4,5-d] pyridazine |
| 102 | 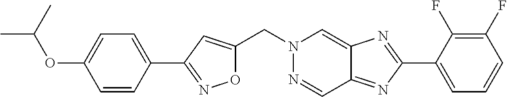 | 2-(2,3- Difluoro- phenyl)- 5-[3-(4- isopropoxy- phenyl)- isoxazol-5- ylmethyl]- 5H- imidazo [4,5-d] pyridazine |
| 103 | 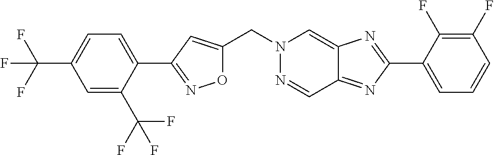 | 5-[3-(2,4-Bis- trifluoro- methyl- phenyl)- isoxazol-5- ylmethyl]- 2-(2,3- difluoro- phenyl)-5H- imidazo [4,5-d] pyridazine |
| 104 | 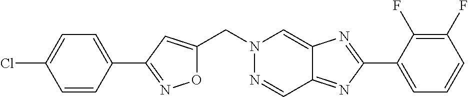 | 5-[3-(4- Chloro- phenyl)- isoxazol-5- ylmethyl]-2- (2,3-difluoro- phenyl)-5H- imidazo[4,5-d] pyridazine |
| 105 | 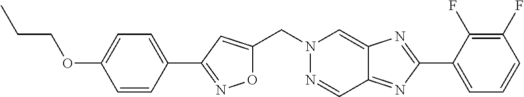 | 2-(2,3- Difluoro- phenyl)- 5-[3-(4- propoxy- phenyl)- isoxazol-5- ylmethyl]- 5H-imidazo [4,5-d] pyridazine |
| 106 | 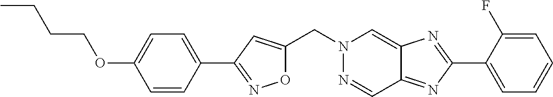 | 5-[3-(4- Butoxy- phenyl)- isoxazol-5- ylmethyl]- 2-(2-fluoro- phenyl)-5H- imidazo[4,5-d] pyridazine |
| 107 | 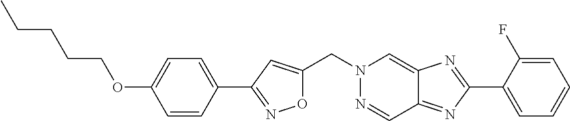 | 2-(2- Fluoro- phenyl)-5- [3-(4- pentyloxy- phenyl)- isoxazol- 5- ylmethyl]-5H- imidazo[4,5-d] pyridazine |
| 108 | 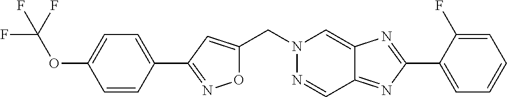 | 2-(2-Fluoro- phenyl)- 5-[3-(4- trifluoro- methoxy- phenyl)- isoxazol- 5- ylmethyl]-5H- imidazo [4,5-d] pyridazine |
| 109 | 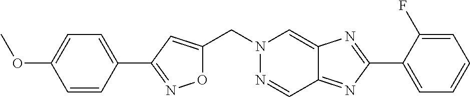 | 2-(2-Fluoro- phenyl)- 5-[3-(4- methoxy- phenyl)- isoxazol- 5-ylmethyl]- 5H- imidazo [4,5-d] pyridazine |
| 110 | 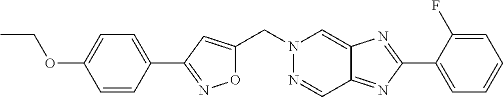 | 5-[3-(4- Ethoxy- phenyl)- isoxazol-5- ylmethyl]-2- (2-fluoro- phenyl)-5H- imidazo [4,5-d] pyridazine |
| 111 | 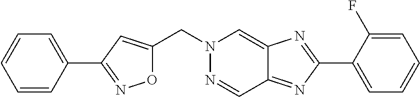 | 2-(2-Fluoro- phenyl)- 5-(3-phenyl- isoxazol-5- ylmethyl)-5H- imidazo [4,5-d] pyridazine |
| 112 | 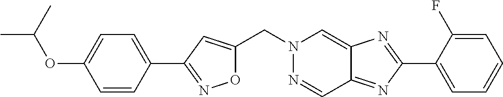 | 2-(2-Fluoro- phenyl)- 5-[3-(4- isopropoxy- phenyl)- isoxazol- 5- ylmethyl]-5H- imidazo [4,5-d] pyridazine |
| 113 | 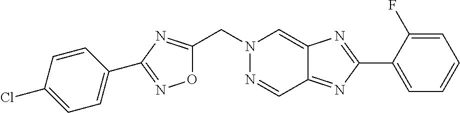 | 5-[3-(4- Chloro- phenyl)- [1,2,4] oxadiazol-5- ylmethyl]- 2-(2-fluoro- phenyl)- 5H-imidazo [4,5-d] pyridazine |
| 114 | 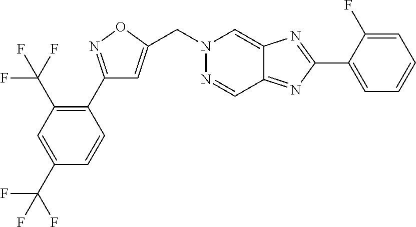 | 5-[3-(2,4- Bis- trifluoro- methyl- phenyl)- isoxazol-5- ylmethyl]- 2- (2-fluoro- phenyl)- 5H- imidazo[4,5-d] pyridazine |
| 115 | 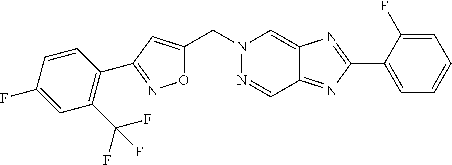 | 2-(2-Fluoro- phenyl)- 5-[3-(4-fluoro- 2- trifluoro- methyl- phenyl)- isoxazol-5- ylmethyl]-5H- imidazo [4,5-d] pyridazine |
| 116 | 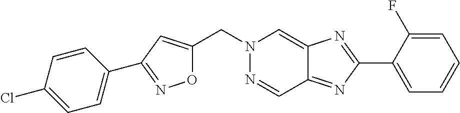 | 5-[3-(4- Chloro- phenyl)- isoxazol-5- ylmethyl]-2-(2- fluoro- phenyl)-5H- imidazo[4,5-d] pyridazine |
| 117 | 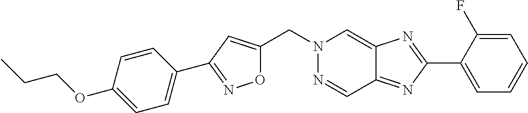 | 2-(2-Fluoro- phenyl)- 5-[3-(4- propoxy- phenyl)- isoxazol- 5-ylmethyl]- 5H- imidazo [4,5-d] pyridazine |
| 119 | 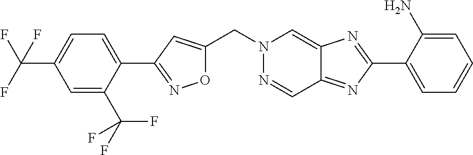 | 2-{5-[3-(2,4-Bis- trifluoro- methyl- phenyl)- isoxazol- 5-ylmethyl]- 5H- imidazo [4,5-d] pyridazin- 2-yl}- phenylamine |
| 120 | 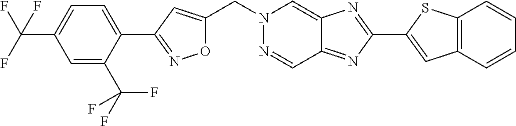 | 2-Benzo[b] thiophen- 2-yl-5-[3- (2,4-bis- trifluoro- methyl- phenyl)- isoxazol- 5-ylmethyl]- 5H- imidazo [4,5-d] pyridazine |
| 121 | 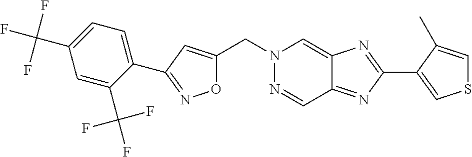 | 5-[3-(2,4-Bis- trifluoro- methyl- phenyl)- isoxazol-5- ylmethyl]- 2-(4-methyl- thiophen-3- yl)-5H- imidazo[4,5- d]pyridazine |
| 122 | 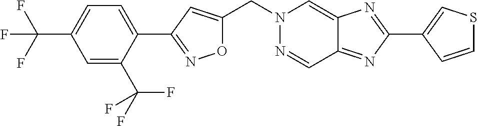 | 5-[3-(2,4- Bis- trifluoro- methyl- phenyl)- isoxazol-5- ylmethyl]- 2-thiophen- 3-yl- 5H-imidazo [4,5-d] pyridazine |
| 123 | 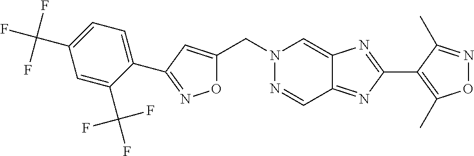 | 5-[3-(2,4-Bis- trifluoro- methyl- phenyl)- isoxazol-5- ylmethyl]- 2-(3,5- dimethyl- isoxazol-4- yl)-5H- imidazo[4,5- d]pyridazine |
| 124 | 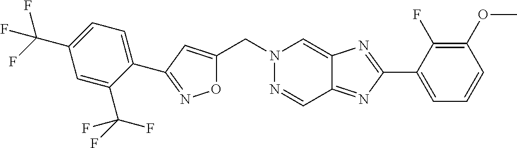 | 5-[3-(2,4-Bis- trifluoro- methyl- phenyl)- isoxazol-5- ylmethyl]- 2-(2-fluoro- 3-methoxy- phenyl)- 5H-imidazo [4,5-d] pyridazine |
| 125 | 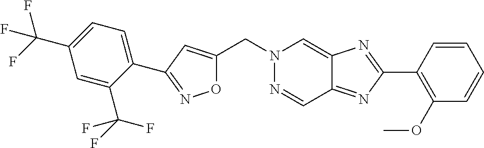 | 5-[3-(2,4-Bis- trifluoro- methyl- phenyl)- isoxazol-5- ylmethyl]-2- (2-methoxy- phenyl)-5H- imidazo[4,5-d] pyridazine |
| 126 | 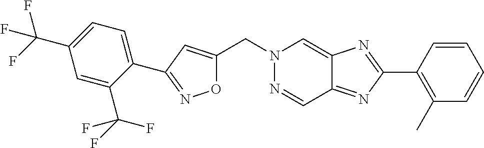 | 5-[3-(2,4-Bis- trifluoro- methyl- phenyl)- isoxazol-5- ylmethyl]-2- o-tolyl-5H- imidazo [4,5-d] pyridazine |
| 127 | 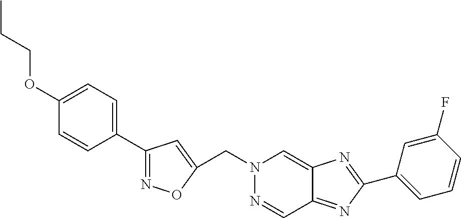 | 2-(3-Fluoro- phenyl)- 5-[3-(4- propoxy- phenyl)- isoxazol- 5- ylmethyl]- 5H- imidazo[4,5-d] pyridazine |
| 128 | 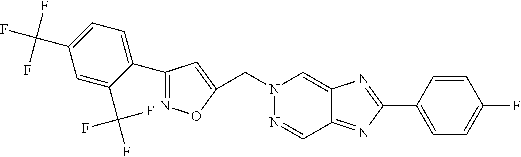 | 5-[3-(2,4-Bis- trifluoro- methyl- phenyl)- isoxazol-5- ylmethyl]-2- (4-fluoro- phenyl)-5H- imidazo[4,5-d] pyridazine |
| 129 | 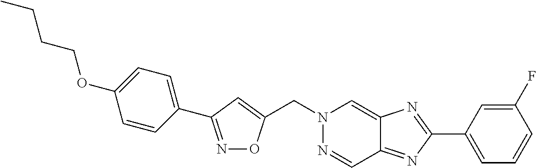 | 5-[3-(4- Butoxy- phenyl)- isoxazol- 5- ylmethyl]-2- (3-fluoro- phenyl)-5H- imidazo[4,5-d] pyridazine |
| 130 | 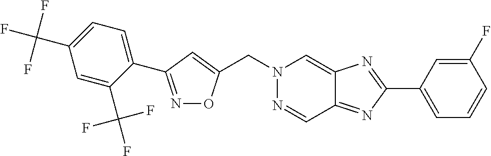 | 5-[3-(2,4-Bis- trifluoro- methyl- phenyl)- isoxazol-5- ylmethyl]- 2-(3-fluoro- phenyl)-5H- imidazo [4,5-d] pyridazine |
| 131 | 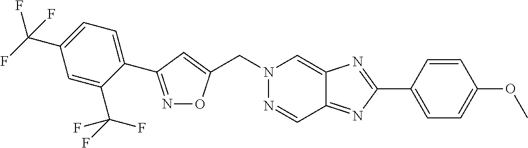 | 5-[3-(2,4-Bis- trifluoro- methyl- phenyl)- isoxazol-5- ylmethyl]- 2-(4-methoxy- phenyl)-5H- imidazo [4,5-d] pyridazine |
| 132 | 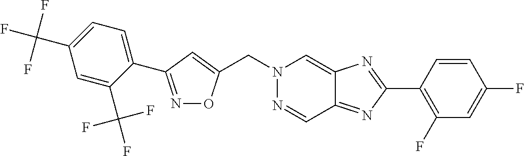 | 5-[3-(2,4-Bis- trifluoro- methyl- phenyl)- isoxazol-5- ylmethyl]- 2-(2,4- difluoro- phenyl)-5H- imidazo [4,5-d] pyridazine |
| 133 | 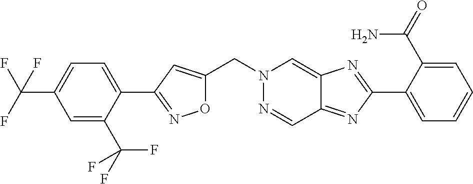 | 2-{5-[3-(2,4-Bis- trifluoro- methyl- phenyl)- isoxazol- 5- ylmethyl]-5H- imidazo[4,5-d] pyridazin- 2-yl}- benzamide |
| 134 | 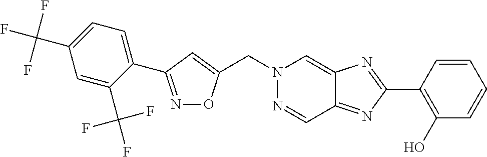 | 2-{5-[3-(2,4-Bis- trifluoro- methyl- phenyl)- isoxazol- 5-ylmethyl]- 5H-imidazo [4,5-d] pyridazin- 2-yl}- phenol |
| 135 | 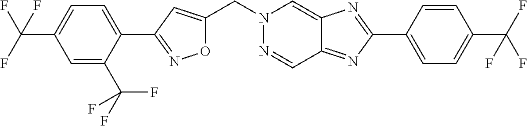 | 5-[3-(2,4-Bis- trifluoro- methyl- phenyl)- isoxazol-5- ylmethyl]- 2-(4- trifluoro- methyl- phenyl)-5H- imidazo[4,5-d] pyridazine |
| 136 | 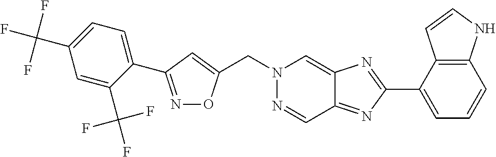 | 5-[3-(2,4-Bis- trifluoro- methyl- phenyl)- isoxazol-5- ylmethyl]-2- (1H-indol-4- yl)-5H- imidazo [4,5-d] pyridazine |
| 137 | 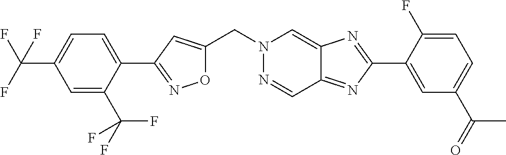 | 1-(3-{5-[3- (2,4-Bis- trifluoro- methyl- phenyl)- isoxazol- 5- ylmethyl]-5H- imidazo[4,5-d] pyridazin-2- yl}-4- fluoro-phenyl)- ethanone |
| 138 | 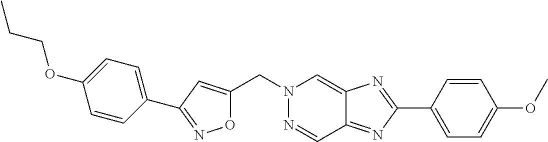 | 2-(4-Methoxy- phenyl)-5-[3- (4-propoxy- phenyl)- isoxazol- 5-ylmethyl]- 5H- imidazo [4,5-d] pyridazine |
| 139 | 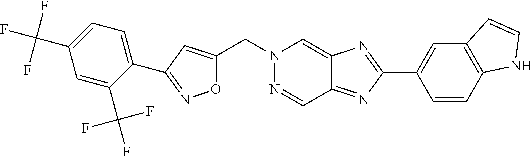 | 5-[3-(2,4-Bis- trifluoro- methyl- phenyl)- isoxazol-5- ylmethyl]-2- (1H-indol- 5-yl)- 5H-imidazo [4,5-d] pyridazine |
| 140 | 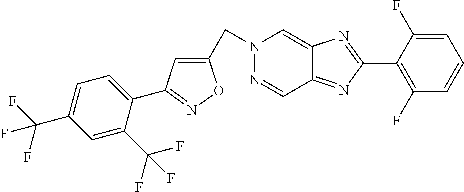 | 5-[3-(2,4-Bis- trifluoro- methyl- phenyl)- isoxazol-5- ylmethyl]-2- (2,6-difluoro- phenyl)-5H- imidazo [4,5-d] pyridazine |
| 141 | 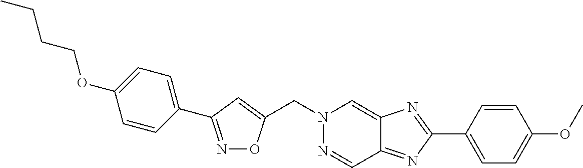 | 5-[3-(4- Butoxy- phenyl)- isoxazol- 5- ylmethyl]-2-(4- methoxy- phenyl)-5H- imidazo[4,5-d] pyridazine |
| 142 | 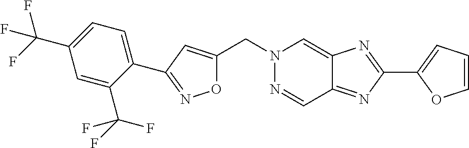 | 5-[3-(2,4-Bis- trifluoro- methyl- phenyl)- isoxazol-5- ylmethyl]-2- furan-2-yl-5H- imidazo[4,5-d] pyridazine |
| 143 | 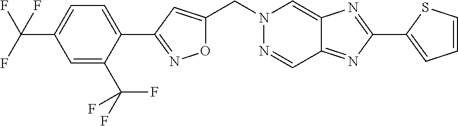 | 5-[3-(2,4-Bis- trifluoro- methyl- phenyl)- isoxazol-5- ylmethyl]-2- thiophen-2-yl- 5H-imidazo [4,5-d] pyridazine |
| 144 | 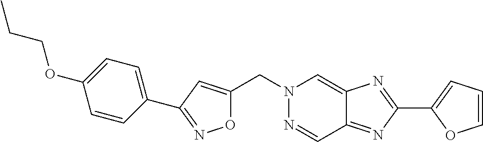 | 2-Furan-2-yl-5- [3-(4-propoxy- phenyl)- isoxazol-5- ylmethyl]-5H- imidazo[4,5- d]pyridazine |
| 145 | 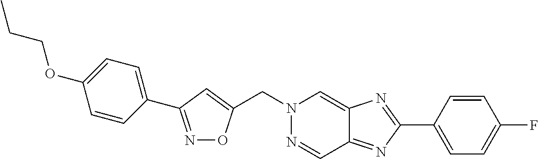 | 2-(4-Fluoro- phenyl)-5- [3-(4-propoxy- phenyl)- isoxazol- 5- ylmethyl]- 5H- imidazo[4,5-d] pyridazine |
| 146 | 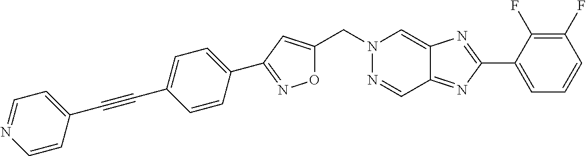 | 2-(2,3- Difluoro- phenyl)-5-[3- (4- pyridin-4- ylethynyl- phenyl)- isoxazol- 5- ylmethyl]-5H- imidazo [4,5-d] pyridazine |
| 147 | 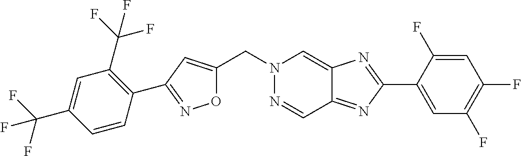 | 5-[3-(2,4-Bis- trifluoro- methyl- phenyl)- isoxazol-5- ylmethyl]-2- (2,4,5-trifluoro- phenyl)-5H- imidazo [4,5-d] pyridazine |
| 148 | 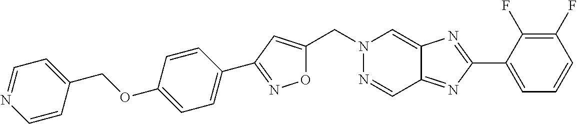 | 2-(2,3-Difluoro- phenyl)- 5-{3-[4- (pyridin-4- ylmethoxy)- phenyl]- isoxazol-5- ylmethyl}- 5H-imidazo [4,5-d] pyridazine |
| 149 | 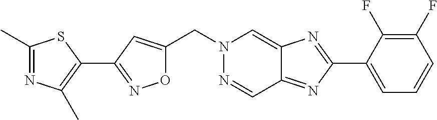 | 2-(2,3-Difluoro- phenyl)-5- [3-(2,4- dimethyl- thiazol- 5-yl)-isoxazol- 5-ylmethyl]- 5H-imidazo [4,5-d] pyridazine |
| 150 | 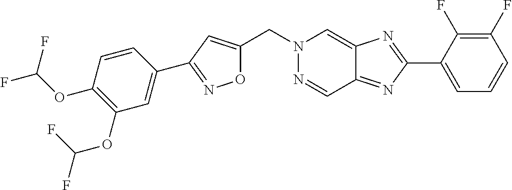 | 5-[3-(3,4-Bis- difluoro- methoxy- phenyl)- isoxazol-5- ylmethyl]- 2-(2,3-difluoro- phenyl)-5H- imidazo [4,5-d] pyridazine |
| 151 | 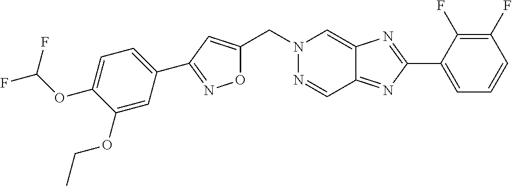 | 5-[3-(4- Difluoro- methoxy- 3- ethoxy-phenyl)- isoxazol-5- ylmethyl]- 2-(2,3-difluoro- phenyl)- 5H-imidazo[4,5- d]pyridazine |
| 152 | 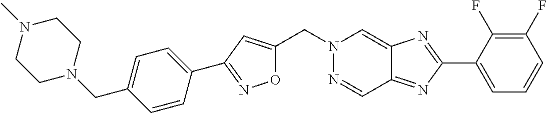 | 2-(2,3-Difluoro- phenyl)-5-{3- [4-(4- methyl- piperazin- 1-ylmethyl)- phenyl]- isoxazol-5- ylmethyl}- 5H-imidazo[4,5- d]pyridazine |
| 153 | 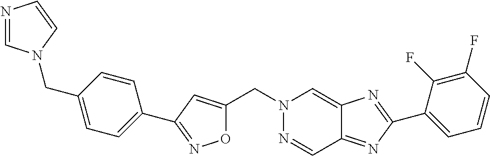 | 2-(2,3-Difluoro- phenyl)-5-[3-(4- imidazol-1- ylmethyl- phenyl)-isoxazol- 5-ylmethyl]- 5H- imidazo [4,5-d] pyridazine |
| 154 | 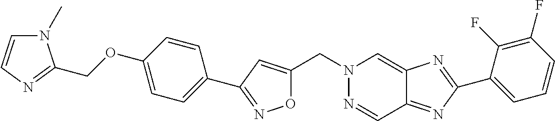 | 2-(2,3-Difluoro- phenyl)-5- {3-[4-(1- methyl-1H- imidazol- 2-ylmethoxy)- phenyl]-isoxazol- 5-ylmethyl}- 5H- imidazo [4,5-d] pyridazine |
| 155 | 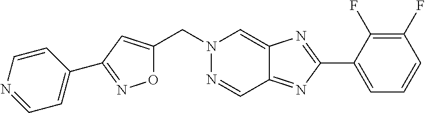 | 2-(2,3-Difluoro- phenyl)-5-(3- pyridin- 4-yl-isoxazol- 5-ylmethyl)- 5H- imidazo [4,5-d] pyridazine |
| 156 | 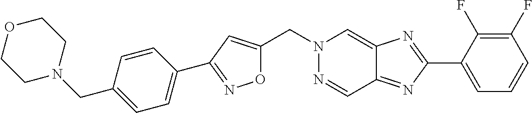 | 2-(2,3-Difluoro- phenyl)-5-[3-(4- morpholin-4- ylmethyl- phenyl)- isoxazol- 5-ylmethyl]-5H- imidazo[4,5- d]pyridazine |
| 157 | 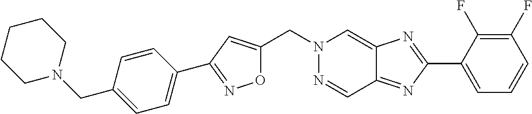 | 2-(2,3-Difluoro- phenyl)-5-[3-(4- piperidin-1- ylmethyl- phenyl)- isoxazol- 5-ylmethyl]- 5H- imidazo[4,5- d]pyridazine |
| 158 | 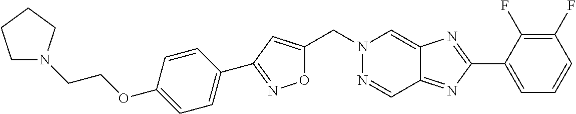 | 2-(2,3-Difluoro- phenyl)-5- {3-[4-(2- pyrrolidin-1-yl- ethoxy)-phenyl]- isoxazol-5- ylmethyl}- 5H-imidazo [4,5- d]pyridazine |
| 159 | 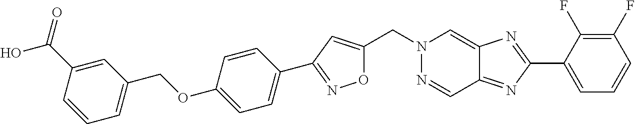 | 3-(4-{5-[2-(2,3- Difluoro- phenyl)- imidazo [4,5-d] pyridazin- 5-ylmethyl]- isoxazol-3-yl}- phenoxymethyl)- benzoic acid |
| 160 | 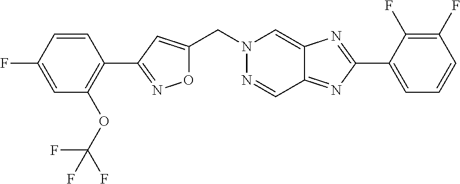 | 2-(2,3-Difluoro- phenyl)-5-[3-(4- fluoro-2- trifluoromethoxy- phenyl)-isoxazol- 5-ylmethyl]-5H- imidazo[4,5-d] pyridazine |
| 161 | 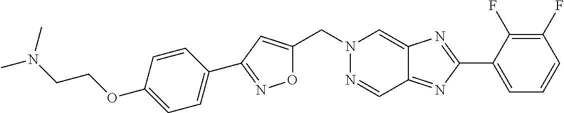 | [2-(4-{5-[2-(2,3- Difluoro- phenyl)- imidazo[4,5-d] pyridazin- 5-ylmethyl]- isoxazol-3-yl}- phenoxy)- ethyl]-dimethyl- amine |
| 162 | 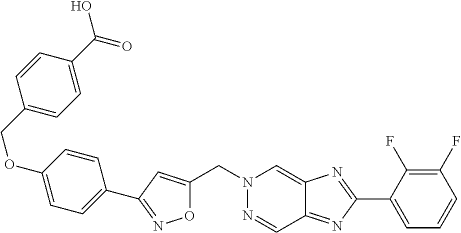 | 4-(4-{5-[2-(2,3- Difluoro- phenyl)- imidazo[4,5-d] pyridazin- 5-ylmethyl]- isoxazol-3-yl}- phenoxy- methyl)- benzoic acid |
| 163 | 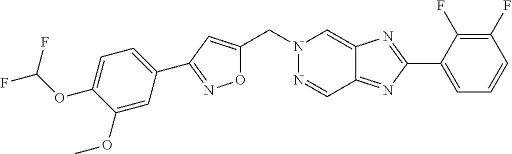 | 5-[3-(4- Difluoro- methoxy- 3-methoxy- phenyl)- isoxazol-5- ylmethyl]-2-(2,3- difluoro-phenyl)- 5H-imidazo[4,5- d]pyridazine |
| 164 | 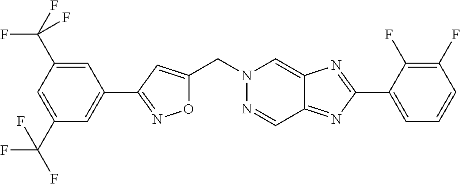 | 5-[3-(3,5-Bis- trifluoro- methyl- phenyl)- isoxazol-5- ylmethyl]- 2-(2,3-difluoro- phenyl)-5H- imidazo [4,5-d] pyridazine |
| 165 | 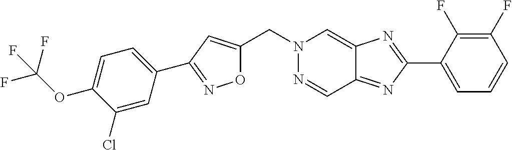 | 5-[3-(3-Chloro- 4- trifluoro- methoxy- phenyl)- isoxazol- 5-ylmethyl]- 2-(2,3- difluoro- phenyl)- 5H-imidazo [4,5-d] pyridazine |
| 166 | 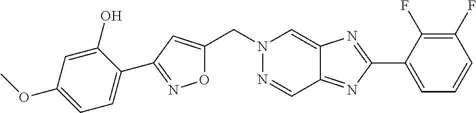 | 2-{5-[2-(2,3- Difluoro- phenyl)- imidazo[4,5-d] pyridazin-5- ylmethyl]- isoxazol- 3-yl}-5- methoxy- phenol |
| 167 | 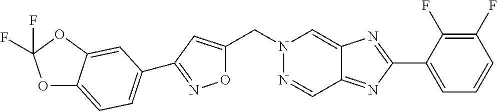 | 5-[3-(2,2- Difluoro- benzo [1,3]dioxol- 5-yl)-isoxazol- 5- ylmethyl]-2- (2,3- difluoro- phenyl)- 5H- imidazo[4,5-d] pyridazine |
| 168 | 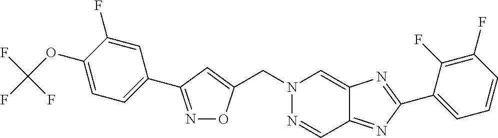 | 2-(2,3- Difluoro- phenyl)-5-[3- (3-fluoro- 4- trifluoro- methoxy- phenyl)- isoxazol- 5- ylmethyl]-5H- imidazo [4,5-d] pyridazine |
| 169 | 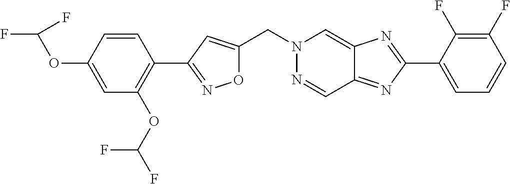 | 5-[3-(2,4-Bis- difluoro- methoxy- phenyl)- isoxazol-5- ylmethyl]- 2-(2,3- difluoro- phenyl)-5H- imidazo [4,5-d] pyridazine |
| 170 | 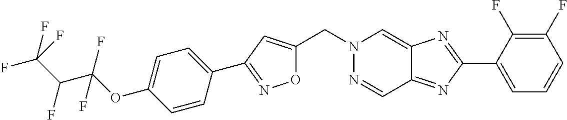 | 2-(2,3- Difluoro- phenyl)-5- {3-[4- (1,1,2,3,3,3- hexafluoro- propoxy)- phenyl]-isoxazol- 5-ylmethyl}-5H- imidazo[4,5-d] pyridazine |
| 171 | 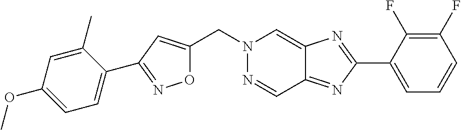 | 2-(2,3- Difluoro- phenyl)- 5-[3-(4- methoxy-2- methyl- phenyl)- isoxazol- 5-ylmethyl]- 5H- imidazo [4,5-d] pyridazine |
| 172 | 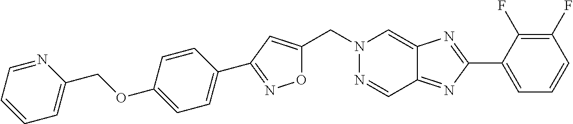 | 2-(2,3- Difluoro- phenyl)-5- {3-[4- (pyridin-2- ylmethoxy)- phenyl]- isoxazol- 5-ylmethyl}- 5H-imidazo [4,5-d] pyridazine |
| 173 | 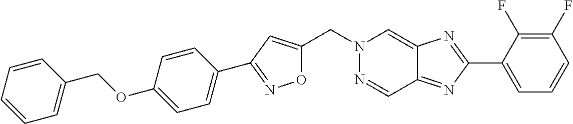 | 5-[3-(4- Benzyloxy- phenyl)- isoxazol- 5-ylmethyl]- 2-(2,3- difluoro- phenyl)- 5H-imidazo [4,5-d] pyridazine |
| 174 | 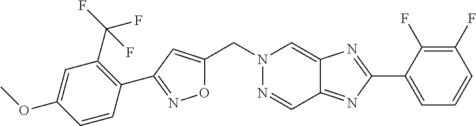 | 2-(2,3-Difluoro- phenyl)-5-[3-(4- methoxy-2- trifluoro- methyl- phenyl)- isoxazol-5- ylmethyl]- 5H-imidazo[4,5- d]pyridazine |
| 175 | 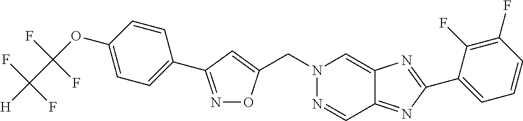 | 2-(2,3- Difluoro- phenyl)-5- {3-[4- (1,1,2,2- tetrafluoro- ethoxy)- phenyl]- isoxazol-5- ylmethyl}- 5H-imidazo [4,5-d] pyridazine |
| 176 | 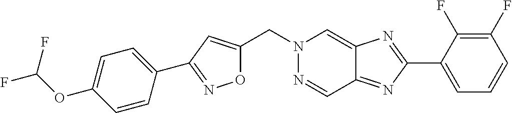 | 5-[3-(4- Difluoro- methoxy- phenyl)- isoxazol-5- ylmethyl]- 2-(2,3-difluoro- phenyl)-5H- imidazo [4,5-d] pyridazine |
| 177 | 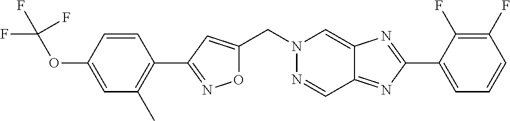 | 2-(2,3- Difluoro- phenyl)-5-[3- (2- methyl-4- trifluoro- methoxy- phenyl)- isoxazol- 5-ylmethyl]- 5H- imidazo [4,5-d] pyridazine |
| 178 | 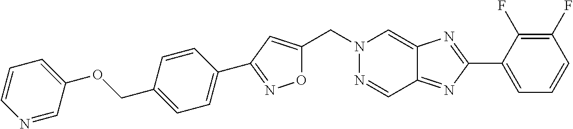 | 2-(2,3- Difluoro- phenyl)- 5-{3-[4- (pyridin- 3- yloxymethyl)- phenyl]- isoxazol-5- ylmethyl}-5H- imidazo[4,5- d]pyridazine |
| 179 | 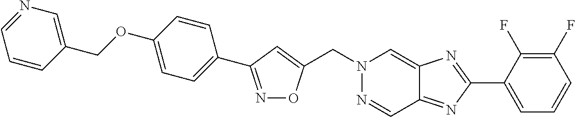 | 2-(2,3- Difluoro- phenyl)-5-{3- [4- (pyridin-3- ylmethoxy)- phenyl]- isoxazol- 5-ylmethyl}- 5H- imidazo[4,5- d]pyridazine |
| 180 | 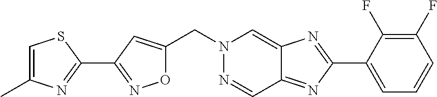 | 2-(2,3- Difluoro- phenyl)-5-[3-(4- methyl-thiazol- 2-yl)- isoxazol-5- ylmethyl]- 5H-imidazo [4,5-d] pyridazine |
| 181 | 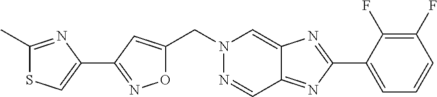 | 2-(2,3-Difluoro- phenyl)-5-[3- (2-methyl- thiazol-4-yl)- isoxazol-5- ylmethyl]-5H- imidazo[4,5-d] pyridazine |
| 182 | 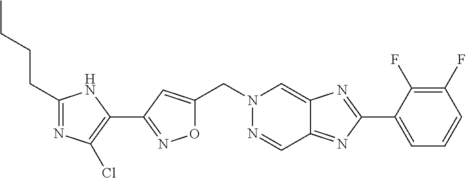 | 5-[3-(2- Butyl-5- chloro-3H- imidazol- 4-yl)-isoxazol-5- ylmethyl]- 2-(2,3-difluoro- phenyl)-5H- imidazo [4,5-d] pyridazine |
| 183 | 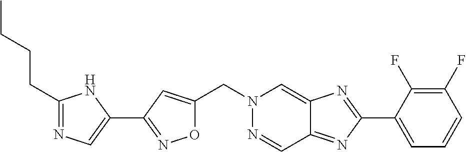 | 5-[3-(2-Butyl- 3H-imidazol- 4-yl)-isoxazol- 5- ylmethyl]-2- (2,3- difluoro-phenyl)- 5H- imidazo [4,5-d] pyridazine |
| 184 | 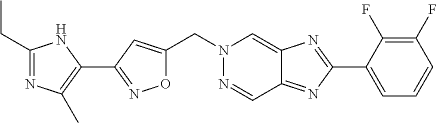 | 2-(2,3- Difluoro- phenyl)- 5-[3-(2- ethyl- 5-methyl-3H- imidazol- 4-yl)-isoxazol- 5-ylmethyl]- 5H- imidazo [4,5-d] pyridazine |
| 185 | 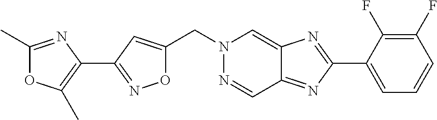 | 2-(2,3- Difluoro- phenyl)-5- [3-(2,5- dimethyl- oxazol- 4-yl)-isoxazol- 5- ylmethyl]- 5H- imidazo [4,5-d] pyridazine |
| 186 | 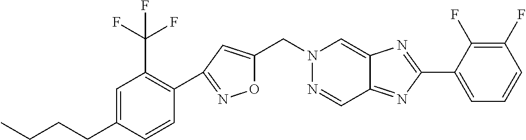 | 5-[3-(4-Butyl- 2- trifluoro- methyl- phenyl)- isoxazol-5- ylmethyl]- 2-(2,3- difluoro- phenyl)-5H- imidazo [4,5-d] pyridazine |
| 187 | 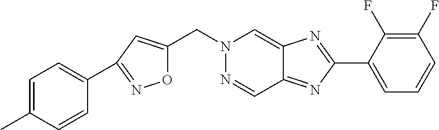 | 2-(2,3-Difluoro- phenyl)-5-(3- p-tolyl- isoxazol-5- ylmethyl)-5H- imidazo[4,5- d]pyridazine |
| 188 | 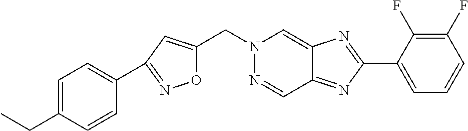 | 2-(2,3-Difluoro- phenyl)-5-[3- (4-ethyl- phenyl)-isoxazol- 5-ylmethyl]-5H- imidazo[4,5-d] pyridazine |
| 189 | 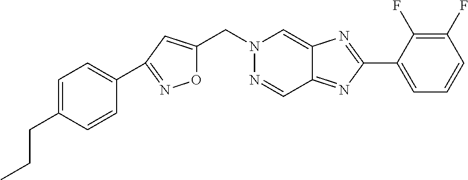 | 2-(2,3-Difluoro- phenyl)-5-[3-(4- propyl-phenyl)- isoxazol- 5-ylmethyl]-5H- imidazo[4,5-d] pyridazine |
| 190 | 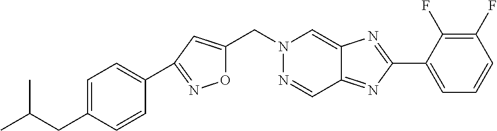 | 2-(2,3-Difluoro- phenyl)-5-[3-(4- isobutyl-phenyl)- isoxazol-5- ylmethyl]- 5H-imidazo[4,5-d] pyridazine |
| 191 | 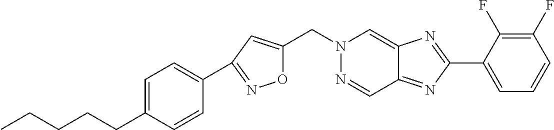 | 2-(2,3-Difluoro- phenyl)-5-[3- (4-pentyl- phenyl)- isoxazol-5- ylmethyl]-5H- imidazo[4,5-d] pyridazine |
| 192 | 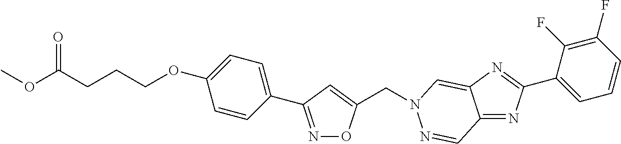 | 4-(4-{5-[2-(2,3- Difluoro- phenyl)- imidazo[4,5-d] pyridazin-5- ylmethyl]- isoxazol-3-yl}- phenoxy)- butyric acid methyl ester |
| 193 | 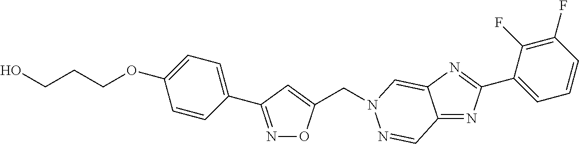 | 3-(4-{5-[2-(2,3- Difluoro- phenyl)- imidazo[4,5-d] pyridazin-5- ylmethyl]- isoxazol-3-yl}- phenoxy)- propan- 1-ol |
| 194 | 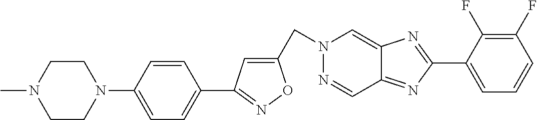 | 2-(2,3- Difluoro- phenyl)-5- {3-[4-(4- methyl- piperazin- 1-yl)-phenyl]- isoxazol-5- ylmethyl}- 5H-imidazo [4,5-d] pyridazine |
| 195 | 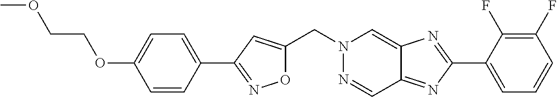 | 2-(2,3-Difluoro- phenyl)-5- {3-[4- (2-methoxy- ethoxy)- phenyl]- isoxazol- 5-ylmethyl}- 5H- imidazo[4,5-d] pyridazine |
| 196 | 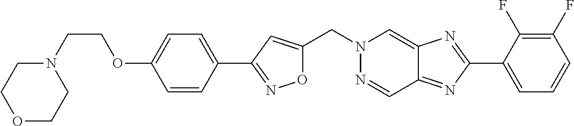 | 2-(2,3-Difluoro- phenyl)-5-{3-[4- (2-morpholin- 4-yl- ethoxy)-phenyl]- isoxazol-5- ylmethyl}- 5H-imidazo[4,5- d]pyridazine |
| 197 | 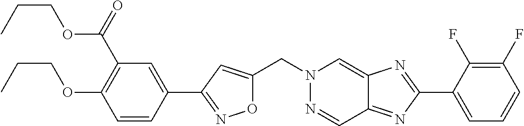 | 5-{5-[2-(2,3- Difluoro- phenyl)- imidazo[4,5-d] pyridazin- 5-ylmethyl]- isoxazol-3-yl}- 2- propoxy- benzoic acid propyl ester |
| 198 | 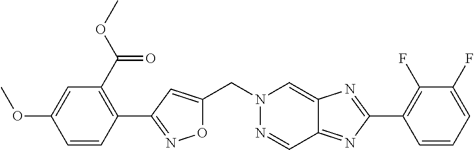 | 2-{5-[2-(2,3- Difluoro- phenyl)- imidazo[4,5-d] pyridazin- 5-ylmethyl]- isoxazol-3-yl}- 5-methoxy- benzoic acid methyl ester |
| 199 | 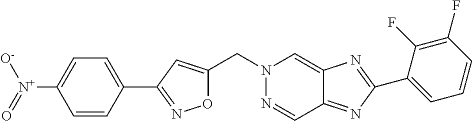 | 2-(2,3-Difluoro- phenyl)-5-[3-(4- nitro-phenyl)- isoxazol-5- ylmethyl]-5H- imidazo[4,5-d] pyridazine |
| 200 | 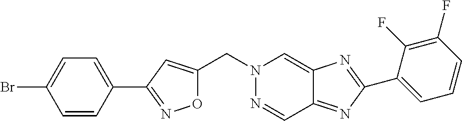 | 5-[3-(4-Bromo- phenyl)-isoxazol- 5- ylmethyl]-2-(2,3- difluoro-phenyl)- 5H- imidazo[4,5-d] pyridazine |
| 201 | 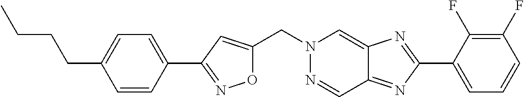 | 5-[3-(4-Butyl- phenyl)-isoxazol- 5- ylmethyl]-2-(2,3- difluoro-phenyl)- 5H- imidazo[4,5-d] pyridazine |
| 202 | 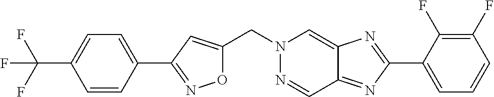 | 2-(2,3-Difluoro- phenyl)-5-[3-(4- trifluoro- methyl- phenyl)-isoxazol- 5- ylmethyl]-5H- imidazo [4,5-d]pyridazine |
| 203 | 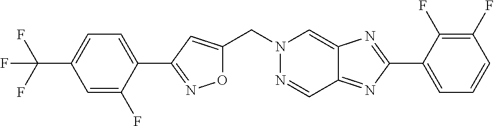 | 2-(2,3-Difluoro- phenyl)-5-[3-(2- fluoro- 4-trifluoro- methyl- phenyl)-isoxazol- 5- ylmethyl]-5H- imidazo [4,5-d]pyridazine |
| 204 | 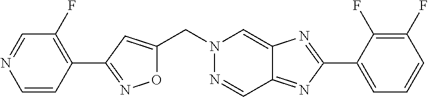 | 2-(2,3-Difluoro- phenyl)-5-[3-(3- fluoro- pyridin-4-yl)- isoxazol- 5-ylmethyl]-5H- imidazo[4,5-d] pyridazine |
| 205 | 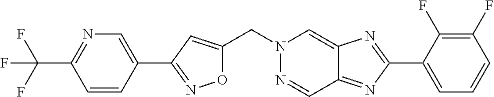 | 2-(2,3-Difluoro- phenyl)-5-[3-(6- trifluoro- methyl- pyridin-3-yl)- isoxazol-5- ylmethyl]-5H- imidazo [4,5-d] pyridazine |
| 206 | 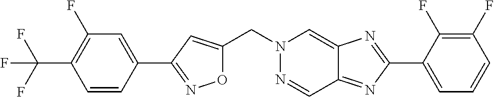 | 2-(2,3-Difluoro- phenyl)-5-[3- (3-fluoro- 4- trifluoro- methyl- phenyl)-isoxazol- 5- ylmethyl]-5H- imidazo [4,5-d] pyridazine |
| 207 | 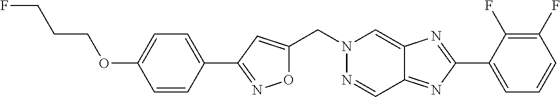 | 2-(2,3- Difluoro- phenyl)-5-{3- [4-(3-fluoro- propoxy)- phenyl]- isoxazol- 5-ylmethyl}- 5H-imidazo [4,5-d] pyridazine |
| 208 | 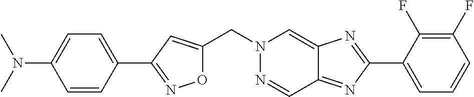 | (4-{5-[2-(2,3- Difluoro- phenyl)- imidazo[4,5-d] pyridazin- 5-ylmethyl]- isoxazol-3-yl}- phenyl)- dimethyl- amine |
| 209 | 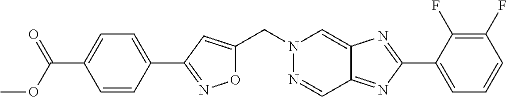 | 4-{5-[2-(2,3- Difluoro- phenyl)- imidazo[4,5-d] pyridazin- 5-ylmethyl]- isoxazol-3-yl}- benzoic acid methyl ester |
| 210 | 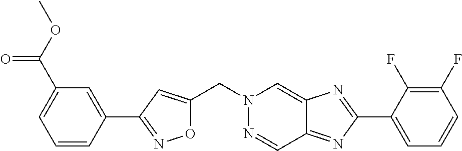 | 3-{5-[2-(2,3- Difluoro- phenyl)- imidazo[4,5-d] pyridazin- 5-ylmethyl]- isoxazol-3-yl}- benzoic acid methyl ester |
| 211 | 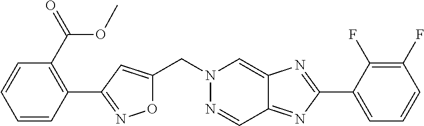 | 2-{5-[2-(2,3- Difluoro- phenyl)- imidazo[4,5-d] pyridazin- 5-ylmethyl]- isoxazol-3-yl}- benzoic acid methyl ester |
| 212 | 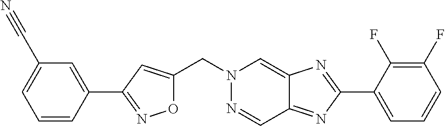 | 3-{5-[2-(2,3- Difluoro- phenyl)- imidazo[4,5-d] pyridazin- 5-ylmethyl]- isoxazol-3-yl}- benzonitrile |
| 213 | 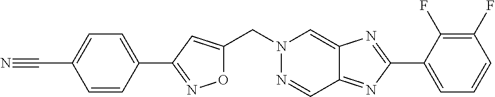 | 4-{5-[2-(2,3- Difluoro- phenyl)- imidazo[4,5-d] pyridazin- 5-ylmethyl]- isoxazol-3-yl}- benzonitrile |
| 214 | 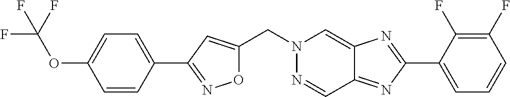 | 2-(2,3-Difluoro- phenyl)-5-[3- (4- trifluoro- methoxy- phenyl)- isoxazol- 5-ylmethyl]- 5H-imidazo [4,5-d] pyridazine |
| 215 | 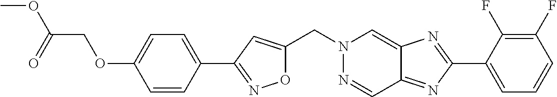 | (4-{5-[2-(2,3- Difluoro- phenyl)- imidazo [4,5-d] pyridazin- 5-ylmethyl]- isoxazol-3-yl}- phenoxy)- acetic acid methyl ester |
| 216 | 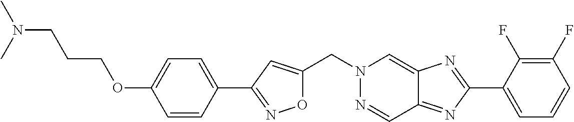 | [3-(4-{5-[2-(2,3- Difluoro- phenyl)- imidazo [4,5-d] pyridazin- 5-ylmethyl]- isoxazol-3-yl}- phenoxy)- propyl]- dimethyl- amine |
| 217 | 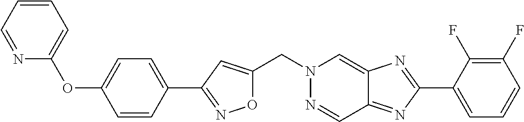 | 2-(2,3-Difluoro- phenyl)-5-{3- [4-(pyridin- 2-yloxy)- phenyl]- isoxazol- 5-ylmethyl}- 5H-imidazo [4,5-d] pyridazine |
| 218 | 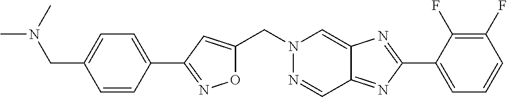 | (4-{5-[2-(2,3- Difluoro- phenyl)- imidazo[4,5-d] pyridazin- 5-ylmethyl]- isoxazol-3-yl}- benzyl)- dimethyl- amine |
| 219 | 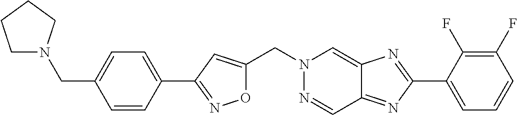 | 2-(2,3-Difluoro- phenyl)-5-[3-(4- pyrrolidin-1- ylmethyl- phenyl)- isoxazol- 5-ylmethyl]- 5H-imidazo [4,5-d] pyridazine |
| 220 | 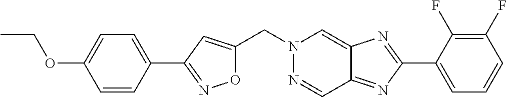 | 2-(2,3-Difluoro- phenyl)-5-[3-(4- ethoxy-phenyl)- isoxazol- 5-ylmethyl]-5H- imidazo[4,5-d] pyridazine |
| 221 | 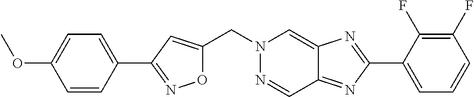 | 2-(2,3-Difluoro- phenyl)-5-[3- (4-methoxy- phenyl)- isoxazol-5- ylmethyl]- 5H-imidazo [4,5-d] pyridazine |
| 222 | 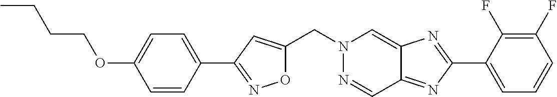 | 5-[3-(4-Butoxy- phenyl)-isoxazol- 5-ylmethyl]-2- (2,3-difluoro- phenyl)- 5H-imidazo [4,5-d] pyridazine |
| 223 | 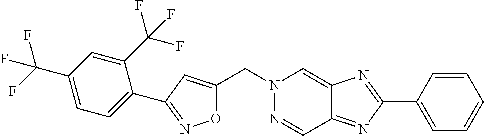 | 5-[3-(2,4-Bis- trifluoro- methyl- phenyl)- isoxazol-5- ylmethyl]- 2-phenyl-5H- imidazo[4,5-d] pyridazine |
| 224 | 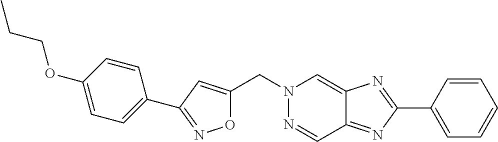 | 2-Phenyl-5-[3-(4- propoxy-phenyl)- isoxazol-5- ylmethyl]- 5H-imidazo [4,5-d] pyridazine |
| 225 | 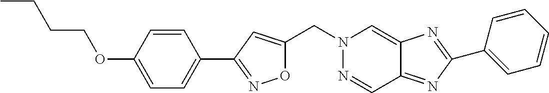 | 5-[3-(4-Butoxy- phenyl)-isoxazol- 5-ylmethyl]- 2-phenyl- 5H-imidazo [4,5-d] pyridazine |
| 226 | 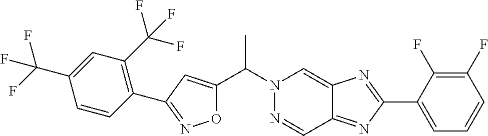 | 5-{1-[3-(2,4-Bis- trifluoro- methyl- phenyl)- isoxazol-5- yl]-ethyl}-2-(2,3- difluoro-phenyl)- 5H-imidazo[4,5- d]pyridazine |
| 227 | 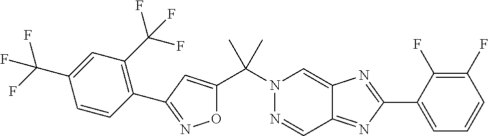 | 5-{1-[3-(2,4- Bis- trifluoro- methyl- phenyl)-isoxazol- 5-yl]-1-methyl- ethyl}- 2-(2,3-difluoro- phenyl)-5H- imidazo[4,5-d] pyridazine |
| 228 | 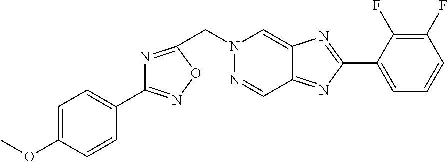 | 2-(2,3-Difluoro- phenyl)-5-[3-(4- methoxy-phenyl)- [1,2,4]oxadiazol- 5-ylmethyl]- 5H-imidazo [4,5-d] pyridazine |
| 229 | 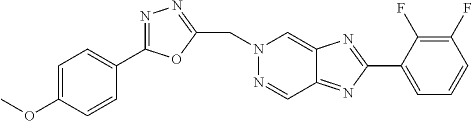 | 2-(2,3-Difluoro- phenyl)-5-[5-(4- methoxy-phenyl)- [1,3,4]oxadiazol- 2-ylmethyl]- 5H-imidazo [4,5-d] pyridazine |
| 230 | 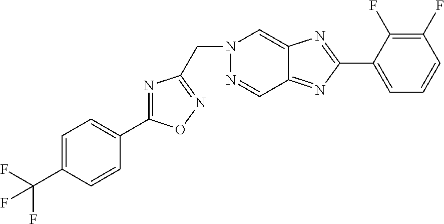 | 2-(2,3-Difluoro- phenyl)-5-[5-(4- trifluoro- methyl- phenyl)- [1,2,4]oxadiazol- 3-ylmethyl]- 5H-imidazo [4,5-d] pyridazine |
| 231 | 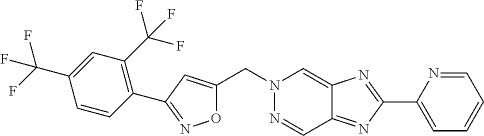 | 5-[3-(2,4-Bis- trifluoro- methyl- phenyl)- isoxazol-5- ylmethyl]- 2-pyridin-2- yl-5H- imidazo [4,5-d] pyridazine |
| 232 | 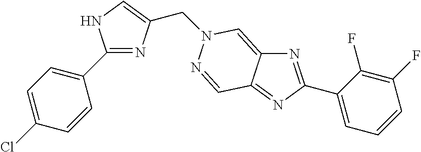 | 5-[2-(4-Chloro- phenyl)-1H- imidazol-4- ylmethyl]-2- (2,3-difluoro- phenyl)- 5H-imidazo [4,5-d] pyridazine |
| 233 | 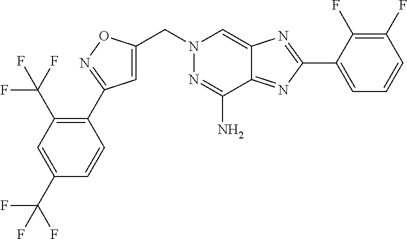 | 6-[3-(2,4- Bis- trifluoro- methyl- phenyl)- isoxazol-5- ylmethyl]- 2-(2,3-difluoro- phenyl)-6H- imidazo [4,5-d] pyridazin-4- ylamine |
| 234 | 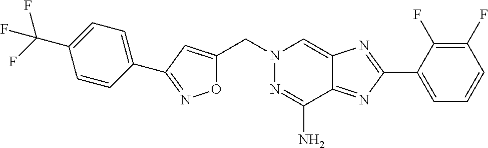 | 2-(2,3- Difluoro- phenyl)- 6-[3-(4- trifluoro- methyl- phenyl)- isoxazol- 5-ylmethyl]- 6H-imidazo [4,5-d] pyridazin- 4-ylamine |
| 235 | 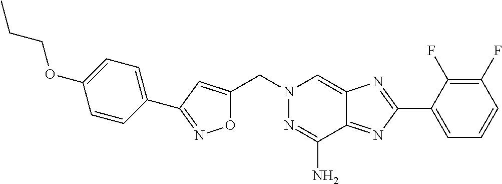 | 2-(2,3-Difluoro- phenyl)-6-[3-(4- propoxy-phenyl)- isoxazol-5- ylmethyl]- 6H-imidazo [4,5-d] pyridazin-4- ylamine |
| 236 | 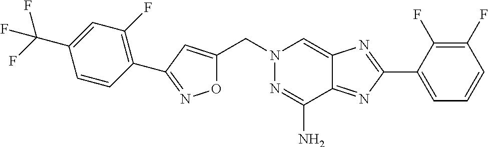 | 2-(2,3-Difluoro- phenyl)-6-[3-(2- fluoro-4- trifluoro- methyl- phenyl)-isoxazol- 5-ylmethyl]-6H- imidazo[4,5-d] pyridazin- 4-ylamine |
| 237 | 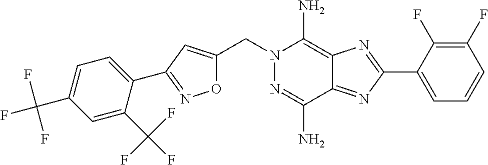 | 5-[3-(2,4-Bis- trifluoro- methyl- phenyl)- isoxazol-5- ylmethyl]- 2-(2,3-difluoro- phenyl)-5H- imidazo [4,5-d]pyridazine- 4,7-diamine |
| 238 | 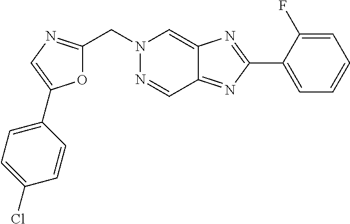 | 5-[5-(4-Chloro- phenyl)-oxazol-2- ylmethyl]-2-(2- fluoro-phenyl)-5H- imidazo[4,5-d] pyridazine |
| 239 | 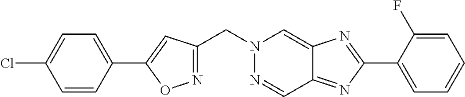 | 5-[5-(4-Chloro- phenyl)-isoxazol- 3-ylmethyl]- 2-(2-fluoro- phenyl)- 5H-imidazo [4,5-d] pyridazine |
| 240 | 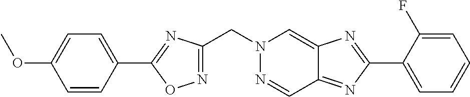 | 2-(2-Fluoro- phenyl)-5-[5-(4- methoxy-phenyl)- [1,2,4]oxadiazol- 3-ylmethyl]- 5H-imidazo [4,5-d] pyridazine |
| 241 | 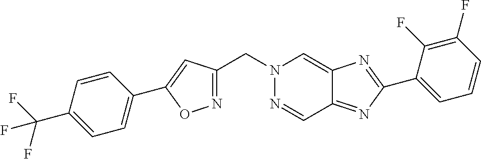 | 2-(2,3-Difluoro- phenyl)-5-[5-(4- trifluoromethyl- phenyl)-isoxazol- 3-ylmethyl]-5H- imidazo [4,5-d] pyridazine |
| 242 | 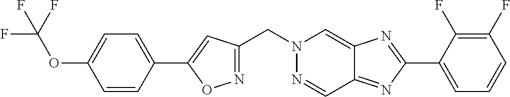 | 2-(2,3-Difluoro- phenyl)-5-[5-(4- trifluoromethoxy- phenyl)-isoxazol- 3-ylmethyl]-5H- imidazo [4,5-d] pyridazine |
| 243 | 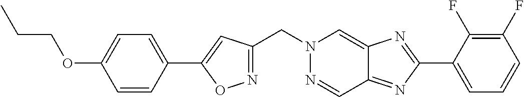 | 2-(2,3-Difluoro- phenyl)-5- [5-(4- propoxy- phenyl)- isoxazol-3- ylmethyl]- 5H-imidazo [4,5-d] pyridazine |
| 244 | 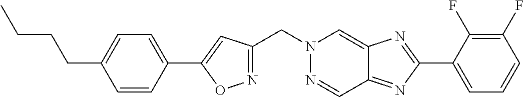 | 5-[5-(4-Butyl- phenyl)-isoxazol- 3-ylmethyl]- 2- (2,3-difluoro- phenyl)- 5H-imidazo [4,5-d] pyridazine |
| 245 | 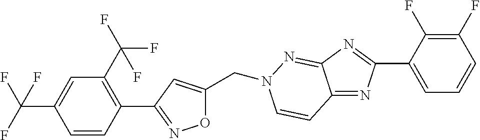 | 2-[3-(2,4-Bis- trifluoro- methyl- phenyl)-isoxazol- 5-ylmethyl]-6- (2,3-difluoro- phenyl)-2H- imidazo [4,5-c] pyridazine |
| 246 | 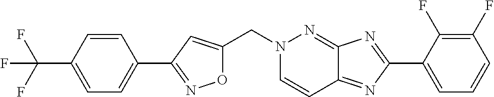 | 6-(2,3-Difluoro- phenyl)-2-[3-(4- trifluoro- methyl- phenyl)-isoxazol- 5-ylmethyl]- 2H-imidazo [4,5-c] pyridazine |
| 247 | 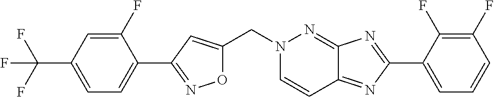 | 6-(2,3-Difluoro- phenyl)-2-[3-(2- fluoro-4- trifluoro- methyl- phenyl)-isoxazol- 5-ylmethyl]-2H- imidazo(4,5-c] pyridazine |
| 248 | 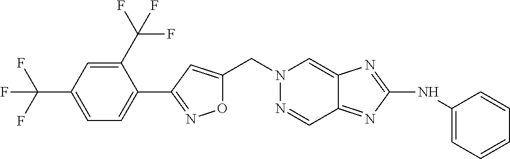 | {5-[3-(2,4-Bis- trifluoro- methyl- phenyl)- isoxazol-5- ylmethyl]-5H- imidazo[4,5- d]pyridazin-2-yl}- phenyl-amine |
| 249 | 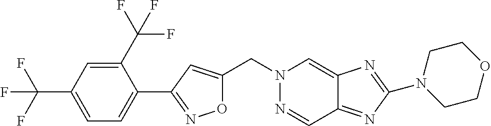 | 5-[3-(2,4-Bis- trifluoro- methyl- phenyl)- isoxazol-5- ylmethyl]-2- morpholin-4-yl- 5H-imidazo [4,5-d] pyridazine |
| 250 | 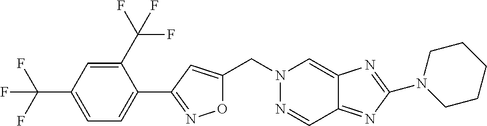 | 5-[3-(2,4-Bis- trifluoro- methyl- phenyl)- isoxazol-5- ylmethyl]-2- piperidin-1-yl- 5H-imidazo [4,5-d] pyridazine |
| 251 | 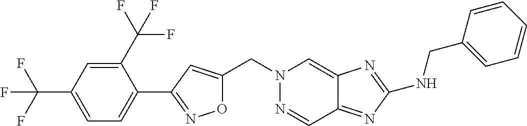 | Benzyl-{5-[3-(2,4- bis-trifluoro- methyl- phenyl)-isoxazol- 5-ylmethyl]-5H- imidazo[4,5-d] pyridazin-2-yl}- amine |
| 252 | 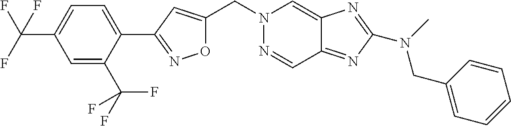 | Benzyl-{5-[3- (2,4-bis- trifluoro- methyl- phenyl)-isoxazol- 5-ylmethyl]-5H- imidazo[4,5-d] pyridazin-2-yl)- methyl-amine |
| 253 | 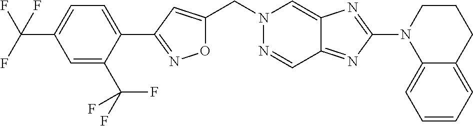 | 1-{5-[3-(2,4- Bis-trifluoro- methyl- phenyl)-isoxazol- 5-ylmethyl]-5H- imidazo[4,5-d] pyridazin-2-yl}- 1,2,3,4- tetrahydro- quinoline |
| 254 | 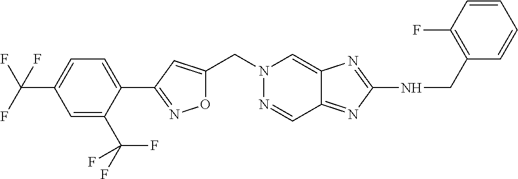 | {5-[3-(2,4-Bis- trifiuoro- methyl- phenyl)- isoxazol-5- ylmethyl]-5H- imidazo[4,5- d]pyridazin- 2-yl}-(2-fluoro- benzyl)-amine |
| 255 | 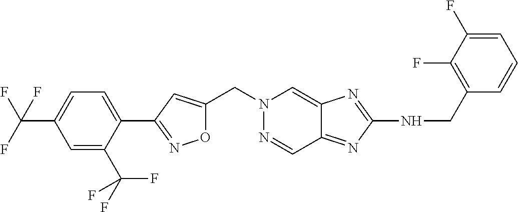 | {5-[3-(2,4-Bis- trifluoro- methyl- phenyl)- isoxazol-5- ylmethyl]-5H- imidazo[4,5- d]pyridazin-2- yl}-(2,3-difluoro- benzyl)-amine |
| 256 | 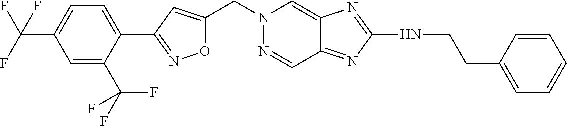 | {5-[3-(2,4-Bis- trifluoro- methyl- phenyl)- isoxazol-5- ylmethyl]-5H- imidazo[4,5- d]pyridazin- 2-yl}- phenethyl- amine |
| 257 | 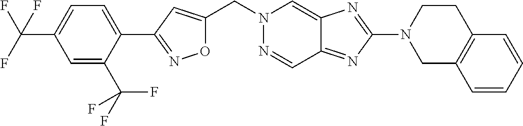 | 2-{5-[3-(2,4- Bis- trifluoro- methyl- phenyl)- isoxazol-5- ylmethyl]-5H- imidazo[4,5-d] pyridazin-2-yl}- 1,2,3,4- tetrahydro- isoquinoline |
| 258 | 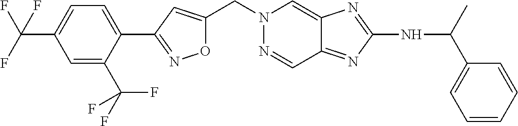 | {5-[3-(2,4-Bis- trifluoro- methyl- phenyl)- isoxazol-5- ylmethyl]-5H- imidazo[4,5- d]pyridazin-2-yl}- (1-phenyl- ethyl)- amine |
| 259 | 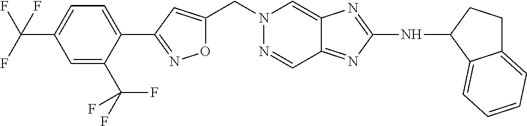 | {5-[3-(2,4-Bis- trifluoro- methyl- phenyl)- isoxazol-5- ylmethyl]-5H- imidazo[4,5- d]pyridazin-2- yl}-indan-1- yl-amine |
| 260 | 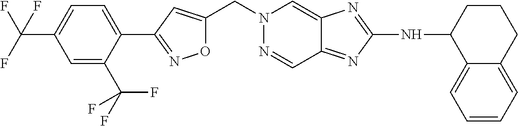 | {5-[3-(2,4-Bis- trifluoro- methyl- phenyl)- isoxazol-5- ylmethyl]-5H- imidazo[4,5- d]pyridazin- 2-yl}-(1,2,3,4- tetrahydro- naphthalen- 1-yl)-amine |
| 261 | 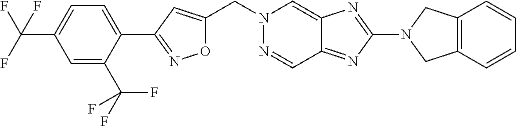 | 5-[3-(2,4-Bis- trifluoro- methyl- phenyl)- isoxazol-5- ylmethyl]-2-(1,3- dihydro- isoindol-2-yl)- 5H-imidazo[4,5- d]pyridazine |
| 262 | 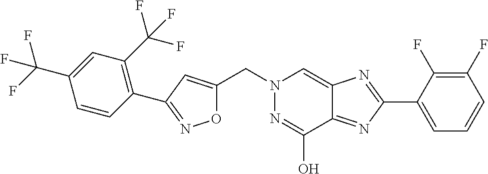 | 6-[3-(2,4-Bis- trifluoro- methyl- phenyl)- isoxazol-5- ylmethyl]-2-(2,3- difluoro- phenyl)-6H- imidazo[4,5-d] pyridazin-4- ol |
| 263 | 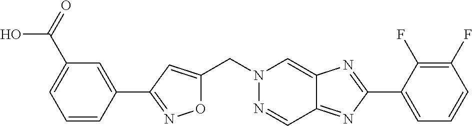 | 3-{5-[2-(2,3- Difluoro- phenyl)- imidazo[4,5-d] pyridazin-5- ylmethyl]- isoxazol-3-yl}- benzoic acid |
| 264 | 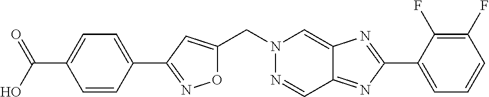 | 4-{5-[2-(2,3- Difluoro- phenyl)- imidazo[4,5-d] pyridazin-5- ylmethyl]- isoxazol-3-yl}- benzoic acid |
| 265 | 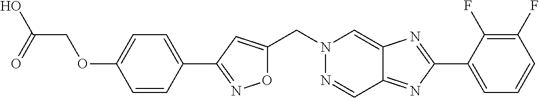 | (4-{5-[2-(2,3- Difluoro- phenyl)- imidazo[4,5-d] pyridazin-5- ylmethyl]- isoxazol-3-yl}- phenoxy)- acetic acid |
| 266 | 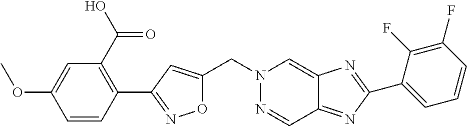 | 2-{5-[2-(2,3- Difluoro- phenyl)- imidazo[4,5-d] pyridazin-5- ylmethyl]- isoxazol-3-yl}- 5-methoxy- benzoic acid |
| 267 | 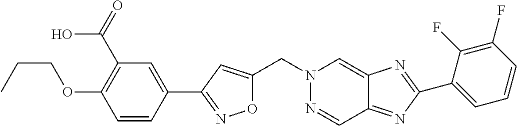 | 5-{5-[2-(2,3- Difluoro- phenyl)- imidazo[4,5-d] pyridazin-5- ylmethyl]- isoxazol-3-yl}- 2-propoxy- benzoic acid |
| 268 | 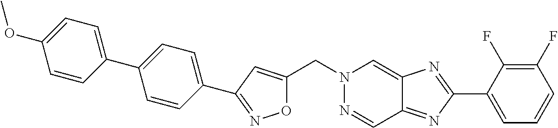 | 2-(2,3-Difluoro- phenyl)-5-[3-(4′- methoxy- biphenyl- 4-yl)-isoxazol-5- ylmethyl]-5H- imidazo[4,5-d] pyridazine |
| 269 | 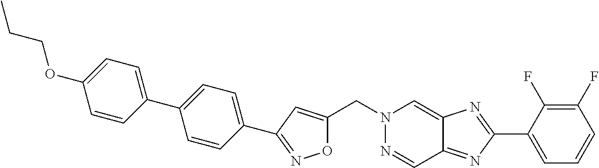 | 2-(2,3-Difluoro- phenyl)-5-[3-(4′- propoxy-biphenyl- 4-yl)-isoxazol-5- ylmethyl]-5H- imidazo [4,5-d]pyridazine |
| 270 | 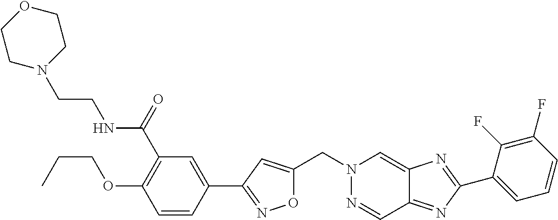 | 5-{5-[2-(2,3- Difluoro- phenyl)- imidazo[4,5-d] pyridazin-5- ylmethyl]- isoxazol-3-yl}- N-(2- morpholin- 4-yl- ethyl)-2- propoxy- benzamide |
| 271 | 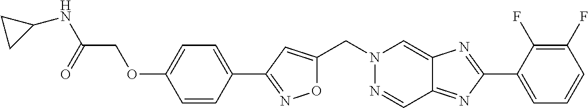 | N- Cyclopropyl-2- (4-{5-[2-(2,3- difluoro- phenyl)- imidazo [4,5-d] pyridazin- 5-ylmethyl]- isoxazol- 3-yl}- phenoxy)- acetamide |
| 272 | 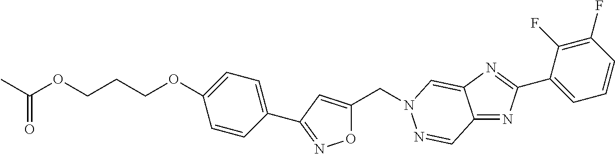 | Acetic acid 3-(4-{5-[2-(2,3- difluoro- phenyl)-imidazo [4,5-d]pyridazin- 5-ylmethyl]- isoxazol-3-yl}- phenoxy)- propyl ester |
| 273 | 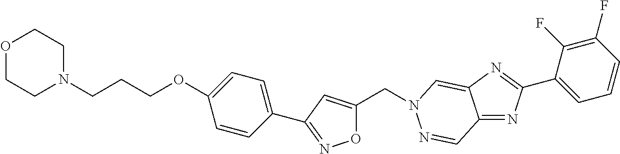 | 2-(2,3-Difluoro- phenyl)-5-{3-[4-(3- morpholin-4- yl-propoxy)- phenyl]- isoxazol-5- ylmethyl}-5H- imidazo[4,5- d]pyridazine |
| 274 | 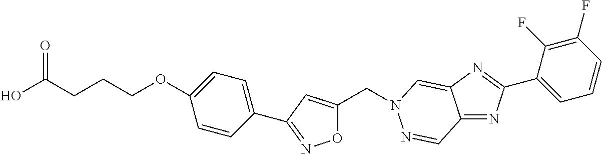 | 4-(4-{5-[2- (2,3-Difluoro- phenyl)- imidazo[4,5-d] pyridazin-5- ylmethyl]- isoxazol-3-yl}- phenoxy)- butyric acid |
| 275 | 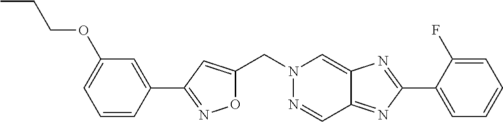 | 2-(2-Fluoro- phenyl)-5-[3-(3- propoxy- phenyl)-isoxazol- 5-ylmethyl]-5H- imidazo[4,5-d] pyridazine |
| 276 | 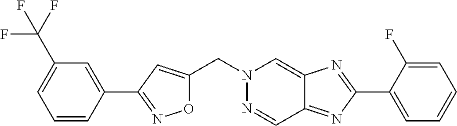 | 2-(2-Fluoro- phenyl)-5-[3-(3- trifluoromethyl- phenyl)-isoxazol- 5-ylmethyl]- 5H-imidazo [4,5-d] pyridazine |
| 277 | 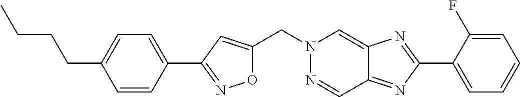 | 5-[3-(4- Butyl-phenyl)- isoxazol-5- ylmethyl]-2-(2- fluoro-phenyl)- 5H-imidazo [4,5-d] pyridazine |
| 278 | 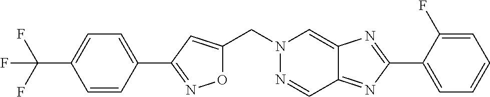 | 2-(2-Fluoro- phenyl)-5-[3-(4- trifluoro- methyl- phenyl)-isoxazol- 5-ylmethyl]- 5H-imidazo [4,5-d] pyridazine |
| 279 | 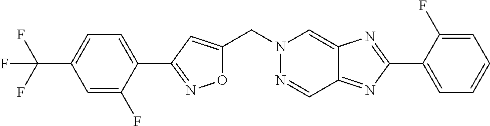 | 2-(2-Fluoro- phenyl)-5-[3- (2-fluoro-4- trifluoro- methyl- phenyl)-isoxazol- 5-ylmethyl]- 5H- imidazo[4,5-d] pyridazine |
| 280 | 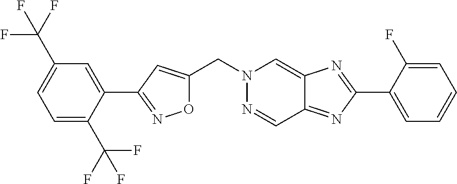 | 5-[3-(2,5-Bis- trifluoro- methyl- phenyl)- isoxazol-5- ylmethyl]-2-(2- fluoro- phenyl)-5H- imidazo[4,5-d] pyridazine |
| 281 | 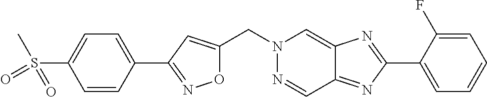 | 2-(2-Fluoro- phenyl)-5- [3-(4- methane- sulfonyl- phenyl)- isoxazol-5- ylmethyl]-5H- imidazo[4,5-d] pyridazine |
| 282 | 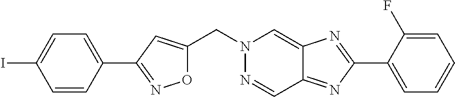 | 2-(2-Fluoro- phenyl)-5-[3- (4-iodo- phenyl)-isoxazol- 5-ylmethyl]-5H- imidazo[4,5-d] pyridazine |
| 283 | 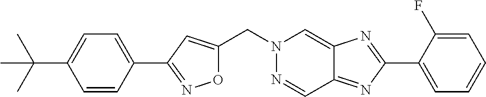 | 5-[3-(4-tert- Butyl-phenyl)- isoxazol-5- ylmethyl]-2-(2- fluoro-phenyl)- 5H- imidazo[4,5-d] pyridazine |
| 284 | 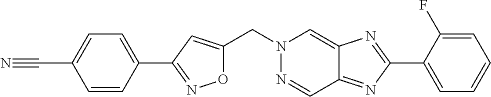 | 4-{5-[2-(2-Fluoro- phenyl)-imidazo [4,5-d]pyridazin- 5-ylmethyl]- isoxazol- 3-yl}- benzonitrile |
| 285 | 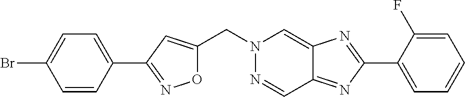 | 5-[3-(4-Bromo- phenyl)-isoxazol- 5-ylmethyl]- 2-(2- fluoro-phenyl)- 5H-imidazo [4,5-d] pyridazine |
| 286 | 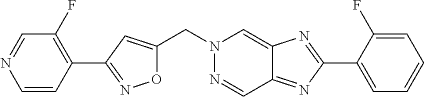 | 2-(2-Fluoro- phenyl)-5-[3-(3- fluoro- pyridin-4-yl)- isoxazol-5- ylmethyl]-5H- imidazo[4,5-d] pyridazine |
| 287 | 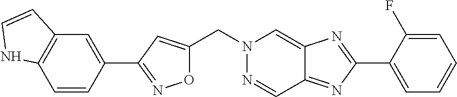 | 2-(2-Fluoro- phenyl)-5-[3-(1H- indol-5- yl)-isoxazol-5- ylmethyl]-5H- imidazo[4,5-d] pyridazine |
| 288 | 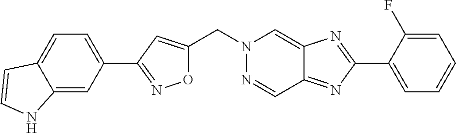 | 2-(2-Fluoro- phenyl)-5-[3-(1H- indol- 6-yl)-isoxazol-5- ylmethyl]-5H- imidazo[4,5-d] pyridazine |
| 289 | 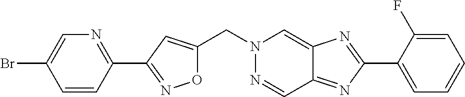 | 5-[3-(5-Bromo- pyridin-2-yl)- isoxazol-5- ylmethyl]-2-(2- fluoro-phenyl)- 5H- imidazo[4,5-d] pyridazine |
| 290 | 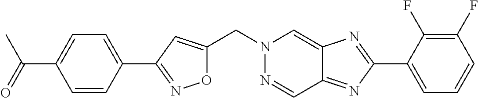 | 1-(4-{5-[2-(2,3- Difluoro-phenyl)- imidazo[4,5-d] pyridazin- 5-ylmethyl]- isoxazol-3-yl}- phenyl)- ethanone |
| 291 | 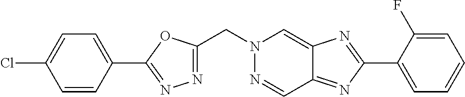 | 5-[5-(4-Chloro- phenyl)- [1,3,4]oxadiazol- 2-ylmethyl]-2-(2- fluoro-phenyl)- 5H-imidazo[4,5- d]pyridazine |
| 292 | 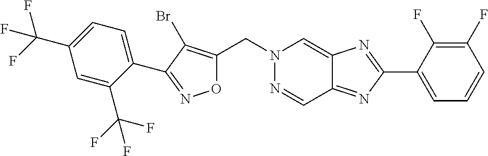 | 5-[3-(2,4-Bis- trifluoromethyl- phenyl)-4- bromo-isoxazol- 5-ylmethyl]-2-(2,3- difluoro-phenyl)- 5H-imidazo[4,5- d]pyridazine |

41. A compound selected from any one or more of the compounds from the Table below or a pharmaceutically acceptable salt or solvate thereof:

| Com- | | |
|---|---|---|
| pound | | |
| # | Structure | Name |
| 293 | 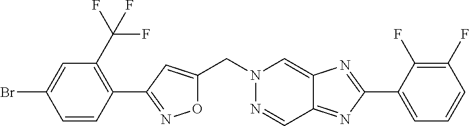 | 5-[3-(4-Bromo-2-trifluoromethyl- phenyl)-isoxazol-5-ylmethyl]-2-(2,3- difluoro-phenyl)-5H-imidazo[4,5- d]pyridazine |
| 294 | 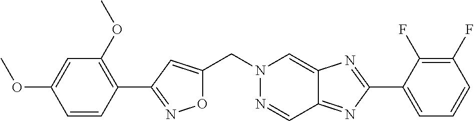 | 2-(2,3-Difluoro-phenyl)-5-[3-(2,4- dimethoxy-phenyl)-isoxazol-5- ylmethyl]-5H-imidazo[4,5-d]pyridazine |
| 295 | 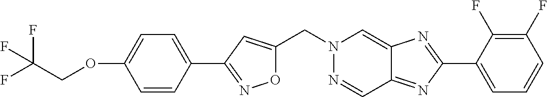 | 2-(2,3-Difluoro-phenyl)-5-{3-[4-(2,2,2- trifluoro-ethoxy)-phenyl]-isoxazol-5- ylmethyl}-5H-imidazo[4,5-d]pyridazine |
| 296 | 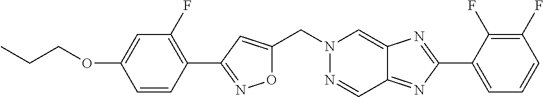 | 2-(2,3-Difluoro-phenyl)-5-[3-(2-fluoro- 4-propoxy-phenyl)-isoxazol-5- ylmethyl]-5H-imidazo[4,5-d]pyridazine |
| 297 | 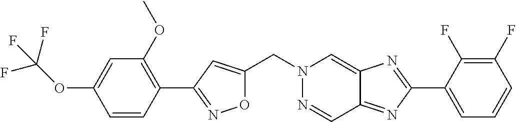 | 2-(2,3-Difluoro-phenyl)-5-[3-(2- methoxy-4-trifluoromethoxy-phenyl)- isoxazol-5-ylmethyl]-5H-imidazo[4,5- d]pyridazine |
| 298 | 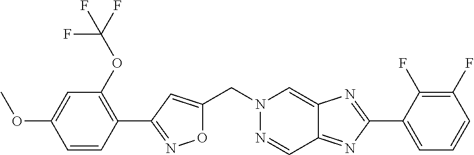 | 2-(2,3-Difluoro-phenyl)-5-[3-(4- methoxy-2-trifluoromethoxy-phenyl)- isoxazol-5-ylmethyl]-5H-imidazo[4,5- d]pyridazine |
| 299 | 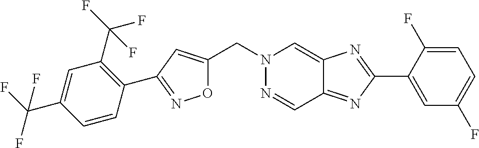 | 5-[3-(2,4-Bis-trifluoromethyl-phenyl)- isoxazol-5-ylmethyl]-2-(2,5-difluoro- phenyl)-5H-imidazo[4,5-d]pyridazine |
| 300 | 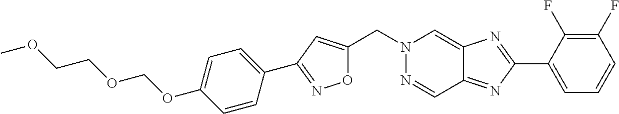 | 2-(2,3-Difluoro-phenyl)-5-{3-[4-(2- methoxy-ethoxymethoxy)-phenyl]- isoxazol-5-ylmethyl}-5H-imidazo[4,5- d]pyridazine |
| 301 | 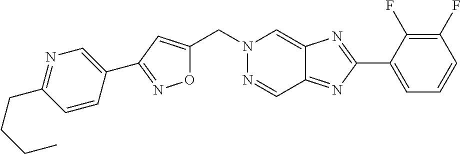 | 5-[3-(6-Butyl-pyridin-3-yl)-isoxazol-5- ylmethyl]-2-(2,3-difluoro-phenyl)-5H- imidazo[4,5-d]pyridazine |
| 302 | 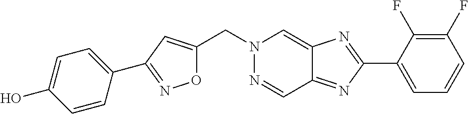 | 4-{5-[2-(2,3-Difluoro-phenyl)- imidazo[4,5-d]pyridazin-5-ylmethyl]- isoxazol-3-yl}-phenol |
| 303 | 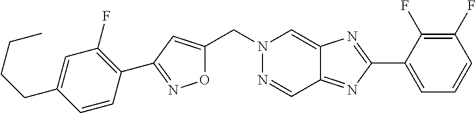 | 5-[3-(4-Butyl-2-fluoro-phenyl)- isoxazol-5-ylmethyl]-2-(2,3-difluoro- phenyl)-5H-imidazo[4,5-d]pyridazine |
| 304 | 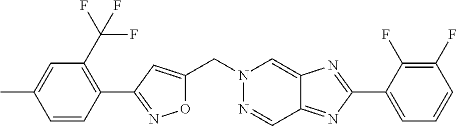 | 2-(2,3-Difluoro-phenyl)-5-[3-(4-methyl- 2-trifluoromethyl-phenyl)-isoxazol-5- ylmethyl]-5H-imidazo[4,5-d]pyridazine |
| 305 | 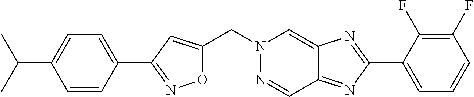 | 2-(2,3-Difluoro-phenyl)-5-[3-(4- isopropyl-phenyl)-isoxazol-5-ylmethyl]- 5H-imidazo[4,5-d]pyridazine |
| 306 | 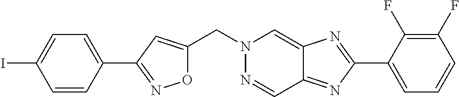 | 2-(2,3-Difluoro-phenyl)-5-[3-(4-iodo- phenyl)-isoxazol-5-ylmethyl]-5H- imidazo[4,5-d]pyridazine |
| 307 | 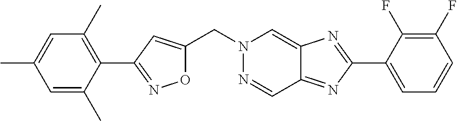 | 2-(2,3-Difluoro-phenyl)-5-[3-(2,4,6- trimethyl-phenyl)-isoxazol-5-ylmethyl]- 5H-imidazo[4,5-d]pyridazine |
| 308 | 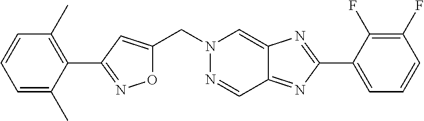 | 2-(2,3-Difluoro-phenyl)-5-[3-(2,6- dimethyl-phenyl)-isoxazol-5-ylmethyl]- 5H-imidazo[4,5-d]pyridazine |
| 309 | 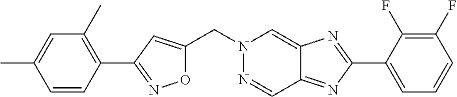 | 2-(2,3-Difluoro-phenyl)-5-[3-(2,4- dimethyl-phenyl)-isoxazol-5-ylmethyl]- 5H-imidazo[4,5-d]pyridazine |
| 310 | 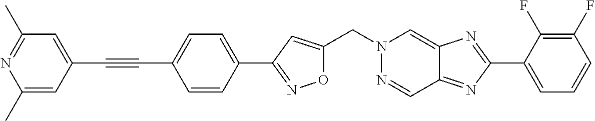 | 2-(2,3-Difluoro-phenyl)-5-{3-[4-(2,6- dimethyl-pyridin-4-ylethynyl)-phenyl]- isoxazol-5-ylmethyl}-5H-imidazo[4,5- d]pyridazine |
| 311 | 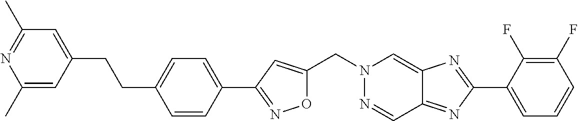 | 2-(2,3-Difluoro-phenyl)-5-(3-{4-[2-(2,6- dimethyl-pyridin-4-yl)-ethyl]-phenyl}- isoxazol-5-ylmethyl)-5H-imidazo[4,5- d]pyridazine |
| 312 | 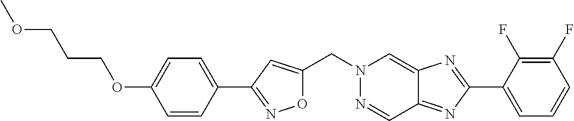 | 2-(2,3-Difluoro-phenyl)-5-{3-[4-(3- methoxy-propoxy)-phenyl]-isoxazol-5- ylmethyl}-5H-imidazo[4,5-d]pyridazine |
| 313 | 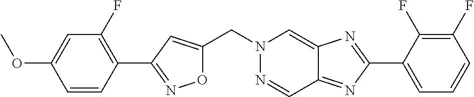 | 2-(2,3-Difluoro-phenyl)-5-[3-(2-fluoro- 4-methoxy-phenyl)-isoxazol-5- ylmethyl]-5H-imidazo[4,5-d]pyridazine |
| 314 | 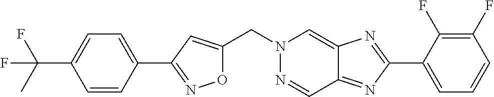 | 5-{3-[4-(1,1-Difluoro-ethyl)-phenyl]- isoxazol-5-ylmethyl}-2-(2,3-difluoro- phenyl)-5H-imidazo[4,5-d]pyridazine |
| 315 | 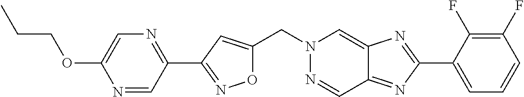 | 2-(2,3-Difluoro-phenyl)-5-[3-(5- propoxy-pyrazin-2-yl)-isoxazol-5- ylmethyl]-5H-imidazo[4,5-d]pyridazine |
| 316 | 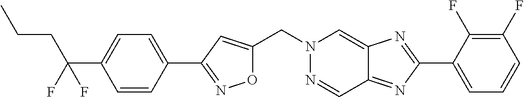 | 5-{3-[4-(1,1-Difluoro-butyl)-phenyl]- isoxazol-5-ylmetyl}-2-(2,3-difluoro- phenyl)-5H-imidazo[4,5-d]pyridazine |
| 317 | 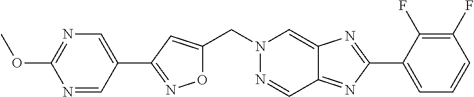 | 2-(2,3-Difluoro-phenyl)-5-[3-(2- methoxy-pyrimidin-5-yl)-isoxazol-5- ylmethyl]-5H-imidazo[4,5-d]pyridazine |
| 318 | 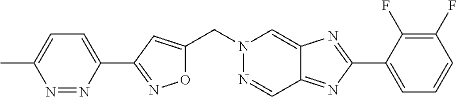 | 2-(2,3-Difluoro-phenyl)-5-[5-(6-methyl- pyridazin-3-yl)-isoxazol-3-ylmethyl]- 5H-imidazo[4,5-d]pyridazine |
| 319 | 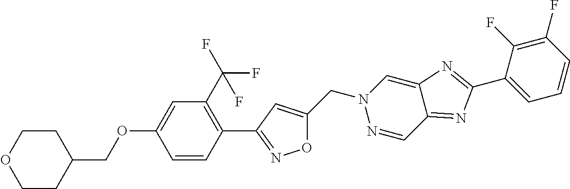 | 2-(2,3-Difluoro-phenyl)-5-{3-[4- (tetrahydro-pyran-4-ylmethoxy)-2- trifluoromethyl-phenyl]-isoxazol-5- ylmethyl}-5H-imidazo[4,5-d]pyridazine |
| 320 | 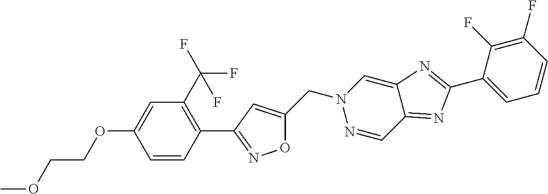 | 2-(2,3-Difluoro-phenyl)-5-{3-[4-(2- methoxy-ethoxy)-2-trifluoromethyl- phenyl]-isoxazol-5-ylmethyl}-5H- imidazo[4,5-d]pyridazine |
| 321 | 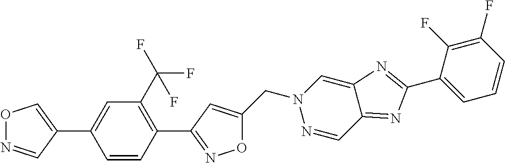 | 2-(2,3-Difluoro-phenyl)-5-[3-(4- isoxazol-4-yl-2-trifluoromethyl-phenyl)- isoxazol-5-ylmethyl]-5H-imidazo[4,5- d]pyridazine |
| 322 | 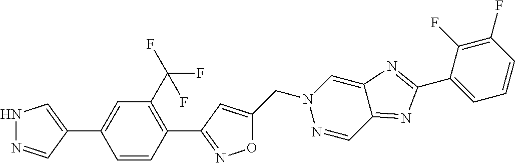 | 2-(2,3-Difluoro-phenyl)-5-{3-[4-(1H- pyrazol-4-yl)-2-trifluoromethyl-phenyl]- isoxazol-5-ylmethyl}-5H-imidazo[4,5- d]pyridazine |
| 323 | 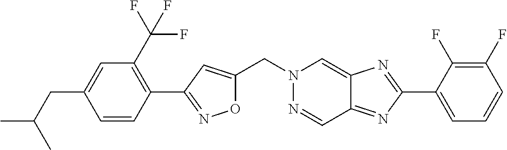 | 2-(2,3-Difluoro-phenyl)-5-[3-(4- isobutyl-2-trifluoromethyl-phenyl)- isoxazol-5-ylmethyl]-5H-imidazo[4,5- d]pyridazine |
| 324 | 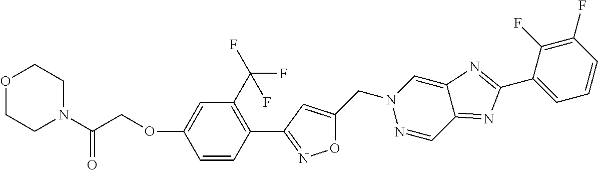 | 2-(4-{5-[2-(2,3-Difluoro-phenyl)- imidazo[4,5-d]pyridazin-5-ylmethyl]- isoxazol-3-yl}-3-trifluoromethyl- phenoxy)-1-morpholin-4-yl-ethanone |
| 325 | 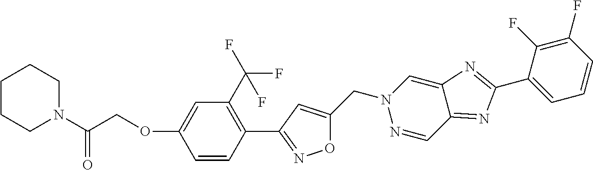 | 2-(4-{5-[2-(2,3-Difluoro-phenyl)- imidazo[4,5-d]pyridazin-5-ylmethyl]- isoxazol-3-yl}-3-trifluoromethyl- phenoxy)-1-piperidin-1-yl-ethanone |
| 326 | 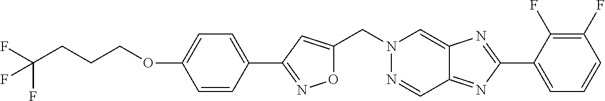 | 2-(2,3-Difluoro-phenyl)-5-{3-[4-(4,4,4- trifluoro-butoxy)-phenyl]-isoxazol-5- ylmethyl}-5H-imidazo[4,5-d]pyridazine |
| 327 | 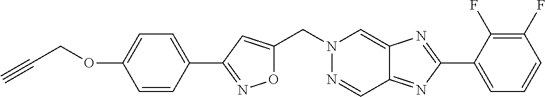 | 2-(2,3-Difluoro-phenyl)-5-[3-(4-prop-2- ynyloxy-2-trifluoromethyl-phenyl)- isoxazol-5-ylmethyl]-5H-imidazo[4,5- d]pyridazine |
| 328 | 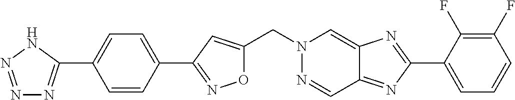 | 2-(2,3-Difluoro-phenyl)-5-{3-[4-(1H- tetrazol-5-yl)-phenyl]-isoxazol-5- ylmethyl}-5H-imidazo[4,5-d]pyridazine |
| 329 | 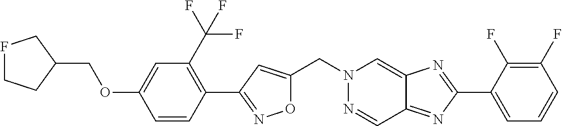 | 2-(2,3-Difluoro-phenyl)-5-{3-(4- (tetrahydro-furan-3-ylmethoxy)-2- trifluoromethyl-phenyl]-isoxazol-5- ylmethyl}-5H-imidazo[4,5-d]pyridazine |
| 330 | 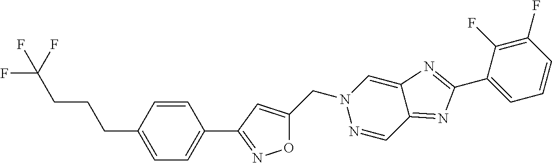 | 2-(2,3-Difluoro-phenyl)-5-{3-[4-(4,4,4- trifluoro-butyl)-phenyl]-isoxazol-5- ylmethyl}-5H-imidazo[4,5-d]pyridazine |
| 331 | 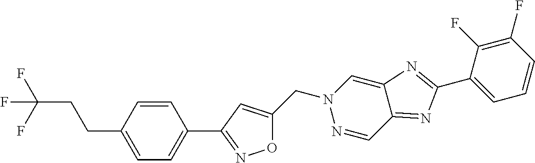 | 2-(2,3-Difluoro-phenyl)-5-{3-[4-(3,3,3- trifluoro-propyl)-phenyl]-isoxazol-5- ylmethyl}-5H-imidazo[4,5-d]pyridazine |
| 332 | 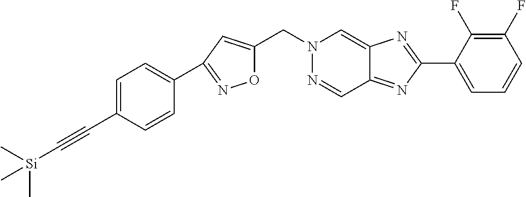 | 2-(2,3-Difluoro-phenyl)-5-[3-(4- trimethylsilanylethynyl-phenyl)- isoxazol-5-ylmethyl]-5H-imidazo[4,5- d]pyridazine |
| 333 | 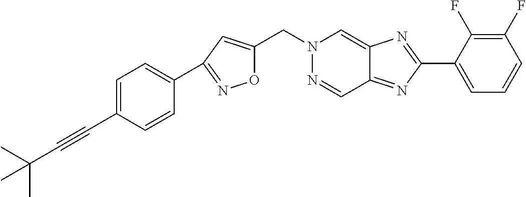 | 2-(2,3-Difluoro-phenyl)-5-{3-[4-(3,3- dimethyl-but-1-ynyl)-phenyl]-isoxazol- 5-ylmethyl}-5H-imidazo[4,5- d]pyridazine |
| 334 | 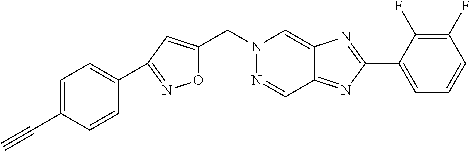 | 2-(2,3-Difluoro-phenyl)-5-[3-(4- ethynyl-phenyl)-isoxazol-5-ylmethyl]- 5H-imidazo[4,5-d]pyridazine |
| 335 | 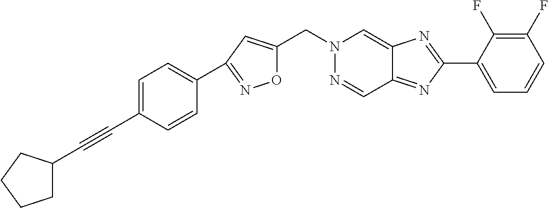 | 5-[3-(4-Cyclopentylethynyl-phenyl)- isoxazol-5-ylmethyl]-2-(2,3-difluoro- phenyl)-5H-iidazo[4,5-d]pyridazine |
| 336 | 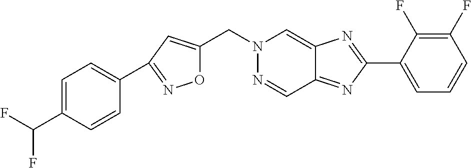 | 5-[3-(4-Difluoromethyl-phenyl)- isoxazol-5-ylmethyl]-2-(2,3-difluoro- phenyl)-5H-imidazo[4,5-d]pyridazine |
| 337 | 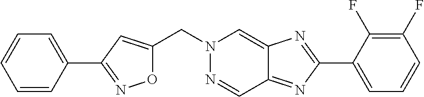 | 2-(2,3-Difluoro-phenyl)-5-(3-phenyl- isoxazol-5-ylmethyl)-5H-imidazo[4,5- d]pyridazine |
| 338 | 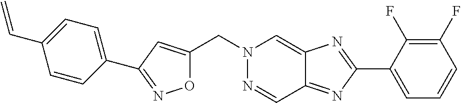 | 2-(2,3-Difluoro-phenyl)-5-[3-(4-vinyl- phenyl)-isoxazol-5-ylmethyl]-5H- imidazo[4,5-d]pyridazine |
| 339 | 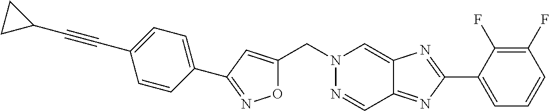 | 5-[3-(4-Cyclopropylethynyl-phenyl)- isoxazol-5-ylmethyl]-2-(2,3-difluoro- phenyl)-5H-imidazo[4,5-d]pyridazine |
| 340 | 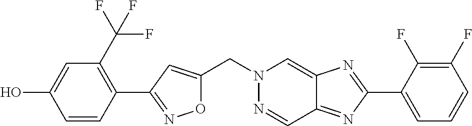 | 4-{5-[2-(2,3-Difluoro-phenyl)- imidazo[4,5-d]pyridazin-5-ylmethyl]- isoxazol-3-yl}-3-trifluoromethyl-phenol |

42. A pharmaceutical composition comprising a pharmaceutically acceptable carrier and a therapeutically effective amount of a compound of claim 1, or a pharmaceutically acceptable salt or solvate thereof.

43. A method for treating a viral infection in a patient mediated at least in part by a virus in the Flaviviridae family of viruses which method comprises administering to the patient a compound of claim 1, or a pharmaceutically acceptable salt or solvate thereof.

44. The method of claim 43 wherein said viral infection is a hepatitis C mediated viral infection.

45. The method of claim 43 in combination with the administration of a therapeutically effective amount of one or more agents active against hepatitis C virus.

46. The method of claim 45 wherein said agent active against hepatitis C virus is an inhibitor of HCV proteases, HCV polymerase, HCV helicase, HCV NS4B protein, HCV entry, HCV assembly, HCV egress, HCV NS5A protein, or inosine 5′-monophosphate dehydrogenase.

47. The method of claim 45 wherein said agent active against hepatitis C virus is interferon.
